(19) United States
(12) Patent Application Publication
(10) Pub. No.: US 2009/0156575 A1
(43) Pub. Date: Jun. 18, 2009

(54) NOVEL DIAZASPIROALKANES AND THEIR USE FOR TREATMENT OF CCR8 MEDIATED DISEASES

(76) Inventors: Lena Borjesson, Lund (SE); Henrik Johansson, Lund (SE); Anna Kristoffersson, Lund (SE); Igor Shamovsky, Lund (SE); Marco Skrinjar, Lund (SE); Stephen Connolly, Leicestershire (GB); Tero Linnanen, Horsholm (DK)

Correspondence Address: MORGAN LEWIS & BOCKIUS LLP, 1111 PENNSYLVANIA AVENUE NW, WASHINGTON, DC, 20004, US

(21) Appl. No.: 12/065,822

(22) PCT Filed: Sep. 4, 2006
(86) PCT No.: PCT/SE2006/001012

§ 371 Date: Sep. 30, 2008

(30) Foreign Application Priority Data
Sep. 6, 2005 (SE) 0501967-4

(51) Int. Cl. C07D 471/10; A61K 31/438; A61K 31/444; A61K 31/501; A61P 11/00

(52) U.S. Cl. 514/210.16; 546/16; 514/278; 514/256; 544/335; 514/274; 514/210.18; 544/312; 544/238; 514/252.04

(57) ABSTRACT

The invention provides compounds of general formula

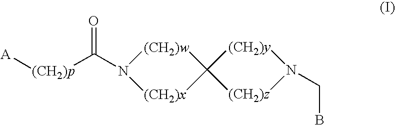

wherein A, B, p, w, x, y, and z are as defined in the specification, processes for their preparation, pharmaceutical compositions containing them and their use in therapy.

The present invention relates to diazaspiro compounds, processes for their preparation, pharmaceutical compositions containing them and their use in therapy.

Both the initial stages of a disease as well as the long-term tissue remodeling and muscle hypotrophy depend on recruitment of leukocytes to the inflammatory lesion. Leukocyte recruitment involves the migration of leukocytes into the diseased tissue from the blood vessel and their activation, which leads to progression of disease. The mechanism underlying this recruitment, chemotaxis, is similar both in classically defined immune mediated pathological conditions (i.e. allergic and autoimmune diseases) as well as others (i.e. atherosclerosis and Parkinson's disease). Thus, intervention of leukocyte recruitment to the inflamed target tissue constitutes an attractive novel therapeutic principle.

The chemokines are a large family (>50 members) of small 8- to 15-kDa secreted, heparin-binding polypeptides with the primary function of controlling trafficking and activation of leukocytes. They are distinct from classical chemoattractants (i.e. bacterial derived N-formyl peptides, complement components, lipid molecules and platelet activating factor) on the basis of shared structural similarities. All chemokines have four conserved cysteines residues that form disulfide bonds, which are critical for the 3-D structure. The chemokines are further subclassed according to the position of the first two cysteines. The two major subclasses are the CC-chemokines, that have the cysteines adjacent, and the CXC-cytokines, that have the cysteines separated by one amino acid. The two other families, the C and the CX3C chemokines, are much smaller and only comprise one or a few members.

The C-X-C chemokines include several potent chemoattractants and activators of neutrophils such as interleukin-8 (IL-8) and neutrophil-activating peptide 2 (NAP-2).

The C-C chemokines include potent chemoattractants of monocytes and lymphocytes, such as human monocyte chemotactic proteins 1-3 (MCP-1, MCP-2 and MCP-3), RANTES (Regulated on Activation, Normal T Expressed and Secreted), eotaxin and the macrophage inflammatory proteins 1α and 1β (MIP-1α and MIP-1β) and CCL1.

Studies have demonstrated that the actions of the chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10, CXCR1, CXCR2, CXCR3 and CXCR4.

The accumulation of immune cells at a site of allergic inflammation occurs within 6-48 hours after allergen challenge and is a hallmark of allergic diseases. Studies have shown that antigen-specific CD4⁺ T cells are detected in lung tissue of asthmatic patients after exposure to the allergen. Although infiltrating T cells are relatively few in number compared to eosinophils, compelling evidence has demonstrated essential roles for T cells in orchestrating the inflammatory process in human asthma. A close correlation exists in humans between the level of TH2 cytokines produced by T cells, serum level of IgE and prevalence of asthma.

The human CCR8 receptor has been shown to interact with the human chemokine CCL1 (I-309). This chemokine is a potent eosinophil, T-, dentritic- and endothelial cell chemoattractant. The receptor has been shown to be transiently upregulated on polarized TH2 cells after optimal TCR cross linkage in the presence of costimulatory signals (i.e. CD28). The coordinated upregulation of CCR8 on activated T cells after antigen challenge indicates that it contributes to redistribution of the activated T cells to the inflammatory foci within the inflamed tissue expressing CCL1. Indeed, in vivo models of allergic airway inflammation using mice deficient in CCR8 expression have shown a profound block in recruitment of effector T cells to the inflamed lung tissue and production of TH2 cytokines. Moreover, T cells infiltrating the human airway subepithelium during allergen challenge have been shown to be CCR8 positive. Importantly, the number of CCR8 positive cells migrating into the airway submucosa following allergen challenge has been shown to correlate with decreases in FEV1.

Considering the significant role CCR8 plays in TH2 cell chemotaxis, and the importance of TH2 cells in allergic conditions such as asthma, CCR8 represents a good target for drug development in the treatment of respiratory diseases such as asthma.

WO2005040167 describes diazaspiro compounds and their use in therapy.

The present inventors have now identified a novel set of compounds which act as CCR8 receptor antagonists.

In accordance with the present invention, there is therefore provided a compound of general formula

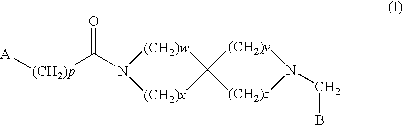

wherein
B represents the group

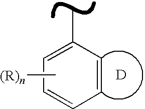

ring D, together with the two benzene carbon atoms to which it is fused, is a 5- or 6-membered, non-aromatic ring containing one or two ring-oxygen atoms, and optionally containing a carbon-carbon double bond between two ring carbon atoms other than said benzene carbon atoms, ring D being optionally substituted with one or more substituents independently selected from C₁-C₆ alkyl, C₃-C₆ cycloalkyl, or phenyl (said phenyl being optionally substituted with one or more substituents independently selected from halogen, hydroxyl or C₁-C₄ alkoxy);
   and additionally wherein when ring D is a 5-membered, non-aromatic ring containing two ring-oxygen atoms that are 1,3 disposed, ring D may be optionally substituted with group E, wherein group E together with a single carbon atom on ring D, represents a 4- to 8-membered cycloalkyl ring, such that group E forms a spiro structure with ring D;
w, x, y and z are independently 1, 2 or 3;
Each R independently represents halogen or C₁-C₄ alkyl;
n is 0, 1 or 2;
A represents a group selected from phenyl, a 5- or 6-membered heteroaromatic ring containing at least one ring heteroatom independently selected from nitrogen, oxygen or sulphur, or pyridine-N-oxide, each group being optionally substituted with one or more substituents independently selected from hydroxyl, —CN, halogen, oxo (═O), C₁-C₆ aminoalkyl, C₁-C₆ alkylamino-C₁-C₆ alkyl, N,N-di(C₁-C₆)alkylamino-C₁-C₆ alkyl, C₁-C₆ alkoxy, C₁-C₆ alkylcarbonyl, —NR¹R², —C(O)—NR³R⁴, —C₁-C₆ alkylenyl-C(O)—NR³R⁴, —C₁-C₄ alkyl-C(O)—NR⁵R⁶, —NHSO₂—R⁷, —NHC(O)R⁸, —SO₂NH₂, carboxyl, carboxyl-C₁-C₆ alkyl, C₁-C₆ alkoxycarbonyl, C₁-C₄ alkoxycarbonyl-C₁-C₄ alkyl, C₃-C₆ cycloalkylamino, phenyl, pyridyl (said phenyl and pyridyl being optionally further substituted with one or more groups independently selected from halogen, hydroxyl, carboxy or C₁-C₄ alkyl), C₁-C₆ alkyl or C₃-C₆ cycloalkyl (said latter two C₁-C₆ alkyl and C₃-C₆ cycloalkyl substituents being optionally further substituted with one or more substituents independently selected from halogen, hydroxyl, or —CN);
or A represents a 9- or 10-membered bicyclic ring system containing one or more ring heteroatoms independently selected from nitrogen, oxygen or sulphur and which is optionally substituted with one or more substituents independently selected from hydroxyl, —CN, halogen, oxo, C₁-C₆ alkoxy, —NR⁹R¹⁰, carboxyl, or C₁-C₆ alkyl;
p is 0, 1 or 2;
R¹ and R² each independently represent a hydrogen atom, a C₁-C₆ alkyl, C₃-C₆ cycloalkyl or R¹ and R² together with the nitrogen atom to which they are attached form a hydantoin group or form a 4- to 7-membered saturated heterocycle, said heterocycle being optionally substituted with hydroxyl, C₁-C₄ alkoxy, or C₁-C₄ alkoxy-C₁-C₄ alkyl;
R³ and R⁴ each independently represent a hydrogen atom, C₁-C₆ alkyl, or C₃-C₆ cycloalkyl, or R³ and R⁴ together with the nitrogen atom to which they are attached form a 4- to 7-membered saturated heterocycle, said heterocycle being optionally substituted with aminocarbonyl;
R⁵ and R⁶ each independently represent a hydrogen atom, C₁-C₆ alkyl, or C₃-C₆ cycloalkyl, or R⁵ and R⁶ together with the nitrogen atom to which they are attached form a 4- to 7-membered saturated heterocycle, said heterocycle being optionally substituted with aminocarbonyl;
R⁷ represents C₁-C₆ alkyl, or a 6-membered saturated or unsaturated heterocyclic ring, the ring containing at least one nitrogen atom, the ring being optionally substituted with one or more substituents independently selected from halogen, oxo, C₁-C₆ alkoxy, or C₁-C₆ alkyl;
R⁸ represents pyridine-N-oxide optionally substituted with one or more substituents independently selected from halogen, or C₁-C₆ alkyl, or R⁸ represents C₁-C₆ alkyl, C₁-C₆ hydroxyalkyl, or a 5- or 6-membered saturated heterocyclic ring containing at least one heteroatom independently selected from nitrogen and oxygen, which ring being optionally substituted with one or more substituents independently selected from halogen, C₁-C₆ alkoxy, oxo, or C₁-C₆ alkyl;
R⁹ and R¹⁰ each independently represent a hydrogen atom or C₁-C₆ alkyl;
   or a pharmaceutically acceptable salt thereof.

The compounds of the present invention act as particularly potent CCR8 antagonists. Furthermore, the compounds of the present invention may also possess properties that render them particular desirable for pharmaceutical compounds, such as low toxicity, good selectivity and/or good metabolic stability.

In the context of the present specification, an alkyl substituent group or an alkyl moiety in a substituent group may be linear or branched. Furthermore, an alkenyl substituent group or an alkenyl moiety in a substituent group may be linear or branched.

When any chemical moiety or group in formula (I) is described as being optionally substituted, it will be appreciated that the moiety or group may be either unsubstituted or substituted by one or more of the specified substituents. A moiety or group where the only substituent present is hydrogen is considered unsubstituted.

It will be appreciated that throughout the specification, the number and nature of substituents on rings in the compounds of the invention will be selected so as to avoid sterically undesirable combinations.

When R¹ and R² (or R³ and R⁴, or R⁵ and R⁶) together represent a saturated heterocycle, it should be understood that the only heteroatom present is the nitrogen atom to which R¹ and R² (or R³ and R⁴, or R⁵ and R⁶) are attached.

When A represents a 9- or 10-membered bicyclic ring system, the two rings in the bicycle are fused together. By fused together is meant that two adjacent atoms in the ring system are shared by both rings. Preferably, the bicyclic ring system is a 9- or 10-membered bicyclic heteroaromatic ring system. It should be noted that one or both of the rings in the bicycle may be aromatic. Furthermore, the one or more heteroatoms in the bicycle may be present in an aromatic part of the bicycle or alternatively may be present in a non-aromatic part of the bicycle.

In the definition of R⁷, it should be noted that the 6-membered saturated or unsaturated heterocyclic ring may have alicyclic or aromatic properties. An unsaturated ring may be partially or fully unsaturated.

w, x, y and z are independently 1, 2 or 3. Example combinations of w+x, and y+z are listed below:

| w + x | | y + z |
|---|---|---|
| 4 | and | 4 |
| 3 | and | 4 |
| 4 | and | 3 |
| 2 | and | 4 |
| 4 | and | 2 |

When w+x is equal to 4, then both w and x may be equal to 2. Alternatively, one of w and x may be 1, and the other of w or x equal to 3. When y+z is equal to 4, then both y and z may be equal to 2. Alternatively, one of y and z may be 1, and the other of y or z equal to 3. When w+x is equal to 3, then one of w and x may be 1, and the other of w or x equal to 2. When y+z is equal to 3, then one of y and z may be 1, and the other of y or z equal to 2.

Combinations of w, x, y and z include: w, x, y and z each equal to 2; or w and x each equal to 2, one of y and z equal to 2 and the other of y and z equal to 1; or y and z each equal to 2, one of w and x equal to 2 and the other of w and x equal to 1; or w and x each equal to 1, and y and z each equal to 2.

In an embodiment of the present invention, the sum of w+x does does not exceed 5, and the sum of y+z does does not exceed 5.

In an embodiment of the present invention, the sum of w+x+y+z is greater than 5.

In an embodiment of the present invention, the sum of w+x does does not exceed 5, the sum of y+z does does not exceed 5, and the sum of w+x+y+z is greater than 5.

In a further embodiment of the present invention, w, x, y and z are each equal to 2.

In a further embodiment of the present invention, w and x are each equal to 1, and y and z are each equal to 2.

In a further embodiment of the present invention, w and x are each equal to 2, and y and z are each equal to 1.

In a further embodiment of the present invention, w, x and y are each equal to 2, and z is equal to 1.

In a further embodiment of the present invention, w is equal to 1, and x, y and z are each equal to 2.

In a further embodiment of the present invention, w and y are each equal to 1, and x and z are each equal to 2.

A represents a group selected from phenyl, a 5- or 6-membered heteroaromatic ring containing at least one ring heteroatom independently selected from nitrogen, oxygen or sulphur, or pyridine-N-oxide, each group being optionally substituted as defined above. The heteroaromatic ring may contain 1, 2, 3 or 4 heteroatoms, typically 1, 2, or 3 heteroatoms, and more typically 1 or 2 heteroatoms.

Examples of such 5- or 6-membered heteroaromatic rings containing at least one ring heteroatom are pyridyl, pyrazolyl, thiadiazolyl, isoxazolyl, imidazolyl, pyrrolyl, pyridazinyl, pyrazinyl, oxadiazolyl, furyl, pyrimidinyl, thiazolyl, oxazolyl, isothiazolyl, triazolyl, tetrazolyl or thienyl.

It will be appreciated that the definition of ring A (and other heterocyclic groups referred to in formula (I)) is not intended to include unstable structures and is not intended to include any 0-0, O—S or S—S bonds.

When A represents a group selected from phenyl, a 5- or 6-membered heteroaromatic ring containing at least one ring heteroatom independently selected from nitrogen, oxygen or sulphur, or pyridine-N-oxide, each group may be substituted with one or more (e.g. 1, 2 or 3, preferably 1 or 2) substituent(s) independently selected from hydroxyl; —CN; halogen (e.g. chlorine, fluorine, bromine or iodine); oxo (i.e. ═O); C₁-C₆ aminoalkyl, preferably C₁-C₄ aminoalkyl (e.g. aminomethyl and aminoethyl); C₁-C₆ alkylamino-C₁-C₆ alkyl, preferably C₁-C₄ alkylamino-C₁-C₄ alkyl (e.g. CH₃—NH—CH₂—); N,N-di(C₁-C₆)alkylamino-C₁-C₆ alkyl, preferably di(C₁-C₄)alkylamino-C₁-C₄ alkyl; C₁-C₆ alkoxy, preferably C₁-C₄ alkoxy (e.g. methoxy, ethoxy, n-propoxy or n-butoxy); C₁-C₆ alkylcarbonyl, preferably C₁-C₄ alkylcarbonyl (e.g. methoxycarbonyl or ethoxycarbonyl); —NR¹R²; —C(O)—NR³R⁴; —C₁-C₆ alkylenyl-C(O)—NR³R⁴, preferably —C₁-C₄ alkylenyl-C(O)—NR³R⁴; —C₁-C₄ alkyl-C(O)—NR⁵R⁶ (e.g. —CH₂—C(O)—NR⁵R⁶, —CH₂—CH₂—C(O)—NR⁵R⁶); —NHSO₂—R⁷; —NHC(O)R⁸; —SO₂NH₂; carboxyl; carboxyl-C₁-C₆ alkyl, preferably carboxyl-C₁-C₄ alkyl (e.g. carboxymethyl, carboxyethyl, carboxypropyl, carboxypropyl, carboxybutyl, more typically —CH₂—COOH, —(CH₂)₂—COOH); C₁-C₆, preferably C₁-C₄ alkoxycarbonyl (e.g. methoxycarbonyl or ethoxycarbonyl); C₁-C₄ alkoxycarbonyl-C₁-C₄ alkyl, preferably C₁-C₂ alkoxycarbonyl-C₁-C₂ alkyl (e.g. CH₃—O—C(O)—CH₂—); C₃-C₆ cycloalkylamino (e.g. cyclopropylamino, cyclobutylamino, cyclopentylamino or cyclohexylamino); phenyl or pyridyl (said phenyl and pyridyl being optionally further substituted with one or more groups independently selected from halogen (e.g. chlorine or fluorine), hydroxyl, carboxy or C₁-C₄ alkyl (e.g. methyl)); C₁-C₆ alkyl, preferably C₁-C₄ alkyl (e.g. methyl, ethyl, n-propyl, isopropyl, n-butyl, isobutyl, tert-butyl, n-pentyl or n-hexyl); C₃-C₆ cycloalkyl (e.g. cyclopropyl, cyclobutyl, cyclopentyl or cyclohexyl); the latter two C₁-C₆ alkyl and C₃-C₆ cycloalkyl substituents being optionally further substituted with one or more substituents independently selected from halogen (e.g. chlorine or fluorine), hydroxyl, or —CN.

When A is a 5- or 6-membered heteroaromatic ring substituted by oxo, an example is pyridone (e.g. pyridin-2(1H)-one).

In an embodiment of the present invention, A is phenyl, a 5- or 6-membered heteroaromatic ring containing at least one ring heteroatom independently selected from nitrogen, oxygen or sulphur, or pyridine-N-oxide, and A is either unsubstituted or is substituted with a single substituent as defined above.

In another embodiment of the invention, A is phenyl, pyridyl or pyrimidinyl, each of which may be optionally substituted.

In a further embodiment, A is phenyl, pyridyl or pyrimidinyl substituted with 0, 1 or 2 substituents independently selected from hydroxyl, cyano, halogen, C₁-C₆ alkyl, NH₂, C₁-C₄ alkoxycarbonyl, C₁-C₄ alkoxycarbonyl-C₁-C₄ alkyl, —C(O)—NR³R⁴, —C₁-C₄ alkyl-C(O)—NR⁵R⁶, or —NHC(O)R⁸.

In a further embodiment, A is pyridyl or pyrimidinyl, each substituted with NH₂.

When A is optionally substituted pyridine-N-oxide, the pyridine-N-oxide is preferably either unsubstituted or substituted with C₁-C₄ alkyl.

A also represents an optionally substituted 9- or 10-membered bicyclic ring system containing one or more (e.g. 1, 2 or 3) ring heteroatoms independently selected from nitrogen, oxygen or sulphur.

Examples of such 9- or 10-membered bicyclic heteroaromatic ring systems are indolyl, indazolyl, quinolinyl, naphthyridinyl (e.g. 1,8-naphthyridinyl, 2,7-naphthyridinyl), benzimidazolyl, isoindolyl, indolinyl, benzofuranyl, benzothiophenyl, benzimidazolyl, benzthiazolyl, purinyl, isoquinolinyl, cinnolinyl, quinazolinyl and quinoxalinyl.

When A is a 9- or 10-membered bicyclic ring system containing one or more heteroatoms independently selected from nitrogen, oxygen or sulphur, the bicyclic ring system may be substituted with one or more (e.g. 1, 2 or 3) substituent(s) independently selected from hydroxyl; —CN; halogen (e.g. chlorine or fluorine); oxo; C₁-C₆ alkoxy, preferably C₁-C₄ alkoxy (e.g. methoxy, ethoxy, n-propoxy or n-butoxy); —NR⁹R¹⁰, carboxyl, or C₁-C₆ alkyl, preferably C₁-C₄ alkyl (e.g. methyl, ethyl, n-propyl, isopropyl, n-butyl, isobutyl, tert-butyl, n-pentyl or n-hexyl).

In an embodiment of the present invention, when A is a 9- or 10-membered bicyclic ring system containing one or more heteroatoms independently selected from nitrogen, oxygen or sulphur, A is either unsubstituted or is substituted with a single substituent.

When A is substituted with one or more substituents, the substituents may be present on any suitable available position. Preferably, the substituents are attached on a suitable ring carbon atom.

R¹ and R² each independently represent a hydrogen atom or a C₁-C₆ alkyl group, preferably C₁-C₄, alkyl group (e.g. methyl, ethyl, n-propyl, isopropyl, n-butyl, isobutyl, tert-butyl, n-pentyl or n-hexyl) or a C₃-C₆ cycloalkyl group (e.g. cyclopropyl, cyclobutyl, cyclopentyl or cyclohexyl); or R¹ and R² together with the nitrogen atom to which they are attached form a hydantoin group or form a 4- to 7-membered saturated heterocycle (e.g pyrrolidinyl or piperidinyl), said heterocycle being optionally substituted with hydroxyl, C₁-C₄ alkoxy (e.g. methoxy), or C₁-C₄ alkoxy-C₁-C₄ alkyl (e.g. methoxymethyl). The heterocycle will typically be unsubstituted or substituted with one or more (e.g. 1 or 2) of said substitutents.

In an embodiment of the present invention, R¹ and R² each independently represent a hydrogen atom, a C₁-C₆ alkyl, or R¹ and R² together with the nitrogen atom to which they are attached form a 4- to 7-membered saturated heterocycle, said heterocycle being optionally substituted with hydroxyl, C₁-C₄ alkoxy, or C₁-C₄ alkoxy-C₁-C₄ alkyl;

In a further embodiment of the present invention —NR¹R² is —NH₂, methylamino, dimethylamino, or pyrrolidinyl, the pyrrolidinyl being optionally substituted by hydroxyl or methoxymethyl.

R³ and R⁴ each independently represent a hydrogen atom, C₁-C₆ alkyl, preferably C₁-C₄ alkyl group (e.g. methyl, ethyl, n-propyl, isopropyl, n-butyl, isobutyl, tert-butyl, n-pentyl or n-hexyl) or C₃-C₆ cycloalkyl (e.g. cyclopropyl, cyclobutyl, cyclopentyl or cyclohexyl), or R³ and R⁴ together with the nitrogen atom to which they are attached form a 4- to 7-membered saturated heterocycle (e.g. pyrrolidinyl or piperidinyl), said heterocycle being optionally substituted with aminocarbonyl.

R⁵ and R⁶ each independently represent a hydrogen atom, C₁-C₆ alkyl, preferably C₁-C₄, alkyl group (e.g. methyl, ethyl, n-propyl, isopropyl, n-butyl, isobutyl, tert-butyl, n-pentyl or n-hexyl), or C₃-C₆ cycloalkyl (e.g. cyclopropyl, cyclobutyl, cyclopentyl or cyclohexyl), or R⁵ and R⁶ together with the nitrogen atom to which they are attached form a 4- to 7-membered saturated heterocycle (e.g pyrrolidinyl or piperidinyl), said heterocycle being optionally substituted with aminocarbonyl.

R⁷ represents C₁-C₆ alkyl (e.g. methyl), or a 6-membered saturated or unsaturated heterocyclic ring, the ring containing at least one, typically one or two, nitrogen atoms (e.g. pyridinyl, pyrimidinyl or piperidinyl), the ring being optionally substituted with one or more (e.g. 1 or 2) substituents independently selected halogen (e.g. chlorine or fluorine), oxo, C₁-C₆ alkoxy (e.g. methoxy), or C₁-C₆ alkyl such as C₁-C₄ alkyl group (e.g. methyl).

In an embodiment of the present invention, R⁷ represents C₁-C₆ alkyl, or a 6-membered saturated or unsaturated heterocyclic ring, the ring containing at least one nitrogen atom, the ring being optionally substituted with one or more substituents independently selected from oxo or methyl.

R⁸ represents pyridine-N-oxide optionally substituted with one or more substituents independently selected from halogen (e.g. chlorine or fluorine), or C₁-C₆ alkyl (e.g. methyl), or R⁸ represents C₃-C₆ cycloalkyl (e.g. cyclopropyl, cyclobutyl, cyclopentyl or cyclohexyl), C₁-C₆ hydroxyalkyl (e.g. hydroxycyclopropyl, hydroxycyclobutyl, hydroxycyclopentyl or hydroxycyclohexyl), or a 5- or 6-membered saturated heterocyclic ring containing at least one (e.g. 1 or 2) heteroatom selected from nitrogen and oxygen (e.g. pyrrolidinyl, tetrahydrofuranyl, or piperidinyl), which ring being optionally substituted with one or more (e.g. 1 or 2) substituents independently selected from halogen (e.g. chlorine or fluorine), C₁-C₆ alkoxy (e.g. methoxy), oxo, or C₁-C₆ alkyl (e.g. methyl). In an embodiment of the present invention, the 5- or 6-membered saturated heterocyclic ring is substituted with one or more substituents independently selected from halogen, oxo, or C₁-C₆ alkyl.

R⁹ and R¹⁰ each independently represent a hydrogen atom or C₁-C₆ alkyl, preferably C₁-C₄, alkyl group (e.g. methyl, ethyl, n-propyl, isopropyl, n-butyl, isobutyl, tert-butyl, n-pentyl or n-hexyl). In an embodiment of the present invention, R¹ and R¹⁰ are both hydrogen.

p is 0, 1 or 2. In an embodiment of the present invention, p is 0.

Each R group independently represents halogen (e.g. chlorine, fluorine, bromine or iodine), typically chlorine, or C₁-C₄ alkyl (e.g. methyl, ethyl, n-propyl, isopropyl, n-butyl, isobutyl, tert-butyl), typically methyl.

n represents 0, 1 or 2, typically 0 or 1. In an embodiment of the present invention, n is 0.

Ring D contains one or two ring-oxygen atoms. By ring-atom is meant an atom that is present in ring D (rather than being present in any substituents on ring D). Ring D does not contain any other ring atoms apart from oxygen and carbon. It will be appreciated that the definition of ring D is not intended to include unstable structures and is not intended to include any O—O bonds.

Ring D may be unsubstituted (i.e. the only substituent on ring D being hydrogen) or may be substituted with one or more substituents selected from C₁-C₆ alkyl, preferably C₁-C₄ alkyl (e.g. methyl, ethyl, n-propyl, isopropyl, n-butyl, or isobutyl, preferably methyl), C₃-C₆ cycloalkyl, or phenyl (said phenyl being optionally substituted with one or more substituents independently selected from halogen (e.g. chlorine or fluorine), hydroxyl or C₁-C₄ alkoxy). Additionally, when ring D is a 5-membered, non-aromatic ring containing two ring-oxygen atoms that are 1,3 disposed (i.e. where the two ring oxygen atoms in ring D have a 1,3 positional relationship relative to each other), ring D may also be optionally substituted with group E.

In a further embodiment of the present invention, ring D together with the two benzene carbon atoms to which it is fused, is a 5- or 6-membered, non-aromatic ring containing one or two ring-oxygen atoms, and optionally containing a carbon-carbon double bond between two ring carbon atoms other than said benzene carbon atoms, ring D being optionally substituted with one or more (e.g. 1, 2, 3 or 4) C₁-C₄ alkyl groups, typically one or more (e.g. 1, 2, 3 or 4) methyl groups.

In an embodiment of the present invention, ring D, together with the two benzene carbon atoms to which it is fused, is a 5-membered, non-aromatic ring containing one or two ring-oxygen atoms and which does not contain any double bonds other than that between said benzene carbon atoms, or ring D, together with the two benzene carbon atoms to which it is fused, is a 6-membered, non-aromatic ring containing one or two ring-oxygen atoms optionally containing a carbon-carbon double bond between two ring carbon atoms other than said benzene carbon atoms, each ring D being optionally substituted as defined herein.

In a further embodiment of the present invention, ring D, together with the two benzene carbon atoms to which it is fused, is a 5- or 6-membered, non-aromatic ring containing one or two ring-oxygen atoms wherein each ring D does not contain any double bonds other than that between said benzene carbon atoms, each ring D being optionally substituted as defined herein.

In an embodiment of the present invention, ring D is substituted by C₁-C₆ alkyl, preferably C₁-C₄ alkyl.

In a further embodiment of the present invention, ring D is substituted by at least two C₁-C₆ alkyl groups, preferably at least two C₁-C₄ alkyl (e.g. methyl) groups.

Group E, together with a single carbon atom on ring D, represents a 4- to 8-membered (e.g. 4, 5, 6, 7 or 8) cycloalkyl ring, such that group E forms a spiro structure with ring D.

In a further embodiment of the present invention, when ring D is other than a 5-membered non-aromatic ring containing two ring oxygen atoms that are 1,3 disposed, ring D is optionally substituted with methyl, and when ring is a 5-membered non-aromatic ring containing two ring oxygen atoms that are 1,3 disposed, ring D is optionally substituted with C₁-C₆ alkyl, C₃-C₆ cycloalkyl, optionally substituted phenyl or group E.

In one aspect of the invention, group B represents the group

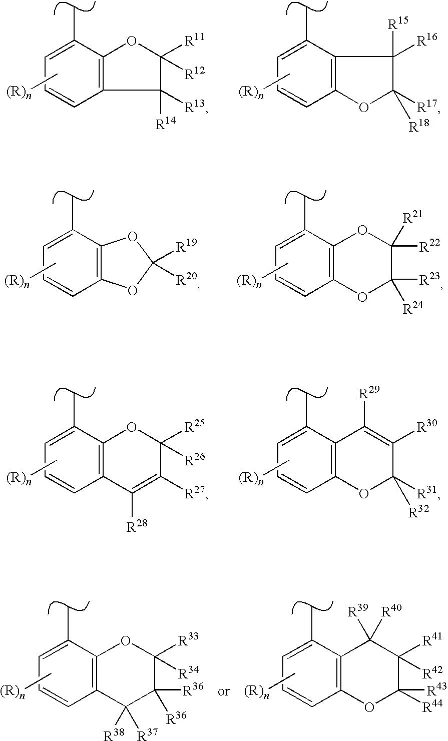

wherein each R¹¹, R¹², R¹³, R¹⁴, R¹⁵, R¹⁶, R¹⁷, R¹⁸, R²¹, R²², R²³, R²⁴, R²⁵, R²⁶, R²⁷, R²⁸, R²⁹, R³⁰ R³¹, R³², R³³, R³⁴, R³⁵, R³⁶, R³⁷, R³⁸, R³⁹, R⁴⁰, R⁴¹, R⁴², R⁴³, and R⁴⁴ independently represent a hydrogen atom or C₁-C₆ alkyl;

R¹⁹ and R²⁰ each independently represent hydrogen, C₁-C₆ alkyl, C₃-C₆ cycloalkyl, or optionally substituted phenyl; or R¹⁹ and R²⁰ together with the carbon to which they are attached form a 4- to 8-membered cycloalkyl ring;

n is 0, 1 or 2, and each R represents a group independently selected from halogen or C₁-C₄ alkyl.

In this aspect, R¹¹ and R¹² independently represent a hydrogen atom or C₁-C₆ alkyl, preferably C₁-C₄ alkyl e.g. methyl. In an embodiment of this aspect, R¹¹ and R¹² are both methyl.

R¹³ and R¹⁴ independently represent a hydrogen atom or C₁-C₆ alkyl, preferably C₁-C₄ alkyl e.g. methyl. In an embodiment of this aspect, R¹³ and R¹⁴ are both hydrogen.

R¹⁵ and R¹⁶ independently represent a hydrogen atom or C₁-C₆ alkyl, preferably C₁-C₄ alkyl e.g. methyl. In an embodiment of this aspect, R¹⁵ and R¹⁶ are both hydrogen.

R¹⁷ and R¹⁸ independently represent a hydrogen atom or C₁-C₆ alkyl, preferably C₁-C₄ alkyl e.g. methyl. In an embodiment of this aspect, R¹⁷ and R¹⁸ are both methyl.

R²¹ and R²² independently represent a hydrogen atom or C₁-C₆ alkyl, preferably C₁-C₄ alkyl e.g. methyl. In an embodiment of this aspect, R²¹ and R²² are both hydrogen, or R²¹ and R²² are both methyl.

R²³ and R²⁴ independently represent a hydrogen atom or C₁-C₆ alkyl, preferably C₁-C₄ alkyl e.g. methyl. In an embodiment of this aspect, R²³ and R²⁴ are both hydrogen or R²³ and R²⁴ are both methyl.

R²⁵, R²⁶, R³¹, and R³² independently represent a hydrogen atom or C₁-C₆ alkyl, preferably C₁-C₄ alkyl e.g. methyl. In an embodiment of this aspect, R²⁵, R²⁶, R³¹, and R³² are all methyl.

R¹⁹ and R²⁰ independently represent a hydrogen atom or C₁-C₆ alkyl, preferably C₁-C₄ alkyl (e.g. methyl, ethyl, n-propyl, isopropyl, n-butyl, isobutyl, tert-butyl, n-pentyl or n-hexyl), C₃-C₆ cycloalkyl (e.g. cyclopropyl, cyclobutyl, cyclopentyl or cyclohexyl) or phenyl, or R¹⁹ and R²⁰ together with the carbon to which they are attached form a 4- to 8-membered cycloalkyl ring (e.g. a 4, 5, 6, 7 or 8-membered cycloalkyl ring). In an embodiment of this aspect, R¹⁹ and R²⁰ are both C₁-C₄ alkyl (e.g. methyl) or R¹⁹ and R²⁰ together with the carbon to which they are attached form a 4- to 8-membered cycloalkyl ring.

R²⁷ and R²⁸ independently represent hydrogen or C₁-C₆ alkyl, preferably C₁-C₄ alkyl (e.g. methyl). In an embodiment of this aspect, R²⁷ and R²⁸ are both hydrogen.

R²⁹ and R³⁰ independently represent hydrogen or C₁-C₆ alkyl, preferably C₁-C₄ alkyl (e.g. methyl). In an embodiment of this aspect, R²⁹ and R³⁰ are both hydrogen.

R³³, R³⁴, R³⁵, R³⁶, R³⁷, R³⁸, R³⁹, R⁴⁰, R⁴¹, R⁴², R⁴³, and R⁴⁴ independently represent a hydrogen atom or C₁-C₆ alkyl, preferably C₁-C₄ alkyl. Preferably, R³³, R³⁴, R³⁵, R³⁶, R³⁷, R³⁸, R³⁹, R⁴⁰, R⁴¹, R⁴², R⁴³, and R⁴⁴ independently represent hydrogen or methyl.

In a further embodiment of this aspect, each R¹¹, R¹², R¹³, R¹⁴, R¹⁵, R¹⁶, R¹⁷, R¹⁸, R²¹, R²², R²³, R²⁴, R²⁵, R²⁶, R²⁷, R²⁸, R²⁹, R³⁰ R³¹, R³², R³³, R³⁴, R³⁵, R³⁶, R³⁷, R³⁸, R³⁹, R⁴⁰, R⁴¹, R⁴², R⁴³, and R⁴⁴ independently represent hydrogen or methyl.

Examples of group B include

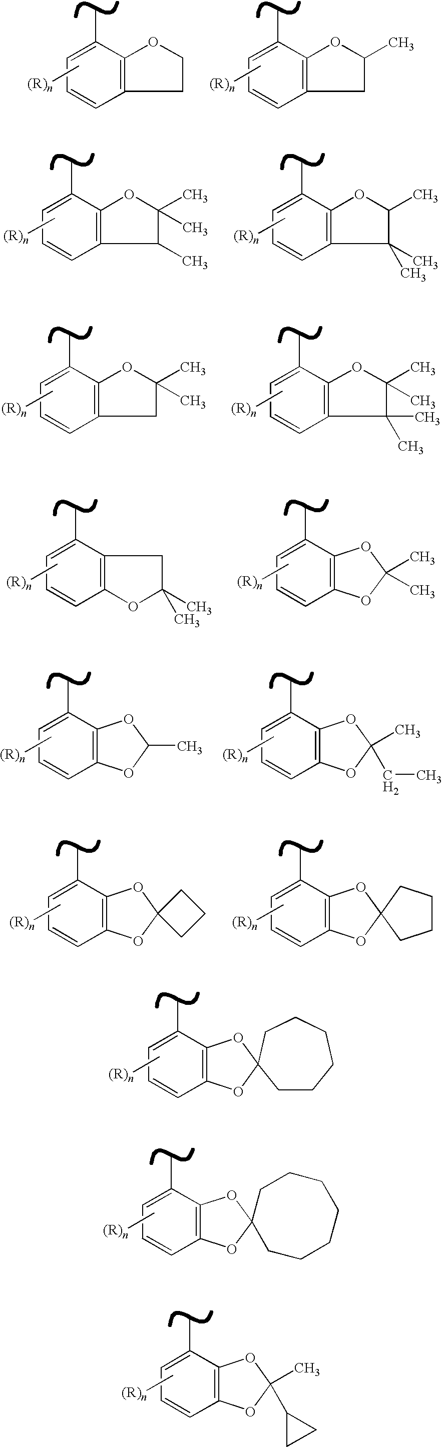

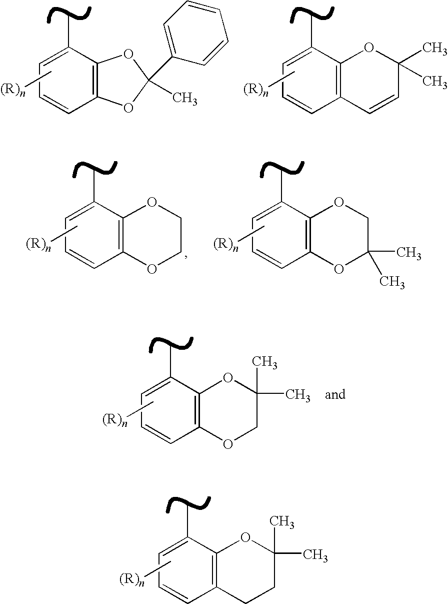

wherein each R represents a group independently selected from halogen or C₁-C₄ alkyl, and n is 0, 1 or 2.

In one embodiment of the present invention, group B is selected from the group consisting of

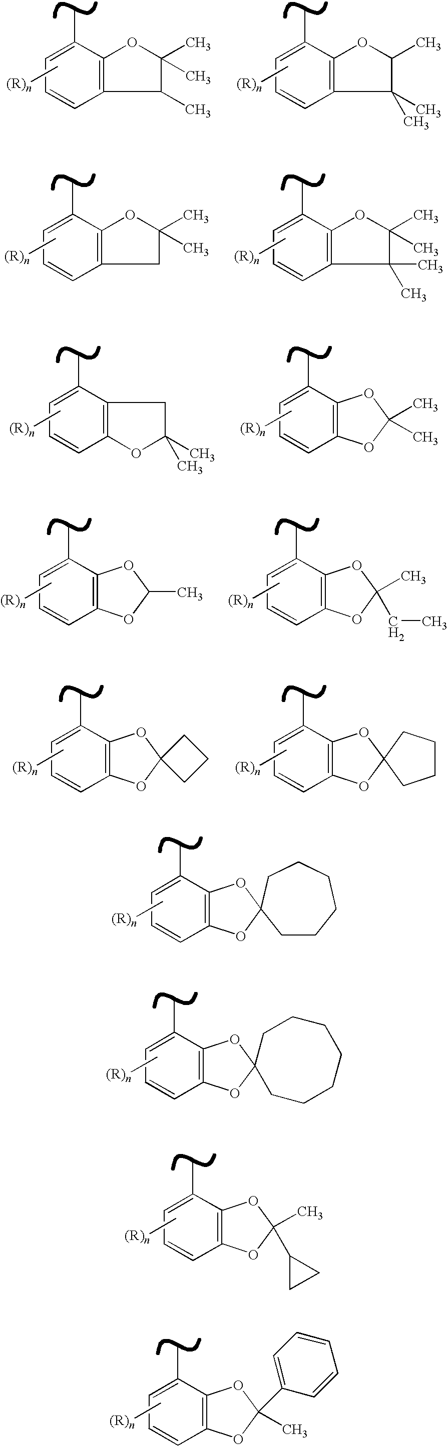

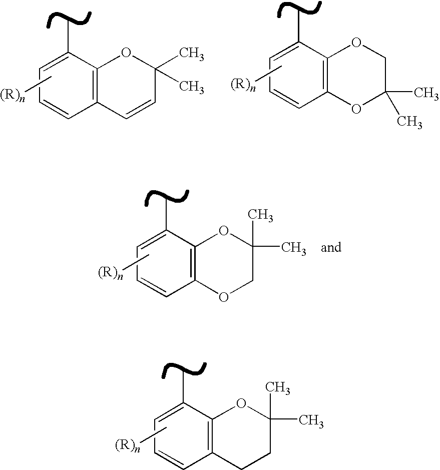

In a further embodiment of the present invention, group B is

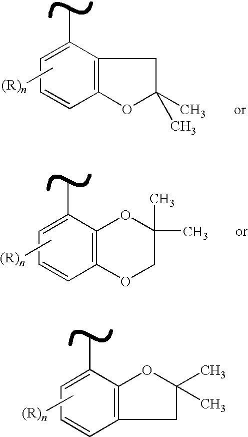

wherein each R represents a group independently selected from halogen or C₁-C₄ alkyl, and n is 0, 1 or 2.

In yet a further embodiment of the present invention, group B is

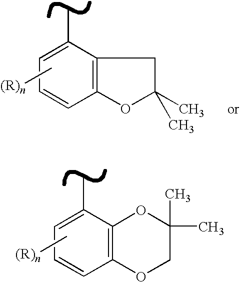

wherein each R represents a group independently selected from halogen or C₁-C₄ alkyl, and n is 0, 1 or 2.

In a further embodiment of the present invention, group B has structure (X) below:

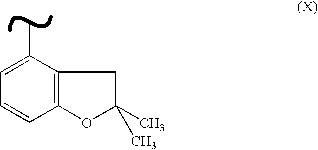

Without being bound by theory, the use of oxygen-containing bicycles in position B of formula (I) are considered to be advantageous, for example, in allowing for particularly potent CCR8 antagonism.

For example, group B being of structure (X) allows for very good CCR8 potency, and in addition, is particularly stable to oxidation thereby allowing enhanced metabolic stability. In this regard, stability against human microsomal metabolism in vitro is indicative of stability towards metabolism in vivo.

In a further aspect, the present invention provides a compound of general formula

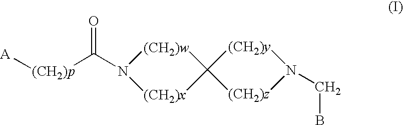

wherein
B represents the group

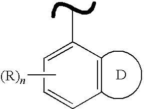

ring D, together with the two benzene carbon atoms to which it is fused, is a 5- or 6-membered, non-aromatic ring containing one or two ring-oxygen atoms, and optionally containing a carbon-carbon double bond between two ring carbon atoms other than said benzene carbon atoms, ring D being optionally substituted with one or more substituents independently selected from C₁-C₆ alkyl, C₃-C₆ cycloalkyl, or phenyl;
   and additionally wherein when ring D is a 5-membered, non-aromatic ring containing two ring-oxygen atoms that are 1,3 disposed, ring D may be optionally substituted with group E, wherein group E together with a single carbon atom on ring D, represents a 4- to 8-membered cycloalkyl ring, such that group E forms a spiro structure with ring D;
w, x, y and z are independently 1, 2 or 3;
Each R independently represents halogen or C₁-C₄ alkyl;
n is 0, 1 or 2;
A represents a group selected from phenyl, a 5- or 6-membered heteroaromatic ring containing at least one ring heteroatom independently selected from nitrogen, oxygen or sulphur, or pyridine-N-oxide, each group being optionally substituted with one or more substituents independently selected from hydroxyl, —CN, halogen, oxo (═O), C₁-C₆ aminoalkyl, C₁-C₆ alkoxy, C₁-C₆ alkylcarbonyl, —NR¹R², —C(O)—NR³R⁴, —C₁-C₄ alkyl-C(O)—NR⁵R⁶, —NHSO₂—R⁷, —NHC(O)R⁹, —SO₂NH₂, carboxyl, carboxyl-C₁-C₆ alkyl, C₁-C₆ alkoxycarbonyl, C₁-C₄ alkoxycarbonyl-C₁-C₄ alkyl, C₃-C₆ cycloalkylamino, phenyl, pyridyl (said phenyl and pyridyl being optionally further substituted with one or more groups independently selected from halogen, hydroxyl, carboxy or C₁-C₄ alkyl), C₁-C₆ alkyl or C₃-C₆ cycloalkyl (said latter two C₁-C₆ alkyl and C₃-C₆ cycloalkyl substituents being optionally further substituted with one or more substituents independently selected from halogen, hydroxyl, or —CN);
or A represents a 9- or 10-membered bicyclic ring system containing one or more ring heteroatoms independently selected from nitrogen, oxygen or sulphur and which is optionally substituted with one or more substituents independently selected from hydroxyl, —CN, halogen, oxo, C₁-C₆ alkoxy, —NR⁹R¹⁰, carboxyl, or C₁-C₆ alkyl;
p is 0, 1 or 2;
R¹ and R² each independently represent a hydrogen atom, a C₁-C₆ alkyl, or R¹ and R² together with the nitrogen atom to which they are attached form a 4- to 7-membered saturated heterocycle, said heterocycle being optionally substituted with hydroxyl, C₁-C₄ alkoxy, or C₁-C₄ alkoxy-C₁-C₄ alkyl;
R³ and R⁴ each independently represent a hydrogen atom, C₁-C₆ alkyl, or C₃-C₆ cycloalkyl, or R³ and R⁴ together with the nitrogen atom to which they are attached form a 4- to 7-membered saturated heterocycle, said heterocycle being optionally substituted with aminocarbonyl;
R⁵ and R⁶ each independently represent a hydrogen atom, C₁-C₆ alkyl, or C₃-C₆ cycloalkyl, or R⁵ and R⁶ together with the nitrogen atom to which they are attached form a 4- to 7-membered saturated heterocycle, said heterocycle being optionally substituted with aminocarbonyl;
R⁷ represents C₁-C₆ alkyl, or a 6-membered saturated or unsaturated heterocyclic ring, the ring containing at least one nitrogen atom, the ring being optionally substituted with one or more substituents independently selected from oxo, or methyl;
R⁸ represents pyridine-N-oxide optionally substituted with one or more substituents independently selected from halogen, or C₁-C₆ alkyl, or R⁸ represents C₁-C₆ alkyl, C₁-C₆ hydroxyalkyl, or a 5- or 6-membered saturated heterocyclic ring containing at least one heteroatom independently selected from nitrogen and oxygen, which ring being optionally substituted with one or more substituents independently selected from halogen, oxo, or C₁-C₆ alkyl;
R⁹ and R¹⁰ each independently represent a hydrogen atom or C₁-C₆ alkyl; or a pharmaceutically acceptable salt thereof.

In a further aspect, the present invention provides a compound according to formula (I) or a pharmaceutically acceptable salt thereof, wherein A represents pyridyl or pyrimidinyl each substituted with NH₂; w, x, y and z are independently 1, 2, or 3; p is 0, and B represents the group:

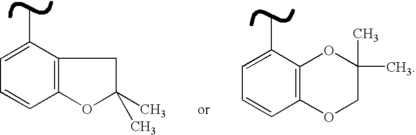

In a further aspect, the present invention provides a compound according to formula (I) or a pharmaceutically acceptable salt thereof, wherein A is pyridyl substituted with at least one (e.g. one) group independently selected from NR¹R², or —C₁-C₂-alkyl-C(O)—NR³R⁴; R¹ and R² each independently represent hydrogen or —C₁-C₄-alkyl; R³ and R⁴ each independently represent hydrogen or —C₁-C₄-alkyl; w, x, y and z are independently 1, 2, or 3 with the proviso that w+x is not greater than 5 and y+z is not greater than 5 and that the sum of w+x+y+z is greater 5; p is 0, and B represents the group:

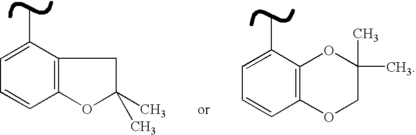

For compounds of formula (I) and salts thereof which are capable of existing in stereoisomeric forms, it will be understood that the invention encompasses all geometric and optical isomers of the compounds/salts of formula (I) and mixtures thereof including racemates. Tautomers and mixtures thereof also form an aspect of the present invention.

Particular compounds of the present invention include the following or pharmaceutically acceptable salts thereof:
3-[(2,2-dimethyl-2,3-dihydro-1-benzofuran-7-yl)methyl]-9-isonicotinoyl-3,9-diazaspiro[5.5]undecane;
3-[(2,2-dimethyl-2,3-dihydro-1-benzofuran-7-yl)methyl]-9-[(1-oxidopyridin-2-yl)carbonyl]-3,9-diazaspiro[5.5]undecane;
3-({9-[(2,2-dimethyl-2,3-dihydro-1-benzofuran-7-yl)methyl]-3,9-diazaspiro[5.5]undec-3-yl}carbonyl)pyridin-2(1H)-one;
[2-({9-[(2,2-dimethyl-2,3-dihydro-1-benzofuran-7-yl)methyl]-3,9-diazaspiro[5.5]undec-3-yl}carbonyl)phenyl]acetic acid;
methyl [2-({9-[(2,2-dimethyl-2,3-dihydro-1-benzofuran-7-yl)methyl]-3,9-diazaspiro[5.5]undec-3-yl}carbonyl)phenyl]acetate;
3-({9-[(2,2-dimethyl-2,3-dihydro-1-benzofuran-7-yl)methyl]-3,9-diazaspiro[5.5]undec-3-yl}carbonyl)-1-methylpyridin-2(1H)-one;
3-[(2,2-dimethyl-2,3-dihydro-1-benzofuran-7-yl)methyl]-9-(pyrimidin-4-ylcarbonyl)-3,9-diazaspiro[5.5]undecane;
4-({9-[(2,2-dimethyl-2,3-dihydro-1-benzofuran-7-yl)methyl]-3,9-diazaspiro[5.5]undec-3-yl}carbonyl)pyridin-3-ol;
2-({9-[(2,2-dimethyl-2,3-dihydro-1-benzofuran-7-yl)methyl]-3,9-diazaspiro[5.5]undec-3-yl}carbonyl)pyridin-3-ol;
5-({9-[(2,2-dimethyl-2,3-dihydro-1-benzofuran-7-yl)methyl]-3,9-diazaspiro[5.5]undec-3-yl}carbonyl)pyridin-2-amine;
3-({9-[(2,2-dimethyl-2,3-dihydro-1-benzofuran-7-yl)methyl]-3,9-diazaspiro[5.5]undec-3-yl}carbonyl)pyridin-2-amine;
2-({9-[(2,2-dimethyl-2,3-dihydro-1-benzofuran-7-yl)methyl]-3,9-diazaspiro[5.5]undec-3-yl}carbonyl)pyridin-4-ol;
3-({9-[(2,2-dimethyl-2,3-dihydro-1-benzofuran-7-yl)methyl]-3,9-diazaspiro[5.5]undec-3-yl}carbonyl)pyridin-4-ol;
3-(1H-1,2,3-benzotriazol-5-ylcarbonyl)-9-[(2,2-dimethyl-2,3-dihydro-1-benzofuran-7-yl)methyl]-3,9-diazaspiro[5.5]undecane;
5-({9-[(2,2-dimethyl-2,3-dihydro-1-benzofuran-7-yl)methyl]-3,9-diazaspiro[5.5]undec-3-yl}carbonyl)pyridine-2-carbonitrile;
2′-({9-[(2,2-dimethyl-2,3-dihydro-1-benzofuran-7-yl)methyl]-3,9-diazaspiro[5.5]undec-3-yl}carbonyl)biphenyl-2-carboxylic acid;
2-[2-({9-[(2,2-dimethyl-2,3-dihydro-1-benzofuran-7-yl)methyl]-3,9-diazaspiro[5.5]undec-3-yl}carbonyl)phenyl]acetamide;
1-{[2-({9-[(2,2-dimethyl-2,3-dihydro-1-benzofuran-7-yl)methyl]-3,9-diazaspiro[5.5]undec-3-yl}carbonyl)phenyl]acetyl}-D-prolinamide;
N-cyclopropyl-2-[2-({9-[(2,2-dimethyl-2,3-dihydro-1-benzofuran-7-yl)methyl]-3,9-diazaspiro[5.5]undec-3-yl}carbonyl)phenyl]acetamide;
3-[2-(2-azetidin-1-yl-2-oxoethyl)benzoyl]-9-[(2,2-dimethyl-2,3-dihydro-1-benzofuran-7-yl)methyl]-3,9-diazaspiro[5.5]undecane;
[5-chloro-2-({9-[(2,2-dimethyl-2,3-dihydro-1-benzofuran-7-yl)methyl]-3,9-diazaspiro[5.5]undec-3-yl}carbonyl)phenyl]acetic acid;
3-[2-({9-[(2,2-dimethyl-2,3-dihydro-1-benzofuran-7-yl)methyl]-3,9-diazaspiro[5.5]undec-3-yl}carbonyl)phenyl]propanoic acid;
4-({9-[(2,2-dimethyl-2,3-dihydro-1-benzofuran-7-yl)methyl]-3,9-diazaspiro[5.5]undec-3-yl}carbonyl)pyridin-2-amine;
4-({9-[(2,2-dimethyl-2,3-dihydro-1-benzofuran-7-yl)methyl]-3,9-diazaspiro[5.5]undec-3-yl}carbonyl)pyridin-3-amine;
N-[2-({9-[(2,2-dimethyl-2,3-dihydro-1-benzofuran-7-yl)methyl]-3,9-diazaspiro[5.5]undec-3-yl}carbonyl)phenyl]methanesulfonamide;
4-({9-[(2,2-dimethyl-2,3-dihydro-1-benzofuran-7-yl)methyl]-3,9-diazaspiro[5.5]undec-3-yl}carbonyl)-1H-pyrazol-3-amine;
3-[(2,2-dimethyl-2,3-dihydro-1-benzofuran-7-yl)methyl]-9-(1,2,3-thiadiazol-4-ylcarbonyl)-3,9-diazaspiro[5.5]undecane;
3-[(2,2-dimethyl-2,3-dihydro-1-benzofuran-7-yl)methyl]-9-[(3-methylisoxazol-4-yl)carbonyl]-3,9-diazaspiro[5.5]undecane;
3-[(2,2-dimethyl-2,3-dihydro-1-benzofuran-7-yl)methyl]-9-(1H-pyrazol-4-ylcarbonyl)-3,9-diazaspiro[5.5]undecane;
3-[(2,2-dimethyl-2,3-dihydro-1-benzofuran-7-yl)methyl]-9-(3-furoyl)-3,9-diazaspiro[5.5]undecane;
3-[(2,2-dimethyl-2,3-dihydro-1-benzofuran-7-yl)methyl]-9-(isoxazol-5-ylcarbonyl)-3,9-diazaspiro[5.5]undecane;
3-[(2,2-dimethyl-2,3-dihydro-1-benzofuran-7-yl)methyl]-9-[(1-methyl-1H-imidazol-4-yl)carbonyl]-3,9-diazaspiro[5.5]undecane;
1-[5-({9-[(2,2-dimethyl-2,3-dihydro-1-benzofuran-7-yl)methyl]-3,9-diazaspiro[5.5]undec-3-yl}carbonyl)-1H-pyrrol-3-yl]ethanone;
3-[(2,2-dimethyl-2,3-dihydro-1 benzofuran-7-yl)methyl]-9-(1H-pyrazol-3-ylcarbonyl)-3,9-diazaspiro[5.5]undecane;
3-[(2,2-dimethyl-2,3-dihydro-1-benzofuran-7-yl)methyl]-9-(1H-indol-3-ylcarbonyl)-3,9-diazaspiro[5.5]undecane;
3-[(2,2-dimethyl-2,3-dihydro-1-benzofuran-7-yl)methyl]-9-(1H-indazol-3-ylcarbonyl)-3,9-diazaspiro[5.5]undecane;
3-[(2,2-dimethyl-2,3-dihydro-1 benzofuran-7-yl)methyl]-9-(1H-indol-2-ylcarbonyl)-3,9-diazaspiro[5.5]undecane;
3-(2-chloroisonicotinoyl)-9-[(2,2-dimethyl-2,3-dihydro-1-benzofuran-7-yl)methyl]-3,9-diazaspiro[5.5]undecane;
[2-({9-[(2,2-dimethyl-2,3-dihydro-1-benzofuran-7-yl)methyl]-3,9-diazaspiro[5.5]undec-3-yl}carbonyl)phenyl]amine;
N-[2-({9-[(2,2-dimethyl-2,3-dihydro-1-benzofuran-7-yl)methyl]-3,9-diazaspiro[5.5]undec-3-yl}carbonyl)phenyl]acetamide;
N-[2-({9-[(2,2-dimethyl-2,3-dihydro-1-benzofuran-7-yl)methyl]-3,9-diazaspiro[5.5]undec-3-yl}carbonyl)phenyl]-2-hydroxyacetamide;
1-[4-({9-[(2,2-dimethyl-2,3-dihydro-1-benzofuran-7-yl)methyl]-3,9-diazaspiro[5.5]undec-3-yl}carbonyl)pyridin-2-yl]pyrrolidin-3-ol;
3-[(2,2-dimethyl-2,3-dihydro-1-benzofuran-7-yl)methyl]-9-{2-[(2S)-2-(methoxymethyl)pyrrolidin-1-yl]isonicotinoyl}-3,9-diazaspiro[5.5]undecane;
4-({9-[(2,2-dimethyl-2,3-dihydro-1-benzofuran-7-yl)methyl]-3,9-diazaspiro[5.5]undec-3-yl}carbonyl)-N-methylpyridin-2-amine;
N-[2-({9-[(2,2-dimethyl-2,3-dihydro-1-benzofuran-7-yl)methyl]-3,9-diazaspiro[5.5]undec-3-yl}carbonyl)phenyl]-6-methyl-2,4-dioxo-1,2,3,4-tetrahydropyrimidine-5-sulfonamide;
1-[2-({9-[(2,2-dimethyl-2,3-dihydro-1-benzofuran-7-yl)methyl]-3,9-diazaspiro[5.5]undec-3-yl}carbonyl)phenyl]imidazolidine-2,4-dione;
N-[2-({9-[(2,2-dimethyl-2,3-dihydro-1-benzofuran-7-yl)methyl]-3,9-diazaspiro[5.5]undec-3-yl}carbonyl)phenyl]nicotinamide 1-oxide;
N-[2-({9-[(2,2-dimethyl-2,3-dihydro-1-benzofuran-7-yl)methyl]-3,9-diazaspiro[5.5]undec-3-yl}carbonyl)phenyl]-1-methyl-L-prolinamide;
N-[2-({9-[(2,2-dimethyl-2,3-dihydro-1-benzofuran-7-yl)methyl]-3,9-diazaspiro[5.5]undec-3-yl}carbonyl)phenyl]tetrahydrofuran-2-carboxamide;
N-[2-({9-[(2,2-dimethyl-2,3-dihydro-1-benzofuran-7-yl)methyl]-3,9-diazaspiro[5.5]undec-3-yl}carbonyl)phenyl]-5-oxoprolinamide;
[4-({9-[(2,2-dimethyl-2,3-dihydro-1-benzofuran-7-yl)methyl]-3,9-diazaspiro[5.5]undec-3-yl}carbonyl)phenyl]amine;
3-[(2,2-dimethyl-2,3-dihydro-1-benzofuran-7-yl)methyl]-9-(3-methylisonicotinoyl)-3,9-diazaspiro[5.5]undecane;
2-({9-[(2,2-dimethyl-2,3-dihydro-1-benzofuran-7-yl)methyl]-3,9-diazaspiro[5.5]undec-3-yl}carbonyl)pyridin-4-amine;
3-[(2,2-dimethyl-2,3-dihydro-1-benzofuran-7-yl)methyl]-9-(2-methylisonicotinoyl)-3,9-diazaspiro[5.5]undecane;
6-({9-[(2,2-dimethyl-2,3-dihydro-1-benzofuran-7-yl)methyl]-3,9-diazaspiro[5.5]undec-3-yl}carbonyl)pyridin-3-amine;
4-({9-[(2,2-dimethyl-2,3-dihydro-1-benzofuran-7-yl)methyl]-3,9-diazaspiro[5.5]undec-3-yl}carbonyl)pyridazin-3-amine;
{[2-({9-[(2,2-dimethyl-2,3-dihydro-1-benzofuran-7-yl)methyl]-3,9-diazaspiro[5.5]undec-3-yl}carbonyl)pyridin-4-yl]methyl}amine;
4-({9-[(2,2-dimethyl-2,3-dihydro-1-benzofuran-7-yl)methyl]-3,9-diazaspiro[5.5]undec-3-yl}carbonyl)quinolin-2-ol;
3-[(2,2-dimethyl-2,3-dihydro-1-benzofuran-7-yl)methyl]-9-(1,8-naphthyridin-2-ylcarbonyl)-3,9-diazaspiro[5.5]undecane;
3-[(2,2-dimethyl-2,3-dihydro-1-benzofuran-7-yl)methyl]-9-(1,6-naphthyridin-2-ylcarbonyl)-3,9-diazaspiro[5.5]undecane;
4-({9-[(2,2-dimethyl-2,3-dihydro-1-benzofuran-7-yl)methyl]-3,9-diazaspiro[5.5]undec-3-yl}carbonyl)-6-methoxypyridin-3-amine;
4-({9-[(2,2-dimethyl-2,3-dihydro-1 benzofuran-7-yl)methyl]-3,9-diazaspiro[5.5]undec-3-yl}carbonyl)-2-methylquinolin-3-amine;
7-({9-[(2,2-dimethyl-2,3-dihydro-1-benzofuran-7-yl)methyl]-3,9-diazaspiro[5.5]undec-3-yl}carbonyl)-1H-indole-2,3-dione;
3-({9-[(2,2-dimethyl-2,3-dihydro-1-benzofuran-7-yl)methyl]-3,9-diazaspiro[5.5]undec-3-yl}carbonyl)pyridin-4-amine;
5-({9-[(2,2-dimethyl-2,3-dihydro-1-benzofuran-7-yl)methyl]-3,9-diazaspiro[5.5]undec-3-yl}carbonyl)pyridin-3-amine;
3-[(2,2-dimethyl-2,3-dihydro-1-benzofuran-7-yl)methyl]-9-(1H-indol-7-ylcarbonyl)-3,9-diazaspiro[5.5]undecane;
3-[(2,2-dimethyl-2,3-dihydro-1-benzofuran-7-yl)methyl]-9-(1H-indol-5-ylcarbonyl)-3,9-diazaspiro[5.5]undecane;
3-[(2,2-dimethyl-2,3-dihydro-1-benzofuran-7-yl)methyl]-9-(1H-indol-6-ylcarbonyl)-3,9-diazaspiro[5.5]undecane;
3-(1H-benzimidazol-6-ylcarbonyl)-9-[(2,2-dimethyl-2,3-dihydro-1-benzofuran-7-yl)methyl]-3,9-diazaspiro[5.5]undecane;
4-({9-[(3,3-dimethyl-2,3-dihydro-1,4-benzodioxin-5-yl)methyl]-3,9-diazaspiro[5.5]undec-3-yl}carbonyl)pyrimidin-2-amine;
3-({9-[(2,2-dimethyl-2,3-dihydro-1-benzofuran-7-yl)methyl]-3,9-diazaspiro[5.5]undec-3-yl}carbonyl)benzonitrile;
4-({9-[(2,2-dimethyl-2,3-dihydro-1-benzofuran-7-yl)methyl]-3,9-diazaspiro[5.5]undec-3-yl}carbonyl)benzonitrile;
4-({9-[(2,2-dimethyl-2,3-dihydro-1-benzofuran-7-yl)methyl]-3,9-diazaspiro[5.5]undec-3-yl}carbonyl)benzenesulfonamide;
[3-({9-[(2,2-dimethyl-2,3-dihydro-1-benzofuran-7-yl)methyl]-3,9-diazaspiro[5.5]undec-3-yl}carbonyl)phenyl]amine;
5-({9-[(2,2-dimethyl-2,3-dihydro-1-benzofuran-7-yl)methyl]-3,9-diazaspiro[5.5]undec-3-yl}carbonyl)pyrazin-2(1H)-one;
5-({9-[(2,2-dimethyl-2,3-dihydro-1-benzofuran-7-yl)methyl]-3,9-diazaspiro[5.5]undec-3-yl}carbonyl)pyridin-2(1H)-one;
3-isonicotinoyl-9-[(2-methyl-2,3-dihydro-1-benzofuran-7-yl)methyl]-3,9-diazaspiro[5.5]undecane;
3-isonicotinoyl-9-[(2,3,3-trimethyl-2,3-dihydro-1-benzofuran-7-yl)methyl]-3,9-diazaspiro[5.5]undecane;
3-isonicotinoyl-9-[(2,2,3-trimethyl-2,3-dihydro-1-benzofuran-7-yl)methyl]-3,9-diazaspiro[5.5]undecane;
3-(2,3-dihydro-1-benzofuran-7-ylmethyl)-9-isonicotinoyl-3,9-diazaspiro[5.5]undecane;
3-isonicotinoyl-9-[(2,2,3,3-tetramethyl-2,3-dihydro-1-benzofuran-7-yl)methyl]-3,9-diazaspiro[5.5]undecane;
3-[(5-chloro-2,2-dimethyl-2,3-dihydro-1-benzofuran-7-yl)methyl]-9-isonicotinoyl-3,9-diazaspiro[5.5]undecane;
3-isonicotinoyl-9-[(2,2,4-trimethyl-2,3-dihydro-1-benzofuran-7-yl)methyl]-3,9-diazaspiro[5.5]undecane;
3-[(4-chloro-2,2-dimethyl-2,3-dihydro-1-benzofuran-7-yl)methyl]-9-isonicotinoyl-3,9-diazaspiro[5.5]undecane;
3-[(2,2-dimethyl-2,3-dihydro-1-benzofuran-4-yl)methyl]-9-isonicotinoyl-3,9-diazaspiro[5.5]undecane;
4-({9-[(2,2-dimethyl-2,3-dihydro-1-benzofuran-4-yl)methyl]-3,9-diazaspiro[5.5]undec-3-yl}carbonyl)pyridin-3-amine;
6-({9-[(2,2-dimethyl-2,3-dihydro-1-benzofuran-4-yl)methyl]-3,9-diazaspiro[5.5]undec-3-yl}carbonyl)pyridin-3-amine;
2-[2-({9-[(2,2-dimethyl-2,3-dihydro-1-benzofuran-4-yl)methyl]-3,9-diazaspiro[5.5]undec-3-yl}carbonyl)phenyl]acetamide;
3-(1,3-benzodioxol-4-ylmethyl)-9-isonicotinoyl-3,9-diazaspiro[5.5]undecane;
3-[(2,2-dimethyl-1,3-benzodioxol-4-yl)methyl]-9-isonicotinoyl-3,9-diazaspiro[5.5]undecane;
4-({9-[(2,2-dimethyl-1,3-benzodioxol-4-yl)methyl]-3,9-diazaspiro[5.5]undec-3-yl}carbonyl)pyridin-3-amine;
4-({9-[(2,2-dimethyl-1,3-benzodioxol-4-yl)methyl]-3,9-diazaspiro[5.5]undec-3-yl}carbonyl)pyridin-2-amine;
2-({9-[(2,2-dimethyl-1,3-benzodioxol-4-yl)methyl]-3,9-diazaspiro[5.5]undec-3-yl}carbonyl)pyridin-3-amine;
2-[2-({9-[(2,2-dimethyl-1,3-benzodioxol-4-yl)methyl]-3,9-diazaspiro[5.5]undec-3-yl}carbonyl)phenyl]acetamide;
4-({9-[(2,2-dimethyl-1,3-benzodioxol-4-yl)methyl]-3,9-diazaspiro[5.5]undec-3-yl}carbonyl)pyridazin-3-amine;
4-({9-[(2-ethyl-2-methyl-1,3-benzodioxol-4-yl)methyl]-3,9-diazaspiro[5.5]undec-3-yl}carbonyl)pyridin-3-amine;
4-{[9-(spiro[1,3-benzodioxole-2,1′-cyclobutan]-4-ylmethyl)-3,9-diazaspiro[5.5]undec-3-yl}carbonyl]pyridin-3-amine;
4-{[9-(spiro[1,3-benzodioxole-2,1′-cyclopentan]-4-ylmethyl)-3,9-diazaspiro[5.5]undec-3-yl]carbonyl}pyridin-3-amine;
4-{[9-(spiro[1,3-benzodioxole-2,1′-cyclopentan]-4-ylmethyl)-3,9-diazaspiro[5.5]undec-3-yl]carbonyl}pyridin-2-amine;
4-{[9-(spiro[1,3-benzodioxole-2,1′-cycloheptan]-4-ylmethyl)-3,9-diazaspiro[5.5]undec-3-yl]carbonyl}pyridin-3-amine;
3-[(2-ethyl-2-methyl-1,3-benzodioxol-4-yl)methyl]-9-[(1-oxidopyridin-2-yl)carbonyl]-3,9-diazaspiro[5.5]undecane;
3-[(1-oxidopyridin-2-yl)carbonyl]-9-(spiro[1,3-benzodioxole-2,1′-cyclobutan]-4-ylmethyl)-3,9-diazaspiro[5.5]undecane;
3-[(1-oxidopyridin-2-yl)carbonyl]-9-(spiro[1,3-benzodioxole-2,1′-cyclooctan]-4-ylmethyl)-3,9-diazaspiro[5.5]undecane;
3-[(2-methyl-2-phenyl-1,3-benzodioxol-4-yl)methyl]-9-[(1-oxidopyridin-2-yl)carbonyl]-3,9-diazaspiro[5.5]undecane;
3-[(2-cyclopropyl-2-methyl-1,3-benzodioxol-4-yl)methyl]-9-[(1-oxidopyridin-2-yl)carbonyl]-3,9-diazaspiro[5.5]undecane;
3-[(2,2-dimethyl-2H-chromen-8-yl)methyl]-9-isonicotinoyl-3,9-diazaspiro[5.5]undecane;
4-({9-[(2,2-dimethyl-2H-chromen-8-yl)methyl]-3,9-diazaspiro[5.5]undec-3-yl}carbonyl)pyridin-3-amine;
6-({9-[(2,2-dimethyl-2H-chromen-8-yl)methyl]-3,9-diazaspiro[5.5]undec-3-yl}carbonyl)pyridin-3-amine;
2-[2-({9-[(2,2-dimethyl-2H-chromen-8-yl)methyl]-3,9-diazaspiro[5.5]undec-3-yl}carbonyl)phenyl]acetamide;
3-(2,3-dihydro-1,4-benzodioxin-5-ylmethyl)-9-isonicotinoyl-3,9-diazaspiro[5.5]undecane;
3-[(2,2-dimethyl-2,3-dihydro-1,4-benzodioxin-5-yl)methyl]-9-isonicotinoyl-3,9-diazaspiro[5.5]undecane;
3-[(2,2-dimethyl-2,3-dihydro-1-benzofuran-7-yl)methyl]-9-[3-(3-pyridin-2-yl-1,2,4-oxadiazol-5-yl)propanoyl]-3,9-diazaspiro[5.5]undecane;
4-({9-[(2,2-dimethyl-2H-chromen-8-yl)methyl]-3,9-diazaspiro[5.5]undec-3-yl}carbonyl)pyridin-2-amine;
6-({9-[(2,2-dimethyl-2H-chromen-8-yl)methyl]-3,9-diazaspiro[5.5]undec-3-yl}carbonyl)pyridin-2(1H)-one;
2-({9-[(2,2-dimethyl-2,3-dihydro-1-benzofuran-7-yl)methyl]-3,9-diazaspiro[5.5]undec-3-yl}carbonyl)benzonitrile;
4-({9-[(2,2-dimethyl-2,3-dihydro-1-benzofuran-7-yl)methyl]-3,9-diazaspiro[5.5]undec-3-yl}carbonyl)pyridine-2,6-diol;
3-[(6-fluoro-4H-1,3-benzodioxin-8-yl)methyl]-9-isonicotinoyl-3,9-diazaspiro[5.5]undecane;
4-({9-[(2,2-dimethyl-2H-chromen-8-yl)methyl]-3,9-diazaspiro[5.5]undec-3-yl}carbonyl)pyridazin-3-amine;
5-chloro-4-({9-[(2,2-dimethyl-1,3-benzodioxol-4-yl)methyl]-3,9-diazaspiro[5.5]undec-3-yl}carbonyl)pyrimidin-2-amine;
4-({9-[(2,2-dimethyl-2,3-dihydro-1-benzofuran-7-yl)methyl]-3,9-diazaspiro[5.5]undec-3-yl}carbonyl)pyrimidin-2-amine;
4-({9-[(2,2-dimethyl-1,3-benzodioxol-4-yl)methyl]-3,9-diazaspiro[5.5]undec-3-yl}carbonyl)pyrimidin-2-amine;
4-({9-[(2,2-dimethyl-2H-chromen-8-yl)methyl]-3,9-diazaspiro[5.5]undec-3-yl}carbonyl)pyrimidin-2-amine;
8-[(2,2-dimethyl-2,3-dihydro-1-benzofuran-7-yl)methyl]-2-(pyridin-4-ylacetyl)-2,8-diazaspiro[4.5]decane;
4-({9-[(2,2-dimethyl-2,3-dihydro-1-benzofuran-4-yl)methyl]-3,9-diazaspiro[5.5]undec-3-yl}carbonyl)pyridazin-3-amine;
4-({9-[(2,2-dimethyl-2,3-dihydro-1-benzofuran-4-yl)methyl]-3,9-diazaspiro[5.5]undec-3-yl}carbonyl)pyrimidin-2-amine;
4-({9-[(3,3-dimethyl-2,3-dihydro-1,4-benzodioxin-5-yl)methyl]-3,9-diazaspiro[5.5]undec-3-yl}carbonyl)pyridin-3-amine;
4-({9-[(3,3-dimethyl-2,3-dihydro-1,4-benzodioxin-5-yl)methyl]-3,9-diazaspiro[5.5]undec-3-yl}carbonyl)pyridin-2-amine;
4-({9-[(2,2,3,3-tetramethyl-2,3-dihydro-1-benzofuran-7-yl)methyl]-3,9-diazaspiro[5.5]undec-3-yl}carbonyl)pyrimidin-2-amine;
6-({9-[(2,2,3,3-tetramethyl-2,3-dihydro-1-benzofuran-7-yl)methyl]-3,9-diazaspiro[5.5]undec-3-yl}carbonyl)pyridin-3-amine;
4-({9-[(2,2,3,3-tetramethyl-2,3-dihydro-1-benzofuran-7-yl)methyl]-3,9-diazaspiro[5.5]undec-3-yl}carbonyl)pyridin-2-amine;
4-({9-[(2,2,3,3-tetramethyl-2,3-dihydro-1-benzofuran-7-yl)methyl]-3,9-diazaspiro[5.5]undec-3-yl}carbonyl)pyridin-3-amine;
4-({9-[(2,2-dimethyl-2H-chromen-5-yl)methyl]-3,9-diazaspiro[5.5]undec-3-yl}carbonyl)pyrimidin-2-amine;
4-({9-[(2,2-dimethyl-3,4-dihydro-2H-chromen-8-yl)methyl]-3,9-diazaspiro[5.5]undec-3-yl}carbonyl)pyrimidin-2-amine;
6-amino-3-({9-[(2,2-dimethyl-1,3-benzodioxol-4-yl)methyl]-3,9-diazaspiro[5.5]undec-3-yl}carbonyl)pyridin-2(1H)-one;
2-({9-[(2,2-dimethyl-2,3-dihydro-1-benzofuran-4-yl)methyl]-3,9-diazaspiro[5.5]undec-3-yl}carbonyl)pyridin-3-amine;
8-[(2,2-dimethyl-2,3-dihydro-1-benzofuran-7-yl)methyl]-2-isonicotinoyl-2,8-diazaspiro[4.5]decane;
8-[(2,2-dimethyl-2H-chromen-8-yl)methyl]-2-isonicotinoyl-2,8-diazaspiro[4.5]decane;
2-[(2,2-dimethyl-2,3-dihydro-1-benzofuran-7-yl)methyl]-8-isonicotinoyl-2,8-diazaspiro[4.5]decane;
7-[(2,2-dimethyl-2,3-dihydro-1-benzofuran-7-yl)methyl]-2-isonicotinoyl-2,7-diazaspiro[3.5]nonane;
7-[(2,2-dimethyl-2H-chromen-8-yl)methyl]-2-isonicotinoyl-2,7-diazaspiro[3.5]nonane;
2-[(2,2-dimethyl-2,3-dihydro-1-benzofuran-7-yl)methyl]-8-(pyridin-4-ylacetyl)-2,8-diazaspiro[4.5]decane;
7-[(2,2-dimethyl-2H-chromen-8-yl)methyl]-2-(pyridin-4-ylacetyl)-2,7-diazaspiro[3.5]nonane;
7-[(2,2-dimethyl-2,3-dihydro-1-benzofuran-7-yl)methyl]-2-(pyridin-4-ylacetyl)-2,7-diazaspiro[3.5]nonane;
2-[4-({9-[(3,3-dimethyl-2,3-dihydro-1,4-benzodioxin-5-yl)methyl]-3,9-diazaspiro[5.5]undec-3-yl}carbonyl)pyridin-3-yl]acetamide;
2-[(2,2-dimethyl-2H-chromen-8-yl)methyl]-8-isonicotinoyl-2,8-diazaspiro[4.5]decane;
8-[(2,2-dimethyl-2H-chromen-8-yl)methyl]-2-(pyridin-4-ylacetyl)-2,8-diazaspiro[4.5]decane;
3-[4-({9-[(2,2-dimethyl-2,3-dihydro-1-benzofuran-4-yl)methyl]-3,9-diazaspiro[5.5]undec-3-yl}carbonyl)pyridin-3-yl]propanamide;
4-({9-[(3,3-dimethyl-2,3-dihydro-1,4-benzodioxin-5-yl)methyl]-3,9-diazaspiro[5.5]undec-3-yl}carbonyl)pyridine-2-carbonitrile;
4-({9-[(3,3-dimethyl-2,3-dihydro-1,4-benzodioxin-5-yl)methyl]-3,9-diazaspiro[5.5]undec-3-yl}carbonyl)pyridine-2-carboxamide;
(2E)-3-[2-({9-[(3,3-dimethyl-2,3-dihydro-1,4-benzodioxin-5-yl)methyl]-3,9-diazaspiro[5.5]undec-3-yl}carbonyl)phenyl]acrylamide;
6-({9-[(2,2-dimethyl-2,3-dihydro-1-benzofuran-4-yl)methyl]-3,9-diazaspiro[5.5]undec-3-yl}carbonyl)pyridazin-3(2H)-one;
5-({9-[(2,2-dimethyl-2,3-dihydro-1-benzofuran-4-yl)methyl]-3,9-diazaspiro[5.5]undec-3-yl}carbonyl)pyridin-3-ol;
3-({9-[(2,2-dimethyl-2,3-dihydro-1-benzofuran-4-yl)methyl]-3,9-diazaspiro[5.5]undec-3-yl}carbonyl)pyridin-4(1H)-one;
3-({9-[(2,2-dimethyl-2,3-dihydro-1-benzofuran-4-yl)methyl]-3,9-diazaspiro[5.5]undec-3-yl}carbonyl)pyrazin-2(1H)-one;
6-({9-[(2,2-dimethyl-2,3-dihydro-1-benzofuran-4-yl)methyl]-3,9-diazaspiro[5.5]undec-3-yl}carbonyl)pyridin-2-amine;
6-({9-[(2,2-dimethyl-2,3-dihydro-1-benzofuran-4-yl)methyl]-3,9-diazaspiro[5.5]undec-3-yl}carbonyl)pyridin-3-ol;
6-({9-[(3,3-dimethyl-2,3-dihydro-1,4-benzodioxin-5-yl)methyl]-3,9-diazaspiro[5.5]undec-3-yl}carbonyl)pyridin-2-amine;
4-({7-[(2,2-dimethyl-2,3-dihydro-1-benzofuran-4-yl)methyl]-2,7-diazaspiro[3.5]non-2-yl}carbonyl)pyrimidin-2-amine;
6-({7-[(2,2-dimethyl-2,3-dihydro-1-benzofuran-4-yl)methyl]-2,7-diazaspiro[3.5]non-2-yl}carbonyl)pyridin-3-amine;
2-[4-({9-[(2,2-dimethyl-2,3-dihydro-1-benzofuran-4-yl)methyl]-3,9-diazaspiro[5.5]undec-3-yl}carbonyl)pyridin-3-yl]acetamide;
2-[4-({9-[(2,2,3,3-tetramethyl-2,3-dihydro-1-benzofuran-7-yl)methyl]-3,9-diazaspiro[5.5]undec-3-yl}carbonyl)pyridin-3-yl]acetamide;
N-cyclopropyl-2-[4-({9-[(2,2-dimethyl-2,3-dihydro-1-benzofuran-4-yl)methyl]-3,9-diazaspiro[5.5]undec-3-yl}carbonyl)pyridin-3-yl]acetamide;
[4-({9-[(3,3-dimethyl-2,3-dihydro-1,4-benzodioxin-5-yl)methyl]-3,9-diazaspiro[5.5]undec-3-yl}carbonyl)pyridin-3-yl]acetic acid;
[4-({9-[(2,2,3,3-tetramethyl-2,3-dihydro-1-benzofuran-7-yl)methyl]-3,9-diazaspiro[5.5]undec-3-yl}carbonyl)pyridin-3-yl]acetic acid;
[4-({9-[(2,2-dimethyl-2,3-dihydro-1-benzofuran-4-yl)methyl]-3,9-diazaspiro[5.5]undec-3-yl}carbonyl)pyridin-3-yl]acetic acid;
6-({7-[(2,2-dimethyl-2,3-dihydro-1-benzofuran-4-yl)methyl]-2,7-diazaspiro[4.4]non-2-yl}carbonyl)pyridin-3-amine;
5-chloro-4-({9-[(2,2-dimethyl-2,3-dihydro-1-benzofuran-4-yl)methyl]-3,9-diazaspiro[5.5]undec-3-yl}carbonyl)pyridin-2-amine;
2-[3-({9-[(2,2-dimethyl-2,3-dihydro-1-benzofuran-4-yl)methyl]-3,9-diazaspiro[5.5]undec-3-yl}carbonyl)phenyl]acetamide;
4-({9-[(2,2-dimethyl-2,3-dihydro-1-benzofuran-4-yl)methyl]-3,9-diazaspiro[5.5]undec-3-yl}carbonyl)benzamide;
2-[4-({9-[(3,3-dimethyl-2,3-dihydro-1,4-benzodioxin-5-yl)methyl]-3,9-diazaspiro[5.5]undec-3-yl}carbonyl)phenyl]acetamide;
5-chloro-4-({9-[(3,3-dimethyl-2,3-dihydro-1,4-benzodioxin-5-yl)methyl]-3,9-diazaspiro[5.5]undec-3-yl}carbonyl)pyrimidin-2-amine;
6-({9-[(3,3-dimethyl-2,3-dihydro-1,4-benzodioxin-5-yl)methyl]-3,9-diazaspiro[5.5]undec-3-yl}carbonyl)pyridin-3-amine;
2-({9-[(3,3-dimethyl-2,3-dihydro-1,4-benzodioxin-5-yl)methyl]-3,9-diazaspiro[5.5]undec-3-yl}carbonyl)pyridin-3-amine;
6-({9-[(2,2-dimethyl-2,3-dihydro-1,4-benzodioxin-5-yl)methyl]-3,9-diazaspiro[5.5]undec-3-yl}carbonyl)pyridin-3-amine;
4-({9-[(2,2-dimethyl-2,3-dihydro-1,4-benzodioxin-5-yl)methyl]-3,9-diazaspiro[5.5]undec-3-yl}carbonyl)pyridin-2-amine;
4-({9-[(2,2-dimethyl-2,3-dihydro-1,4-benzodioxin-5-yl)methyl]-3,9-diazaspiro[5.5]undec-3-yl}carbonyl)pyridin-3-amine;
4-({9-[(2,2-dimethyl-2,3-dihydro-1,4-benzodioxin-5-yl)methyl]-3,9-diazaspiro[5.5]undec-3-yl}carbonyl)pyrimidin-2-amine;
4-({9-[(3,3-dimethyl-2,3-dihydro-1,4-benzodioxin-5-yl)methyl]-3,9-diazaspiro[5.5]undec-3-yl}carbonyl)pyrimidin-2-amine;
8-[(2,2-dimethyl-2,3-dihydro-1-benzofuran-7-yl)methyl]-2-(pyridin-4-ylacetyl)-2,8-diazaspiro[4.5]decane;
6-({9-[(2,2-dimethyl-1,3-benzodioxol-4-yl)methyl]-3,9-diazaspiro[5.5]undec-3-yl}carbonyl)pyrimidin-4-amine;
6-({9-[(3,3-dimethyl-2,3-dihydro-1,4-benzodioxin-5-yl)methyl]-3,9-diazaspiro[5.5]undec-3-yl}carbonyl)pyrimidin-4-amine; or
methyl [2-({9-[(2,2-dimethyl-2,3-dihydro-1-benzofuran-7-yl)methyl]-3,9-diazaspiro[5.5]undec-3-yl}carbonyl)phenyl]acetate.

It should be noted that each exemplified compound above represents a particular and independent aspect of the invention.

In a further aspect, the present invention include the following or pharmaceutically acceptable salts thereof:
2-[2-({9-[(2,2-dimethyl-2,3-dihydro-1-benzofuran-7-yl)methyl]-3,9-diazaspiro[5.5]undec-3-yl}carbonyl)phenyl]acetamide;
N-cyclopropyl-2-[2-({9-[(2,2-dimethyl-2,3-dihydro-1-benzofuran-7-yl)methyl]-3,9-diazaspiro[5.5]undec-3-yl}carbonyl)phenyl]acetamide;
4-({9-[(2,2-dimethyl-2,3-dihydro-1-benzofuran-7-yl)methyl]-3,9-diazaspiro[5.5]undec-3-yl}carbonyl)pyridin-3-amine;
3-[(2,2-dimethyl-2,3-dihydro-1-benzofuran-4-yl)methyl]-9-isonicotinoyl-3,9-diazaspiro[5.5]undecane;
6-({9-[(2,2-dimethyl-2,3-dihydro-1-benzofuran-7-yl)methyl]-3,9-diazaspiro[5.5]undec-3-yl}carbonyl)pyridin-3-amine;
4-({9-[(2,2-dimethyl-1,3-benzodioxol-4-yl)methyl]-3,9-diazaspiro[5.5]undec-3-yl}carbonyl)pyridin-3-amine;
2-({9-[(2,2-dimethyl-1,3-benzodioxol-4-yl)methyl]-3,9-diazaspiro[5.5]undec-3-yl}carbonyl)pyridin-3-amine;
4-({9-[(2,2-dimethyl-2,3-dihydro-1-benzofuran-4-yl)methyl]-3,9-diazaspiro[5.5]undec-3-yl}carbonyl)pyridin-3-amine;
4-({9-[(2,2-dimethyl-2,3-dihydro-1-benzofuran-4-yl)methyl]-3,9-diazaspiro[5.5]undec-3-yl}carbonyl)pyridin-2-amine;
6-({9-[(2,2-dimethyl-2H-chromen-8-yl)methyl]-3,9-diazaspiro[5.5]undec-3-yl}carbonyl)pyridin-3-amine;
6-({9-[(2,2-dimethyl-2,3-dihydro-1-benzofuran-4-yl)methyl]-3,9-diazaspiro[5.5]undec-3-yl}carbonyl)pyridin-3-amine;
2-[2-({9-[(2,2-dimethyl-1,3-benzodioxol-4-yl)methyl]-3,9-diazaspiro[5.5]undec-3-yl}carbonyl)phenyl]acetamide;
4-({9-[(2,2-dimethyl-2,3-dihydro-1-benzofuran-7-yl)methyl]-3,9-diazaspiro[5.5]undec-3-yl}carbonyl)pyrimidin-2-amine;
4-({9-[(2,2-dimethyl-1,3-benzodioxol-4-yl)methyl]-3,9-diazaspiro[5.5]undec-3-yl}carbonyl)pyrimidin-2-amine;
4-({9-[(2,2-dimethyl-2,3-dihydro-1-benzofuran-4-yl)methyl]-3,9-diazaspiro[5.5]undec-3-yl}carbonyl)pyrimidin-2-amine;
4-({9-[(3,3-dimethyl-2,3-dihydro-1,4-benzodioxin-5-yl)methyl]-3,9-diazaspiro[5.5]undec-3-yl}carbonyl)pyridin-2-amine;
4-({9-[(2,2-dimethyl-2,3-dihydro-1,4-benzodioxin-5-yl)methyl]-3,9-diazaspiro[5.5]undec-3-yl}carbonyl)pyrimidin-2-amine;
6-({9-[(2,2-dimethyl-2,3-dihydro-1,4-benzodioxin-5-yl)methyl]-3,9-diazaspiro[5.5]undec-3-yl}carbonyl)pyridin-3-amine;
6-({9-[(3,3-dimethyl-2,3-dihydro-1,4-benzodioxin-5-yl)methyl]-3,9-diazaspiro[5.5]undec-3-yl}carbonyl)pyridin-3-amine; or
2-[4-({9-[(3,3-dimethyl-2,3-dihydro-1,4-benzodioxin-5-yl)methyl]-3,9-diazaspiro[5.5]undec-3-yl}carbonyl)pyridin-3-yl]acetamide.

It should also be noted that each of the exemplified compounds in Examples 1 to 181 represents an independent aspect of the present invention.

According to the present invention there is also provided a process for the preparation of compounds of formula (I) which comprises:

(a) reaction of a compound of formula (II):

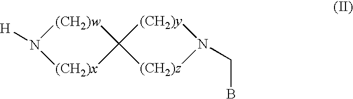

where w, x, y, z and B are as defined in formula (I), with a compound of formula (III)

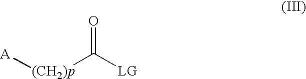

wherein p is as defined in formula (I) and A is as defined in formula (I) or a protected derivative thereof, and LG is a leaving group, or

(b) reaction of a compound of formula (IV):

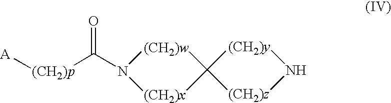

wherein p, w, x, y, and Z are as defined in formula (I) and A is as defined in formula (I) or a protected derivative thereof, with an aldehyde compound of formula (V):

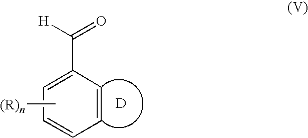

wherein D, n and R are as defined in formula (I), or

(c) reaction of a compound of formula (IV) defined above with a compound of formula (VI)

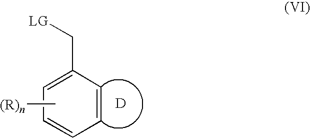

wherein D, n and R are as defined in formula (I), and LG is a suitable leaving group and optionally thereafter (a), (b) or (c):
converting a compound of formula (I) into another compound of formula (I),
removing any protecting groups, and/or
forming a pharmaceutically acceptable salt.

A compound of formula (II) can be prepared by process (d) by reacting a compound of formula (VII):

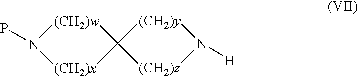

wherein w, x, y and z are as defined in formula (I) and P is a suitable protecting group, with a compound of formula (V), to form a compound of formula (II)′

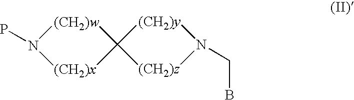

wherein B, w, x, y and z are as defined in formula (I) and P is a suitable protecting group, and subsequently removing the protecting group P.

A compound of formula (II) can be also be prepared by process (e) by reacting a compound of formula (VII) with a compound of (VI), and removing the protecting group P.

A compound of formula (IV) can be prepared by process (f) by reacting a compound of formula (VIII):

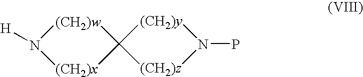

wherein w, x, y and z are as defined in formula (I) and P is a suitable protecting group, with a compound of formula (III) as defined above, to form a compound of formula (IV)′

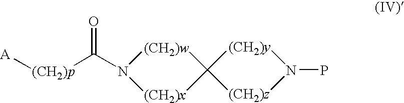

wherein A, p, w, x, y and z are as defined in formula (I) and P is a suitable protecting group, and subsequently removing the protecting group P.

Process (a) may be carried out using standard coupling reactions that are well know in the art. A suitable leaving group LG is, for example OH or chlorine. The coupling reaction may typically be carried out using activating reagents such as N-[(1H-1,2,3-benzotriazol-1-yloxy)(dimethylamino)methylene]-N-methylmethanaminium hexafluorophosphate (HBTU), N-[(dimethylamino)(3H-[1,2,3]triazolo[4,5-b]pyridin-3-yloxy)methylene]-N-methylmethanaminium hexafluorophosphate (HATU), or (benzotriazol-1-yloxy)tripyrrolidinophosphonium hexafluorophosphate (PYBOP). Typically, the reaction is carried out in the presence of a suitable base (e.g. triethylamine) and an organic solvent (e.g. dichloromethane) at a suitable temperature (e.g. room temperature).

Process (b) may be carried out using standard reductive amination procedures which are well known in the art. Typically, the reaction is carried out in the presence of a reducing agent, typically sodium triacetoxyborohydride [NaBH(OAc)₃]. Typically, the reaction is carried out in the presence of a suitable base (e.g. triethylamine) and an organic solvent (e.g. dichloromethane) at a suitable temperature (e.g. room temperature).

Process (c) may be carried out in a suitable organic solvent (e.g. DMF) at a suitable temperature (e.g. room temperature). The use of leaving groups are well known in the art for this type of reaction. Examples of typical leaving groups are halo, alkoxy, trifluoromethanesulfonyloxy, methanesulfonyloxy, or p-toluenesulfonyloxy. Typically, the leaving group is a halogen such as chlorine or bromine.

The coupling step of process (d) may be carried out according to the conditions described for process (b) above. The coupling step of process (e) may be carried out according to the conditions described for process (c) above. The coupling step of process (f) may be carried out according to the conditions described for process (a) above. An example of a typical protecting group P used in processes (d), (e) and (f) is tert-butyloxycarbonyl (t-boc). However, other suitable protecting groups may be used as described hereinafter.

Compounds of formulae (III), (V), (VI), (V), (VII) and (VIII) are either commercially available, are well known in the literature or may be prepared easily using known techniques, for example as shown in the accompanying Examples. The synthesis of diazaspiro intermediates are well known in the art (and are described for example, in WO 97/11940, U.S. Pat. No. 5,451,578, WO2005/084667, WO2005/044978, WO2005/080376, WO9711940, J. Comb. Chem. 2006, 8, 132-140, Bioorganic and Medicinal Chemistry Letters 12 (2203), 1103-1107) and analogous methods may be used to synthesise suitable spiro intermediates/starting reagents.

It will be appreciated by those skilled in the art that in the process of the present invention certain functional groups such as hydroxyl or amino groups in the starting reagents or intermediate compounds may need to be protected by protecting groups. Thus, the preparation of the compounds of formula (I) may involve, at an appropriate stage, the removal of one or more protecting groups.

The protection and deprotection of functional groups is well known in the art, and is described, for example, in ‘Protective Groups in Organic Chemistry’, edited by J. W. F. McOmie, Plenum Press (1973), and ‘Protective Groups in Organic Synthesis’, 2nd edition, T. W. Greene & P. G. M. Wuts, Wiley-Interscience (1991).

It should be noted that reference to intermediate compounds of formula (II), (II)′, (III), (IV), (V), (VI), (VII), (VIII) and (IV)′ encompasses free base forms and any suitable salts thereof.

Intermediates of formula (II) and (II)′ or salts thereof are believed to be novel and comprise an independent aspect of the invention.

Accordingly, the present invention also provides an intermediate of formula (II) or salt thereof

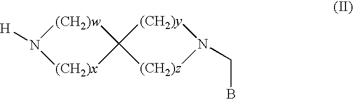

wherein B, w, x, y and z are as hereinbefore defined with respect to formula (I).

Furthermore, the present invention also provides an intermediate of formula (II)′ or salt thereof

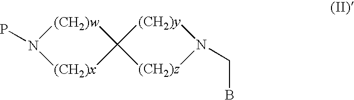

wherein B, w, x, y and z are as hereinbefore defined with respect to formula (I), and P is a suitable amino protecting group, for example t-boc.

For compounds of formulae (II) and (II)′, embodiments of the invention include those wherein each of B, w, x, y and z are as defined herein above in embodiments of the invention concerning compound of formula (I).

The compounds of formula (I) above may be converted to a pharmaceutically acceptable salt or solvate thereof, preferably a basic addition salt such as sodium, potassium, calcium, aluminium, lithium, magnesium, zinc, benzathine, chloroprocaine, choline, diethanolamine, ethanolamine, ethyldiamine, meglumine, tromethamine or procaine, or an acid addition salt such as a hydrochloride, hydrobromide, phosphate, acetate, benzenesulfonate, fumarate, maleate, tartrate, citrate, oxalate, methanesulphonate or p-toluenesulphonate.

The compounds of formula (I) and pharmaceutically acceptable salts thereof may exist in solvated, for example hydrated, as well as unsolvated forms, and the present invention encompasses all such solvated forms.

It will be appreciated that the compounds of formula (I) and pharmaceutically acceptable salts thereof may exist as zwitterions. In this regard, the representation of formula (I) and the examples of the present invention covers zwitterionic forms and mixtures thereof in all proportions.

The compounds of formula (I) have activity as pharmaceuticals, in particular as modulators of chemokine receptor (especially CCR8) activity, and may be used in the treatment (therapeutic or prophylactic) of conditions/diseases in human and non-human animals which are exacerbated or caused by excessive or dysregulated production of chemokines.

In an embodiment of the present invention, the compounds of the present invention have an IC50 value of less than 5 μM, or less than 2 μM, or less than 1 μM, or less than 0.1 μM or less than 0.05 μM when measured in the CCL1 SPA binding assay described herein.

A compound of the invention, or a pharmaceutically acceptable salt thereof, can be used in the treatment of:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus;

2. bone and joints: arthritis associated with or including osteoarthritis/osteoarthrosis, both primary and secondary to, for example, congenital hip dysplasia; cervical and lumbar spondylitis, and low back and neck pain; rheumatoid arthritis and Still's disease; seronegative spondyloarthropathies including ankylosing spondylitis, psoriatic arthritis, reactive arthritis and undifferentiated spondarthropathy; septic arthritis and other infection-related arthropathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositis and polymyositis; polymyalgia rheumatica; juvenile arthritis including idiopathic inflammatory arthritis of whatever joint distribution and associated syndromes, and rheumatic fever and its systemic complications; vasculitis including giant cell arteritis, Takayasu's arteritis, Churg-Strauss syndrome, polyarteritis nodosa, microscopic polyarteritis, and vasculitis associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthralgias, tendonitis, and myopathies;

3. pain and connective tissue remodelling of musculoskeletal disorders due to injury [for example sports injury] or disease: arthritis (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritis, scleroderma, mixed connective tissue disorder, spondyloarthropathies or periodontal disease (such as periodontitis);

4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosis et atrophica, pyoderma gangrenosum, skin sarcoid, discoid lupus erythematosus, pemphigus, pemphigoid, epidermolysis bullosa, urticaria, angioedema, vasculitis, toxic erythemas, cutaneous eosinophilias, alopecia greata, male-pattern baldness, Sweet's syndrome, Weber-Christian syndrome, erythema multiform; cellulitis, both infective and non-infective; panniculitis; cutaneous lymphomas, non-melanoma skin cancer and other dysplastic lesions; drug-induced disorders including fixed drug eruptions;

5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;

6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritus ani; coeliac disease, irritable bowel syndrome, and food-related allergies which may have effects remote from the gut (for example migraine, rhinitis or eczema);

7. abdominal: hepatitis, including autoimmune, alcoholic and viral; fibrosis and cirrhosis of the liver; cholecystitis; pancreatitis, both acute and chronic;

8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvovaginitis; Peyronie's disease; erectile dysfunction (both male and female);

9. allograft rejection: acute and chronic following, for example, transplantation of kidney, heart, liver, lung, bone marrow, skin or cornea or following blood transfusion; or chronic graft versus host disease;

10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;

11. other auto-immune and allergic disorders including Hashimoto's thyroiditis, Graves' disease, Addison's disease, diabetes mellitus, idiopathic thrombocytopenic purpura, eosinophilic fascitis, hyper-IgE syndrome, antiphospholipid syndrome;

12. other disorders with an inflammatory or immunological component; including acquired immune deficiency syndrome (AIDS), leprosy, Sezary syndrome, and paraneoplastic syndromes;

13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitis; disorders of the proximal and peripheral veins including phlebitis and thrombosis, including deep vein thrombosis and complications of varicose veins;

14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes;

15. gastrointestinal tract: Coeliac disease, proctitis, eosinophilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema; and

16. other disorders: sepsis.

Thus, in a further aspect, the present invention provides a compound of formula (I) or a pharmaceutically-acceptable salt thereof, as hereinbefore defined for use in therapy.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

In a further aspect, the present invention provides a method of treating a respiratory disease in a patient suffering from, or at risk of, said disease, which comprises administering to the patient a therapeutically effective amount of a compound of formula (I) or a pharmaceutically acceptable salt thereof, as hereinbefore defined.

In a further aspect, present invention provides the use of a compound of formula (I) or a pharmaceutically-acceptable salt thereof, as hereinbefore defined in the manufacture of a medicament for use in treating a respiratory disease.

In a further aspect, the present invention provides a method of treating an airways disease in a patient suffering from, or at risk of, said disease, which comprises administering to the patient a therapeutically effective amount of a compound of formula (I) or a pharmaceutically acceptable salt thereof, as hereinbefore defined.

In a further aspect, the present invention provides the use of a compound of formula (I) or a pharmaceutically-acceptable salt thereof, as hereinbefore defined in the manufacture of a medicament for use in treating an airways disease.

In a further aspect, the present invention provides a method of treating asthma, chronic obstructive pulmonary disease or rhinitis in a patient suffering from, or at risk of, said disease, which comprises administering to the patient a therapeutically effective amount of a compound of formula (I) or a pharmaceutically acceptable salt thereof, as hereinbefore defined.

In a further aspect, the present invention provides the use of a compound of formula (I) or a pharmaceutically-acceptable salt thereof, as hereinbefore defined in the manufacture of a medicament for use in treating asthma, chronic obstructive pulmonary disease or rhinitis.

In a further aspect, the present invention provides the use of a compound of formula (I) or a pharmaceutically-acceptable salt thereof, as hereinbefore defined in the manufacture of a medicament for the treatment of human diseases or conditions in which modulation of CCR8 activity is beneficial.

For the above-mentioned therapeutic uses the dosage administered will, of course, vary with the compound employed, the mode of administration, the treatment desired and the disorder indicated.

The compounds of formula (I) and pharmaceutically acceptable salts and solvates thereof may be used on their own but will generally be administered in the form of a pharmaceutical composition in which the formula (I) compound/salt/solvate (active ingredient) is in association with a pharmaceutically acceptable adjuvant, diluent or carrier.

Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I) or a pharmaceutically acceptable salt thereof, as hereinbefore defined, in association with a pharmaceutically acceptable adjuvant, diluent or carrier.

The invention further provides a process for the preparation of a pharmaceutical composition of the invention which comprises mixing a compound of formula (I) or a pharmaceutically acceptable salt thereof, as hereinbefore defined, with a pharmaceutically acceptable adjuvant, diluent or carrier.

The pharmaceutical compositions may be administered topically (e.g. to the lung and/or airways or to the skin) in the form of solutions, suspensions, heptafluoroalkane aerosols and dry powder formulations, or systemically, e.g. by oral administration in the form of tablets, capsules, syrups, powders or granules, or by parenteral administration in the form of solutions or suspensions, or by subcutaneous administration or by rectal administration in the form of suppositories or transdermally.

Dry powder formulations and pressurized HFA aerosols of the compounds of the invention may be administered by oral or nasal inhalation. For inhalation, the compound is desirably finely divided. The finely divided compound preferably has a mass median diameter of less than 10 μm, and may be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

The compounds of the invention may also be administered by means of a dry powder inhaler. The inhaler may be a single or a multi dose inhaler, and may be a breath actuated dry powder inhaler.

One possibility is to mix the finely divided compound of the invention with a carrier substance, for example, a mono-, di- or polysaccharide, a sugar alcohol, or another polyol. Suitable carriers are sugars, for example, lactose, glucose, raffinose, melezitose, lactitol, maltitol, trehalose, sucrose, mannitol; and starch. Alternatively the finely divided compound may be coated by another substance. The powder mixture may also be dispensed into hard gelatine capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient. With this system the active ingredient, with or without a carrier substance, is delivered to the patient.

For oral administration the compound of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compound of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules.

Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

The invention further relates to combination therapies wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, the compounds of the invention may be combined with agents listed below.

Non-steroidal anti-inflammatory agents (hereinafter NSAIDs) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerin; intra-articular therapies such as hyaluronic acid derivatives; and nutritional supplements such as glucosamine.

The present invention still further relates to the combination of a compound of the invention, or a pharmaceutically acceptable salt thereof, together with a cytokine or agonist or antagonist of cytokine function, (including agents which act on cytokine signalling pathways such as modulators of the SOCS system) including alpha-, beta-, and gamma-interferons; insulin-like growth factor type I (IGF-1); interleukins (IL) including IL1 to 17, and interleukin antagonists or inhibitors such as anakinra; tumour necrosis factor alpha (TNF-α) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxifylline.

In addition the invention relates to a combination of a compound of the invention, or a pharmaceutically acceptable salt thereof, with a monoclonal antibody targeting B-Lymphocytes (such as CD20 (rituximab), MRA-aIL16R and T-Lymphocytes, CTLA4-Ig, HuMax Il-15).

The present invention still further relates to the combination of a compound of the invention, or a pharmaceutically acceptable salt thereof, with a modulator of chemokine receptor function such as an antagonist of CCR1, CCR2, CCR2A, CCR2B, CCR3, CCR4, CCR5, CCR6, CCR7, CCR8, CCR9, CCR10 and CCR11 (for the C-C family); CXCR1, CXCR2, CXCR3, CXCR4 and CXCR5 (for the C-X-C family) and CX₃CR1 for the C-X₃-C family.

The present invention further relates to the combination of a compound of the invention, or a pharmaceutically acceptable salt thereof, with an inhibitor of matrix metalloprotease (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12, including agents such as doxycycline.

The present invention still further relates to the combination of a compound of the invention, or a pharmaceutically acceptable salt thereof, and a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY x 1005.

The present invention further relates to the combination of a compound of the invention, or a pharmaceutically acceptable salt thereof, and a receptor antagonist for leukotrienes (LT) B4, LTC4, LTD4, and LTE4. selected from the group consisting of the phenothiazine-3-1s such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of the invention, or a pharmaceutically acceptable salt thereof, and a phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

The present invention further relates to the combination of a compound of the invention, or go a pharmaceutically acceptable salt thereof, and a histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a compound of the invention, or a pharmaceutically acceptable salt thereof, and a proton pump inhibitor (such as omeprazole) or a gastroprotective histamine type 2 receptor antagonist.

The present invention further relates to the combination of a compound of the invention, or a pharmaceutically acceptable salt thereof, and an antagonist of the histamine type 4 receptor.

The present invention still further relates to the combination of a compound of the invention, or a pharmaceutically acceptable salt thereof, and an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention further relates to the combination of a compound of the invention, or a pharmaceutically acceptable salt thereof, and an anticholinergic agents including muscarinic receptor (M1, M2, and M3) antagonist such as atropine, hyoscine, glycopyrrolate, ipratropium bromide, tiotropium bromide, oxitropium bromide, pirenzepine or telenzepine.

The present invention still further relates to the combination of a compound of the invention, or a pharmaceutically acceptable salt thereof, and a beta-adrenoceptor agonist (including beta receptor subtypes 1-4) such as isoprenaline, salbutamol, formoterol, salmeterol, terbutaline, orciprenaline, bitolterol mesylate, or pirbuterol, or a chiral enantiomer thereof.

The present invention further relates to the combination of a compound of the invention, or a pharmaceutically acceptable salt thereof, and a chromone, such as sodium cromoglicate or nedocromil sodium.

The present invention still further relates to the combination of a compound of the invention, or a pharmaceutically acceptable salt thereof, with a glucocorticoid, such as flunisolide, triamcinolone acetonide, beclomethasone dipropionate, budesonide, fluticasone propionate, ciclesonide or mometasone furoate.

The present invention further relates to the combination of a compound of the invention, or a pharmaceutically acceptable salt thereof, with an agent that modulates a nuclear hormone receptor such as PPARs.

The present invention still further relates to the combination of a compound of the invention, or a pharmaceutically acceptable salt thereof, together with an immunoglobulin (Ig) or Ig preparation or an antagonist or antibody modulating Ig function such as anti-IgE (for example omalizumab).

The present invention further relates to the combination of a compound of the invention, or a pharmaceutically acceptable salt thereof, and another systemic or topically-applied anti-inflammatory agent, such as thalidomide or a derivative thereof, a retinoid, dithranol or calcipotriol.

The present invention still further relates to the combination of a compound of the invention, or a pharmaceutically acceptable salt thereof, and combinations of aminosalicylates and sulfapyridine such as sulfasalazine, mesalazine, balsalazide, and olsalazine; and immunomodulatory agents such as the thiopurines, and corticosteroids such as budesonide.

The present invention further relates to the combination of a compound of the invention, or a pharmaceutically acceptable salt thereof, together with an antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

The present invention still further relates to the combination of a compound of the invention, or a pharmaceutically acceptable salt thereof, and a cardiovascular agent such as a calcium channel blocker, a beta-adrenoceptor blocker, an angiotensin-converting enzyme (ACE) inhibitor, an angiotensin-2 receptor antagonist; a lipid lowering agent such as a statin or a fibrate; a modulator of blood cell morphology such as pentoxifylline; thrombolytic, or an anticoagulant such as a platelet aggregation inhibitor.

The present invention further relates to the combination of a compound of the invention, or a pharmaceutically acceptable salt thereof, and a CNS agent such as an antidepressant (such as sertraline), an anti-Parkinsonian drug (such as deprenyl, L-dopa, ropinirole, pramipexole, a MAOB inhibitor such as selegine and rasagiline, a comP inhibitor such as tasmar, an A-2 inhibitor, a dopamine reuptake inhibitor, an NMDA antagonist, a nicotine agonist, a dopamine agonist or an inhibitor of neuronal nitric oxide synthase), or an anti-Alzheimer's drug such as donepezil, rivastigmine, tacrine, a COX-2 inhibitor, propentofylline or metrifonate.

The present invention still further relates to the combination of a compound of the invention, or a pharmaceutically acceptable salt thereof, and an agent for the treatment of acute or chronic pain, such as a centrally or peripherally-acting analgesic (for example an opioid or derivative thereof), carbamazepine, phenyloin, sodium valproate, amitryptyline or other anti-depressant agent-s, paracetamol, or a non-steroidal anti-inflammatory agent.

The present invention further relates to the combination of a compound of the invention, or a pharmaceutically acceptable salt thereof, together with a parenterally or topically-applied (including inhaled) local anaesthetic agent such as lignocaine or a derivative thereof.

A compound of the present invention, or a pharmaceutically acceptable salt thereof, can also be used in combination with an anti-osteoporosis agent including a hormonal agent such as raloxifene, or a biphosphonate such as alendronate.

The present invention still further relates to the combination of a compound of the invention, or a pharmaceutically acceptable salt thereof, together with a: (i) tryptase inhibitor; (ii) platelet activating factor (PAF) antagonist; (iii) interleukin converting enzyme (ICE) inhibitor; (iv) IMPDH inhibitor; (v) adhesion molecule inhibitors including VLA-4 antagonist; (vi) cathepsin; (vii) kinase inhibitor such as an inhibitor of tyrosine kinase (such as Btk, Itk, Jak3 or MAP, for example Gefitinib or Imatinib mesylate), a serine/threonine kinase (such as an inhibitor of a MAP kinase such as p38, JNK, protein kinase A, B or C, or IKK), or a kinase involved in cell cycle regulation (such as a cyclin dependent kinase); (viii) glucose-6 phosphate dehydrogenase inhibitor; (ix) kinin-B₁- or B₂-receptor antagonist; (x) anti-gout agent, for example colchicine; (xi) xanthine oxidase inhibitor, for example allopurinol; (xii) uricosuric agent, for example probenecid, sulfinpyrazone or benzbromarone; (xiii) growth hormone secretagogue; (xiv) transforming growth factor (TGFβ); (xv) platelet-derived growth factor (PDGF); (xvi) fibroblast growth factor for example basic fibroblast growth factor (bFGF); (xvii) granulocyte macrophage colony stimulating factor (GM-CSF); (xviii) capsaicin cream; (xix) tachykinin NK₁ or NK.sub3. receptor antagonist such as NKP-608C, SB-233412 (talnetant) or D-4418; (xx) elastase inhibitor such as UT-77 or ZD-0892; (xxi) TNF-alpha converting enzyme inhibitor (TACE); (xxii) induced nitric oxide synthase (iNOS) inhibitor; (xxiii) chemoattractant receptor-homologous molecule expressed on TH2 cells, (such as a CRTH2 antagonist); (xxiv) inhibitor of P38; (xxv) agent modulating the function of Toll-like receptors (TLR), (xxvi) agent modulating the activity of purinergic receptors such as P2×7; or (xxvii) inhibitor of transcription factor activation such as NFκB, API, or STATS.

A compound of the invention, or a pharmaceutically acceptable salt thereof, can also be used in combination with an existing therapeutic agent for the treatment of cancer, for example suitable agents include:

(i) an antiproliferative/antineoplastic drug or a combination thereof, as used in medical oncology, such as an alkylating agent (for example cis-platin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin);

(ii) a cytostatic agent such as an antiestrogen (for example tamoxifen, toremifene, raloxifene, droloxifene or iodoxyfene), an estrogen receptor down regulator (for example fulvestrant), an antiandrogen (for example bicalutamide, flutamide, nilutamide or cyproterone acetate), a LHRH antagonist or LHRH agonist (for example goserelin, Leuprorelin or buserelin), a progestogen (for example megestrol acetate), an aromatase inhibitor (for example as anastrozole, letrozole, vorazole or exemestane) or an inhibitor of 5α-reductase such as finasteride;

(iii) an agent which inhibits cancer cell invasion (for example a metalloproteinase inhibitor like marimastat or an inhibitor of urokinase plasminogen activator receptor function);

(iv) an inhibitor of growth factor function, for example: a growth factor antibody (for example the anti-erbb2 antibody trastuzumab, or the anti-erbb1 antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) or 6-acrylamido-N-(3-chloro-4-fluorophenyl)-7-(3-morpholinopropoxy)quinazolin-4-amine (CI 1033)), an inhibitor of the platelet-derived growth factor family, or an inhibitor of the hepatocyte growth factor family;

(v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin αvβ3 function or an angiostatin);

(vi) a vascular damaging agent such as combretastatin A4, or a compound disclosed in WO 99/02166, WO 00/40529, WO 00/41669, WO 01/92224, WO 02/04434 or WO 02/08213;

(vii) an agent used in antisense therapy, for example one directed to one of the targets listed above, such as ISIS 2503, an anti-ras antisense;

(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; or

(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell energy, approaches using transfected immune cells such as cytokine-transfected dendritic cells, approaches using cytokine-transfected tumour cell lines and approaches using anti-idiotypic antibodies.

The invention will now be further explained by reference to the following illustrative examples.

The following abbreviations are used in the examples.

HATU—N-[(dimethylamino)(3H-[1,2,3]triazolo[4,5-b]pyridin-3-yloxy)methylene]-N-methylmethanaminium hexafluorophosphate;

HBTU—N-[(1H-1,2,3-benzotriazol-1-yloxy)(dimethylamino)methylene]-N-methylmethanaminium hexafluorophosphate;

HOBT—1-Hydroxybenzotriazole;

PYBOP—benzotriazol-1-yloxy)tripyrrolidinophosphonium hexafluorophosphate;

AIBN—2,2′-(E)-diazene-1,2-diylbis(2-methylpropanenitrile);

NMP—1-methyl-2-pyrrolidinone;

Boc—tert-butoxycarbonyl;

DBU—1,8-diazabicyclo[5.4.0]undec-7-ene;

THF—tetrahydrofuran;

DIBAL—H-diisobutylaluminium hydride;

TBME—tert-butyl methyl ether;

EtOAc—ethyl acetate;

RP—18-reversed phase C18;

SCX—strong cation-exchange.

HPLC Method A

HPLC method A was performed with an Agilent 1100 series machine on Kromasil©D C18 5 μm 3.0×100 mm column. The aqueous phase was water/TFA (99.8/0.1) and the organic phase was acetonitrile/TFA (99.92/0.08). Flow was 1 ml/min and the gradient was set from 10 to 100% of organic phase over 20 min. Detection was carried out at 220, 254 and 280 nm.

HPLC Method B

HPLC method B was performed with an Agilent 1100 series machine on XTerra® RP₈ 5 μm 3.0×100 mm column. The aqueous phase was 15 mM NH3 in water and the organic phase was acetonitrile. Flow was 1 ml/min and the gradient was set from 10 to 100% of organic phase over 20 min. Detection was carried out at 220, 254 and 280 nm.

HPLC Method C

HPLC method C was performed with an Agilent 1100 series machine on BDS C-18 5 μm 4.6×250 mm column. The aqueous phase was 20 mM NH4OAc in water and the organic phase was acetonitrile. Flow was 0.7 ml/min and the gradient was set from 50 to 100% of organic phase over 10 min. Detection was carried out at 220, 254 and 280 nm.

Intermediate A

### 3-[(2,2-dimethyl-2,3-dihydro-1-benzofuran-7-yl)methyl]-3,9-diazaspiro[5.5]undecane

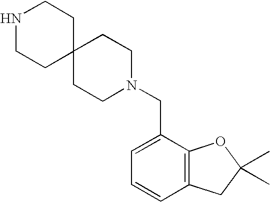

A mixture of tert-butyl 3,9-diazaspiro[5.5]undecane-3-carboxylate hydrochloric acid salt (5.00 g, 17.2 mmol), 2,2-dimethyl-2,3-dihydro-1-benzofuran-7-carbaldehyde (3.26 g, 18.5 mmol), sodium triacetoxyborohydride (5.97 g, 28.2 mmol) and acetonitrile was stirred at room temperature for 3 h. The reaction mixture was applied to silica and eluted first with 20% EtOAc in heptane, and then with EtOAc/MeOH/triethylamine (90/515). The fraction containing the crude product was evaporated and to this Intermediate tert-butyl 9-[(2,2-dimethyl-2,3-dihydro-1-benzofuran-7-yl)methyl]-3,9-diazaspiro[5.5]undecane-3-carboxylate was added 1M methanolic hydrochloric acid (50 ml) and the mixture was stirred at room temperature for 1 h then evaporated. The residue was purified by acidic ion-exchange resin to yield the product as a off-white solid (4.71 g, 71%).

¹H NMR (399.989 MHz, D2O) δ 7.12 (d, 1H), 7.01 (d, 1H), 6.80 (t, 1H), 3.63-3.56 (m, 2H), 3.08-2.99 (m, 4H), 2.95 (s, 2H), 2.66-2.50 (m, 4H), 1.68-1.42 (m, 8H), 1.39-1.30 (m, 6H)

APCI-MS m/z: 315.3 [MH+]

HPLC (Method A) Retention time: 4.23 min

HPLC (Method B) Retention time: 8.07 min

Intermediate B

### 3-[(2,2-dimethyl-1,3-benzodioxol-4-yl)methyl]-3,9-diazaspiro[5.5]undecane

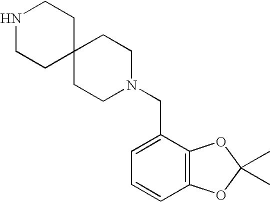

The compound was prepared by the procedure of Intermediate A using tert-butyl 3,9-diazaspiro[5.5]undecane-3-carboxylate hydrochloric acid salt and 2,2-dimethyl-1,3-benzodioxole-4-carbaldehyde as starting materials to give the product as a yellow oily solid (0.9 g, 51%).

¹H NMR (399.99 MHz, DMSO-D6) δ 6.78-6.64 (m, 3H), 3.40-3.23 (m, 2H), 2.65-2.55 (m, 4H), 2.35-2.26 (m, 4H), 1.61 (s, 6H), 1.40 (t, 4H), 1.28 (t, 4H)

APCI-MS m/z: 317.2 [MH+]

HPLC (Method A) Retention time: 6.58 min

HPLC (Method B) Retention time: 2.00 min

Intermediate C

### 3-[(2,2-dimethyl-2,3-dihydro-1-benzofuran-4-yl)methyl]-3,9-diazaspiro[5.5]undecane Dihydrochloride

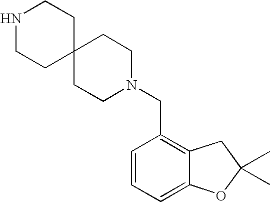

A mixture of tert-butyl 3,9-diazaspiro[5.5]undecane-3-carboxylate (0.56 g, 2.0 mmol), Intermediate W (0.35 g, 2.0 mmol), sodium triacetoxyborohydride (0.84 g, 4.0 mmol) in acetonitrile was stirred at 40° C. for 3 h. Aqueous sodium hydrogen carbonate was added, and the mixture extracted with ethyl acetate. The organic layer was evaporated, the residue was dissolved in methanol, 4M hydrochloric acid in dioxane (5 ml) was added and the mixture was stirred for 1 h. The reaction mixture was evaporated to yield the product as an off-white solid (0.4 g, 52%).

APCI-MS m/z: 315.3 [MH+]

Intermediate D

### 2-methyl-2,3-dihydro-1-benzofuran-7-carbaldehyde

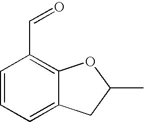

The title compound was prepared by the procedure described in Intermediate T using 3-bromoprop-1-ene and salicylaldehyde to afford the product (3 g, 75%).

¹H NMR (299.944 MHz, CDCl3) δ 10.22 (s, 1H), 7.61-7.57 (m, 1H), 7.39-7.35 (m, 1H), 6.91 (t, J=7.6 Hz, 1H), 5.17-5.05 (m, 1H), 3.41-3.30 (m, 1H), 2.90-2.79 (m, 1H), 1.59-1.53 (m, 3H)

Intermediate F

### 5-chloro-2,2-dimethyl-2,3-dihydro-1-benzofuran-7-carbaldehyde

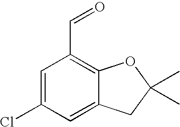

The title compound was prepared by the procedure described in Intermediate T using 3-chloro-2-methylprop-1-ene and 5-chloro-2-hydroxybenzaldehyde (0.8 g, 37%).

¹H NMR (399.99 MHz, CDCl3) δ 10.16 (s, 1H), 7.57-7.56 (m, 1H), 7.31-7.29 (m, 1H), 3.03 (s, 2H), 1.55 (s, 6H)

Intermediate G

### 2,2,3,3-tetramethyl-2,3-dihydro-1-benzofuran-7-carbaldehyde

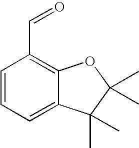

The title compound was prepared by the procedure described in Intermediate T using salicylaldehyde and 1-bromo-2,3-dimethylbut-2-ene (3 g, 34%)

¹H NMR (399.99 MHz, CDCl3) δ 10.25 (s, 1H), 7.61-7.57 (m, 1H), 7.28-7.26 (m, 1H), 6.93 (t, J=7.5 Hz, 1H), 1.40 (s, 6H), 1.23 (s, 6H)

Intermediate H

### 2,2,4-trimethyl-2,3-dihydro-1-benzofuran-7-carbaldehyde

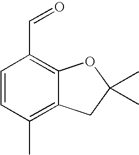

The title compound was prepared by the procedure described in Intermediate T using 3-chloro-2-methylprop-1-ene and 2-hydroxy-4-methylbenzaldehyde (1.1 g, 47%)

¹H NMR (399.99 MHz, CDCl3) δ 10.16 (s, 1H), 7.52 (d, J=8.0 Hz, 1H), 6.71 (d, J=8.0 Hz, 1H), 2.94 (s, 2H), 2.26 (s, 3H), 1.55 (s, 6H)

Intermediate J

### 2,2-dimethyl-2,3-dihydro-1,4-benzodioxin-5-carbaldehyde

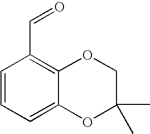

A mixture of 2,3-dihydroxybenzaldehyde (4.0 g, 29 mmol), 3-chloro-2-methylprop-1-ene (2.8 ml, 29 mmol), potassium carbonate (4.4 g, 32 mmol) and NMP (15 ml) were heated at 40° C. for 10 h. The mixture was diluted with ethyl acetate, washed with water and then aqueous potassium carbonate. The organic layer was evaporated and the residue was purified on silica. The resulting intermediate 3-hydroxy-2-[(2-methylprop-2-en-1-yl)oxy]benzaldehyde (1.8 g, 9.4 mmol) was dissolved in formic acid and the mixture heated at reflux for 2 h and then evaporated. The residue was dissolved in ethyl acetate, washed with sodium hydrogen carbonate and purified on silica to obtain the product in 3% yield.

¹H NMR (399.99 MHz, CDCl3) δ 10.41 (d, J=0.5 Hz, 1H), 7.40-7.37 (m, 1H), 7.08-7.04 (m, 1H), 6.92 (t, J=7.9 Hz, 1H), 4.01 (s, 2H), 1.39 (s, 6H)

Intermediate K

### 3-[(2,2-dimethyl-2H-chromen-8-yl)methyl]-3,9-diazaspiro[5.5]undecane Dihydrochloride

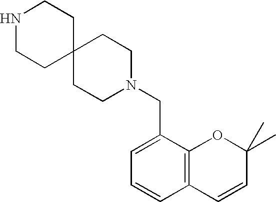

The title compound was synthesised by the procedure of Intermediate A using tert-butyl 3,9-diazaspiro[5.5]undecane-3-carboxylate and Intermediate 0 as starting materials to afford the product as a yellow sticky solid (400 mg, 57%).

APCI-MS m/z: 327.3 [MH+]

Intermediate L

### -(3,9-diazaspiro[5.5]undec-3-ylcarbonyl)pyridin-2-amine

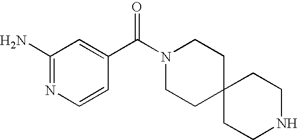

The title compound was prepared by the procedure of Intermediate S using 2-aminoisonicotinic acid and tert-butyl 3,9-diazaspiro[5.5]undecane-3-carboxylate as starting materials to give the product as a white solid (1.7 g, 29%).

APCI-MS m/z: 275.1 [MH+]

Intermediate M

### 4-(3,9-diazaspiro[5.5]undec-3-ylcarbonyl)pyridin-3-amine

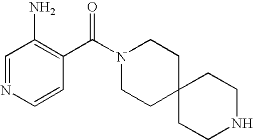

The compound was prepared by the amide coupling procedure of Example 8 and the Boc cleaving procedure of Intermediate A using tert-butyl 3,9-diazaspiro[5.5]undecane-3-carboxylate hydrochloric acid salt and 3-aminoisonicotinic acid as starting materials to give the product as a yellow oily solid (3.00 g, 66%).

¹H NMR (399.99 MHz, DMSO-D6) δ 8.06 (s, 1H), 7.76 (d, 1H), 6.92 (d, 1H), 5.28 (d, 1H), 3.71-3.49 (m, 2H), 3.25-3.09 (m, 2H), 2.63 (s, 4H), 1.57-1.24 (m, 8H)

APCI-MS m/z: 275.2 [MH+]

Intermediate N

### 3-[(1-oxidopyridin-2-yl)carbonyl]-3,9-diazaspiro[5.5]undecane

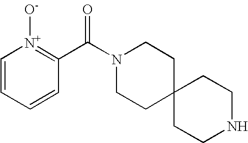

The compound was prepared by the amide coupling procedure of Example 8 and the Boc cleaving procedure of Intermediate A using tert-butyl 3,9-diazaspiro[5.5]undecane-3-carboxylate hydrochloric acid salt and pyridine-2-carboxylic acid 1-oxide as starting materials to give the product as a yellow oily solid (1.99 g, 70%).

¹H NMR (399.99 MHz, DMSO-D6) δ 8.26 (d, 1H), 7.52-7.36 (m, 3H), 3.71-3.42 (m, 2H), 3.16-2.94 (m, 2H), 2.88-2.70 (m, 4H), 1.59-1.41 (m, 6H), 1.39-1.28 (m, 2H)

APCI-MS m/z: 276.2 [MH+]

Intermediate O

### 2,2-dimethyl-2H-chromene-8-carbaldehyde

### a) 2-[(1,1-dimethylprop-2-yn-1-yl)oxy]benzaldehyde

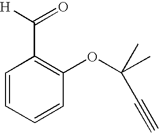

Salicylaldehyde (0.86 ml, 8.19 mmol) was dissolved in dry CH₃CN (20 ml). CuCl (4 mg, 0.04 mmol) and DBU (1.34 ml, 9.01 mmol) were added. The mixture was cooled to 0° C. under argon. 3-chloro-3-methylbut-1-yne (0.92 ml, 8.19 mmol) was added and the mixture was stirred at 0° C. to room temperature for 4 h. The mixture was evaporated and the residue was dissolved in toluene, washed with 1M Hydrochloric acid, 1M NaOH, saturated aqueous sodium bicarbonate solution and brine, dried over sodium sulphate and evaporated. The crude product was purified using column chromatography on silica eluting with heptane:EtOAc 10:1 to afford the title compound as a yellow oil (1.17 g, 76%).

¹H NMR (399.99 MHz, CDCl3) δ 10.45 (s, 1H), 7.89-7.85 (m, 1H), 7.57-7.50 (m, 2H), 7.14 (ddd, J=13.7, 2.3, 0.8 Hz, 1H), 2.62 (s, 1H), 1.74 (s, 9H)

### b) 2,2-dimethyl-2H-chromene-8-carbaldehyde

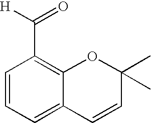

2-[(1,1-dimethylprop-2-yn-1-yl)oxy]benzaldehyde (1.10 g, 5.84 mmol) was dissolved in diethylaniline (10 ml) and the mixture heated at 190° C. for 1 h. After cooling the mixture was diluted with heptane, washed with 1M hydrochloric acid and water, dried over sodium sulphate and evaporated. The crude product was purified using column chromatography on silica eluting with heptane:EtOAc 16:1 to afford the title compound as an orange oil (0.54 g, 49%).

¹H NMR (399.99 MHz, CDCl3) δ 10.48 (s, 1H), 7.65 (d, J=7.6 Hz, 1H), 7.18 (d, J=7.1 Hz, 1H), 6.89 (t, J=7.4 Hz, 1H), 6.35 (d, J=9.8 Hz, 1H), 5.71 (d, J=9.8 Hz, 1H), 1.51 (s, 9H)

Intermediate P

### 4-(3,9-diazaspiro[5.5]undec-3-ylcarbonyl)pyrimidin-2-amine

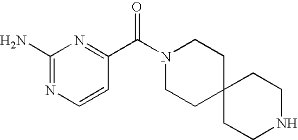

The compound was prepared by the amide coupling procedure of Example 119 and the Boc cleaving procedure of Intermediate A using tert-butyl 3,9-diazaspiro[5.5]undecane-3-carboxylate hydrochloric acid salt and 2-aminopyrimidine-4-carboxylic acid as starting materials to give the product as a yellow oily solid (3.00 g, 45%).

¹H NMR (299.946 MHz, DMSO-D6) δ 8.31 (d, J=4.8 Hz, 1H), 6.81 (s, 2H), 6.55 (d, J=5.0 Hz, 1H), 3.54 (t, J=5.8 Hz, 2H), 3.33-3.19 (m, 4H), 2.67-2.59 (m, 4H), 1.48-1.28 (m, 6H)

APCI-MS m/z: 276.2 [MH+]

Intermediate Q

### 3-[(2,2,3,3-tetramethyl-2,3-dihydro-1-benzofuran-7-yl)methyl]-3,9-diazaspiro[5.5]undecane Dihydrogen Chloride

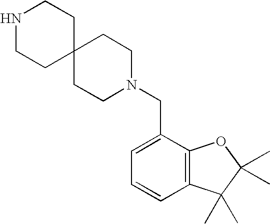

The title compound was prepared by the procedure of Intermediate C using tert-butyl 3,9-diazaspiro[5.5]undecane-3-carboxylate and intermediate G as starting materials to give the product as a gum (800 mg, 100%).

APCI-MS m/z: 343.1 [MH+]

Intermediate S

### 3-isonicotinoyl-3,9-diazaspiro[5.5]undecane

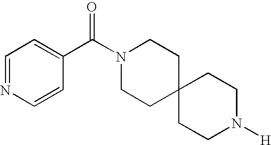

A mixture of isonicotinic acid (2.1 g, 17 mmol), HBTU (7.2 g, 19 mmol), triethylamine (1.9 g, 19 mmol) and dichloromethane (90 ml) was stirred overnight at room temperature. The mixture was washed with aqueous sodium hydrogen carbonate, the organic layer was evaporated and the residue purified on silica to obtain the intermediate tert-butyl 9-isonicotinoyl-3,9-diazaspiro[5.5]undecane-3-carboxylate. This Intermediate was dissolved in methanol and added to a 2M methanolic hydrochloric acid (100 ml) solution, the reaction mixture was stirred at room temperature for 1 h and evaporated. The residue was purified by acidic ion-exchange resin to yield the product as a white solid (2.8 g, 62%).

APCI-MS m/z: 260.4 [MH+]

Intermediate T

### 2,2,3-trimethyl-2,3-dihydro-1-benzofuran-7-carbaldehyde and 2,3,3-trimethyl-2,3-dihydro-1-benzofuran-7-carbaldehyde

### 1:1 Mixture

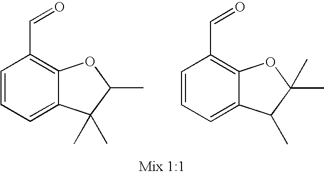

A mixture of salicylaldehyde (5 g, 41 mmol), 1-bromo-3-methylbut-2-ene (6.1 g, 41 mmol), potassium carbonate (5.7 g, 41 mmol), and NMP (25 ml) were stirred at 40° C. overnight then diluted with ethyl acetate and washed with water. The organic layer was evaporated and the residue purified on silica (0% to 100% EtOAc in heptane). The intermediate (2-[(3-methylbut-2-en-1-yl)oxy]benzaldehyde) was dissolved in NMP (25 ml) and heated at reflux for 8 h. The mixture was diluted with ethyl acetate and washed with water. The organic layer was evaporated and the residue was purified on silica (0% to 100% EtOAc in heptane). The resulting (3-(1,1-dimethylprop-2-en-1-yl)-2-hydroxybenzaldehyde) was dissolved in formic acid (40 ml) and heated at reflux for 8 h. The formic acid was evaporated and the residue was dissolved in ethyl acetate and washed with aqueous sodium hydrogen carbonate. The organic layer was evaporated and the residue was purified on silica to obtain the title compound as a 1:1 isomeric mixture (1 g, 13%).

¹H NMR (499.879 MHz, CDCl3) δ 10.24 (d, J=3.7 Hz, 2H), 7.62-7.59 (m, 2H), 7.31-7.29 (m, 2H), 6.96 (t, J=7.5 Hz, 1H), 6.92 (t, J=7.5 Hz, 1H), 4.58-4.54 (m, 1H), 3.20-3.15 (m, 1H), 1.55 (s, 3H), 1.45 (d, J=6.6 Hz, 3H), 1.36 (s, 6H), 1.26 (d, J=7.2 Hz, 3H), 1.15 (s, 3H)

Intermediate U

### 4-chloro-2,2-dimethyl-2,3-dihydro-1-benzofuran-7-carbaldehyde

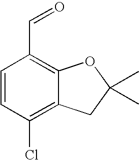

A mixture of methyl 4-chloro-2-hydroxybenzoate (5 g, 27 mmol), methallyl chloride 2.4 g, 27 mmol), potassium carbonate (4 g, 29 mmol) and NMP (25 ml) were stirred at 40 C overnight then diluted with ethyl acetate and washed with water. The organic layer was evaporated and the residue purified on silica (0% to 100% EtOAc in heptane). The resulting (methyl 4-chloro-2-[(2-methylprop-2-en-1-yl)oxy]benzoate (3.5 g, 15 mmol)) was dissolved in NMP (25 ml), heated at reflux for 8 h then diluted with ethyl acetate and washed with water. The organic layer was evaporated and the residue purified on silica (0% to 100% EtOAc in heptane). The resulting methyl 4-chloro-2-hydroxy-3-(2-methylprop-2-en-1-yl)benzoate (3 g, 12 mmol)) was dissolved in formic acid (25 ml) and heated at reflux for 8 h. The formic acid was evaporated and the residue was dissolved in ethyl acetate and washed with aqueous sodium hydrogen carbonate. The organic layer was evaporated and the residue purified on silica (0% to 100% EtOAc in heptane). The resulting methyl 4-chloro-2,2-dimethyl-2,3-dihydro-1-benzofuran-7-carboxylate (0.2 g, 0.8 mmol)) was dissolved in THF (4 ml) and 1M DIBAL-H in THF (2.2 ml, 2 mmol) was added. The mixture was heated at 40° C. for 4 h, quenched with 2M hydrochloric acid and extracted with ethyl acetate. The organic layer was evaporated, the residue was dissolved in diethyl ether (10 ml) and manganese dioxide (360 mg, 4 mmol) was added. The mixture was stirred at room temperate overnight, then filtered. The organic layer was evaporated and the residue was purified on silica to obtain the title compound (77 mg, 13%).

¹H NMR (399.99 MHz, CDCl3) δ 10.16 (s, 1H), 7.56 (d, J=8.5 Hz, 1H), 6.88 (d, J=8.5 Hz, 1H), 3.07 (s, 2H), 1.58 (s, 6H)

Intermediate V

### 2-[2-(3,9-diazaspiro[5.5]undec-3-ylcarbonyl)phenyl]acetamide

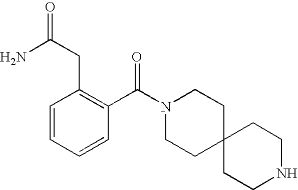

The compound was prepared by the amide coupling procedure of Intermediate S, using 2-(2-methoxy-2-oxoethyl)benzoic acid and tert-butyl 3,9-diazaspiro[5.5]undecane-3-carboxylate as starting materials. The Intermediate tert-butyl 9-[2-(2-methoxy-2-oxoethyl)benzoyl]-3,9-diazaspiro[5.5]undecane-3-carboxylate was dissolved in 7M ammonia in methanol, then stirred for 4 days and evaporated. Using the same Boc cleaving procedure and purification as for Intermediate S gave the product as a white solid (0.4 g, 37%).

APCI-MS m/z: 316.2 [MH+]

Intermediate W

### 2,2-dimethyl-2,3-dihydro-1-benzofuran-4-carbaldehyde

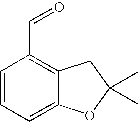

Method 1

The title compound was prepared by the procedure described in Intermediate T using 3-hydroxybenzaldehyde and 3-chloro-2-methylprop-1-ene to afford the product (50 mg, 7%).

¹H NMR (499.879 MHz, CDCl3) δ 10.05 (s, 1H), 7.34-7.29 (m, 2E), 7.00-6.97 (m, 1H), 3.36 (s, 2H), 1.51 (s, 6H)

Method 2

### a) 3-(2-Methylallyloxy)benzoic Acid Methyl Ester

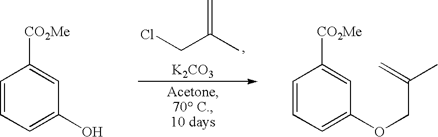

To a solution of 3-hydroxybenzoic acid methyl ester (0.668 moles) in acetone (670 ml) was added K₂CO₃ (0.835 moles, 1.2 eq) followed by 3-chloro-2-methylpropene (75.59 g, 82.5 ml, 1.2 eq). The mixture was heated at 70° C. for 10 days, cooled to room temp and partitioned between EtOAc (500 ml) and water (1000 ml). The aqueous layer was washed with EtOAc (2×250 ml) and the combined organics were washed with water (2×500 ml) and brine (100 ml); dried (MgSO₄), filtered and concentrated in vacuo to leave 136.4 g (99%) of a very pale yellow oil.

¹H NMR (300 MHz, CDCl₃) δ 7.63 (dd, 8.1 and 2.7 Hz, 1H), 7.58 (d, 2.7 Hz, 1H), 7.34 (t, 8.1 Hz, 1H), 7.12 (dd, 8.1 and 2.7 Hz, 1H), 5.11 (s, 1H), 5.00 (s, 1H), 4.48 (s, 2H), 3.91 (s, 3H) and 1.84 (s, 3H).

### b) 3-Hydroxy-2-(2-methylallyl)benzoic acid methyl ester and 3-hydroxy-4-(2-methylallyl)benzoic Acid Methyl Ester

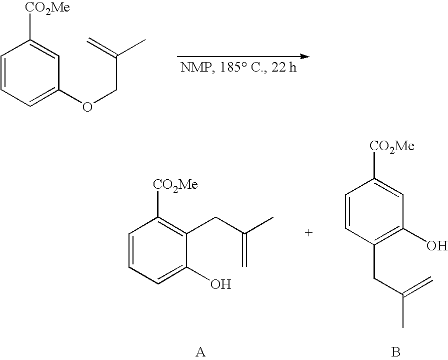

A solution of 3-(2-methylallyloxy)benzoic acid methyl ester (103.12 g, 0.5 moles) in NMP (103 ml) under nitrogen was heated at 185° C. for 22 hours. The mixture was cooled to room temperature and partitioned between EtOAc (500 ml) and water (1000 ml). The organic layer was washed with water (2×500 ml) and brine (100 ml); dried (MgSO₄), filtered and concentrated in vacuo to leave 106.2 g of a crude brown oil. Flash chromatography (2×53 g, Biotage 75 L, neat DCM) afforded 67 g of a crude yellow oil (mostly 2-regioisomer, A, Rf=0.48) and 23.5 g (23%) of a pink solid (4-regioisomer, B, Rf=0.23). The crude yellow oil was rechromatographed (2×33.5 g, Biotage 75 L, neat DCM) to give 42.0 g (41%) of a pale yellow oil. TBME (t-butyl methyl ether) may also be used instead of EtOAc as extraction solvent.

2-Regioisomer, A, ¹H NMR (400 MHz, CDCl₃) δ 7.44 (dd, 7.6 and 0.8 Hz, 1H), 7.19 (t, 7.6 Hz, 1H), 7.01 (dd, 7.6 and 0.8 Hz, 1H), 5.46 (s, 1H), 4.89 (s, 1H), 4.69 (s, 1H), 3.87 (s, 3H), 3.77 (s, 2) and 1.80 (s, 3H).

APCI-MS m/z ˜205 [M(−H)]+.

### c) 2,2-Dimethyl-2,3-dihydrobenzofuran-4-carboxylic Acid Methyl Ester

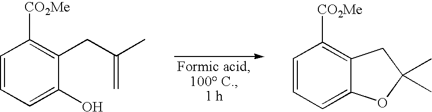

A solution of 3-hydroxy-2-(2-methylallyl)benzoic acid methyl ester (42.0 g, 0.2 moles) in 99% formic acid (120 ml) was heated at reflux for 1 hour. The mixture was cooled to room temp and partitioned between EtOAc (250 ml) and water (1000 ml). The aqueous layer was washed with EtOAc (2×100 ml) and the combined organics were washed with water (2×200 ml) and brine (100 ml); dried (MgSO₄), filtered and concentrated in vacuo to leave 40.4 g (96%) of a yellow oil/white solid. One gram of this mixture was placed on a pad of silica (6.5 cm dia×4.5 cm) and eluted with neat DCM (250 ml) to give 850 mg of a yellow oil. The silica pad was flushed with Et₂O (125 ml) to give 110 mg of a pink solid. TBME may also be used instead of EtOAc as extraction solvent

¹H NMR (300 MHz, CDCl₃) δ 7.49 (d, 7.8 Hz, 1H), 7.17 (t, 7.8 Hz, 1H), 6.90 (d, 7.8 Hz, 1H), 3.89 (s, 3H), 3.35 (s, 2H) and 1.49 (s, 6H).

APCI-MS m/z=207 [M(+H)]+.

### d) (2,2-Dimethyl-2,3-dihydrobenzofuran-4-yl)methanol

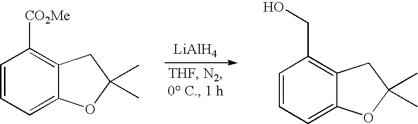

To a solution of 2,2-dimethyl-2,3-dihydrobenzofuran-4-carboxylic acid methyl ester (39.4 g, 0.19 moles) in dry THF (300 ml) at 0° C. under nitrogen was added lithium aluminium hydride (1M solution in THF, 287 ml, 0.287 moles, 1.5 eq) dropwise over 30 mins. The mixture was allowed to warm up to room temperature and stirred (using an overhead mechanical stirrer) for a further 18 hours. The mixture was cooled to 0° C. and water (11 ml, equivalent to 10.87 g of LiAlH₄ used) was added dropwise, followed by 15% NaOH solution (11 ml) and followed by water (33 ml). The resulting precipitate was removed by filtration through a Celite (bottom)/Na₂SO₄ (top) pad. The pad was washed with EtOAc (500 ml) and the filtrate was concentrated in vacuo to leave 32.6 g (93%) of a crude red/pink solid.

¹H NMR (400 MHz, CDCl₃) δ 7.12 (t, 7.6 Hz, 1H), 6.84 (d, 7.6 Hz, 1H), 6.69 (d, 7.6 Hz, 1H), 4.61 (d, 4.5 Hz, 2H), 3.04 (s, 2H) and 1.48 (s, 6H).

### e) (2,2-Dimethyl-2,3-dihydrobenzofuran-4-carboxaldehyde

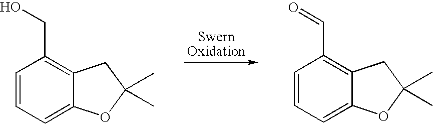

To a solution of oxalyl chloride (27.87 g, 19.2 ml, 0.22 moles, 1.2 eq) in dry DCM (250 ml) at −78° C. under nitrogen was added dropwise a solution of dimethyl sulphoxide (32.88 g, 29.9 ml, 0.42 moles, 2.3 eq) in dry DCM (35 ml). After 30 mins, a solution of (2,2-dimethyl-2,3-dihydrobenzofuran-4-yl)methanol (32.6 g, 0.183 moles) in dry DCM (75 ml) was also added dropwise. After 1 hour at −78° C., triethylamine (92.6 g, 127.5 ml, 0.915 moles, 5 eq) was added dropwise and the reaction mixture was allowed to warm up to room temp overnight. The yellow suspension was washed with saturated NH₄Cl solution (250 ml) and brine (250 ml). The organic layer was dried (Na₂SO₄), filtered and concentrated in vacuo to leave 30.5 g (95%) of an orange oil.

H NMR (400 MHz, CDCl₃) δ 10.04 (s, 1H), 7.33-7.26 (m, 2H), 6.99-6.96 (m, 1H), 3.35 (s, 2H) and 1.50 (s, 6H).

Intermediate X

### 2,2-dim ethyl-2H-chromene-5-carbaldehyde

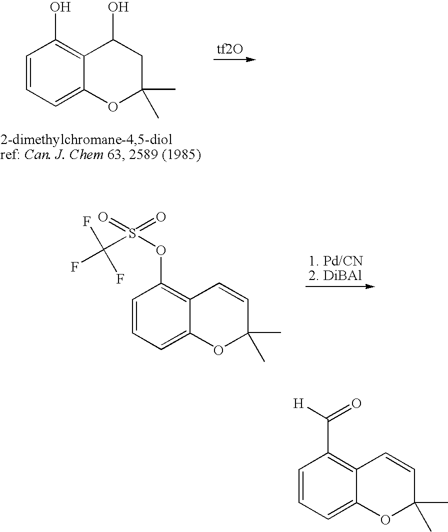

### (a) 2,2-dimethyl-2H-chromen-5-yl trifluoromethanesulfonate

A mixture of 2-dimethylchroman-4,5-diol (1.04 g, 5.38 mmol), triethylamine (2.2 ml, 16 mmol), potassium carbonate (2.2 g, 16 mmol) and dichloromethane (30 ml) was stirred under argon at 0° C. and triflic anhydride (2.0 ml, 11.8 mmol) was added. After 30 min the mixture was passed through silica, evaporated and purified on a silica column (0% to 30% EtOAc in heptane) to yield the subtitle compound (593 mg, 36%).

### (b) 2,2-dimethyl-2H-chromene-5-carbonitrile

A mixture of 2,2-dimethyl-2H-chromen-5-yl trifluoromethanesulfonate (520 mg, 1.70 mmol), zinc dicyanide (150 mg, 1.23 mmol), 1,1′-bis(diphenylphosphino)ferrocene-palladium (II) dichloride dichloromethane complex (51 mg, 0.06 mmol) and NMP (3 ml) was heated in microwave reactor at 150° C. for 20 min. The reaction mixture was paRTioned between water (50 ml) and heptane/TBME (1/1, 50 ml), and the evaporated organic layer was purified by silica chromatography (0% to 30% EtOAc in heptane to yield the subtitle compound (148 mg, 47%).

### (c) 2,2-dimethyl-2H-chromene-5-carbaldehyde

A mixture 2,2-dimethyl-2H-chromene-5-carbonitrile (140 mg, 0.76 mmol), DiBAL (1M solution in THF, 2 ml, 2 mmol) and THF (3 ml) was stirred under argon at 40° C. After for 6 h 1M hydrochloric acid (10 ml) and heptane/TBME (1/1, 20 ml) was added and organic layers filtered through silica to yield the title compound (43 mg, 30%).

¹H NMR (399.99 MHz, CDCl₃) δ 10.14 (s, 1H), 7.41 (d, J=10.2 Hz, 1H), 7.35-7.22 (m, 2H), 7.06-7.01 (m, 1H), 5.83 (dd, J=10.1, 3.1 Hz, 1H), 1.48-1.39 (m, 6H)

EI-MS m/z: 188.0 [M+]

Intermediate Y

### 2,2-dimethylchroman-8-carbaldehyde

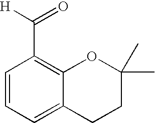

A mixture of Intermediate O (404 mg, 2.1 mmol), 10% palladium on activated carbon (32 mg) and ethanol (5 ml) was stirred under an atmosphere of hydrogen at 50 psi. After 60 min the mixture was filtered and evaporated. Residue was rapidly stirred with 2,2,6,6-tetramethylpiperidin-1-yloxy free radical (58 mg, 0.37 mmol), sodium bromide (478 mg, 4.6 mmol), aqueous sodium bicarbonate (5 ml), EtOAc (3 ml), toluene (3 ml) and water (1 ml). To this mixture was added 10% sodium hypochlorite solution in water (1.5 ml) portionwise. After 1 h the organic layer was separated and filtered through silica to yield the title compound (360 mg, 88%)

EI-MS m/z: 190.0 [M+]

Intermediate Z

### 3,3-dimethyl-2,3-dihydro-1,4-benzodioxin-5-carbaldehyde

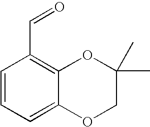

The title compound was prepared by the procedure of Intermediate J using methyl 2,3-dihydroxybenzoate and 3-chloro-2-methylprop-1-ene as starting materials and using the reduction and oxidation procedures from Intermediate U to give the product (300 mg, 5%).

¹H NMR (399.99 MHz, CDCL3) δ 10.42 (d, J=0.6 Hz, 1H), 7.44-7.41 (m, 1H), 7.12-7.10 (m, 1H), 6.92-6.86 (m, 1H), 3.96 (s, 2H), 1.43 (s, 6H)

Intermediate AA

### 3-[(3,3-dimethyl-2,3-dihydro-1,4-benzodioxin-5-yl)methyl]-3,9-diazaspiro[5.5]undecane

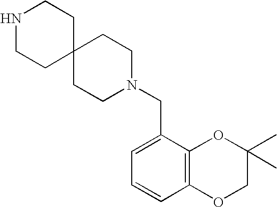

The title compound was prepared by the procedure of Intermediate C using tert-butyl 3,9-diazaspiro[5.5]undecane-3-carboxylate and intermediate Z as starting materials followed by ion-exchange chromatography on SCX to give the product as a gum (1.10 g, 48%).

APCI-MS m/z: 331.2 [MH+]

Intermediate AB

### 3-[2-ethoxy-1-(ethoxycarbonyl)-2-oxoethyl]isonicotinic acid

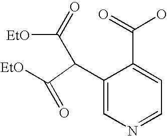

3-bromoisonicotinic acid (730 mg, 3.61 mmol) and Cuprous Bromide (31 mg, 0.22 mmol) was suspended in an excess of diethyl malonate (30 ml). Sodium hydride (631 mg, 26.3 mmol, 55% in oil) was added in portions under argon. After addition the mixture was stirred for 2 hrs at 80° C. The mixture was diluted with H2O and washed with TBDME (3×30 ml). The aqueous phase was acidified to pH 4 using conc. HCl and extracted with TBDME (3×30 ml). The combined organic layers (from 2^{nd} extraction) were dried over Na2SO4 and evaporated. The crude product was recrystallized from TBDME and Heptane affording 668 mg (66%) the title compound as a green solid.

APCI-MS m/z: 282.1 [MH+]

Intermediate AC

### 2-[(2,2-dimethyl-2,3-dihydro-1-benzofuran-4-yl)methyl]-2,7-diazaspiro[4.4]nonane

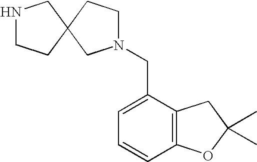

The title compound was prepared by the procedure of Intermediate C using tert-butyl 2,7-diazaspiro[4.4]nonane-2-carboxylate and intermediate W as starting materials followed by ion-exchange chromatography on SCX to give the product as a gum (410 mg, 46%).

APCI-MS i/z: 287.1 [MH+]

Intermediate AD

### 7-[(2,2-dimethyl-2,3-dihydro-1-benzofuran-4-yl)methyl]-2,7-diazaspiro[3.5]nonane

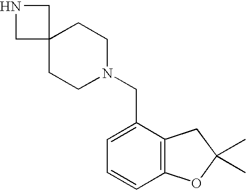

The title compound was prepared by the procedure of Intermediate C using tert-butyl 2,7-diazaspiro[3.5]nonane-2-carboxylate and intermediate W as starting materials followed by ion-exchange chromatography on SCX to give the product as a gum (410 mg, 46%).

APCI-MS m/z: 287.1 [MH+]

Intermediate AE

### 2-[(2,2-dimethyl-2,3-dihydro-1-benzofuran-4-yl)methyl]-2,7-diazaspiro[3.5]nonane

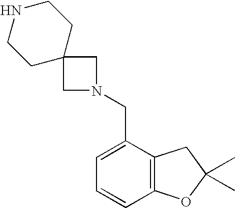

The title compound was prepared by the procedure of Intermediate C using tert-butyl 2,7-diazaspiro[3.5]nonane-7-carboxylate and intermediate W as starting materials followed by ion-exchange chromatography on SCX to give the product as a colourless oil (1.29 g, 95%).

APCI-MS m/z: 287.1 [MH+]

Intermediate AF

### 3-(2-amino-2-oxoethyl)benzoic Acid

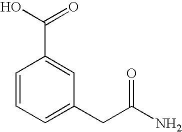

### a) methyl 3-(cyanomethyl)benzoate

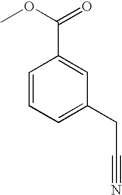

A slurry of methyl 3-(bromomethyl)benzoate (4.0 g, 17.5 mmol) and potassium cyanide (1.2 g, 18.4 mmol) in ethanol (40 ml) was heated at 60° C. over night, filtrated and evaporated. The residue was purified using column chromatography on SiO₂ affording 2.1 g (68%) of the title compound.

¹H NMR (399.988 MHz, CDCl3) δ 8.06-7.98 (m, 2H), 7.56 (d, J=8.0 Hz, 1H), 7.51-7.46 (m, 1H), 3.94 (s, 3H), 3.82 (s, 2H)

### b) methyl 3-(2-amino-2-oxoethyl)benzoate

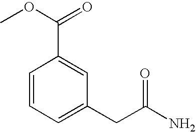

Methyl 3-(cyanomethyl)benzoate (2.1 g, 12 mmol) was dissolved in THF (50 ml) and concentrated HCl (50 ml) and stirred over night. The solution was basified with 1M NaOH, washed with EtOAc, the water phase was acidified with concentrated HCl and extracted with EtOAc. The organic layer was dried over sodium sulphate and evaporated to afford 1.6 g (70%) of the title compound.

APCI-MS m/z: 194.0 [MH+]

### c) 3-(2-amino-2-oxoethyl)benzoic Acid

To a solution of methyl 3-(2-amino-2-oxoethyl)benzoate (1.6 g, 8.3 mmol) in MeOH/THF/water 1:1:1 (30 ml) was lithium hydroxide (2 g, 83 mmol) added and stirred over night. The solution was diluted with water, acidified with HCl, extracted with EtOAc, dried over sodium sulphate and evaporated. The crude product was purified by preparative HPLC (RP-18) to afford 0.6 g (40%) of the title compound as a white solid.

¹H NMR (399.99 MHz, DMSO-D6) δ 7.86 (s, 1H), 7.80 (d, J=7.6 Hz, 1H), 7.50 (d, J=7.6 Hz, 1H), 7.42 (t, J=7.6 Hz, 1H), 3.44 (s, 2H)

APCI-MS m/z: 180.1 [MH+]

Intermediate AG

### 4-(2-amino-2-oxoethyl)benzoic Acid

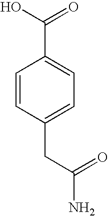

The title compound was prepared by the procedure of Intermediate AF using methyl 4-(bromomethyl)benzoate as starting material to give the product as a white solid (1.0 g, 67%).

¹H NMR (399.99 MHz, DMSO-D6) δ 7.86 (d, J=8.1 Hz, 2H), 7.37 (d, J=8.1 Hz, 2H), 3.46 (s, 2H)

APCI-MS m/z: 180.1 [MH+]

Intermediate AH

### 3-[(2,2-dimethyl-2,3-dihydro-1,4-benzodioxin-5-yl)methyl]-3,9-diazaspiro[5.5]undecane

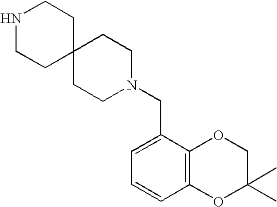

The title compound was prepared by the procedure of Intermediate C using Intermediate J as starting material and purified by acidic ion-exchange resin to yield the product as a white solid (0.4 g, 23%)

¹H NMR (399.99 MHz, CD3OD) δ 6.84-6.76 (m, 3H), 6.74-6.70 (m, 1H), 3.91 (s, 2H), 3.58 (s, 2H), 2.74 (t, J=5.7 Hz, 4H), 2.51 (t, J=5.2 Hz, 4H), 1.55 (t, J=5.5 Hz, 4H), 1.43 (t, J=5.5 Hz, 4H), 1.31 (s, 6H)

APCI-MS m/z: 330.9 [MH+]

Spiro Intermediates

### Preparation of 2,8-Diaza-spiro[4.5]decane-8-carboxylic Acid Tert-Butyl Ester

(also referred to as tert-butyl 2,8-diazaspiro[4.5]decane-8-carboxylate)

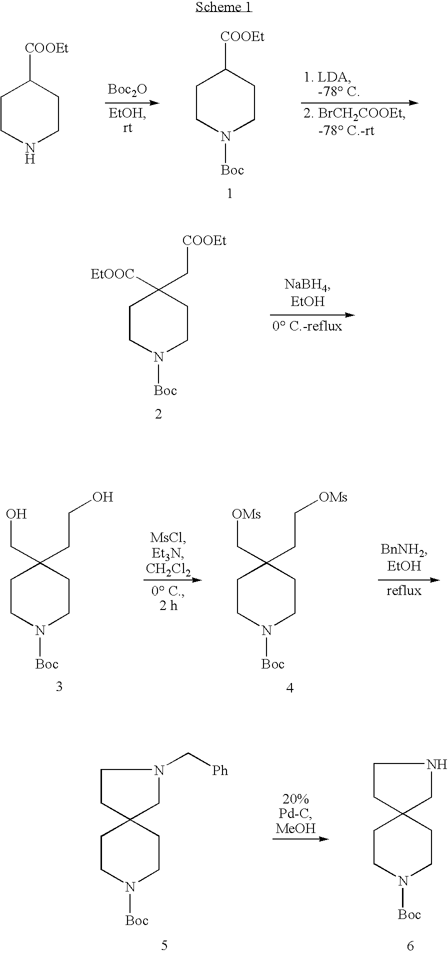

It should be noted that in the following synthetic summary, the intermediate compounds are referred to by their number in scheme 1.

Preparation of Compound 1

To a solution of ethyl piperidine-4-carboxylate (500 g, 3.18 mol) in absolute ethanol (3000 mL) was added dropwise Boc₂O (715 g, 3.28 mol) over 1 hour under water bath (note: the reaction was exothermic, ice bath should be added to calm down the reaction if needed). The mixture was stirred at room temperature for 2 h and concentrated under reduced pressure to give crude compound 1 (˜825 g), which was used for next step without further purification.

Preparation of Compound 2

n-BuLi (280 mL, 2.5 M in TBF, 0.70 mol) was added dropwise to a solution of freshly-distilled diisopropyl amine (98 mL) in dry THF (100 mL) at −78° C. over 1˜2 h under N₂. After the addition, the mixture was stirred at −78° C. for 1 h, then a solution of ethyl bromoacetate (146 g, 0.87 mol) in THF (100 mL) was added dropwise over 1˜2 h. The resulting mixture was stirred at −78° C. for 2 h and then at room temperature overnight. The reaction was quenched with sat. aq. NH₄Cl. The layers were separated, and the aqueous layer was extracted with EtOAc three times. The combined organic layers were washed with 1 N aq. HCl to pH<7, then washed with sat. aq. NaHCO₃ and brine, dried ver MgSO₄ and concentrated to give an oil (220 g)

Preparation of Compound 3

The above oil (100 g) was dissolved in dry ethanol (1500 mL), and NaBH₄ (90 g, 2.4 mol) was added in a ice bath. The mixture was stirred at the same temperature for 4 h and then stirred at room temperature overnight, followed by refluxed for 4 h. The mixture was taken up with 500 mL of H₂O, and the mixture was adjusted to pH=5˜6 with aq. 6 N HCl. The mixture was filtered and the filtrate was concentrated to remove organic solvent. The aqueous layer was extracted with CH₂Cl₂ three times. The combined organic layers were dried over MgSO₄ and purified by chromatography (petroleum ether:EtOAc 2:1, then EtOAc) to give compound 3 (25-30 g).

Preparation of Compound 4

To a solution of compound 3 (110 g, 0.43 mol) and Et₃N (300 g, 412 mL, 3.0 mol) in CH₂Cl₂ (1100 mL) was added dropwise MsCl (170 g, 117 mL, 1.49 mol) in an ice bath. After the addition, the mixture was stirred at the same temperature for 2 h. TLC showed the reaction was complete. The mixture was poured into ice-water (200 mL) and stirred for 10 min. The organic layer was separated and the aqueous layer was extracted with CH₂Cl₂ two times. The combined organic layers were washed with 1 N aq. HCl (200 mL×3) and brine, dried over MgSO₄ and concentrated to give compound 4 (170 g, 96%) as brown syrup.

Preparation of Compound 5

A solution of compound 4 (170 g, 0.41 mol) and benzylamine (176 g, 180 mL, 1.64 mol) in absolute ethanol (1700 mL) was refluxed for 20 h, then concentrated to dryness. EtOAc (1500 mL) was added, the mixture was filtered, the filter cake was washed with EtOAc. The filtrate was concentrated, and the residue was purified by chromatography (petroleum ether/EtOAc 5:1˜2:1) to give compound 5 (90-103 g).

Preparation of Compound 6

A mixture of compound 5 (82 g), 20% Pd(OH)₂/C (15 g) and methanol (1 L) was stirred under 85 Psi of H₂ overnight. The mixture was filtered to remove catalyst. Another 16 g of 20% Pd(OH)₂/C was added and the mixture was stirred under 85 Psi of H2 until the reaction was complete. The mixture was filtered and the filtrate was concentrated to give 59 g of crude product, which was purified by chromatography (CH₂Cl₂, then 5% NH₃·H₂O/MeOH) to give compound 6 (30-45 g). ¹H NMR (CD₄O, HCl salt) δ: 3.58-3.35 (m, 6H), 3.13 (s, 2H), 1.96 (t, 2H), 1.59 (t, 4H), 1.45 (9H).

### Preparation of 2,8-Diaza-spiro[4.5]decane-2-carboxylic Acid Tert-Butyl Ester

(also referred to as tert-butyl 2,8-diaza-spiro[4.5]decane-2-carboxylate)

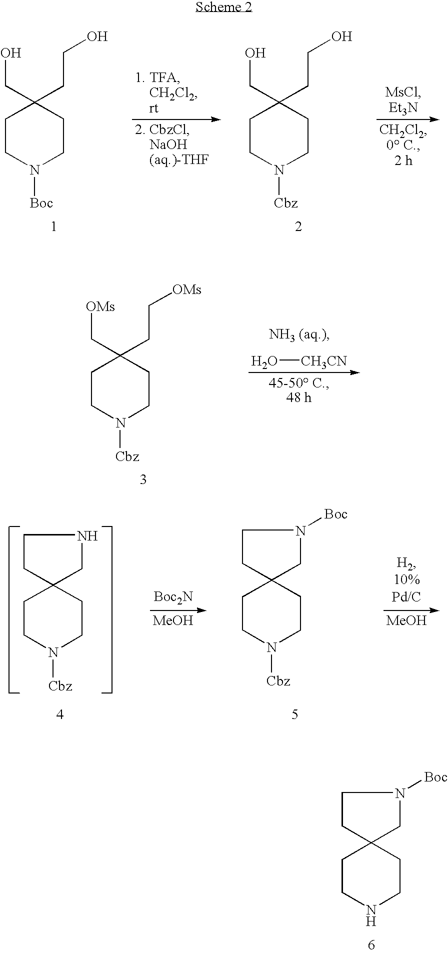

It should be noted that in the following synthetic summary, the intermediate compounds are referred to by their number in scheme 2.

Preparation of Compound 2

A solution of compound 1 (70 g, 0.27 mol) and 200 mL of TFA in CH₂Cl₂ (400 mL) was stirred at room temperature overnight and concentrated. The residue was taken up with H₂O (400 mL) and the mixture was basified with NaOH. THF (400 mL) was added. The mixture was cooled in an ice bath and Cbz-Cl (0.3 mol) was added dropwise with stirring, then 5 N NaOH was added to keep the mixture was basic, and the mixture was stirred at same temperature until the reaction was complete. The mixture was poured into ice-water, the organic layer was separated and the aqueous layer was extracted with CH₂CL₂ (50 mL×3). The combined organic layers were washed with 1 N aq HCl until pH<7, then washed with sat. aq. NaCl, dried (MgSO₄) and concentrated to give compound 2 (35 g).

Preparation of Compound 3

To a solution of compound 2 (35 g, 0.119 mol) and Et₃N (50 mL) in CH₂Cl₂ (150 mL) was added dropwise a solution of MsCl (35 mL) in CH₂Cl₂ (50 mL) in an ice bath under N₂. After the addition, the mixture was stirred at the same temperature for 2 h, then allowed to warm to room temperature and stirred until TLC showed the starting material was consumed completely. The mixture was poured into ice-water (200 mL) and stirred for 10 min. The organic layer was separated and the aqueous layer was extracted with CH₂Cl₂ (50 mL×3). The combined organic layers were washed with 1 N aq. HCl to pH<7, then washed with brine, dried over MgSO₄ and concentrated to give compound 3 (45 g, 84%) as yellowish syrup.

Preparation of Compound 5

A mixture of compound 3 (80 g, 0.178 mol), NH₃·H₂O (2500 mL) and MeOH (250 mL) was sealed in autoclave and stirred at 42° C. for 24 h, then concentrated to give compound 4. The compound 4 was dissolved in anhydrous MeOH (300 mL), Boc₂O (45 g, 0.206 mol) was added. The mixture was stirred at rt for 6 h. The mixture was subjected to silica gel column chromatography (EtOAc/petroleum ether 1:5) to give compound 5 (30 g, two steps yield: 45%).

Preparation of Compound 6

Compound 5 (30 g, 0.08 mol) in MeOH (100 mL) was hydrogenated at the exist of 20% Pd(OH)₂/C (5 g) under 76 cmHg of H₂ at room temperature until the reaction was complete. The mixture was filtered and the filtrate was concentrated. The residue was subjected to chromatography to give compound 6 (11 g, 57%). ¹H NMR (DMSO, HCl salt) δ: 8.88 (br, 2), 3.28-3.23 (m, 2H), 3.10 (d, 2H), 2.99 (br, 2H), 1.68-1.61 (m, 2H), 1.63-1.59 (m, 4H), 1.36 (9H).

### Preparation of 2,7-Diaza-spiro[4.4]nonane-2-carboxylic Acid Tert-Butyl Ester

(also referred to as tert-butyl 2,7-diazaspiro[4.4]nonane-2-carboxylate)

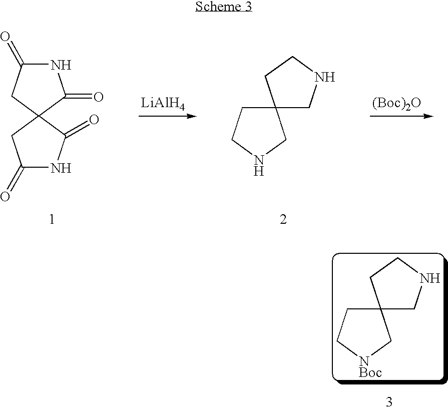

It should be noted that in the following synthetic summary, the intermediate compounds are referred to by their number in scheme 3.

Preparation of Compound 2

To a suspension of 30 g of LiAlH₄ in 900 mL of dry THF was slowly added 27 g of compound 1 (J. Org. Chem. 1981, 2757) under nitrogen atmosphere. The mixture was refluxed for 40 hours. Then 30 mL of aq. KOH (10%) was slowly added at 0° C. The mixture was filtered and the filtrate was extracted with THF (500 mL×3) and 500 mL of acetone. The combined organic layers were dried over Na₂SO₄ and concentrated to afford 16 g of compound 2 (Yield: 77%).

Preparation of Compound 3

To a solution of 16 g of compound 2 in 150 mL of dry methanol was slowly added 26 g of (Boc)₂O in 75 mL of dry methanol at −3˜−2° C. The reaction solution was warmed to room temperature with stirring for 0.5 hour. Then the solvent was removed and the residue was adjusted to pH=3-4 with 10% aq. HCl, extracted with ethyl ether (100 mL×2) to remove impurities. The aqueous phase was basified to pH=10 with K₂CO₃ and extracted with DCM. The organic phase was washed with brine, dried over with Na₂SO₄ and concentrated to give 10 g of compound 3 (Yield: 35%). ¹H NMR (DMSO, HCl salt) δ: 9.52-9.22 (br, 2H), 3.29-3.18 (m, 6H), 3.10-2.98 (m, 2H), 1.93-1.72 (m, 4H), 1.37 (9H).

### Preparation of 2,7-Diaza-spiro[3.5]nonane-2-carboxylic Acid Tert-Butyl Ester

(also referred to as tert-butyl 2,7-diazaspiro[3.5]nonane-2-carboxylate)

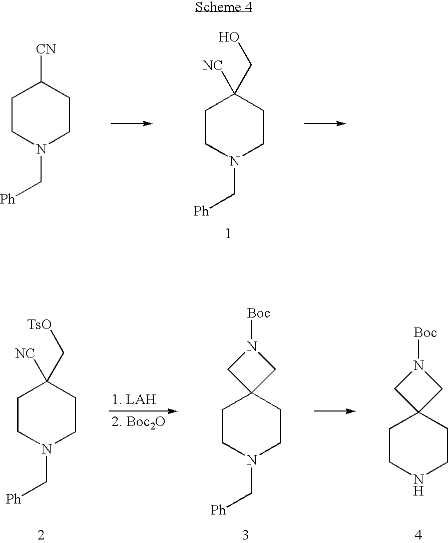

It should be noted that in the following synthetic summary, the intermediate compounds are referred to by their number in scheme 4.

Preparation of Compound 1

To a solution of LDA (12 mmol) in THF cooled to −78° C. was added dropwise the solution of 1-benzyl-piperidine-4-carbonitrile (40 g, 20 mmol) (J. Med. Chem. 1983, 1433-1438) in dry THF (30 mL). After 1 h, gaseous formaldehyde (40 mmol) was passed at −60° C. through this solution, followed by stirring for additional 2 h. Then most of the solvent was removed in vacuo and to the mixture was added saturated NH₄Cl. The mixture was extracted by methylene chloride. The combined organic layers were dried by MgSO₄ and evaporated. The residue was purified by column to give the compound 1 (2 g).

Preparation of Compound 2

To a solution of compound 1 (1.8 g, 8 mmol) in 30 mL of CH₂Cl₂ was added TsCl (20 g, 10.5 mmol), then NaOH (5 g, 0.125 mol) was added slowly while keeping the temperature below 20° C. After the addition, the mixture was stirred at room temperature overnight. Water was added to dissolve the formed solid. The mixture was separated. The organic layer was washed with water, dried over Na₂SO₄ and concentrated to remove most of solvent to give the crude product compound 2, which was used for further step without purification.

Preparation of Compound 3

THF (20 mL) was added dropwise to LiAlH₄ (400 mg) below 0° C., then a solution of the crude compound 2 (3.1 g, 1.82 mol) in THF (15 mL) was added dropwise while keeping the temperature below 10° C. After the addition, the mixture was stirred at room temperature overnight. H₂O (1 mL) was added dropwise to quench the reaction below 10° C. After the mixture was stirred for 1 h, (Boc)₂O (2 g) was added, the resulting mixture was stirred overnight at rt. The mixture was filtered. After evaporation, the residue was purified by column to give compound 3 (1.4 g, 57%).

Preparation of Compound 4

A mixture of compound 3 (1.3 g, 4.11 mmol) and Pd(OH)₂ (200 mg) in 15 mL of MeOH was stirred under 55 psi of H₂ at 35° C. overnight. The mixture was filtered and the filtrate was evaporated. The residue was dissolved in DCM, dried over anhydrous Na₂SO₄ and concentrated. The residue was dissolved in anhydrous ether, then HCl(g)/MeOH was added dropwise until pH=6˜7. The mixture was filtered to afford compound 4 (0.95 g).

¹H NMR(C₄O, HCl salt) δ: 3.72 (s, 4H), 3.30 (t, 4H), 1.99 (t 4H), 1.43 (9H).

### Preparation of 2,7-Diaza-spiro[3.5]nonane-7-carboxylic Acid Tert-Butyl Ester

(also referred to as tert-butyl 2,7-diaza-spiro[3.5]nonane-7-carboxylate)

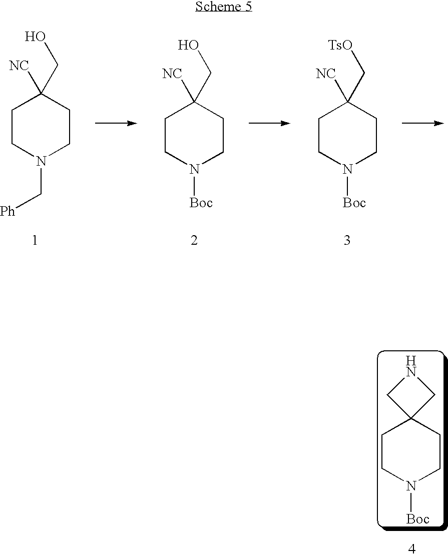

It should be noted that in the following synthetic summary, the intermediate compounds are referred to by their number in scheme 5.

Preparation of Compound 2

To a mixture of compound 1 (6 g, 26 mmol), Boc₂O (6.3 g, 27 mmol) and Pd(OH)₂/C (1.2 g, 5%) in 200 mL autoclave was added 60 mL of MeOH. The mixture was stirred under 0.3 Mpa of H₂ at 20° C. until the reaction was complete. The mixture was filtered and the filtrate was evaporated to give compound 2 (6.3 g), which was used for next step without purification.

Preparation of Compound 3

To a solution of compound 2 (6.3 g) in 60 mL of DCM were added Et₃N (4 mL) and DMAP (0.2 g). The mixture was cooled to 5° C.-10° C. and a solution of TsCl (5.5 g, 9 mmol) in 40 mL of DCM was added dropwise. After the addition, water was added to dissolve some dissoluble material. Then the mixture was adjusted to pH 5˜6 with aq. HCl. The organic layer was separated, washed to pH 7 with water, dried over Na₂SO₄ and concentrated. The residue was purified by column to give the compound 3 (7 g, 68%).

Preparation of Compound 4

THF (40 L) was added to LiAlH₄ (810 mg, 2.13 mol) at 0° C. under N₂. Then a solution of compound 3 (5 g, 1.37 mol) in 30 L of THF was added dropwise at rt over 4 h. After the addition, the mixture was stirred overnight. To the mixture was added dropwise 1.6 mL of 10% aq. NaOH and 0.8 mL of H₂O and stirred for 0.5 h and filtered. The cake was washed with DCM. The filtrate was concentrated, the residue was dissolved in DCM, dried over Na₂SO₄ and concentrated. 2 mL of MeOH was added, followed by 5 L of ether. The mixture was adjusted to pH 6 with HCl/MeOH. The precipitate was filtered and washed with ether to give 1.70 g of compound 4. ¹H NMR (CDCl₃, HCl salt) δ: 9.71 (br, 2H), 3.83 (t, 4H), 3.34-3.31 (m, 4H), 1.86-1.83 (m, 4H), 1.43 (9H).

### Preparation of 3,9-Diaza-spiro[5.5]undecane-3-carboxylic Acid Tert-Butyl Ester

(also referred to as tert-butyl 3,9-diazaspiro[5.5]undecane-3-carboxylate)

U.S. Pat. No. 5,451,578 (Claremon et al.) describes, under example 1 of the patent, a process for synthesising tert-butyl 3,9-diazaspiro[5.5]undecane-3-carboxylate.

A preparation method is also described below, with reference to scheme 6.

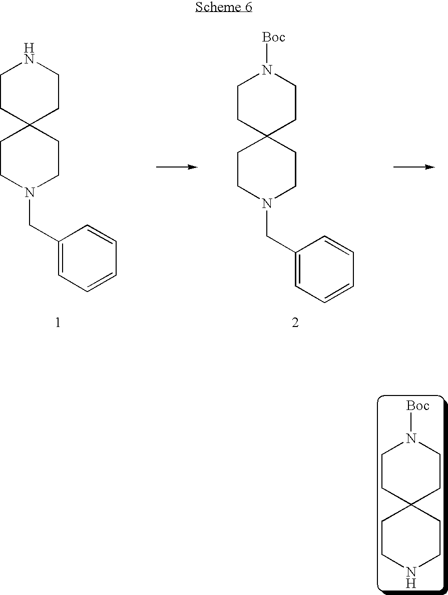

Preparation of Compound 2

The mixture of the compound 1 (2.44 g) (U.S. Pat. No. 6,291,469, page 62, column 123), Boc₂O (2 g) in MeOH (30 mL) was stirred overnight. After evaporation, the residue was purified by column to give the compound 2 (3 g, 87%).

### Preparation of Compound 3,9-Diaza-spiro[5.5]undecane-3-carboxylic Acid Tert-Butyl Ester

A mixture of compound 2 (3 g) and Pd(OH)₂ (300 mg) in 15 mL of MeOH was stirred under 55 psi of H₂ at 35° C. overnight. The mixture was filtered and the filtrate was evaporated. The residue was dissolved in anhydrous ether, then HCl(g)/MeOH was added to pH=67. The mixture was filtered to afford compound 3,9-diaza-spiro[5.5]undecane-3-carboxylic acid tert-butyl ester (2.2 g). ¹H NMR (CDCl₃, HCl salt) δ: 9.49 (br, 2H), 3.39-3.36 (m, 4H), 3.16 (br, 4H), 1.82-1.79 (m, 4H), 1.51-1.48 (m, 4H), 1.44 (9H).

### EXAMPLE 1

### 3-[(2,2-dimethyl-2,3-dihydro-1-benzofuran-7-yl)methyl]-9-isonicotinoyl-3,9-diazaspiro[5.5]undecane Trifluoroacetate

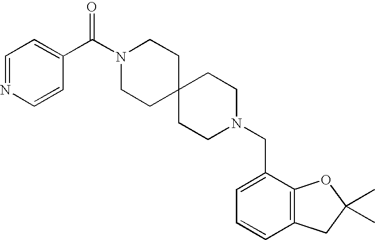

A mixture of 2,2 dimethyl-2,3-dihydro-1-benzofuran-7-carbaldehyde (102 mg, 0.58 mmol), Intermediate S (100 mg, 0.38 mmol), sodium triacetoxyborohydride (200 mg, 0.94 mmol) and acetonitrile (4 ml) were stirred for 18 h at room temperature, diluted with ethyl acetate and washed with aqueous sodium hydrogen carbonate, the organic layer was evaporated and the residue was purified by preparative HPLC (RP-18, gradient acetonitrile/water/TFA from 10/90/0.1 to 95/5/0.1) to give the product as a white solid (94 mg, 46%).

¹H NMR (399.99 MHz, CD3OD) δ 8.85 (d, J=3.9 Hz, 2H), 7.95-7.71 (m, 2H), 7.33-7.25 (m, 1H), 7.24-7.17 (m, 1H), 7.04-6.74 (m, 1H), 4.26 (d, J=11.3 Hz, 2H), 3.78 (m, 2H), 3.45-3.32 (m, 6H), 3.26-3.07 (m, 5H), 2.03 (d, J=14.8 Hz, 2H), 1.88-1.77 (m, 1H), 1.61-1.55 (m, 3H), 1.53-1.43 (m, 1H), 1.42 (s, 3H), 1.40 (s, 3H)

APCI-MS m/z: 420.4 [MH+]

HPLC (Method A) Retention time: 5.03 min

HPLC (Method B) Retention time: 7.95 min

### EXAMPLE 2

### 3-[(2,2-dimethyl-2,3-dihydro-1-benzofuran-7-yl)methyl]-9-[(1-oxidopyridin-2-yl)carbonyl]-3,9-diazaspiro[5.5]undecane

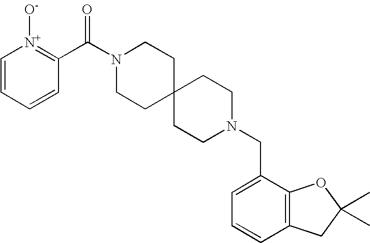

A mixture of Intermediate A (63.3 mg, 0.201 mmol), HATU (76.6 mg, 0.201 mmol), pyridine-2-carboxylic acid 1-oxide (34 mg, 0.24 mmol), triethylamine (49 μl, 0.36 mmol) and dichloromethane (4 mL) was stirred at room temperature for 1.5 h. The reaction mixture was diluted with EtOAc and washed with sodium hydrogen carbonate solution. The organic layer was isolated, evaporated to dryness and the residue was purified by preparative HPLC (RP-18) to give the product as a white solid (65 mg, 74%).

¹H NMR (399.99 MHz, CD3OD) δ 8.36-8.29 (m, 1H), 7.70-7.53 (m, 3H), 7.34-7.27 (m, 1H), 7.24-7.16 (m, 1H), 4.30-4.23 (m, 2H), 3.86-3.46 (m, 2H), 3.46-3.38 (m, 3H), 3.39-3.08 (m, 5H), 2.10-1.97 (m, 2H), 1.93-1.56 (m, 6H), 1.56-1.47 (m, 6H)

APCI-MS m/z: 435.9 [MH+]

HPLC (Method A) Retention time: 5.29 min

HPLC (Method B) Retention time: 6.58 min

### EXAMPLE 3

### 3-({9-[(2,2-dimethyl-2,3-dihydro-1-benzofuran-7-yl)methyl]-3,9-diazaspiro[5.5]undec-3-yl}carbonyl)pyridin-2(1H)-one

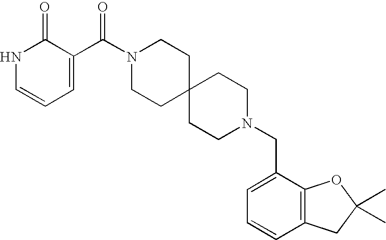

A mixture of Intermediate A (68.8 mg, 0.22 mmol), HATU (83.3 mg, 0.22 mmol), 2-hydroxy nicotinic acid (37 mg, 0.27 mmol), triethylamine (54 μl, 0.39 mmol) and dichloromethane (4 mL) was stirred at room temperature for 1.5 h. The reaction mixture was diluted with EtOAc and washed with sodium hydrogen carbonate solution. The organic layer was isolated, evaporated to dryness and the residue was purified by preparative HPLC (RP-18) to give the product as a white solid (46 mg, 48%).

¹H NMR (399.99 MHz, CD3OD) δ 7.64-7.59 (m, 1H), 7.53-7.49 (m, 1H), 7.16-7.08 (m, 2H), 6.86-6.78 (m, 1H), 6.47-6.40 (m, 1H), 3.84-3.60 (m, 4H), 3.10-2.99 (m, 2H), 2.87-2.62 (m, 4H), 1.79-1.25 (m, 16H)

APCI-MS m/z: 435.9 [MH+]

HPLC (Method A) Retention time: 5.39 min

HPLC (Method B) Retention time: 6.63 min

### EXAMPLE 4

### [2-({9-[(2,2-dimethyl-2,3-dihydro-1-benzofuran-7-yl)methyl]-3,9-diazaspiro[5.5]undec-3-yl}carbonyl)phenyl]acetic Acid

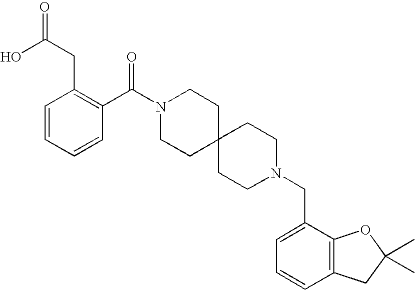

A mixture of methyl [2-({9-[(2,2-dimethyl-2,3-dihydro-1-benzofuran-7-yl)methyl]-3,9-diazaspiro[5.5]undec-3-yl}carbonyl)phenyl]acetate (90 mg, 0.18 mmol), LiOH (4.8 mg, 0.2 mmol), THF (1 ml), MeOH (1 ml) and water (1 ml) was stirred at room temperature for 3 h, acetic acid (1 ml) was added and the product was purified by preparative HPLC (RP-18, gradient acetonitrile/water/NH₄OAc 20% to 70%) to give the title compound as a white solid (14.5 mg, 16%).

¹H NMR (399.99 MHz, DMSO-D6) δ 7.38-7.23 (m, 3H), 7.20-7.13 (m, 1H), 7.07-7.00 (m, 2H), 6.74 (t, J=18.1 Hz, 1H), 3.68-3.40 (m, 4H), 3.40-3.33 (m, 2H), 3.00-2.92 (m, 2H), 2.43-2.21 (m, 4H), 1.92-1.85 (m, 2H), 1.58-1.17 (m, 14H)

APCI-MS m/z: 477.3 [MH+]

HPLC (Method A) Retention time: 3.83 min

HPLC (Method B) Retention time: 7.09 min

### EXAMPLE 5

### Methyl [2-({9-[(2,2-dimethyl-2,3-dihydro-1-benzofuran-7-yl)methyl]-3,9-diazaspiro[5.5]undec-3-yl}carbonyl)phenyl]acetate

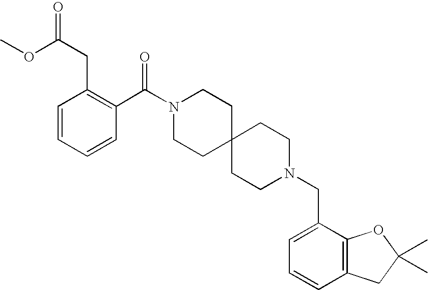

A mixture of Intermediate A (45 mg, 0.23 mmol), HATU (73 mg, 0.19 mmol), 2-(2-methoxy-2-oxoethyl)benzoic acid (43 mg, 0.22 mmol), triethylamine (47 μl, 0.34 mmol) and dichloromethane (4 mL) was stirred at room temperature for 1.5 h. The crude methyl ester was evaporated and purified by preparative HPLC (RP-18) to give the product as a white solid (93 mg, 81%).

¹H NMR (499.881 MHz, DMSO-D6) δ 7.42-7.26 (m, 4H), 7.25-7.19 (m, 2H), 6.94-6.87 (m, 1H), 4.26-4.11 (m, 2H), 3.70-3.49 (m, 3H), 3.30-2.95 (m, 10H), 1.95-1.19 (m, 16H)

APCI-MS m/z: 490.9 [MH+]

HPLC (Method A) Retention time: 8.31 min

HPLC (Method B) Retention time: 10.04 min

### EXAMPLE 6

### 3-({9-[(2,2-dimethyl-2,3-dihydro-1-benzofuran-7-yl)methyl]-3,9-diazaspiro[5.5]undec-3-yl}carbonyl)-1-methylpyridin-2(1H)-one

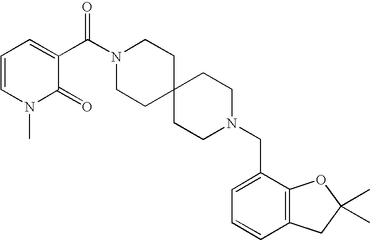

### a) N-methyl-2-hydroxynicotinic Acid

2-Hydroxynicotinic acid (75 mg, 0.54 mmol) was dissolved in MeOH (0.75 mL) and H₂O (0.112 mL). Ground KOH (60 mg g, 1.07 mmol) was added and the reaction mixture was refluxed for 15 min. Methyl iodide (0.389 mL, 6.03 mmol) was added and the reaction mixture was heated to reflux for 2 h. After evaporation to half of the volume and addition of 10% hydrochloric acid (0.075 mL) white crystals of the title compound were obtained by filtration (38 mg).

¹H NMR (399.99 MHz, D₂O): δ 8.35 (dd, 1H), 7.93 (dd, 1H), 6.64 (t, 1H), 3.59 (s, 3H)

### b) 3-({9-[(2,2-dimethyl-2,3-dihydro-1-benzofuran-7-yl)methyl]-3,9-diazaspiro[5.5]undec-3-yl}carbonyl)-1-methylpyridin-2(1H)-one

N-methyl-2-hydroxynicotinic acid (45 mg, 0.29 mmol), Intermediate A (76 mg, 0.24 mmol), HATU (91.9 mg, 0.24 mmol) and triethylamine (43 mg, 0.43 mmol) in CH₂Cl₂ (4 mL) were mixed and stirred for 1 h. The reaction mixture was diluted with saturated aqueous sodium carbonate (2 mL) and the product was extracted with dichloromethane and dried. The pure title compound was obtained by preparative HPLC.

¹H NMR (399.99 MHz, CD3OD) δ 7.81-7.71 (m, 1H), 7.60-7.52 (m, 1H), 7.32-7.26 (m, 1H), 7.23-7.16 (m, 1H), 6.97-6.88 (m, 1H), 6.48-6.36 (m, 1H), 4.32-4.19 (m, 2H), 3.79-3.67 (m, 2H), 3.62-3.55 (m, 3H), 3.45-3.35 (m, 2H), 3.26-3.06 (m, 4H), 2.08-1.96 (m, 2H), 1.84-1.37 (m, 14H)

APCI-MS m/z: 450.5 [MH+]

HPLC (Method A) Retention time: 5.63 min

HPLC (Method B) Retention time: 7.07 min

### EXAMPLE 7

### 3-[(2,2-dimethyl-2,3-dihydro-1-benzofuran-7-yl)methyl]-9-(pyrimidin-4-ylcarbonyl)-3,9-diazaspiro[5.5]undecane

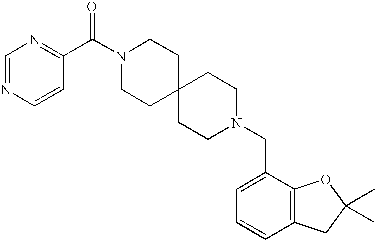

A mixture of Intermediate A (14 mg, 0.44 mmol), HATU (17 mg, 0.44 mmol), pyridimin-4-carboxylic acid (7 mg, 0.53 mmol), triethylamine (11 μl, 0.78 mmol) and dichloromethane (1 mL) was stirred at room temperature for 1.5 h. The reaction mixture was diluted with EtOAc and washed with sodium hydrogen carbonate solution. The organic layer was isolated, evaporated to dryness and the residue was purified by preparative HPLC (RP-18) to give the product as a white solid (6 mg, 32%).

¹H NMR (399.99 MHz, CD3OD) δ 9.25-9.18 (m, 1H), 8.99-8.90 (m, 1H), 7.68-7.62 (m, 1H), 7.32-7.25 (m, 1H), 7.24-7.16 (m, 1H), 6.98-6.88 (m, 1H), 4.32-4.21 (m, 2H), 3.83-3.72 (m, 2H), 3.48-3.35 (m, 4H), 3.28-3.08 (m, 4H), 2.11-1.99 (m, 2H), 1.85-1.56 (m, 6H), 1.54-1.45 (m, 6H

APCI-MS m/z: 421.2 [MH+]

HPLC (Method A) Retention time: 5.97 min

HPLC (Method B) Retention time: 7.70 min

### EXAMPLE 8

### 4-({9-[(2,2-dimethyl-2,3-dihydro-1-benzofuran-7-yl)methyl]-3,9-diazaspiro[5.5]undec-3-yl}carbonyl)pyridin-3-ol

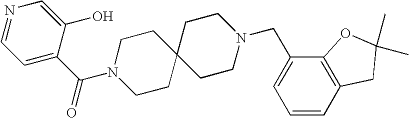

A mixture of Intermediate A (50 mg, 0.13 mmol), HBTU (50 mg, 0.15 mmol), 3-hydroxyisonicotinic acid (21 mg, 0.15 mmol), triethylamine (100 μl, 0.7 mmol) and acetonitrile (1 ml) was stirred at room temperature until the reaction was complete then acidified with TFA. The mixture was evaporated and the residue purified by preparative HPLC (RP-18, gradient acetonitrile/water/TFA from 10/90/0.1 to 95/5/0.1 and acetonitrile/water/NH₄OAc 10/90/0.1 to 95/5/0.1) to give the product as a white solid (2 mg, 3%).

APCI-MS m/z: 436.1 [MH+]

HPLC (Method A) Retention time: 5.34 min

HPLC (Method B) Retention time: 3.87 min

### EXAMPLE 9

### 2-({9-[(2,2-dimethyl-2,3-dihydro-1-benzofuran-7-yl)methyl]-3,9-diazaspiro[5.5]undec-3-yl}carbonyl)pyridin-3-ol

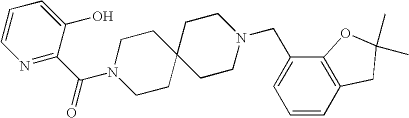

The title compound was prepared by the procedure of Example 8 using Intermediate A and 3-hydroxypyridine-2-carboxylic acid as starting materials to give the product as a white solid (2 mg, 3%).

APCI-MS m/z: 436.5 [MH+]

HPLC (Method A) Retention time: 6.58 min

HPLC (Method B) Retention time: 2.00 min

### EXAMPLE 10

### 5-({9-[(2,2-dimethyl-2,3-dihydro-1-benzofuran-7-yl)methyl]-3,9-diazaspiro[5.5]undec-3-yl}carbonyl)pyridin-2-amine

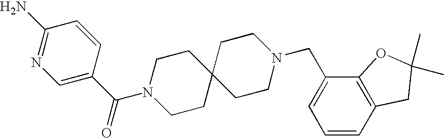

The title compound was prepared by the procedure of Example 8 using Intermediate A and 6-aminonicotinic acid as starting materials to give the product as a white solid (4 mg, 6%).

APCI-MS m/z: 435.5 [MH+]

HPLC (Method A) Retention time: 5.24 min

HPLC (Method B) Retention time: 7.89 min

### EXAMPLE 11

### 3-({9-[(2,2-dimethyl-2,3-dihydro-1-benzofuran-7-yl)methyl]-3,9-diazaspiro[5.5]undec-3-yl}carbonyl)pyridin-2-amine Trifluoroacetate

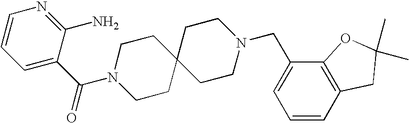

The title compound was prepared by the synthesis procedure of Example 8 using Intermediate A and 2-aminonicotinic acid as starting materials. The crude product was purified by preparative HPLC (RP-18, gradient acetonitrile/water/TFA from 10/90/0.1 to 95/5/0.1) to give the product as a white solid (47 mg, 57%).

¹H NMR (299.946 MHz, DMSO-D6) δ 9.30-9.09 (m, 1H), 8.03 (dd, 1H), 7.74-7.64 (m, 1H), 7.26 (dd, 1H), 6.90 (t, 1H), 6.81 (s, 1H), 4.21 (s, 2H), 3.25 (d, J=11.7 Hz, 4H), 3.13-3.00 (m, 4H), 1.92-1.79 (m, 2H), 1.70-1.31 (m, 12H). The signals of two protons in the aliphatic region are missing due to overlapping resonances with solvent.

APCI-MS m/z: 435.1 [MH+]

HPLC (Method A) Retention time: 5.31 min

HPLC (Method B) Retention time: 8.09 min

### EXAMPLE 12

### 2-({9-[(2,2-dimethyl-2,3-dihydro-1-benzofuran-7-yl)methyl]-3,9-diazaspiro[5.5]undec-3-yl}carbonyl)pyridin-4-ol

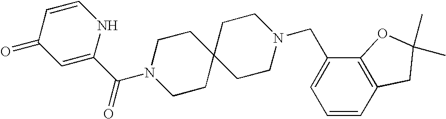

The title compound was prepared by the procedure of Example 8 using Intermediate A and 4-hydroxypyridine-2-carboxylic acid as starting materials to give the product as a white solid (2 mg, 3%).

APCI-MS m/z: 436.1 [MH+]

HPLC (Method B) Retention time: 3.01 min

### EXAMPLE 13

### 3-({9-[(2,2-dimethyl-2,3-dihydro-1-benzofuran-7-yl)methyl]-3,9-diazaspiro[5.5]undec-3-yl}carbonyl)pyridin-4-ol

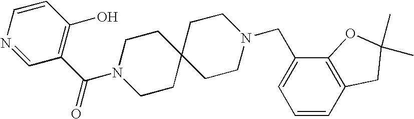

The title compound was prepared by the procedure of Example 8 using Intermediate A and 4-hydroxynicotinic acid as starting materials to give the product as a white solid (4 mg, 6%).

APCI-MS m/z: 436.1 [MH+]

HPLC (Method A) Retention time: 5.38 min

HPLC (Method B) Retention time: 4.71 min

### EXAMPLE 14

### 3-(1H-1,2,3-benzotriazol-5-ylcarbonyl)-9-[(2,2-dimethyl-2,3-dihydro-1-benzofuran-7-yl)methyl]-3,9-diazaspiro[5.5]undecane Trifluoroacetate

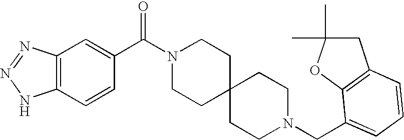

The title compound was prepared by the procedure of Example 8 using Intermediate A and 1H-1,2,3-benzotriazole-5-carboxylic acid as starting materials to give the product as a white solid (39 mg, 45%).

¹H NMR (299.946 MHz, DMSO-D6) δ 9.21 (s, 1H), 7.95 (s, 1H), 7.44 (d, 1H), 7.26 (dd, 2H), 6.90 (t, 1H), 4.20 (s, 2H), 3.62 (s, 2H), 3.25 (d, 4H), 3.06 (s, 4H), 1.91 (d, 2H), 1.78-1.25 (m, 12H)

APCI-MS m/z: 460.5 [MH+]

HPLC (Method A) Retention time: 6.42 min

HPLC (Method B) Retention time: 4.40 min

### EXAMPLE 15

### 5-({9-[(2,2-dimethyl-2,3-dihydro-1-benzofuran-7-yl)methyl]-3,9-diazaspiro[5.5]undec-3-yl}carbonyl)pyridine-2-carbonitrile Trifluoroacetate

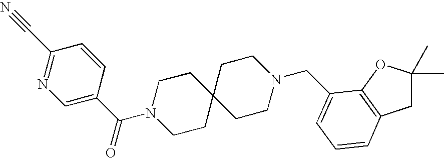

The title compound was prepared by the synthesis procedure of Example 8 using Intermediate A and 6-cyanonicotinic acid as starting materials to give the product as a white solid (22 mg, 26%).

¹H NMR (299.946 MHz, DMSO-D6) δ 9.29-9.03 (m, 1H), 8.77 (s, 1H), 8.21-8.01 (m, 1H), 7.26 (dd, 2H), 6.90 (t, 1H), 4.20 (s, 2H), 3.62 (s, 4H), 3.34-2.95 (m, 6H), 1.96-1.80 (m, 2H), 1.77-1.28 (m, 12H)

APCI-MS m/z: 445.5 [MH+]

HPLC (Method A) Retention time: 7.42 min

HPLC (Method B) Retention time: 9.33 min

### EXAMPLE 16

### 2′-({9-[(2,2-dimethyl-2,3-dihydro-1-benzofuran-7-yl)methyl]-3,9-diazaspiro[5.5]undec-3-yl}carbonyl)biphenyl-2-carboxylic Acid Trifluoroacetate

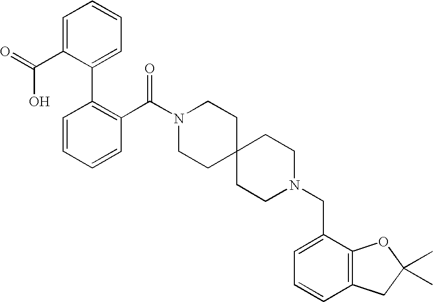

The title compound was prepared by the synthesis procedure of Example 8 using Intermediate A and biphenyl-2,2′-dicarboxylic acid as starting materials to give the product as a white solid (22 mg, 22%).

APCI-MS m/z: 539.2 [MH+]

HPLC (Method A) Retention time: 8.52 min

HPLC (Method B) Retention time: 4.01 min

### EXAMPLE 17

### 2-[2-({9-[(2,2-dimethyl-2,3-dihydro-1-benzofuran-7-yl)methyl]-3,9-diazaspiro[5.5]undec-3-yl}carbonyl)phenyl]acetamide Trifluoroacetate

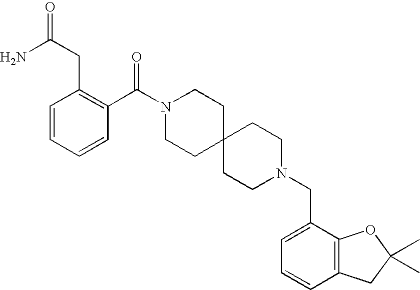

The lithium salt (51 mg, 0.11 mmol) of Example 4 was stirred with sodium bicarbonate (121 mg, 1.44 mmol) in acetonitrile (1 ml) and 1-methyl-2-pyrrolidone (0.5 ml) for 15 min. HBTU (60 mg, 0.18 mmol) and 7M methanolic solution of NH3 (200 μL) were added and the reaction mixture was stirred at room temperature for 1 h. An additional batch of HBTU (40 mg, 0.12 mmol) and 7M methanolic solution of NH3 (200 μL) was added and reaction mixture was stirred at room temperature for a further 16 h. The crude product was purified by preparative HPLC (RP-18, gradient acetonitrile/water/TFA from 10/90/0.1 to 95/5/0.1) to give the product as a white solid (19 mg, 29%).

¹H NMR (499.881 MHz, DMSO-D6) δ 7.39 (s, 1H), 7.36-7.23 (m, 2H), 7.17-7.13 (m, 1H), 7.03 (s, 1H), 6.87 (s, 1H), 6.74 (t, 1H), 3.59 (t, 2H), 3.49-3.40 (m, 2H), 3.34 (d, 2H), 3.09 (d, 2H), 2.97 (s, 2H), 2.40-2.26 (m, 4H), 1.52-1.23 (m, 14H)

APCI-MS m/z: 476.4 [MH+]

HPLC (Method A) Retention time: 6.48 min

HPLC (Method B) Retention time: 8.10 min

### EXAMPLE 18

### 1-{[2-({9-[(2,2-dimethyl-2,3-dihydro-1-benzofuran-7-yl)methyl]-3,9-diazaspiro[5.5]undec-3-yl}carbonyl)phenyl]acetyl}-D-prolinamide

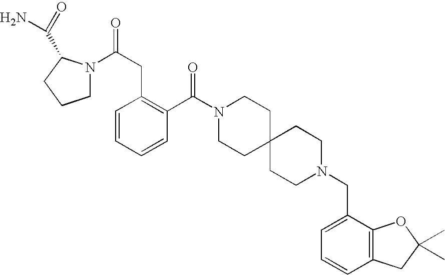

A mixture of Example 4 hydrochloric acid salt (92 mg, 0.18 mmol), HATU (71 mg, 0.19 mmol), D-prolinamide (31 mg, 0.27 mmol), triethylamine (150 μl, 1.0 mmol) and acetonitrile (2 ml) was stirred at room temperature for 1 h then evaporated. The crude product was purified by preparative HPLC (RP-18, gradient acetonitrile/water/TFA 10/90/0.1 and acetonitrile/water/NH4OAc 10:90:0.1 to 95:5:0.1) to give the product as a white solid (13 mg, 13%).

¹H NMR (499.881 MHz, DMSO-D6) δ 7.42-6.85 (m, 7H), 4.20 (d, 2H), 3.29 (s, 2H), 3.26-3.18 (m, 2H), 3.18-2.96 (m, 6H), 2.04-1.71 (m, 6H), 1.62-1.21 (m, 110H), 1.17 (q, 2H), 3.76-3.36 (m, 5H)

APCI-MS m/z: 573.5 [MH+]

HPLC (Method A) Retention time: 6.56 min

HPLC (Method B) Retention time: 7.88 min

### EXAMPLE 19

### N-cyclopropyl-2-[2-({9-[(2,2-dimethyl-2,3-dihydro-1-benzofuran-7-yl)methyl]-3,9-diazaspiro[5.5]undec-3-yl}carbonyl)phenyl]acetamide

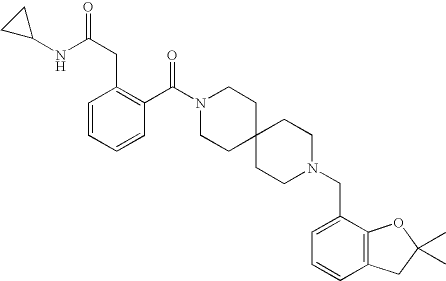

The title compound was prepared by the synthesis procedure of Example 18 using Example 4 hydrochloric acid salt and cyclopropylamine as starting materials to give the product as a white solid (15 mg, 16%).

¹H NMR (499.881 MHz, DMSO-D6) δ 8.06 (d, 1H), 7.35-7.23 (m, 3H), 7.14 (d, 1H), 7.03 (dd, 2H), 6.74 (t, 1H), 3.64-3.51 (m, 2H), 3.36-3.34 (m, 2H), 3.10-3.04 (m, 2H), 2.97 (s, 2H), 2.61-2.55 (m, 1H), 2.39-2.27 (m, 4H), 1.49-1.40 (m, 6H), 1.38 (s, 6H), 1.34-1.27 (m, 2H), 0.60-0.55 (m, 2H), 0.38-0.34 (m, 2H), 3.30-3.26 (m, 2H)

APCI-MS m/z: [MH+] 516.5

HPLC (Method A) Retention time: 7.35 min

HPLC (Method B) Retention time: 9.16 min

### EXAMPLE 20

### 3-[2-(2-azetidin-1-yl-2-oxoethyl)benzoyl]-9-[(2,2-dimethyl-2,3-dihydro-1-benzofuran-7-yl)methyl]-3,9-diazaspiro[5.5]undecane Acetate

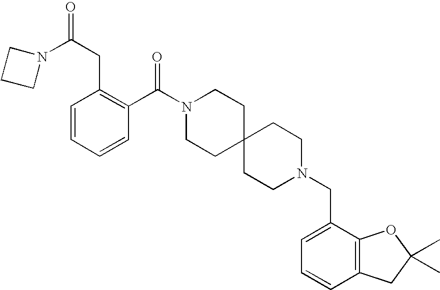

The title compound was prepared by the synthesis procedure of Example 18 using Example 4 hydrochloric acid salt and azetidine as starting materials to give the product as a white solid (13 mg, 14%).

¹H NMR (499.881 MHz, DMSO-D6) δ 7.35-7.24 (m, 3H), 7.16 (s, 1H), 7.05-7.02 (m, 2H), 6.74 (t, 1H), 4.11 (d, 2H), 3.81 (s, 2H), 3.59 (t, 4H), 3.12-3.01 (m, 2H), 2.97 (s, 2H), 2.40-2.26 (m, 4H), 2.22-2.12 (m, 2H), 1.52-1.34 (m, 14H), 1.29 (s, 2H)

APCI-MS m/z: [MH+] 516.5

HPLC (Method A) Retention time: 6.93 min

HPLC (Method B) Retention time: 8.48 min

### EXAMPLE 21

### [5-chloro-2-({9-[(2,2-dimethyl-2,3-dihydro-1-benzofuran-7-yl)methyl]-3,9-diazaspiro[5.5]undec-3-yl}carbonyl)phenyl]acetic Acid Trifluoroacetate

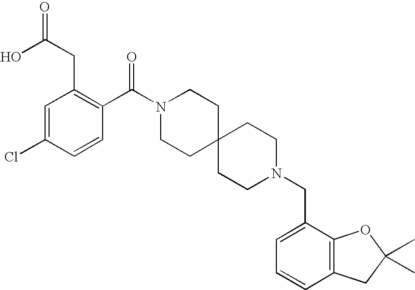

The title compound was prepared by the conditions described in the amide coupling procedure of Example 8 using Intermediate A (55 mg, 0.13 mmol) and 4-chloro-2-[2-methoxy-1-(methoxycarbonyl)-2-oxoethyl]benzoic acid (34 mg, 0.16 mmol) as starting materials. The crude product obtained from the amide coupling was treated with LiOH (80 mg, 3.3 mmol), THF (1 ml), MeOH (1 ml) and water (1 ml). The mixture was stirred at 50° C. for 2 h, acetic acid (1 ml) was added and the crude product was purified by preparative HPLC (RP-18, gradient acetonitrile/water/TFA from 10/90/0.1 to 95/5/0.1) to give the product as a white solid (32 mg, 40%).

¹H NMR (499.881 MHz, DMSO-D6) δ 12.69-12.20 (m, 1H), 9.17 (d, 1H), 7.45 (dd, 1H), 7.39 (dd, 1H), 7.31-7.20 (m, 2H), 6.90 (td, 1H), 4.19 (dd, 2H), 3.74-3.50 (m, 4H), 3.29-3.19 (m, 2H), 3.18-2.97 (m, 6H), 1.96-1.74 (m, 2H), 1.72-1.31 (m, 12H)

APCI-MS m/z: [MH+] 511.1

HPLC (Method A) Retention time: min 9.34

### EXAMPLE 22

### 3-[2-({9-[(2,2-dimethyl-2,3-dihydro-1-benzofuran-7-yl)methyl]-3,9-diazaspiro[5.5]undec-3-yl}carbonyl)phenyl]propanoic Acid Trifluoroacetate

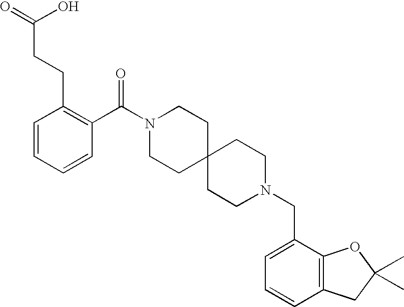

The title compound was prepared by the synthesis procedure of Example 21 using Intermediate A and 2-(3-methoxy-3-oxopropyl)benzoic acid as starting materials to give the product as a white solid (29 mg, 37%).

¹H NMR (499.881 MHz, DMSO-D6) δ 12.32-11.91 (m, 1H), 9.15 (d, 1H), 7.38-7.20 (m, 4H), 7.15 (dd, 1H), 6.90 (td, 1H), 4.19 (dd, 2H), 3.91-3.43 (m, 4H), 3.29-3.19 (m, 2H), 3.18-2.97 (m, 4H), 2.86-2.65 (m, 2H), 1.98-1.81 (m, 2H), 1.73-1.62 (m, 2H), 1.61-1.12 (m, 12H)

APCI-MS m/z: [MH+] 491.4

HPLC (Method A) Retention time: 7.60 min

### EXAMPLE 23

### 4-({9-[(2,2-dimethyl-2,3-dihydro-1-benzofuran-7-yl)methyl]-3,9-diazaspiro[5.5]undec-3-yl}carbonyl)pyridin-2-amine

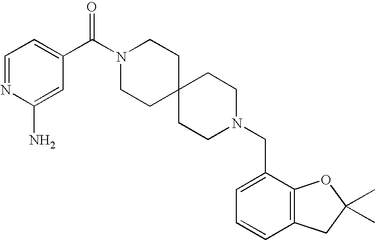

The title compound was prepared by the procedure of Example 8 using Intermediate A and 2-aminopyridine-4-carboxylic acid as starting materials to give the product as a white solid (5 mg, 5%).

¹H NMR (499.881 MHz, DMSO-D6) δ 7.92 (d, 1H), 7.03 (d, 2H), 6.74 (t, 1H), 6.38 (dd, 1H), 6.32 (s, 1H), 6.08 (s, 2H), 3.53 (s, 2H), 3.29 (s, 2H), 3.22 (s, 2H), 2.97 (s, 2H), 2.40-2.26 (m, 4H), 1.52-1.30 (m, 14H)

APCI-MS m/z: [MH+] 435.4

HPLC (Method A) Retention time: 5.17 min

HPLC (Method B) Retention time: 7.88 min

### EXAMPLE 24

### 4-({9-[(2,2-dimethyl-2,3-dihydro-1-benzofuran-7-yl)methyl]-3,9-diazaspiro[5.5]undec-3-yl}carbonyl)pyridin-3-amine Ditrifluoroacetate

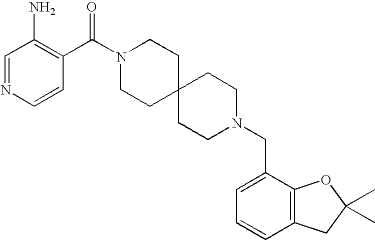

Intermediate A (60 mg, 0.16 mmol), 3-aminoisonicotinic acid (26 mg, 0.19 mmol), HBTU (72 mg, 0.19 mmol) and triethylamine (86 μl, 0.62 mmol) were dissolved in dichloromethane (3 ml) and stirred at room temperature for 1 h. The mixture was diluted with dichloromethane and washed with saturated aqueous Sodium bicarbonate. The organic layer was evaporated and purified by preparative HPLC (RP-18, gradient acetonitrile/water/TFA 10/90/0.1 to 70/30/0.1) to afford the title compound as a clear gum (20 mg, 20%).

¹H NMR (499.881 MHz, CD3OD) δ 8.15 (s, 1H), 8.00 (d, J=3.5 Hz, 1H), 7.63 (d, J=5.6 Hz, 1H), 7.29 (d, J=7.2 Hz, 1H), 7.20 (d, J=7.5 Hz, 1H), 6.92 (t, J=7.6 Hz, 1H), 4.26 (d, J=10.9 Hz, 2H), 3.78 (s, 2H), 3.46-3.34 (m, 18H), 3.26-3.13 (m, 9H), 3.11 (s, 3H), 2.03 (d, J=14.3 Hz, 2H), 1.83 (s, 1H), 1.73-1.59 (m, 4H), 1.50 (d, J=9.8 Hz, 7H)

APCI-MS m/z: 435.2 [MH+]

HPLC (Method A) Retention time: 5.01 min

### EXAMPLE 25

### N-[2-({9-[(2,2-dimethyl-2,3-dihydro-1-benzofuran-7-yl)methyl]-3,9-diazaspiro[5.5]undec-3-yl}carbonyl)phenyl]methanesulfonamide Trifluoroacetate

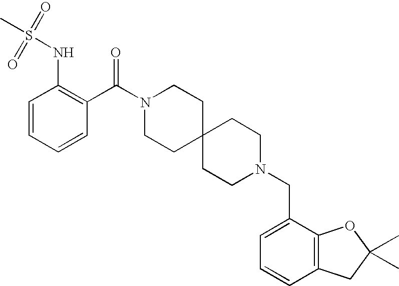

The title compound was prepared by the procedure of Example 24 using Intermediate A (60 mg, 0.16 mmol) and 2-[(methylsulfonyl)amino]benzoic acid (41 mg, 0.19 mmol) as starting materials. The crude product was purified by preparative HPLC (RP-18, gradient acetonitrile/water/TFA 10/90/0.1 to 80/20/0.1) to afford the title compound as a white solid (17 mg, 17%).

¹H NMR (499.881 MHz, CD3OD) δ 7.48 (d, J=2.5 Hz, 2H), 7.36-7.27 (m, 3H), 7.20 (d, J=7.8 Hz, 1H), 6.92 (t, J=7.5 Hz, 1H), 4.26 (s, 2H), 3.75 (s, 2H), 3.44-3.33 (m, 6H), 3.26-3.13 (m, 2H), 3.11 (s, 2H), 3.06 (s, 3H), 2.04 (d, J=14.5 Hz, 2H), 1.85-1.54 (m, 6H), 1.50 (s, 6H)

APCI-MS m/z: 512.2 [MH+]

HPLC (Method A) Retention time: 7.44 min

Examples 26-37 were all synthesised according to example 24, using appropriate acids and Intermediate A, and purified by preparative HPLC (RP-18, gradient acetonitrile/water/TFA 10/90/0.1 to 70/30/0.1).

### EXAMPLE 26

### 4-({9-[(2,2-dimethyl-2,3-dihydro-1-benzofuran-7-yl)methyl]-3,9-diazaspiro[5.5]undec-3-yl}carbonyl)-1H-pyrazol-3-amine Trifluoroacetate

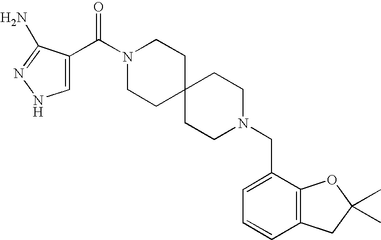

¹H NMR (499.881 MHz, CD3OD) δ 7.72 (s, 1H), 7.30 (d, J=7.3 Hz, 1H), 7.20 (d, J=7.5 Hz, 1H), 6.93 (t, J=7.5 Hz, 1H), 4.27 (s, 2H), 3.70 (q, J=5.9 Hz, 4H), 3.41 (d, J=12.3 Hz, 2H), 3.23-3.15 (m, 2H), 3.11 (s, 2H), 2.03 (d, J=15.5 Hz, 2H), 1.73 (t, J=5.7 Hz, 2H), 1.65 (dd, J=28.1, 4.3 Hz, 3H), 1.54-1.48 (m, 2H), 1.51 (s, 6H)

APCI-MS m/z: 524.2 [MH+]

HPLC (Method A) Retention time: 5.07 min

### EXAMPLE 27

### 3-[(2,2-dimethyl-2,3-dihydro-1-benzofuran-7-yl)methyl]-9-(1,2,3-thiadiazol-4-ylcarbonyl)-3,9-diazaspiro[5.5]undecane Trifluoroacetate

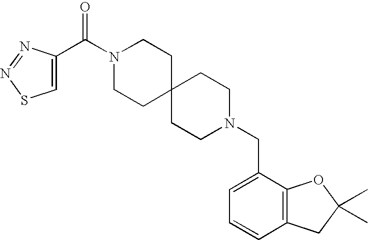

¹H NMR (499.881 MHz, CD3OD) δ 9.34 (d, J=4.2 Hz, 1H), 7.32-7.27 (m, 1H), 7.23-7.18 (m, 1H), 6.93 (td, J=7.5, 3.7 Hz, 1H), 4.27 (d, J=11.4 Hz, 2H), 3.84 (dd, J=11.9, 8.1 Hz, 2H), 3.74-3.68 (m, 2H), 3.42 (d, J=12.5 Hz, 2H), 3.21 (dt, J=25.1, 12.6 Hz, 4H), 3.11 (d, J=5.7 Hz, 2H), 2.07 (d, J=15.0 Hz, 2H), 1.84 (t, J=5.8 Hz, 1H), 1.79 (t, J=5.6 Hz, 1H), 1.73-1.63 (m, 2H), 1.61 (t, J=5.9 Hz, 1H), 1.56 (t, J=5.7 Hz, 1H), 1.50 (d, J=9.9 Hz, 6H) (rotamers)

APCI-MS m/z: 427.1 [MH+]

HPLC (Method A) Retention time: 6.81 min

### EXAMPLE 28

### 3-[(2,2-dimethyl-2,3-dihydro-1-benzofuran-7-yl)methyl]-9-[(3-methylisoxazol-4-yl)carbonyl]-3,9-diazaspiro[5.5]undecane Trifluoroacetate

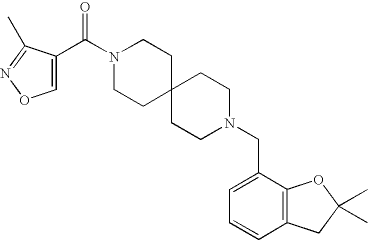

APCI-MS m/z: 424.2 [MH+]

HPLC (Method A) Retention time: 7.28 min

### EXAMPLE 29

### 3-[(2,2-dimethyl-2,3-dihydro-1-benzofuran-7-yl)methyl]-9-(1H-pyrazol-4-ylcarbonyl)-3,9-diazaspiro[5.5]undecane Trifluoroacetate

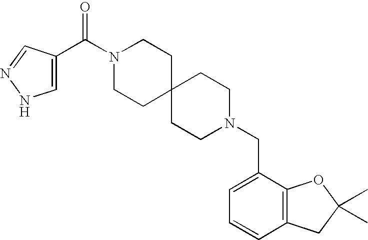

¹H NMR (499.881 MHz, CD3OD) δ 7.89 (s, 2H), 7.30 (dd, J=7.3, 1.0 Hz, 1H), 7.20 (d, J=7.7 Hz, 1H), 6.93 (t, J=7.5 Hz, 1H), 4.27 (s, 2H), 3.72 (dd, J=11.6, 7.2 Hz, 4H), 3.41 (d, J=13.1 Hz, 2H), 3.24-3.15 (m, 2H), 3.11 (s, 2H), 2.04 (d, J=14.4 Hz, 2H), 1.74 (t, J=5.3 Hz, 2H), 1.65 (dd, J=28.1, 3.8 Hz, 3H), 1.54-1.48 (m, 2H), 1.50 (s, 6H)

APCI-MS m/z: 409.2 [MH+]

HPLC (Method A) Retention time: 5.69 min

### EXAMPLE 30

### 3-[(2,2-dimethyl-2,3-dihydro-1-benzofuran-7-yl)methyl]-9-(3-furoyl)-3,9-diazaspiro[5.5]undecane Trifluoroacetate

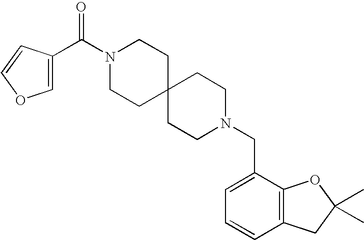

APCI-MS m/z: 409.2 [MH+]

HPLC (Method A) Retention time: 6.98 min

### EXAMPLE 31

### 3-[(2,2-dimethyl-2,3-dihydro-1-benzofuran-7-yl)methyl]-9-(isoxazol-5-ylcarbonyl)-3,9-diazaspiro[5.5]undecane Trifluoroacetate

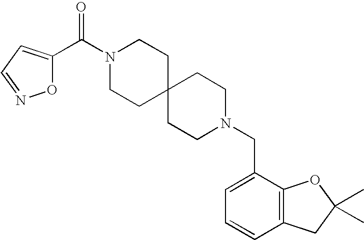

¹H NMR (499.881 MHz, CD3OD) δ 8.51 (d, J=4.3 Hz, 1H), 7.30 (d, J=7.5 Hz, 1H), 7.20 (d, J=7.4 Hz, 1H), 6.93 (t, J=7.5 Hz, 1H), 6.81 (s, 1H), 4.27 (d, J=6.1 Hz, 2H), 3.79-3.72 (m, 2H), 3.67-3.61 (m, 2H), 3.41 (d, J=13.3 Hz, 2H), 3.26-3.15 (m, 2H), 3.11 (s, 2H), 2.06 (d, J=14.9 Hz, 2H), 1.82-1.74 (m, 2H), 1.70-1.60 (m, 2H), 1.59-1.53 (m, 2H), 1.51 (d, J=4.6 Hz, 6H)

APCI-MS m/z: 410.1 [MH+]

HPLC (Method A) Retention time: 6.90 min

### EXAMPLE 32

### 3-[(2,2-dimethyl-2,3-dihydro-1-benzofuran-7-yl)methyl]-9-[(1-methyl-1H-imidazol-4-yl)carbonyl]-3,9-diazaspiro[5.5]undecane Trifluoroacetate

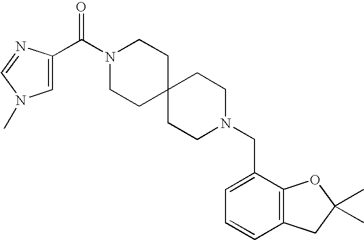

¹H NMR (499.881 MHz, CD3OD) δ 8.70 (s, 1H), 7.90 (s, 1H), 7.30 (dd, J=7.3, 1.1 Hz, 1H), 7.21 (d, J=7.5 Hz, 1H), 6.93 (t, J=7.5 Hz, 1H), 4.28 (s, 2H), 3.92 (s, 3H), 3.77 (bs, 4H), 3.42 (d, J=13.2 Hz, 2H), 3.19 (t, J=12.6 Hz, 2H), 3.11 (s, 2H), 2.04 (d, J=14.0 Hz, 2H), 1.77 (t, J=5.7 Hz, 2H), 1.68 (dd, J=28.2, 4.0 Hz, 2H), 1.55 (t, J=5.8 Hz, 2H), 1.50 (s, 7H)

APCI-MS m/z: 423.2 [MH+]

HPLC (Method A) Retention time: 4.96 min

### EXAMPLE 33

### 1-[5-({9-[(2,2-dimethyl-2,3-dihydro-1-benzofuran-7-yl)methyl]-3,9-diazaspiro[5.5]undec-3-yl}carbonyl)-1H-pyrrol-3-yl]ethanone Trifluoroacetate

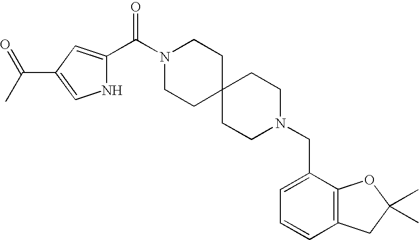

APCI-MS m/z: 450.2 [MH+]

HPLC (Method A) Retention time: 6.67 min

### EXAMPLE 34

### 3-[(2,2-dimethyl-2,3-dihydro-1-benzofuran-7-yl)methyl]-9-(1H-pyrazol-3-ylcarbonyl)-3,9-diazaspiro[5.5]undecane

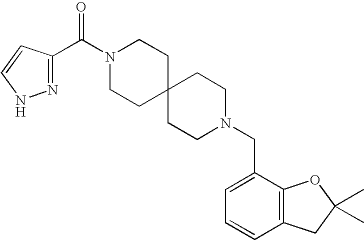

APCI-MS m/z: 409.2 [MH+]

HPLC (Method A) Retention time: 5.73 min

### EXAMPLE 35

### 3-[(2,2-dimethyl-2,3-dihydro-1-benzofuran-7-yl)methyl]-9-(1H-indol-3-ylcarbonyl)-3,9-diazaspiro[5.5]undecane Trifluoroacetate

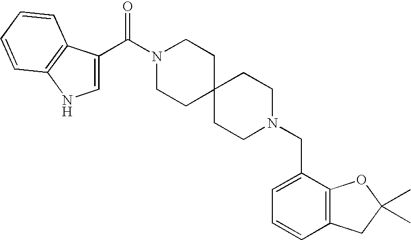

¹H NMR (499.881 MHz, CD3OD) δ 7.64 (d, J=8.0 Hz, 1H), 7.60 (d, J=3.1 Hz, 1H), 7.44 (d, J=8.0 Hz, 1H), 7.29 (d, J=7.3 Hz, 1H), 7.21-7.17 (m, 2H), 7.14 (t, J=7.5 Hz, 1H), 6.93 (t, J=7.5 Hz, 1H), 4.26 (s, 2H), 3.75 (dd, J=11.8, 8.2 Hz, 4H), 3.40 (d, J=13.3 Hz, 2H), 3.23-3.15 (m, 4H), 3.11 (s, 2H), 2.07 (d, J=14.1 Hz, 2H), 1.77-1.72 (m, 2H), 1.69-1.61 (m, 2H), 1.55-1.50 (m, 2H), 1.50 (s, 6H)

APCI-MS m/z: 458.2 [MH+]

HPLC (Method A) Retention time: 7.86 min

### EXAMPLE 36

### 3-[(2,2-dimethyl-2,3-dihydro-1-benzofuran-7-yl)methyl]-9-(1H-indazol-3-ylcarbonyl)-3,9-diazaspiro[5.5]undecane Trifluoroacetate

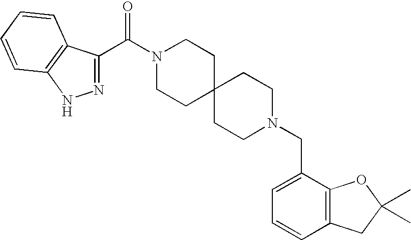

¹H NMR (499.881 MHz, CD3OD) δ 7.92 (d, J=8.3 Hz, 1H), 7.58 (d, J=8.5 Hz, 1H), 7.43 (t, J=7.7 Hz, 1H), 7.30 (d, J=7.1 Hz, 1H), 7.24 (t, J=7.5 Hz, 3H), 7.21 (d, J=7.4 Hz, 2H), 6.93 (t, J=7.5 Hz, 1H), 4.27 (d, J=10.7 Hz, 2H), 3.97 (s, 2H), 3.85 (s, 2H), 3.66 (s, 2H), 3.41 (d, J=13.2 Hz, 2H), 3.28-3.15 (m, 2H), 3.11 (s, 2H), 2.08 (d, J=15.0 Hz, 2H), 1.86-1.74 (m, 2H), 1.72-1.53 (m, 4H), 1.51 (d, J=7.7 Hz, 6H)

APCI-MS m/z: 459.2 [MH+]

HPLC (Method A) Retention time: 7.50 min

### EXAMPLE 37

### 3-[(2,2-dimethyl-2,3-dihydro-1-benzofuran-7-yl)methyl]-9-(1H-indol-2-ylcarbonyl)-3,9-diazaspiro[5.5]undecane Trifluoroacetate

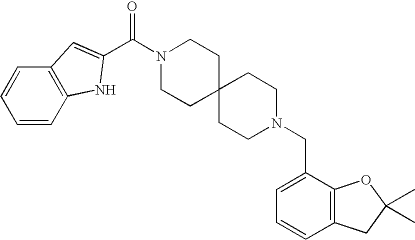

¹H NMR (499.881 MHz, CD3OD) δ 7.60 (d, J=8.0 Hz, 1H), 7.43 (d, J=8.3 Hz, 1H), 7.30 (dd, J=0.5, 6.8 Hz, 1H), 7.24-7.19 (m, 2H), 7.07 (t, J=7.5 Hz, 1H), 6.93 (t, J=7.5 Hz, 1H), 6.80 (s, 1H), 4.28 (s, 2H), 3.94-3.77 (m, 4H), 3.42 (d, J=13.2 Hz, 2H), 3.21 (t, J=12.1 Hz, 2H), 3.12 (s, 2H), 2.07 (d, J=13.9 Hz, 2H), 1.79 (t, J=5.7 Hz, 2H), 1.71-1.63 (m, 2H), 1.57 (t, J=5.7 Hz, 2H), 1.51 (s, 6H)

APCI-MS m/z: 458.2 [MH+]

HPLC (Method A) Retention time: 8.36 min

### EXAMPLE 38

### 3-(2-chloroisonicotinoyl)-9-[(2,2-dimethyl-2,3-dihydro-1-benzofuran-7-yl)methyl]-3,9-diazaspiro[5.5]undecane

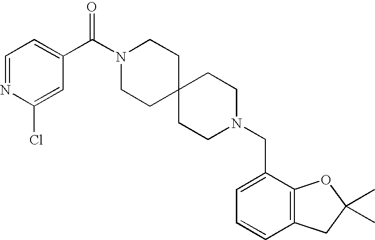

The title compound was prepared by the procedure of Example 8 using Intermediate A and 2-chloropyridine-4-carboxylic acid as starting materials to give the product as a white solid (53 mg, 55%).

¹H NMR (399.99 MHz, DMSO-D6) δ 9.24 (s, 1H), 8.50 (t, 1H), 7.54 (s, 1H), 7.40 (t, 1H), 7.25 (d, 1H), 6.90 (t, 1H), 4.24-4.16 (m, 2H), 3.65-3.52 (m, 2H), 3.31-2.97 (m, 8H), 1.89 (d, J=14.0 Hz, 2H), 1.76-1.25 (m, 12H)

APCI-MS m/z: [MH+] 454.3

HPLC (Method A) Retention time: 7.55 min

HPLC (Method B) Retention time: 9.73 min

### EXAMPLE 39

### [2-({9-[(2,2-dimethyl-2,3-dihydro-1-benzofuran-7-yl)methyl]-3,9-diazaspiro[5.5]undec-3-yl}carbonyl)phenyl]amine Trifluoroacetate

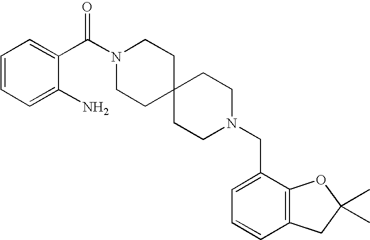

AZ12426941, 793/2102

The title compound was prepared by the synthetic procedure of Example 8 using Intermediate A and 2-[(tert-butoxycarbonyl)amino]benzoic acid as starting materials. The reaction mixture was eluted through silica with EtOAc/Et2NH (95/5), evaporated and treated with 1M methanolic hydrochloric acid (50 ml) for 16 h. The crude product was purified by preparative HPLC (RP-18, gradient acetonitrile/water/TFA from 10/90/0.1 to 95/5/0.1) to give the product as a white solid (76 mg, 60%).

¹H NMR (399.99 MHz, DMSO-D6) δ 7.36 (d, 1H), 7.32-7.23 (m, 2H), 7.16 (d, 1H), 7.09-7.02 (m, 1H), 6.98-6.84 (m, 2H), 4.17 (s, 2H), 3.97-3.30 (m, 4H), 3.21 (d, 2H), 3.12-2.96 (m, 6H), 1.84 (d, 2H), 1.74-1.57 (m, 4H), 1.49-1.33 (m, 8H)

APCI-MS mm/z: [MH+] 434.4

HPLC (Method A) Retention time: 6.81 min

HPLC (Method B) Retention time: 9.65 min

### EXAMPLE 40

### N-[2-({9-[(2,2-dimethyl-2,3-dihydro-1-benzofuran-7-yl)methyl]-3,9-diazaspiro[5.5]undec-3-yl}carbonyl)phenyl]acetamide

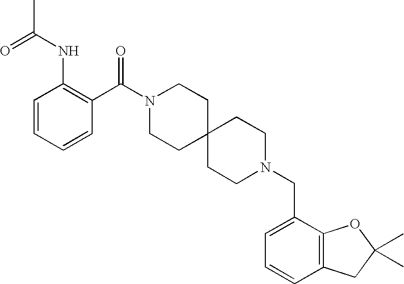

A mixture of Example 39 dihydrochloride salt (70 mg, 0.14 mmol), acetyl chloride (13 μl, 0.17 mmol), N-ethyl-N-isopropylpropan-2-amine (100 μl, 0.58 mmol) and acetonitrile (1 ml) was stirred at room temperature for 1 h then acidified with TFA. The crude product was purified by preparative HPLC (RP-18, gradient acetonitrile/water/aqueous NH₃ from 10/90/0.1 to 95/5/0.1) to give the product as a white solid (28 mg, 41%).

¹H NMR (399.99 MHz, DMSO-D6) δ 9.47 (s, 1H), 7.52 (d, 1H), 7.36 (t, 1H), 7.25-7.14 (m, 2H), 7.06-7.00 (m, 2H), 6.75 (q, 1H), 3.60-3.49 (m, 2H), 3.36 (s, 2H), 3.12 (s, 2H), 3.01-2.94 (m, 2H), 2.40-2.26 (m, 4H), 2.04-1.93 (m, 3H), 1.50-1.40 (m, 6H), 1.40-1.35 (m, 6H), 1.35-1.26 (m, 2H)

APCI-MS m/z: [MH+] 476.2

HPLC (Method A) Retention time: 6.86 min

HPLC (Method B) Retention time: 8.55 min

### EXAMPLE 41

### N-[2-({9-[(2,2-dimethyl-2,3-dihydro-1-benzofuran-7-yl)methyl]-3,9-diazaspiro[5.5]undec-3-yl}carbonyl)phenyl]-2-hydroxyacetamide

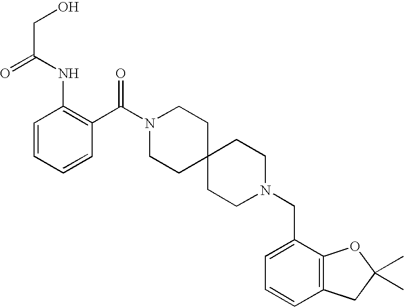

A mixture of Example 39 dihydrochloride salt (70 mg, 0.14 mmol), 2-chloro-2-oxoethyl acetate (13 μl, 0.17 mmol), N-ethyl-N-isopropylpropan-2-amine (100 μl, 0.58 mmol) and acetonitrile (1 ml) was stirred at room temperature for 1 h. Additional 2-chloro-2-oxoethyl acetate (13 μl, 0.17 mmol) was added and mixture heated to 60° C. for 2 h. To the cooled reaction mixture was added water (1 ml) and lithium hydroxide (80 mg, 3.3 mmol) and the reaction mixture was stirred at room temperature for 2 h. The crude product was purified by preparative HPLC (RP-18, gradient acetonitrile/water/aqueous NH₃ from 10/90/0.1 to 95/5/0.1) to give the product as a white solid (34 mg, 50%).

¹H NMR (399.99 MHz, DMSO-D6) δ 8.20 (d, 1H), 7.42 (dd, 1H), 7.32-7.26 (m, 1H), 7.16 (t, 1H), 7.07-7.00 (m, 2H), 6.74 (t, 1H), 3.94 (s, 2H), 3.60 (s, 2H), 3.36 (s, 2H), 3.24 (s, 2H), 2.97 (s, 2H), 2.33 (s, 4H), 1.55-1.28 (m, 14H)

APCI-MS m/z: [MH+] 491.4

HPLC (Method A) Retention time: 6.82 min

HPLC (Method B) Retention time: 8.58 min

### EXAMPLE 42

### 1-[4-({9-[(2,2-dimethyl-2,3-dihydro-1-benzofuran-7-yl)methyl]-3,9-diazaspiro[5.5]undec-3-yl}carbonyl)pyridin-2-yl]pyrrolidin-3-ol

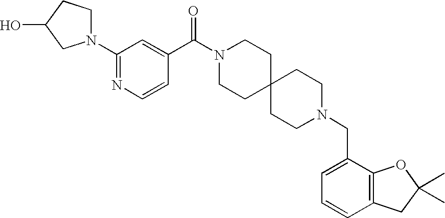

3-(2-chloroisonicotinoyl)-9-[(2,2-dimethyl-2,3-dihydro-1-benzofuran-7-yl)methyl]-3,9-diazaspiro[5.5]undecane (68 mg, 0.15 mmol) and pyrrolidin-3-ol (124 μl, 1.5 mmol) were dissolved in NMP (1 ml) and heated at 200° C. for 10 min in a CEM microwave apparatus. The mixture was diluted with CH3CN and H2O and purified by preparative HPLC (RP-18, gradient acetonitrile/water/TFA 10/90/0.1 to 60/40/0.1) then eluted through a SCX ion exchange column to afford the title compound as a white solid (28 mg, 37%).

¹H NMR (499.881 MHz, CD3OD) δ 8.07 (d, J=5.3 Hz, 1H), 7.08 (d, J=7.7 Hz, 2H), 6.78 (t, J=7.5 Hz, 1H), 6.51 (dd, J=5.3, 1.1 Hz, 1H), 6.42 (s, 1H), 4.53 (dt, J=4.5, 2.2 Hz, 1H), 3.69 (t, J=5.7 Hz, 2H), 3.66 (s, 2H), 3.61-3.51 (m, 5H), 3.43 (d, J=10.9 Hz, 1H), 3.36 (t, J=5.6 Hz, 2H), 3.02 (s, 2H), 2.56 (s, 4H), 2.19-2.10 (m, 1H), 2.07-2.02 (m, 1H), 1.61 (d, J=5.4 Hz, 2H), 1.56 (t, J=5.7 Hz, 2H), 1.47-1.42 (m, 2H), 1.44 (s, 6H)

APCI-MS m/z: 505.2 [MH+]

HPLC (Method A) Retention time: 5.26 min

HPLC (Method B) Retention time: 8.04 min

### EXAMPLE 43

### 3-[(2,2-dimethyl-2,3-dihydro-1-benzofuran-7-yl)methyl]-9-{2-[(2S)-2-(methoxymethyl)pyrrolidin-1-yl]isonicotinoyl}-3,9-diazaspiro[5.5]undecane

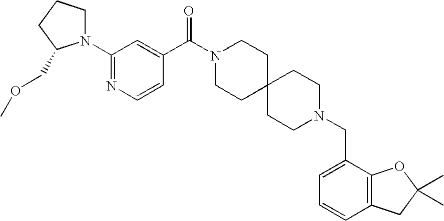

The title compound was prepared according to Example 42 using 3-(2-chloroisonicotinoyl)-9-[(2,2-dimethyl-2,3-dihydro-1-benzofuran-7-yl)methyl]-3,9-diazaspiro[5.5]undecane (68 mg, 0.15 mmol) and (2S)-2-(methoxymethyl)pyrrolidine (173 mg, 1.5 mmol) to afford the product as a white solid (12 mg, 15%).

¹H NMR (499.881 MHz, CD3OD) δ 8.09 (d, J=5.1 Hz, 1H), 7.08 (d, J=7.7 Hz, 2H), 6.78 (t, J=7.4 Hz, 1H), 6.51 (d, J=5.1 Hz, 1H), 6.47 (s, 1H), 4.22-4.16 (m, 1H), 3.76-3.64 (m, 2H), 3.67 (s, 2H), 3.61-3.49 (m, 4H), 3.39-3.32 (m, 5H), 3.02 (s, 2H), 2.57 (s, 4H), 2.16-1.96 (m, 4H), 1.67-1.53 (m, 6H), 1.47-1.42 (m, 2H), 1.45 (s, 6H)

APCI-MS m/z: 533.5 [MH+]

HPLC (Method A) Retention time: 6.20 min

HPLC (Method B) Retention time: 10.22 min

### EXAMPLE 44

### 4-({9-[(2,2-dimethyl-2,3-dihydro-1-benzofuran-7-yl)methyl]-3,9-diazaspiro[5.5]undec-3-yl}carbonyl)-N-methylpyridin-2-amine

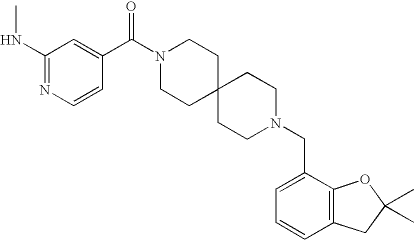

The title compound was prepared according to Example 42 using 3-(2-chloroisonicotinoyl)-9-[(2,2-dimethyl-2,3-dihydro-1-benzofuran-7-yl)methyl]-3,9-diazaspiro[5.5]undecane (68 mg, 0.15 mmol) and methylamine (33% in EtOH) (1.41 ml, 15 mmol) to afford the product as a white solid (12 mg, 18%).

¹H NMR (499.881 MHz, CD3OD) δ 8.00 (d, J=5.3 Hz, 1H), 7.08 (dd, J=7.2, 3.9 Hz, 2H), 6.79 (t, J=7.5 Hz, 1H), 6.47 (dd, J=5.3, 1.3 Hz, 1H), 6.42 (s, 1H), 3.68 (t, J=5.9 Hz, 2H), 3.66 (s, 2H), 3.60 (s, 2H), 3.36 (t, J=5.7 Hz, 2H), 3.03 (s, 2H), 2.87 (s, 3H), 2.59 (s, 4H), 1.68-1.58 (m, 6H), 1.55 (t, J=5.7 Hz, 2H), 1.47-1.42 (m, 2H), 1.44 (s, 6H)

APCI-MS m/z: 449.4 [MH+]

HPLC (Method A) Retention time: 5.32 min

HPLC (Method B) Retention time: 8.52 min

### EXAMPLE 45

### N-[2-({9-[(2,2-dimethyl-2,3-dihydro-1-benzofuran-7-yl)methyl]-3,9-diazaspiro[5.5]undec-3-yl}carbonyl)phenyl]-6-methyl-2,4-dioxo-1,2,3,4-tetrahydropyrimidine-5-sulfonamide

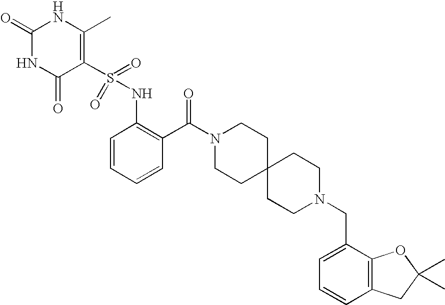

The title compound was prepared by the procedure of Example 40 using Example 39 dihydrochloride salt and 6-methyl-2,4-dioxo-1,2,3,4-tetrahydropyrimidine-5-sulfonyl chloride as starting materials to give the product as a white solid (6 mg, 7%).

¹H NMR (399.99 MHz, DMSO-D6) δ 7.48-7.36 (m, 2H), 7.27 (d, 1H), 7.23-7.16 (m, 1H), 7.14-7.05 (m, 2H), 6.86-6.73 (m, 1H), 3.67-3.38 (m, 4H), 3.24-3.06 (m, 4H), 3.00 (s, 3H), 2.33 (s, 4H), 1.62-1.31 (m, 14H)

APCI-MS m/z: [MH+] 622.2

HPLC (Method A) Retention time: 7.33 min

HPLC (Method B) Retention time: 4.67 min

### EXAMPLE 46

### 1-[2-({9-[(2,2-dimethyl-2,3-dihydro-1-benzofuran-7-yl)methyl]-3,9-diazaspiro[5.5]undec-3-yl}carbonyl)phenyl]imidazolidine-2,4-dione

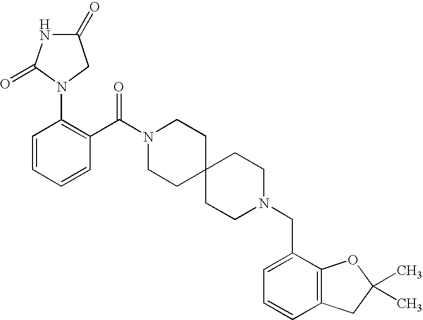

A mixture of Example 39 dihydrochloride salt (70 mg, 0.14 mmol), chloroacetyl isocyanate (14 μl, 0.17 mmol), N-ethyl-N-isopropylpropan-2-amine (100 μl, 0.58 mmol) and tetrahydrofuran (1 ml) was stirred at room temperature for 1 h. Sodium hydride (15 mg, 0.63 mmol) was added and the reaction mixture stirred at 60° C. for 1 h. The crude product was purified by preparative HPLC (RP-18, gradient acetonitrile/water/aqueous NH₃ from 10/90/0.1 to 95/5/0.1) to give the product as a white solid (17 mg, 24%).

¹H NMR (399.99 MHz, DMSO-D6) δ 7.63-7.30 (m, 4H), 7.21-6.99 (m, 2H), 6.88-6.64 (m, 1H), 4.52-4.06 (m, 2H), 3.60-3.47 (m, 2H), 3.26-3.11 (m, 4H), 3.06-2.91 (m, 2H), 2.40-2.21 (m, 4H), 1.62-1.24 (m, 14H)

APCI-MS m/z: [MH+] 517.2

HPLC (Method A) Retention time: 6.81 min

HPLC (Method B) Retention time: 8.63 min

### EXAMPLE 47

### N-[2-({9-[(2,2-dimethyl-2,3-dihydro-1-benzofuran-7-yl)methyl]-3,9-diazaspiro[5.5]undec-3-yl}carbonyl)phenyl]nicotinamide 1-oxide

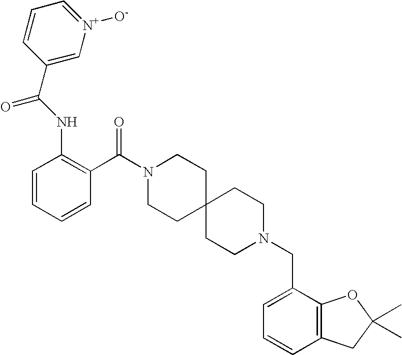

The title compound was prepared by the procedure of Example 8 using Example 39 dihydrochloride salt and nicotinic acid 1-oxide as starting materials to give the product as a white solid (8 mg, 9%).

¹H NMR (399.99 MHz, DMSO-D6) δ 8.60 (s, 1H), 8.40 (d, 1H), 7.74 (d, 1H), 7.61-7.42 (m, 3H), 7.37-7.28 (m, 2H), 7.03 (d, 2H), 6.74 (d, 1H), 3.50 (s, 2H), 3.35 (s, 2H), 3.26-3.16 (m, 2H), 2.97 (s, 2H), 2.36-2.26 (m, 4H), 1.44-1.31 (m, 14H)

APCI-MS m/z: [MH+] 555.0

HPLC (Method A) Retention time: 6.56 min

HPLC (Method B) Retention time: 7.71 min

### EXAMPLE 48

### N-[2-({9-[(2,2-dimethyl-2,3-dihydro-1-benzofuran-7-yl)methyl]-3,9-diazaspiro[5.5]undec-3-yl}carbonyl)phenyl]-1-methyl-L-prolinamide

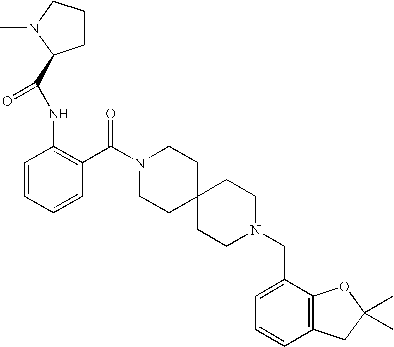

The title compound was prepared by the procedure of Example 8 using Example 39 dihydrochloride salt and 1-methyl-L-proline as starting materials to give the product as a white solid (9 mg, 10%).

¹H NMR (399.99 MHz, DMSO-D6) δ 9.85 (s, 1H), 8.11 (d, 1H), 7.40 (s, 1H), 7.25 (d, 1H), 7.15 (d, 1H), 7.03 (d, 2H), 6.75 (d, 1H), 3.73-3.47 (m, 2H), 3.35 (s, 2H), 3.29 (s, 2H), 3.26-3.17 (m, 2H), 3.11-3.03 (m, 1H), 2.97 (s, 2H), 2.94-2.87 (m, 2H), 2.42-2.11 (m, 7H), 1.75 (s, 2H), 1.51-1.19 (m, 14H)

APCI-MS m/z: [MH+] 545.5

HPLC (Method A) Retention time: 6.31 min

HPLC (Method B) Retention time: 10.61 min

### EXAMPLE 49

### N-[2-({9-[(2,2-dimethyl-2,3-dihydro-1-benzofuran-7-yl)methyl]-3,9-diazaspiro[5.5]undec-3-yl}carbonyl)phenyl]tetrahydrofuran-2-carboxamide

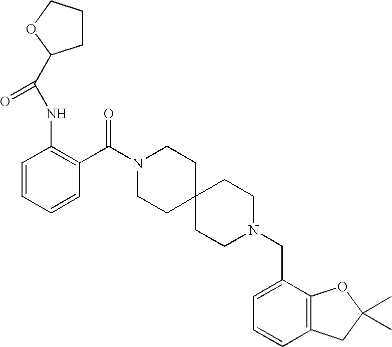

The title compound was prepared by the procedure of Example 8 using Example 39 dihydrochloride salt and tetrahydrofuran-2-carboxylic acid as starting materials to give the product as a white solid (17 mg, 19%).

¹H NMR (399.99 MHz, DMSO-D6) δ 9.61 (s, 1H), 7.45 (d, 1H), 7.40-7.35 (m, 1H), 7.27-7.16 (m, 2H), 7.04 (d, 2H), 6.74 (t, 1H), 3.86 (t, 1H), 3.77-3.64 (m, 3H), 3.54 (s, 2H), 3.29 (s, 2H), 3.15 (t, J=7.5 Hz, 3H), 2.97 (s, 2H), 2.33 (s, 4H), 2.06-1.96 (m, 2H), 1.53-1.21 (m, 14H)

APCI-MS m/z: [MH+] 532.4

HPLC (Method A) Retention time: 7.17 min

HPLC (Method B) Retention time: 8.89 min

### EXAMPLE 50

### N-[2-({9-[(2,2-dimethyl-2,3-dihydro-1-benzofuran-7-yl)methyl]-3,9-diazaspiro[5.5]undec-3-yl}carbonyl)phenyl]-5-oxoprolinamide

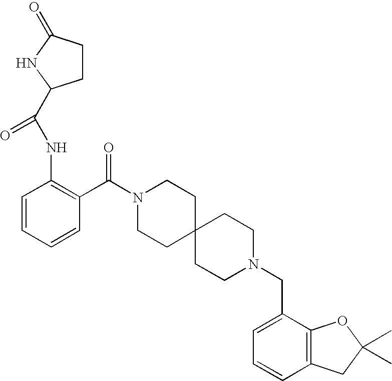

The title compound was prepared by the procedure of Example 8 using Example 39 dihydrochloride salt and 5-oxoproline as starting materials to give the product as a white solid (31 mg, 34%).

¹H NMR (399.99 MHz, DMSO-D6) δ 9.62 (s, 1H), 7.90 (s, 1H), 7.54 (s, 1H), 7.46-7.37 (m, 1H), 7.32-7.17 (m, 2H), 7.10-7.00 (m, 2H), 6.79-6.69 (m, 1H), 4.22-4.14 (m, 1H), 3.62-3.49 (m, 2H), 3.38-3.33 (m, 2H), 3.26-3.10 (m, 2H), 2.97 (s, 2H), 2.41-2.24 (m, 5H), 2.17-2.06 (m, 2H), 2.04-1.92 (m, 1H), 1.55-1.27 (m, 14H)

APCI-MS m/z: [MH+] 545.2

HPLC (Method A) Retention time: 6.51 min

HPLC (Method B) Retention time: 7.93 min

### EXAMPLE 51

### [4-({9-[(2,2-dimethyl-2,3-dihydro-1-benzofuran-7-yl)methyl]-3,9-diazaspiro[5.5]undec-3-yl}carbonyl)phenyl]amine Trifluoroacetate

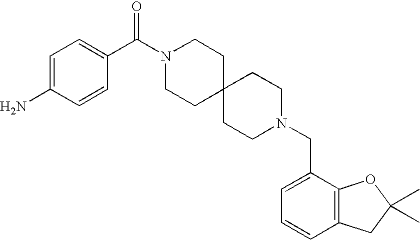

The title compound was prepared by the procedure of Example 39 using Intermediate A dihydrochloride and 4-[(tert-butoxycarbonyl)amino]benzoic acids starting materials to give the product as a white solid (88 mg, 80%).

¹H NMR (399.99 MHz, DMSO-D6) δ 7.29 (d, J=7.6 Hz, 1H), 7.23 (d, J=7.0 Hz, 1H), 7.16 (m, 2H), 6.90 (t, 1H), 6.68 (d, 2H), 4.20 (s, 2H), 3.44 (s, 2H), 3.24 (d, 2H), 3.15-2.98 (m, 4H), 1.89 (d, 2H), 1.66-1.40 (m, 10H), 1.38-1.28 (m, 2H), 1.38-1.28 (m, 2H)

APCI-MS m/z: [MH+] 434.4

HPLC (Method A) Retention time: 5.54 min

HPLC (Method B) Retention time: 8.89 min

### EXAMPLE 52

### 3-[(2,2-dimethyl-2,3-dihydro-1-benzofuran-7-yl)methyl]-9-(3-methylisonicotinoyl)-3,9-diazaspiro[5.5]undecane Trifluoroacetate

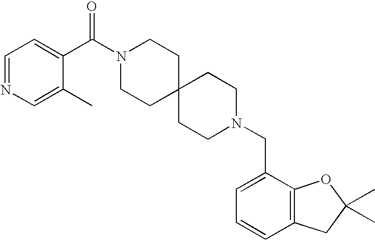

A mixture of Intermediate A (77 mg, 0.20 mmol), HBTU (83 mg, 0.22 mmol), 3-methyl-4-pyridinecarboxylic acid (30 mg, 0.22 mmol), triethylamine (140 μl, 1.0 mmol) and acetonitrile (1 ml) was stirred at room temperature until the reaction was complete, then acidified with acetic acid. The crude product was purified by preparative HPLC (RP-18, gradient acetonitrile/water/TFA from 10/90/0.1 to 95/5/0.1) to give the product as a white solid (13 mg, 14%).

¹H NMR (399.99 MHz, CD3OD) δ 8.81-8.68 (m, 2H), 7.92-7.81 (m, 1H), 7.29 (t, 1H), 7.20 (t, 1H), 6.92 (q, 1H), 4.26 (d, 2H), 3.81 (d, 2H), 3.45-3.36 (m, 2H), 3.28-3.07 (m, 6H), 2.46 (s, 3H), 2.17-1.95 (m, 2H), 1.89-1.37 (m, 12H)

APCI-MS m/z: 434.6 [MH+]

HPLC (Method A) Retention time: 5.30 min

HPLC (Method B) Retention time: 8.34 min

### EXAMPLE 53

### 2-({9-[(2,2-dimethyl-2,3-dihydro-1-benzofuran-7-yl)methyl]-3,9-diazaspiro[5.5]undec-3-yl}carbonyl)pyridin-4-amine Trifluoroacetate

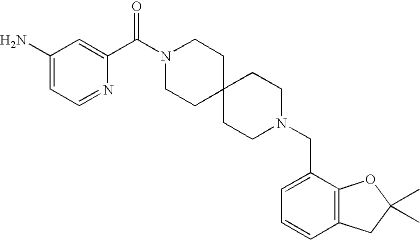

The title compound was prepared by the synthesis procedure of Example 52 using Intermediate A and 4-amino-2-pyridinecarboxylic acid as starting materials. The crude product was purified by preparative HPLC (RP-18, gradient acetonitrile/water/TFA from 10/90/0.1 to 95/5/0.1) to give the product as a white solid (6 mg, 6%).

¹H NMR (399.99 MHz, CD3OD) δ 8.07 (d, 1H), 7.30 (d, 1H), 7.20 (d, 1H), 6.93 (t, 1H), 6.90-6.84 (m, 2H), 4.27 (s, 2H), 3.75 (s, 2H), 3.53-3.37 (m, 4H), 3.27-3.07 (m, 4H), 2.04 (d, 2H), 1.85-1.45 (m, 12H)

APCI-MS m/z: 435.6 [MH+]

HPLC (Method A) Retention time: 5.09 min

HPLC (Method B) Retention time: 7.62 min

### EXAMPLE 54

### 3-[(2,2-dimethyl-2,3-dihydro-1-benzofuran-7-yl)methyl]-9-(2-methylisonicotinoyl)-3,9-diazaspiro[5.5]undecane Trifluoroacetate

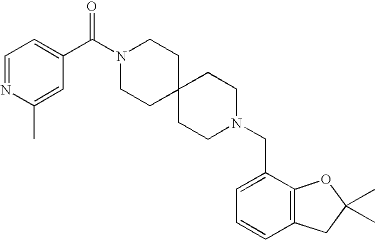

The title compound was prepared by the procedure of Example 52 using Intermediate A and 2-methyl-4-pyridinecarboxylic acid as starting materials to give the product as a white solid (28 mg, 32%).

¹H NMR (399.99 MHz, CD3OD) δ 8.75 (t, 1H), 7.85 (s, 1H), 7.82-7.75 (m, 1H), 7.29 (t, 1H), 7.20 (t, 1H), 6.96-6.86 (m, 1H), 4.26 (d, 2H), 3.84-3.70 (m, 2H), 3.47-3.32 (m, 4H), 3.27-3.04 (m, 4H), 2.79 (m, 3H), 2.03 (d, 2H), 1.87-1.54 (m, 6H), 1.49 (d, 6H)

APCI-MS m/z: 434.6 [MH+]

HPLC (Method A) Retention time: 4.82 min

HPLC (Method B) Retention time: 8.38 min

### EXAMPLE 55

### 6-({9-[(2,2-dimethyl-2,3-dihydro-1-benzofuran-7-yl)methyl]-3,9-diazaspiro[5.5]undec-3-yl}carbonyl)pyridin-3-amine Trifluoroacetate

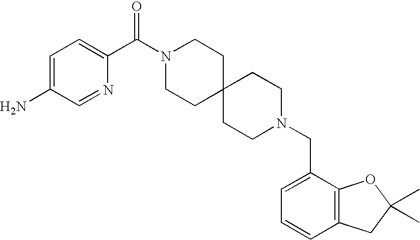

A mixture of Intermediate A (77 mg, 0.20 mmol), 1-ethyl-3-(3-dimethylaminopropyl) carbodiimide hydrochloride (42 mg, 0.22 mmol), 5-aminopicolinic acid (30 mg, 0.22 mmol), triethylamine (140 μl, 1.0 mmol) and acetonitrile (1 ml) was stirred at room temperature overnight and acidified with acetic acid. The crude product was purified by preparative HPLC (RP-18, gradient acetonitrile/water/TFA from 10/90/0.1 to 95/5/0.1) to give the product as a white solid (9 mg, 11%).

¹H NMR (399.99 MHz, CD3OD) δ 7.96 (d, 1H), 7.44 (d, 1H), 7.29 (d, 1H), 7.22-7.17 (m, 2H), 6.93 (t, 1H), 4.28 (s, 2H), 3.75-3.52 (m, 2H), 3.44-3.35 (m, 3H), 3.25-3.13 (m, 2H), 3.11 (m, 3H), 2.04 (d, 2H), 1.78-1.58 (m, 6H), 1.54 (s, 6H)

APCI-MS m/z: 435.6 [MH+]

HPLC (Method A) Retention time: 5.34 min

HPLC (Method B) Retention time: 7.75 min

### EXAMPLE 56

### 4-({9-[(2,2-dimethyl-2,3-dihydro-1-benzofuran-7-yl)methyl]-3,9-diazaspiro[5.5]undec-3-yl}carbonyl)pyridazin-3-amine Trifluoroacetate

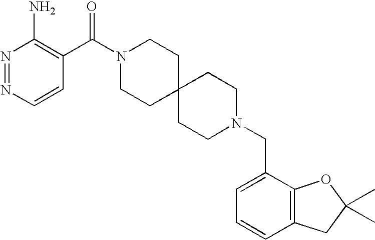

The title compound was prepared by the procedure of Example 52 using Intermediate A and 3-amino-4-pyridazinecarboxylic acid as starting materials to give the product as a white solid (40 mg, 46%).

¹H NMR (399.99 MHz, CD3OD) δ 8.53 (d, 1H), 7.68 (d, 1H), 7.29 (d, 1H), 7.20 (d, 1H), 6.92 (t, 1H), 4.27 (s, 2H), 3.76 (s, 2H), 3.47-3.36 (m, 3H), 3.27-3.14 (m, 2H), 3.11 (m, 3H), 2.03 (d, 2H), 1.89-1.56 (m, 6H), 1.50 (s, 6H)

APCI-MS m/z: 436.6 [MH+]

HPLC (Method A) Retention time: 5.04 min

HPLC (Method B) Retention time: 7.17 min

### EXAMPLE 57

### {[2-({9-[(2,2-dimethyl-2,3-dihydro-1-benzofuran-7-yl)methyl]-3,9-diazaspiro[5.5]undec-3-yl}carbonyl)pyridin-4-yl]methyl}amine Trifluoroacetate

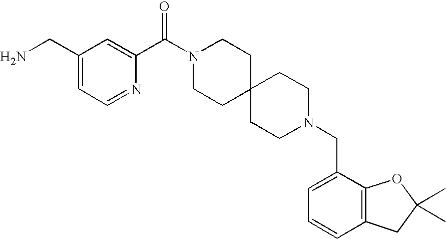

A mixture of Intermediate A (77 mg, 0.20 mmol), HBTU (83 mg, 0.22 mmol), N-Boc-3-(aminomethyl)benzoic acid (56 mg, 0.22 mmol), triethylamine (140 μl, 1.0 mmol) and acetonitrile (1 ml) was stirred at room temperature overnight. The solvent was removed and a solution of methanol and acetyl chloride was added to the light yellow oil. This mixture was stirred overnight, the solvent was removed and the crude product was purified by preparative HPLC (RP-18, gradient acetonitrile/water/TFA from 10/90/0.1 to 95/5/0.1) to give the product as a white solid (9 mg, 10%).

¹H NMR (399.99 MHz, CD3OD) δ 8.66 (t, 1H), 7.64 (s, 1H), 7.55 (t, 1H), 7.32-7.26 (m, 1H), 7.20 (t, 1H), 6.92 (td, 1H), 4.30-4.22 (m, 4H), 3.83-3.74 (m, 2H), 3.49-3.35 (m, 3H), 3.27-3.13 (m, 2H), 3.11 (m, 3H), 2.04 (d, 2H), 1.84-1.55 (m, 6H), 1.50 (d, 6H)

APCI-MS m/z: 449.6 [MH+]

HPLC (Method A) Retention time: 5.04 min

HPLC (Method B) Retention time: 7.17 min

### EXAMPLE 58

### 4-({9-[(2,2-dimethyl-2,3-dihydro-1-benzofuran-7-yl)methyl]-3,9-diazaspiro[5.5]undec-3-yl}carbonyl)quinolin-2-ol Trifluoroacetate (Salt)

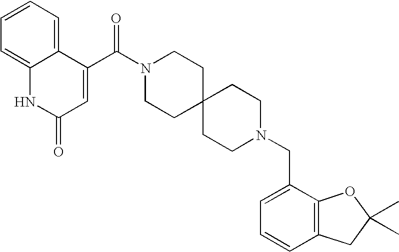

The title compound was prepared by the procedure of Example 52 using Intermediate A and 2-hydroxyquinoline-4-carboxylic acid as starting materials to give the product as a white solid (47 mg, 48%).

¹H NMR (399.99 MHz, CD3OD) δ 7.65-7.59 (m, 1H), 7.52 (d, 1H), 7.44-7.40 (m, 1H), 7.34-7.26 (m, 2H), 7.19 (t, 1H), 6.92 (q, 1H), 6.56 (d, 1H), 4.25 (d, 2H), 3.93-3.77 (m, 2H), 3.44-3.34 (m, 3H), 3.28-3.15 (m, 2H), 3.11 (m, 3H), 2.13-2.00 (m, 2H), 1.93-1.54 (m, 6H), 1.54-1.30 (m, 6H)

APCI-MS m/z: 486.6 [MH+]

HPLC (Method A) Retention time: 6.78 min

HPLC (Method B) Retention time: 8.58 min

### EXAMPLE 59

### 3-[(2,2-dimethyl-2,3-dihydro-1-benzofuran-7-yl)methyl]-9-(1,8-naphthyridin-2-ylcarbonyl)-3,9-diazaspiro[5.5]undecane Trifluoroacetate

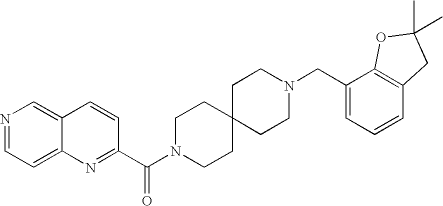

The title compound was prepared by the procedure of Example 52 using Intermediate A and 1,8-naphthyridine-2-carboxylic acid as starting materials to give the product as a white solid (41 mg, 43%).

¹H NMR (399.99 MHz, CD3OD) δ 9.81 (d, 1H), 8.96 (dd, 1H), 8.86 (t, 1H), 8.43 (dd, 1H), 8.05 (dd, 1H), 7.29 (t, 1H), 7.20 (t, 1H), 6.92 (q, 1H), 4.27 (d, 2H), 3.89-3.82 (m, 2H), 3.52-3.37 (m, 3H), 3.29-3.14 (m, 2H), 3.11 (m, 3H), 2.09 (d, 2H), 1.90-1.60 (m, 6H), 1.58-1.45 (m, 6H)

APCI-MS m/z: 471.6 [MH+]

HPLC (Method A) Retention time: 5.88 min

HPLC (Method B) Retention time: 8.31 min

### EXAMPLE 60

### 3-[(2,2-dimethyl-2,3-dihydro-1-benzofuran-7-yl)methyl]-9-(1,6-naphthyridin-2-ylcarbonyl)-3,9-diazaspiro[5.5]undecane Trifluoroacetate

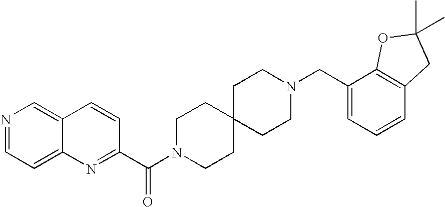

The title compound was prepared by the procedure of Example 52 using Intermediate A and 1,6-naphthyridine-2-carboxylic acid as starting materials to give the product as a white solid (33 mg, 35%).

¹H NMR (399.99 MHz, CD3OD) δ 9.81 (d, 1H), 8.96 (dd, 1H), 8.86 (t, 1H), 8.43 (dd, 1H), 8.05 (dd, 1H), 7.29 (t, 1H), 7.20 (t, 1H), 6.92 (q, 1H), 4.27 (d, 2H), 3.90-3.82 (m, 2H), 3.53-3.37 (m, 3H), 3.29-3.14 (m, 2H), 3.11 (m, 3H), 2.09 (d, 2H), 1.90-1.59 (m, 6H), 1.58-1.45 (m, 6H)

APCI-MS m/z: 471.6 [MH+]

HPLC (Method A) Retention time: 5.35 min

HPLC (Method B) Retention time: 8.44 min

### EXAMPLE 61

### 4-({9-[(2,2-dimethyl-2,3-dihydro-1-benzofuran-7-yl)methyl]-3,9-diazaspiro[5.5]undec-3-yl}carbonyl)-6-methoxypyridin-3-amine

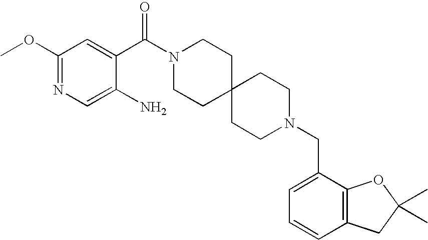

The title compound was prepared by the procedure of Example 52 using Intermediate A and 5-amino-2-methoxy-4-pyridinecarboxylic acid as starting materials. The crude product was purified by preparative HPLC (RP-18, gradient acetonitrile/water/NH4OAc 10:90:0.1 to 95:5:0.1) to give the product as a white solid (39 mg, 42%).

¹H NMR (399.99 MHz, CD3OD) δ 7.71 (s, 1H), 7.08 (d, 2H), 6.78 (t, 1H), 6.54 (s, 1H), 3.82 (s, 3H), 3.71 (s, 2H), 3.57 (s, 2H), 3.37-3.33 (m, 2H), 3.02 (s, 2H), 2.56 (s, 4H), 1.66-1.40 (m, 14H)

APCI-MS m/z: 465.6 [MH+]

HPLC (Method A) Retention time: 5.98 min

HPLC (Method B) Retention time: 8.59 min

### EXAMPLE 62

### 4-({9-[(2,2-dimethyl-2,3-dihydro-1-benzofuran-7-yl)methyl]-3,9-diazaspiro[5.5]undec-3-yl}carbonyl)-2-methylquinolin-3-amine Trifluoroacetate

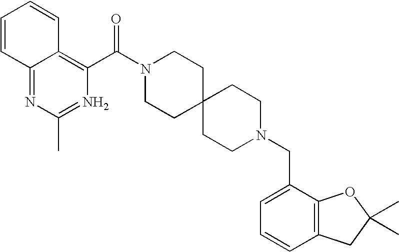

The title compound was prepared by the procedure of Example 52 using Intermediate A and 3-amino-2-methylquinoline-4-carboxylic acid as starting materials. The crude product was purified by preparative HPLC (RP-18, gradient acetonitrile/water/NOAc 10:90:0.1 and acetonitrile/water/TFA from 10/90/0.1 to 95/5/0.1) to give the product as a white solid (12 mg, 12%).

¹H NMR (399.99 MHz, CD3OD) δ 8.00-7.93 (m, 1H), 7.74-7.66 (m, 3H), 7.32-7.14 (m, 2H), 6.92 (q, 1H), 4.25 (d, 2H), 4.08-3.81 (m, 2H), 3.46-3.34 (m, 3H), 3.28-3.17 (m, 2H), 3.10 (d, 3H), 2.82 (d, 3H), 2.12-1.95 (m, 2H), 1.90-1.57 (m, 6H), 1.55-1.26 (m, 6H)

APCI-MS m/z: 499.7 [MH+]

HPLC (Method A) Retention time: 5.89 min

HPLC (Method B) Retention time: 9.11 min

### EXAMPLE 63

### 7-({9-[(2,2-dimethyl-2,3-dihydro-1-benzofuran-7-yl)methyl]-3,9-diazaspiro[5.5]undec-3-yl}carbonyl)-1H-indole-2,3-dione Trifluoroacetate

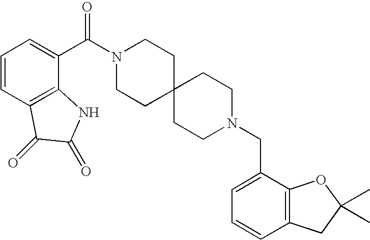

The title compound was prepared by the procedure of Example 52 using Intermediate A and 2,3-dioxoindoline-7-carboxylic acid as starting materials. The crude product was purified by preparative HPLC (RP-18, gradient acetonitrile/water/NH₄OAc 10:90:0.1 and acetonitrile/water/TFA from 10/90/0.1 to 95/5/0.1) to give the product as a light yellow solid (19 mg, 13%).

¹H NMR (399.99 MHz, CD3OD) δ 7.65 (d, 0.5H), 7.54 (d, 0.5H), 7.48 (d, 0.5H) 7.32-7.25 (m, 1.5H), 7.23-7.11 (m, 2H), 6.92 (t, 1H), 4.26 (s, 2H), 3.76 (s, 2H), 3.51-3.36 (m, 3H), 3.26-3.14 (m, 2H), 3.11 (s, 3H), 2.03 (d, 2H), 1.87-1.55 (m, 6H), 1.50 (s, 6H).

APCI-MS m/z: 488.6 [MH+]

HPLC (Method A) Retention time: 7.11 min

HPLC (Method B) Retention time: 5.24 min and 7.43 min

### EXAMPLE 64

### 3-({9-[(2,2-dimethyl-2,3-dihydro-1-benzofuran-7-yl)methyl]-3,9-diazaspiro[5.5]undec-3-yl}carbonyl)pyridin-4-amine Trifluoroacetate

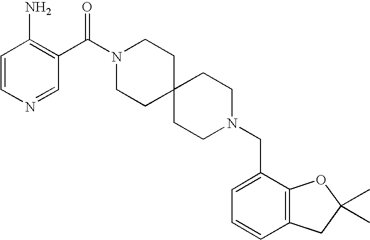

A mixture of Intermediate A (77 mg, 0.20 mmol), PYBOP (114 mg, 0.22 mmol), 4-aminonicotinic acid (37 mg, 0.22 mmol), triethylamine (140 μl, 1.0 mmol) and dichloromethane (1 ml) was stirred at room temperature until the reaction was complete, and then acidified with TFA. The crude product was purified by preparative HPLC (RP-18, gradient acetonitrile/water/TFA from 10/90/0.1 to 95/5/0.1) to give the product as a white solid (41 mg, 31%).

¹H NMR (399.99 MHz, DMSO-D6) δ 8.13-8.01 (m, 2H), 7.22 (dd, 2H), 6.87 (t, 1H), 6.78 (s, 1H), 4.08 (s, 2H), 3.55-3.22 (m, 6H), 3.04 (s, 4H), 1.74-1.36 (m, 14H)

APCI-MS m/z: 435.6 [MH+]

HPLC (Method A) Retention time: 4.92 min

HPLC (Method B) Retention time: 8.05 min

### EXAMPLE 65

### 5-({9-[(2,2-dimethyl-2,3-dihydro-1-benzofuran-7-yl)methyl]-3,9-diazaspiro[5.5]undec-3-yl}carbonyl)pyridin-3-amine

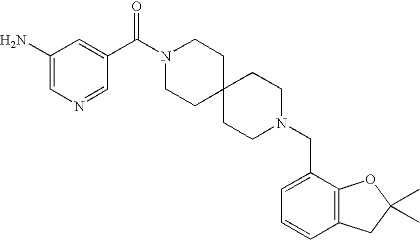

The title compound was prepared by the procedure of Example 52 using Intermediate A and 5-aminonicotinic acid as starting materials to give the product as a white solid (14 mg, 16%).

¹H NMR (399.99 MHz, CD3OD) δ 8.00 (s, 1H), 7.76 (s, 1H), 7.07 (d, 2H), 7.02 (t, 1H), 6.78 (t, 1H), 3.69 (s, 2H), 3.53 (s, 2H), 3.38 (s, 2H), 3.01 (s, 2H), 2.58 (s, 4H), 1.66-1.49 (m, 7H), 1.43 (m, 7H)

APCI-MS m/z: 435.6 [MH+]

HPLC (Method A) Retention time: 5.17 min

HPLC (Method B) Retention time: 7.52 min

### EXAMPLE 66

### 3-[(2,2-dimethyl-2,3-dihydro-1-benzofuran-7-yl)methyl]-9-(1H-indol-7-ylcarbonyl)-3,9-diazaspiro[5.5]undecane Acetate

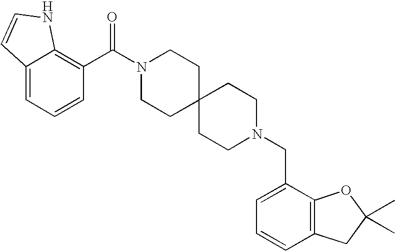

The title compound was prepared by the procedure of Example 52 using Intermediate A and 1H-indole-7-carboxylic acid as starting materials. The crude product was purified by preparative HPLC (RP-18, gradient acetonitrile/water/NH4OAc 10:90:0.1 to 95:5:0.1) to give the product as a white solid (60 mg, 58%).

¹H NMR (399.99 MHz, CD3OD) δ 7.66 (dd, 1H), 7.29 (d, 1H), 7.22 (d, 1H), 7.18-7.05 (m, 3H), 6.87 (t, 1H), 6.52 (d, 1H), 4.04 (s, 2H), 3.93-3.38 (m, 4H), 3.11-3.00 (m, 6H), 1.94 (s, 2H), 1.82-1.41 (m, 12H)

APCI-MS m/z: 458.6 [MH+]

HPLC (Method A) Retention time: 8.81 min

HPLC (Method B) Retention time: 11.00 min

### EXAMPLE 67

### 3-[(2,2-dimethyl-2,3-dihydro-1-benzofuran-7-yl)methyl]-9-(1H-indol-5-ylcarbonyl)-3,9-diazaspiro[5.5]undecane

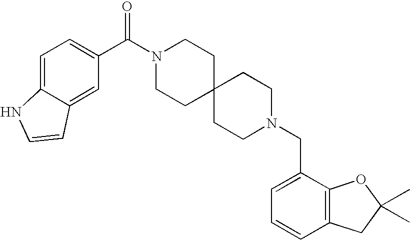

The title compound was prepared by the procedure of Example 52 using Intermediate A and indole-5-carboxylic acid as starting materials. The crude product was purified by preparative HPLC (RP-18, gradient acetonitrile/water/NH4OAc 10:90:0.1 to 95:5:0.1) to give the product as a white solid (63 mg, 69%).

¹H NMR (399.99 MHz, CD3OD) δ 7.63 (s, 1H), 7.43 (d, 1H), 7.31 (d, 1H), 7.15 (dd, 1H), 7.08 (d, 2H), 6.78 (t, 1H), 6.51 (d, 1H), 3.80-3.39 (m, 6H), 3.02 (s, 2H), 2.57 (s, 4H), 1.68-1.39 (m, 14H)

APCI-MS m/z: 458.6 [MH+]

HPLC (Method A) Retention time: 7.66 min

HPLC (Method B) Retention time: 10.26 min

### EXAMPLE 68

### 3-[(2,2-dimethyl-2,3-dihydro-1-benzofuran-7-yl)methyl]-9-(1H-indol-6-ylcarbonyl)-3,9-diazaspiro[5.5]undecane Acetate

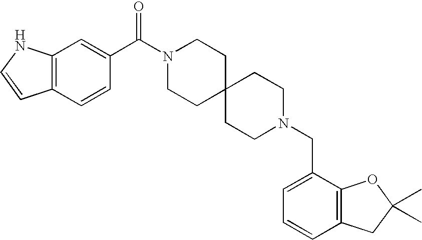

The title compound was prepared by the procedure of Example 52 using Intermediate A and indole-6-carboxylic acid as starting materials. The crude product was purified by preparative HPLC (RP-18, gradient acetonitrile/water/NH4OAc 10:90:0.1 to 95:5:0.1) to give the product as a white solid (58 mg, 56%).

¹H NMR (399.99 MHz, CD3OD) δ 7.61 (d, 1H), 7.47 (s, 1H), 7.36 (d, 1H), 7.22 (d, 1H), 7.17 (d, 1H), 7.05 (dd, 1H), 6.88 (t, 1H), 6.50 (d, 1H), 4.05 (s, 2H), 3.82-3.45 (m, 4H), 3.08 (s, 6H), 1.85-1.42 (m, 14H)

APCI-MS m/z: 458.6 [MH+]

HPLC (Method A) Retention time: 8.35 min

HPLC (Method B) Retention time: 10.51 min

### EXAMPLE 69

### 3-(1H-benzimidazol-6-ylcarbonyl)-9-[(2,2-dimethyl-2,3-dihydro-1-benzofuran-7-yl)methyl]-3,9-diazaspiro[5.5]undecane

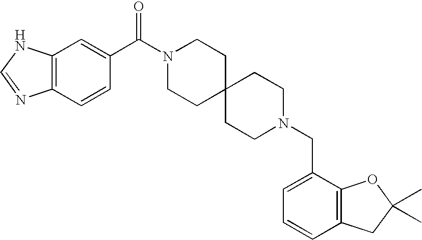

The title compound was prepared by the procedure of Example 52 using Intermediate A and 5-benzimidazolecarboxylic acid as starting materials. The crude product was purified by preparative HPLC (RP-18, gradient acetonitrile/water/NH4OAc 10:90:0.1 to 95:5:0.1) to give the product as a white solid (43 mg, 47%).

¹H NMR (399.99 MHz, CD3OD) δ 8.27 (s, 1H), 7.68 (s, 2H), 7.32 (d, 1H), 7.08 (d, 2H), 6.78 (t, 1H), 3.74 (s, 2H), 3.57 (s, 2H), 3.45 (s, 2H), 3.02 (s, 2H), 2.56 (s, 4H), 1.68-1.39 (m, 14H).

APCI-MS m/z: 459.6 [MH+]

HPLC (Method A) Retention time: 5.52 min

HPLC (Method B) Retention time: 7.98 min

### EXAMPLE 70

### 4-({9-[(3,3-dimethyl-2,3-dihydro-1,4-benzodioxin-5-yl)methyl]-3,9-diazaspiro[5.5]undec-3-yl}carbonyl)pyrimidin-2-amine Trifluoroacetate

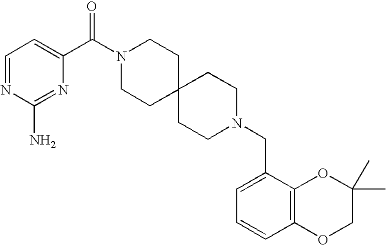

The title compound was prepared by the procedure of Example 77 using intermediate P and intermediate Z as starting materials to give the product as a white solid (65 mg, 32%).

APCI-MS m/z: 452.2 [MH+]

HPLC (Method A) Retention time: 5.14 min

HPLC (Method B) Retention time: 7.82 min

¹H NMR (399.99 MHz, CD3OD) δ 8.38 (t, J=4.8 Hz, 1H), 7.05-6.99 (m, 2H), 6.95-6.90 (m, 1H), 6.78 (d, J=5.2 Hz, 1H), 4.31 (d, J=7.7 Hz, 2H), 3.97 (d, J=4.4 Hz, 2H), 3.75-3.69 (m, 2H), 3.48-3.37 (m, 4H), 3.27-3.12 (m, 2H), 2.06-1.99 (m, 2H), 1.81-1.76 (m, 1H), 1.75-1.61 (m, 3H), 1.59-1.48 (m, 2H), 1.39 (d, J=7.7 Hz, 6H)

### EXAMPLE 71

### 3-({9-[(2,2-dimethyl-2,3-dihydro-1-benzofuran-7-yl)methyl]-3,9-diazaspiro[5.5]undec-3-yl}carbonyl)benzonitrile

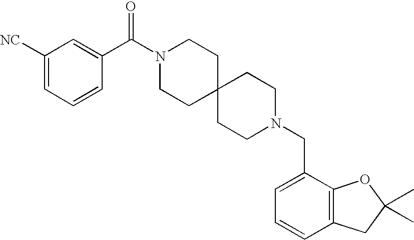

The title compound was prepared by the procedure of Example 52 using Intermediate A and 3-cyanobenzoic acid as starting materials. The crude product was purified by preparative HPLC (RP-18, gradient acetonitrile/water/NH4OAc 10:90:0.1 to 95:5:0.1) to give the product as a white solid (54 mg, 61%).

¹H NMR (399.99 MHz, CD3OD) δ 7.84-7.81 (m, 1H), 7.79 (s, 1H), 7.72-7.61 (m, 2H), 7.07 (d, 2H), 6.78 (t, 1H), 3.76 (s, 2H), 3.56 (s, 2H), 3.38-3.33 (m, 2H), 3.02 (s, 2H), 2.55 (s, 4H), 1.67-1.52 (m, 6H), 1.44 (s, 8H)

APCI-MS m/z: 444.6 [MH+]

HPLC (Method A) Retention time: 7.91 min

HPLC (Method B) Retention time: 10.10 min

### EXAMPLE 72

### 4-({9-[(2,2-dimethyl-2,3-dihydro-1-benzofuran-7-yl)methyl]-3,9-diazaspiro[5.5]undec-3-yl}carbonyl)benzonitrile

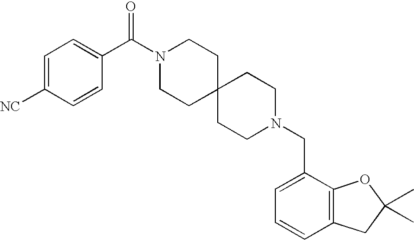

The title compound was prepared by the procedure of Example 52 using Intermediate A and 4-cyanobenzoic acid as starting materials. The crude product was purified by preparative HPLC (RP-18, gradient acetonitrile/water/NH4OAc 10:90:0.1 to 95:5:0.1) to give the product as a white solid (53 mg, 60%).

¹H NMR (399.99 MHz, CD3OD) δ 7.82 (d, 2H), 7.56 (d, 2H), 7.07 (d, 2H), 6.78 (t, 1H), 3.75-3.68 (m, 2H), 3.56 (s, 2H), 3.35-3.33 (m, 2H), 3.02 (s, 2H), 2.54 (s, 4H), 1.65-1.53 (m, 6H), 1.44 (s, 8H)

APCI-MS m/z: 444.6 [MH+]

HPLC (Method A) Retention time: 7.91 min

HPLC (Method B) Retention time: 10.12 min

### EXAMPLE 73

### 4-({9-[(2,2-dimethyl-2,3-dihydro-1-benzofuran-7-yl)methyl]-3,9-diazaspiro[5.5]undec-3-yl}carbonyl)benzenesulfonamide Trifluoroacetate

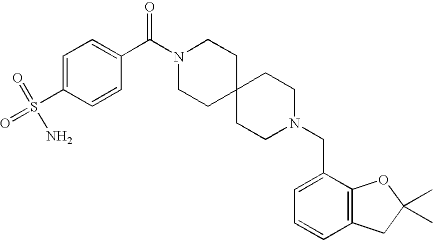

The title compound was prepared by the procedure of Example 52 using Intermediate A and 4-carboxybenzenesulfonamide as starting materials. The crude product was purified by preparative HPLC (RP-18, gradient acetonitrile/water/NH₄OAc 10:90:0.1 and acetonitrile/water/TFA from 10/90/0.1 to 95/5/0.1) to give the product as a white solid (31 mg, 21%).

¹H NMR (399.99 MHz, CD3OD) δ 7.98 (d, 2H), 7.57 (d, 2H), 7.29 (d, 1H), 7.20 (d, 1H), 6.92 (t, 1H), 4.26 (d, 2H), 3.76 (s, 2H), 3.45-3.34 (m, 4H), 3.27-3.07 (m, 4H), 2.04 (d, 2H), 1.84-1.40 (m, 12H).

APCI-MS m/z: 498.6 [MH+]

HPLC (Method A) Retention time: 6.67 min

HPLC (Method B) Retention time: 7.78 min

### EXAMPLE 74

### [3-({9-[(2,2-dimethyl-2,3-dihydro-1-benzofuran-7-yl)methyl]-3,9-diazaspiro[5.5]undec-3-yl}carbonyl)phenyl]amine Trifluoroacetate

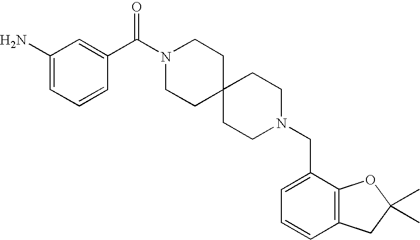

The title compound was prepared by the procedure of Example 57 using Intermediate A and Boc-3-aminobenzoic acid as starting materials to give the product as a white solid (78 mg, 59%).

¹H NMR (399.99 MHz, CD3OD) δ 7.39 (t, 1H), 7.29 (d, 1H), 7.20 (d, 1H), 7.15-7.10 (m, 1H), 7.09-7.03 (m, 2H), 6.92 (t, 1H), 4.26 (s, 2H), 3.74 (s, 2H), 3.49-3.36 (m, 4H), 3.27-3.06 (m, 4H), 2.04 (d, 2H), 1.83-1.38 (m, 12H)

APCI-MS m/z: 434.6 [MH+]

HPLC (Method A) Retention time: 5.48 min

HPLC (Method B) Retention time: 8.93 min

### EXAMPLE 75

### 5-({9-[(2,2-dimethyl-2,3-dihydro-1-benzofuran-7-yl)methyl]-3,9-diazaspiro[5.5]undec-3-yl}carbonyl)pyrazin-2(1H)-one Trifluoroacetate

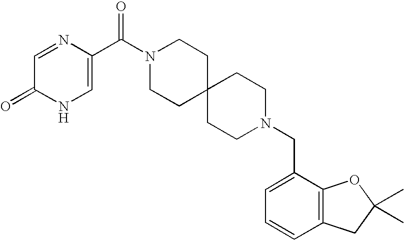

The compound was prepared by the amide coupling procedure of Intermediate S, using Intermediate A and 5-hydroxypyrazine-2-carboxylic acid as starting materials to give the product as a white solid (25 mg, 23%).

¹H NMR (399.99 MHz, CD3OD) δ 8.01 (s, 1H), 7.81 (s, 1H), 7.30 (d, J=6.3 Hz, 1H), 7.20 (d, J=7.3 Hz, 1H), 6.93 (t, J=7.6 Hz, 1H), 4.27 (s, 2H), 3.77-3.67 (m, 4H), 3.45-3.36 (m, 2H), 3.25-3.15 (m, 2H), 3.11 (s, 2H), 2.08-2.00 (m, 2H), 1.79-1.73 (m, 2H), 1.69-1.60 (m, 2H), 1.57-1.52 (m, 2H), 1.50 (s, 6H)

APCI-MS m/z: 437.3 [MH+]

HPLC (Method A) Retention time: 5.55 min

HPLC (Method B) Retention time: 4.90 min

### EXAMPLE 76

### 5-({9-[(2,2-dimethyl-2,3-dihydro-1-benzofuran-7-yl)methyl]-3,9-diazaspiro[5.5]undec-3-yl}carbonyl)pyridin-2(1H)-one Trifluoroacetate

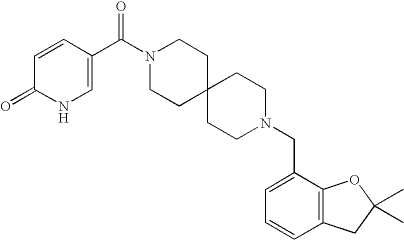

The compound was prepared by the amide coupling procedure of Intermediate S, using Intermediate A and 6-hydroxynicotinic acid as starting materials to give the product as a white solid (65 mg, 61%).

¹H NMR (399.99 MHz, CD3OD) δ 7.74-7.58 (m, 2H), 7.34-7.26 (m, 1H), 7.22-7.14 (m, 1H), 7.01-6.84 (m, 1H), 6.61-6.41 (m, 1H), 4.27 (s, 2H), 3.74-3.52 (m, 4H), 3.46-3.37 (m, 2H), 3.27-3.06 (m, 6H), 2.10-1.99 (m, 2H), 1.77-1.70 (m, 2H), 1.68-1.58 (m, 2H), 1.50 (s, 9H)

APCI-MS m/z: 436.6 [MH+]

HPLC (Method A) Retention time: 5.39 min

HPLC (Method B) Retention time: 7.08 min

### EXAMPLE 77

### 3-isonicotinoyl-9-[(2-methyl-2,3-dihydro-1-benzofuran-7-yl)methyl]-3,9-diazaspiro[5.5]undecane Trifluoroacetate

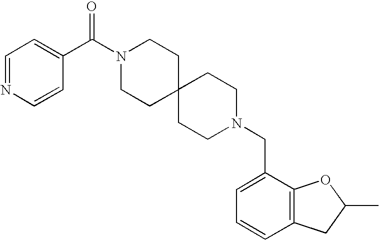

A mixture of Intermediate D (66 mg, 0.41 mmol), Intermediate S (106 mg, 0.41 mmol), sodium triacetoxyborohydride (174 mg, 0.81 mmol) and acetonitrile (2 ml) were heated at 40° C. for 18 h. The mixture was diluted with ethyl acetate and washed with aqueous sodium hydrogen carbonate, the organic layer was evaporated and the residue was purified by preparative HPLC (RP-18, gradient acetonitrile/water/TFA from 10/90/0.1 to 95/5/0.1) to give the product as a white solid (40 mg, 19%)

¹H NMR (399.99 MHz, CD3OD) δ 8.88-8.74 (m, 2H), 7.81-7.64 (m, 2H), 7.34-7.26 (m, 1H), 7.23-7.15 (m, 1H), 6.97-6.87 (m, 1H), 5.10-4.96 (m, 1H), 4.35-4.20 (m, 2H), 3.78 (d, J=4.6 Hz, 2H), 3.46-3.33 (m, 6H), 3.27-3.08 (m, 2H), 2.94-2.83 (m, 1H), 2.04 (d, J=13.4 Hz, 2H), 1.82 (d, J=5.4 Hz, 1H), 1.75-1.55 (m, 4H), 1.51-1.43 (m, 3H)

APCI-MS m/z: 406.3 [MH+]

HPLC (Method A) Retention time: 4.19 min

### EXAMPLE 78

### 3-isonicotinoyl-9-[(2,3,3-trimethyl-2,3-dihydro-1-benzofuran-7-yl)methyl]-3,9-diazaspiro[5.5]undecane and 3-isonicotinoyl-9-[(2,2,3-trimethyl-2,3-dihydro-1-benzofuran-7-yl)methyl]-3,9-diazaspiro[5.5]undecane

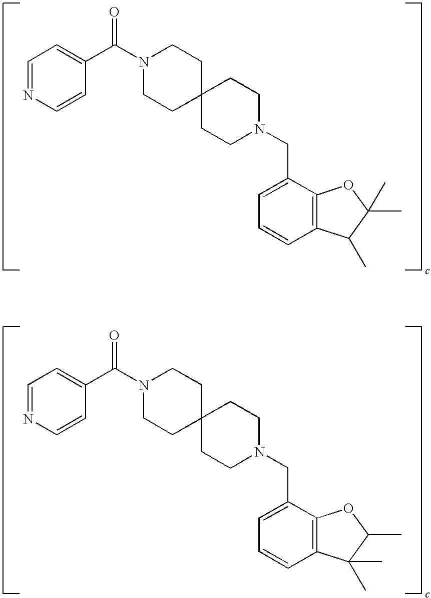

The title compound mixture was prepared by the procedure of Example 77 using Intermediate T and Intermediate S to give a 1:1 isomeric product mixture as a white solid (130 mg, 45%).

¹H NMR (399.99 MHz, CD3OD) δ 8.79 (d, J=4.8 Hz, 2H), 7.72 (d, J=5.9 Hz, 2H), 7.31-7.24 (m, 1H), 7.24-7.18 (m, 1H), 7.02-6.92 (m, 1H), 4.54-4.44 (m, 0.5H), 4.32-4.23 (m, 2H), 3.82-3.74 (m, 2H), 3.46-3.33 (m, 4H), 3.28-3.08 (m, 2.5H), 2.04 (d, J=13.9 Hz, 2H), 1.86-1.77 (m, 1H), 1.74-1.54 (m, 4H), 1.53-1.23 (m, 8.5H), 1.13 (d, J=4.9 Hz, 1.5H)

APCI-MS m/z: 434.4 [MH+]

HPLC (Method A) Retention time: 5.62/5.69 min

HPLC (Method B) Retention time: 8.60/8.65 min

### EXAMPLE 79

### 3-(2,3-dihydro-1-benzofuran-7-ylmethyl)-9-isonicotinoyl-3,9-diazaspiro[5.5]undecane Trifluoroacetate

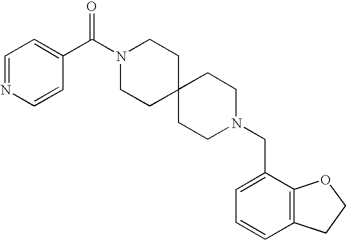

The title compound was prepared by the procedure of Example 77 using 2,3-dihydro-1-benzofuran-7-carbaldehyde and Intermediate S to give the product as a white solid (38 mg, 24%).

¹H NMR (399.99 MHz, CD3OD) δ 8.88-8.74 (m, 2H), 7.84-7.65 (m, 2H), 7.39-7.30 (m, 1H), 7.24-7.15 (m, 1H), 6.99-6.89 (m, 1H), 4.65 (q, J=8.7 Hz, 2H), 4.28 (d, J=11.4 Hz, 2H), 3.78 (d, J=4.2 Hz, 2H), 3.46-3.33 (m, 4H), 3.30-3.09 (m, 4H), 2.03 (d, J=14.8 Hz, 2H), 1.82 (t, J=5.3 Hz, 1H), 1.75-1.54 (m, 4H), 1.50-1.43 (m, 1H)

APCI-MS m/z: 392.3 [MH+]

HPLC (Method A) Retention time: 3.93 min

HPLC (Method B) Retention time: 6.96 min

### EXAMPLE 80

### 3-isonicotinoyl-9-[(2,2,3,3-tetramethyl-2,3-dihydro-1-benzofuran-7-yl)methyl]-3,9-diazaspiro[5.5]undecane Trifluoroacetate

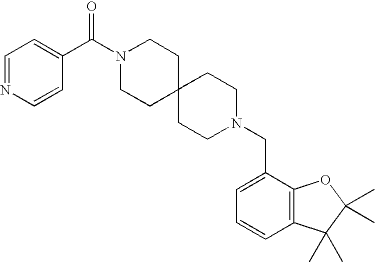

The title compound was prepared by the procedure of Example 77 using Intermediate G and Intermediate S to give the product as a white solid (125 mg, 80%).

¹H NMR (399.99 MHz, CD3OD) δ 8.79 (d, J=2.6 Hz, 2H), 7.75-7.68 (m, 2H), 7.27-7.18 (m, 2H), 7.00-6.94 (m, 1H), 4.27 (d, J=11.5 Hz, 2H), 3.77 (d, J=4.3 Hz, 2H), 3.44-3.33 (m, 4H), 3.26-3.07 (m, 2H), 2.09-1.99 (m, 2H), 1.84-1.78 (m, 1H), 1.74-1.54 (m, 4H), 1.50-1.43 (m, 1H), 1.36 (d, J=11.4 Hz, 6H), 1.22 (d, J=6.0 Hz, 6H)

APCI-MS m/z: 448.4 [MH+]

HPLC (Method A) Retention time: 6.11 min

HPLC (Method B) Retention time: 8.81 min

### EXAMPLE 81

### 3-[(5-chloro-2,2-dimethyl-2,3-dihydro-1-benzofuran-7-yl)methyl]-9-isonicotinoyl-3,9-diazaspiro[5.5]undecane Trifluoroacetate

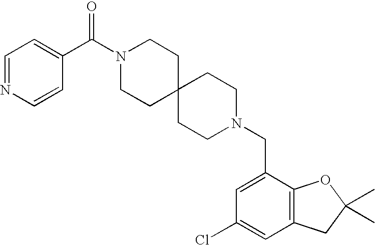

The title compound was prepared by the procedure of Example 77 using Intermediate F and Intermediate S to give the product as a white solid (91 mg, 52%).

¹H NMR (399.99 MHz, CD3OD) δ 8.82 (d, J=5.2 Hz, 2H), 7.79 (d, J=6.3 Hz, 2H), 7.28 (d, J=11.2 Hz, 2H), 4.24 (d, J=11.0 Hz, 2H), 3.82-3.73 (m, 2H), 3.45-3.32 (m, 4H), 3.28-3.09 (m, 5H), 2.04 (d, J=14.9 Hz, 2H), 1.86-1.79 (m, 1H), 1.75-1.55 (m, 4H), 1.50 (d, J=11.0 Hz, 6H)

APCI-MS m/z: 454.3/456.3 [MH+]

HPLC (Method A) Retention time: 6.11 min

HPLC (Method B) Retention time: 9.41 min

### EXAMPLE 82

### 3-isonicotinoyl-9-[(2,2,4-trimethyl-2,3-dihydro-1-benzofuran-7-yl)methyl]-3,9-diazaspiro[5.5]undecane Trifluoroacetate

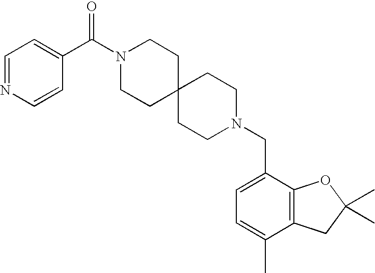

The title compound was prepared by the procedure of Example 77 using Intermediate H and Intermediate S to give the product as a white solid (124 mg, 73%).

¹H NMR (399.99 MHz, CD3OD) δ 8.80 (d, J=5.0 Hz, 2H), 7.74 (d, J=4.7 Hz, 2H), 7.14-7.06 (m, 1H), 6.79-6.71 (m, 1H), 4.21 (d, J=11.5 Hz, 2H), 3.81-3.73 (m, 2H), 3.43-3.32 (m, 5H), 3.24-3.06 (m, 2H), 3.03 (d, J=6.4 Hz, 2H), 2.24 (d, J=4.1 Hz, 3H), 2.08-1.98 (m, 2H), 1.83-1.77 (m, 1H), 1.74-1.55 (m, 4H), 1.50 (d, J=11.3 Hz, 6H)

APCI-MS m/z: 434.3 [MH+]

HPLC (Method A) Retention time: 5.79 min

HPLC (Method B) Retention time: 8.60 min

### EXAMPLE 83

### 3-[(4-chloro-2,2-dimethyl-2,3-dihydro-1-benzofuran-7-yl)methyl]-9-isonicotinoyl-3,9-diazaspiro[5.5]undecane Trifluoroacetate

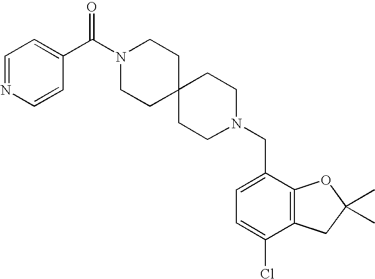

The title compound was prepared by the procedure of Example 77 using Intermediate U and Intermediate S to give the product as a white solid (100 mg, 57%).

¹H NMR (399.99 MHz, CD3OD) δ 8.84 (s, 2H), 7.88-7.77 (m, 2H), 7.27-7.20 (m, 1H), 6.97-6.90 (m, 1H), 4.24 (d, J=10.9 Hz, 2H), 3.78 (d, J=4.5 Hz, 2H), 3.45-3.32 (m, 4H), 3.25-3.07 (m, 4H), 2.03 (d, J=14.7 Hz, 2H), 1.84-1.78 (m, 1H), 1.76-1.61 (m, 3H), 1.62-1.43 (m, 8H)

APCI-MS m/z: 454.1/456.1 [MH+]

HPLC (Method A) Retention time: 9.07 min

HPLC (Method B) Retention time: 9.17 min

### EXAMPLE 84

### 3-[(2,2-dimethyl-2,3-dihydro-1-benzofuran-4-yl)methyl]-9-isonicotinoyl-3,9-diazaspiro[5.5]undecane Trifluoroacetate

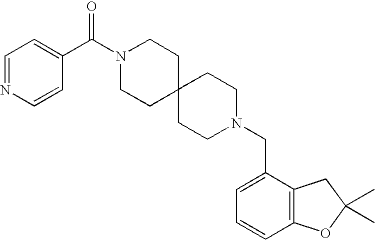

The title compound was prepared by the procedure of Example 77 using Intermediate W and Intermediate S to give the product as a white solid (15 mg, 12%).

¹H NMR (399.99 MHz, CD3OD) δ 8.81-8.73 (m, 2H), 7.73-7.62 (m, 2H), 7.23 (t, J=7.7 Hz, 1H), 6.98-6.91 (m, 1H), 6.78 (d, J=8.1 Hz, 1H), 4.25 (d, J=11.1 Hz, 2H), 3.85-3.73 (m, 2H), 3.46-3.33 (m, 4H), 3.29-3.11 (m, 5H), 2.05 (d, J=14.8 Hz, 2H), 1.89-1.82 (m, 1H), 1.75-1.55 (m, 4H), 1.48 (d, J=6.1 Hz, 6H)

APCI-MS m/z: 420.4 [MH+]

HPLC (Method A) Retention time: 4.87 min

### EXAMPLE 85

### 4-({9-[(2,2-dimethyl-2,3-dihydro-1-benzofuran-4-yl)methyl]-3,9-diazaspiro[5.5]undec-3-yl}carbonyl)pyridin-3-amine Trifluoroacetate

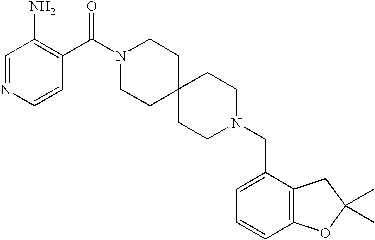

The compound was prepared by the amide coupling procedure of Intermediate S and using Intermediate C and 3-aminoisonicotinic acid as starting materials to give the product as a white solid (60 mg, 41%).

¹H NMR (399.99 MHz, CD3OD) δ 8.15 (s, 1H), 7.98 (d, J=5.1 Hz, 1H), 7.59 (d, J=5.5 Hz, 1H), 7.22 (t, J=7.8 Hz, 1H), 6.95 (d, J=7.8 Hz, 1H), 6.78 (d, J=7.7 Hz, 1H), 4.25 (s, 2H), 3.85-3.72 (m, 2H), 3.48-3.34 (m, 4H), 3.26-3.12 (m, 5H), 2.03 (d, J=14.3 Hz, 2H), 1.91-1.83 (m, 1H), 1.78-1.55 (m, 4H), 1.48 (s, 6H)

APCI-MS m/z: 435.4 [MH+]

HPLC (Method A) Retention time: 4.81 min

HPLC (Method B) Retention time: 7.79 min

### EXAMPLE 86

### 6-({9-[(2,2-dimethyl-2,3-dihydro-1-benzofuran-4-yl)methyl]-3,9-diazaspiro[5.5]undec-3-yl}carbonyl)pyridin-3-amine Trifluoroacetate

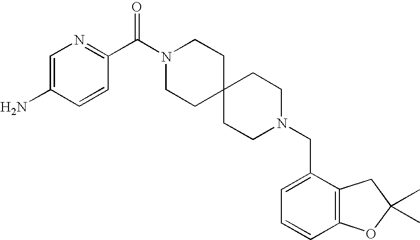

The compound was prepared by the amide coupling procedure of Intermediate S and using Intermediate C and 5-aminopyridine-2-carboxylic acid as starting materials to give the product as a white solid (38 mg, 26%).

¹H NMR (399.99 MHz, CD3OD) δ 7.98 (d, J=2.4 Hz, 1H), 7.56 (d, J=8.7 Hz, 1H), 7.36-7.31 (m, 1H), 7.23 (t, J=7.9 Hz, 1H), 6.95 (d, J=7.6 Hz, 1H), 6.78 (d, J=8.0 Hz, 1H), 4.25 (s, 2H), 3.74-3.57 (m, 4H), 3.46-3.37 (m, 2H), 3.28-3.13 (m, 4H), 2.04 (d, J=14.6 Hz, 2H), 1.84-1.76 (m, 2H), 1.72-1.60 (m, 2H), 1.58-1.50 (m, 2H), 1.48 (s, 6H)

APCI-MS m/z: 435.3 [MH+]

HPLC (Method A) Retention time: 5.03 min

HPLC (Method B) Retention time: 7.68 min

The corresponding benzenesulfonate salt was prepared by heating a 20 mM solution (307.18 ml) of 6-({9-[(2,2-dimethyl-2,3-dihydro-1-benzofuran-4-yl)methyl]-3,9-diazaspiro[5.5]undec-3-yl}carbonyl)pyridin-3-amine in i-PrOH to 40° C. and adding an 80 mM solution (76.79 ml) of Benzene Sulphonic Acid in i-PrOH. The combined solution was stirred at 40° C. for 1 h and then over night at room temperature. The crystals was then filtered of and dried under vacuum. Yield 2.7 g (73%)

¹H NMR (399.99 MHz, DMSO-D6) δ 9.03 (s, 1H), 7.85 (d, J=1.9 Hz, 1H), 7.62-7.57 (m, 2H), 7.36-7.28 (m, 4H), 7.19 (t, J=7.8 Hz, 1H), 6.98-6.92 (m, 2H), 6.78 (d, J=8.0 Hz, 1H), 5.74 (s, 2H), 4.22 (s, 2H), 3.63-3.51 (m, 4H), 3.27-3.07 (m, 6H), 1.90 (d, J=14.3 Hz, 2H), 1.65 (s, 2H), 1.54 (t, J=12.0 Hz, 2H), 1.43 (s, 6H), 1.39-1.31 (m, 2H)

### EXAMPLE 87

### 2-[2-({9-[(2,2-dimethyl-2,3-dihydro-1-benzofuran-4-yl)methyl]-3,9-diazaspiro[5.5]undec-3-yl}carbonyl)phenyl]acetamide Trifluoroacetate

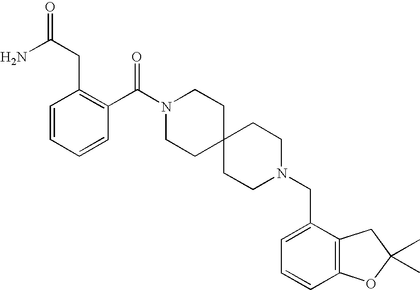

The title compound was prepared by the procedure of Example 77 using Intermediate W and Intermediate V to give the product as a white solid (90 mg, 49%).

¹H NMR (399.99 MHz, CD3OD) δ 7.45-7.38 (m, 2H), 7.37-7.31 (m, 1H), 7.28-7.19 (m, 2H), 6.96 (t, J=6.7 Hz, 1H), 6.81-6.75 (m, 1H), 4.24 (d, J=14.0 Hz, 2H), 3.83-3.72 (m, 2H), 3.70-3.62 (m, 2H), 3.56-3.48 (m, 2H), 3.44-3.36 (m, 2H), 3.28-3.20 (m, 2H), 3.16 (d, J=13.1 Hz, 2H), 2.09-1.96 (m, 2H), 1.87-1.79 (m, 1H), 1.75-1.60 (m, 4H), 1.56 (t, J=5.5 Hz, 1H), 1.48 (d, J=7.7 Hz, 6H)

APCI-MS m/z: 476.3 [MH+]

HPLC (Method A) Retention time: 6.04 min

HPLC (Method B) Retention time: 8.21 min

### EXAMPLE 88

### 3-(1,3-benzodioxol-4-ylmethyl)-9-isonicotinoyl-3,9-diazaspiro[5.5]undecane Trifluoroacetate

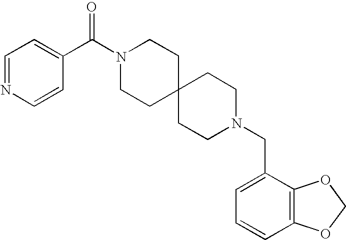

The title compound was prepared by the procedure of Example 1 using 1,3-benzodioxole-4-carbaldehyde and Intermediate S to give the product as a white solid (68 mg, 35%).

¹H NMR (399.99 MHz, CD3OD) δ 8.86 (d, J=5.1 Hz, 2H), 7.98-7.74 (m, 2H), 6.95 (d, J=3.0 Hz, 3H), 6.05 (d, J=8.9 Hz, 2H), 4.31 (d, J=10.4 Hz, 2H), 3.78 (d, J=4.6 Hz, 2H), 3.51-3.39 (m, 2H), 3.38-3.32 (m, 2H), 3.29-3.10 (m, 2H), 2.11-1.96 (m, 2H), 1.88-1.78 (m, 1H), 1.77-1.61 (m, 3H), 1.61-1.54 (m, 1H), 1.50-1.42 (m, 1H)

APCI-MS m/z: 394.3 [MH+]

HPLC (Method A) Retention time: 3.25 min

HPLC (Method B) Retention time: 6.68 min

### EXAMPLE 89

### 3-[(2,2-dimethyl-1,3-benzodioxol-4-yl)methyl]-9-isonicotinoyl-3,9-diazaspiro[5.5]undecane

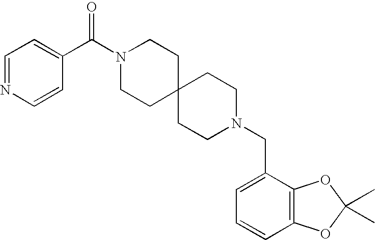

The title compound was prepared by the synthetic procedure of Intermediate A using Intermediate S and 2,2-dimethyl-1,3-benzodioxole-4-carbaldehyde as starting materials. The crude product was purified by preparative HPLC (RP-18, gradient acetonitrile/water/aqueous NH₃ from 10/90/0.1 to 95/5/0.1) to give the product as a white solid (26 mg, 32%).

¹H NMR (399.99 MHz, DMSO-D6) δ 9.32 (s, 1H), 8.69 (s, 2H), 7.41 (s, 2H), 6.93 (s, 3H), 4.29-4.19 (m, 2H), 3.61 (s, 2H), 3.33-2.97 (m, 6H), 1.90 (d, J=14.0 Hz, 2H), 1.78-1.23 (m, 12H)

APCI-MS m/z: [MH+] 422.3

HPLC (Method A) Retention time: 4.51 min

HPLC (Method B) Retention time: 7.44 min

### EXAMPLE 90

### 4-({9-[(2,2-dimethyl-1,3-benzodioxol-4-yl)methyl]-3,9-diazaspiro[5.5]undec-3-yl}carbonyl)pyridin-3-amine

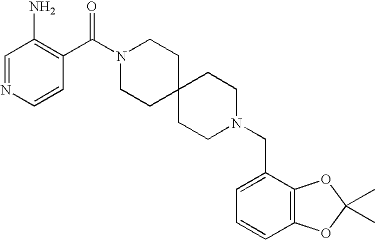

Prepared according to Example 24 using Intermediate B (78 mg, 0.20 mmol) and 3-aminoisoicotinic acid (33 mg, 0.24 mmol) and purified by SCX ion exchange chromatography to afford the title compound as a white solid (50 mg, 57%).

¹H NMR (399.99 MHz, CD3OD) δ 8.07 (s, 1H), 7.82 (d, J=5.0 Hz, 1H), 7.05 (d, J=4.9 Hz, 1H), 6.81-6.72 (m, 2H), 6.65 (dd, J=7.0, 1.9 Hz, 1H), 3.71 (s, 2H), 3.66 (s, 2H), 3.52 (s, 2H), 3.35 (s, 2H), 2.52 (s, 4H), 1.64 (s, 6H), 1.62-1.42 (m, 8H)

APCI-MS m/z: 437.1 [MH+]

HPLC (Method A) Retention time: 4.79 min

### EXAMPLE 91

### 4-({9-[(2,2-dimethyl-1,3-benzodioxol-4-yl)methyl]-3,9-diazaspiro[5.5]undec-3-yl}carbonyl)pyridin-2-amine

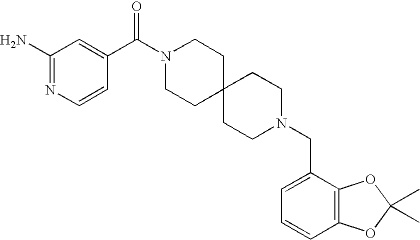

The title compound was prepared by the synthetic procedure of Intermediate A using Intermediate L and 2,2-dimethyl-1,3-benzodioxole-4-carbaldehyde as starting materials. The crude product was purified by preparative HPLC (RP-18, gradient acetonitrile/water/aqueous NH₃ from 10:90:0.1 to 95:5:0.1) to give the product as a white solid (30 mg, 15%).

¹H NMR (399.99 MHz, DMSO-D6) δ 7.93 (d, 1H), 6.80-6.65 (m, 2H), 6.37 (d, 1H), 6.32 (s, 1H), 6.07 (d, 1H), 3.53 (s, 2H), 3.38 (s, 2H), 3.22 (s, 2H), 2.33 (s, 4H), 1.61 (s, 6H), 1.51-1.27 (m, 8H)

APCI-MS m/z: [MH+] 437.1

HPLC (Method A) Retention time: 4.81 min

HPLC (Method B) Retention time: 7.63 min

### EXAMPLE 92

### 2-({9-[(2,2-dimethyl-1,3-benzodioxol-4-yl)methyl]-3,9-diazaspiro[5.5]undec-3-yl}carbonyl)pyridin-3-amine

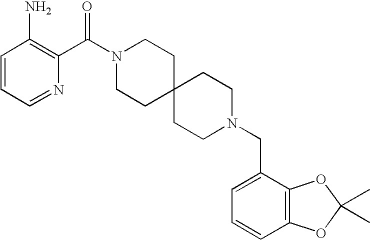

The title compound was prepared according to Example 24 using intermediate B (78 mg, 0.20 mmol) and 3-aminopyridine-2-carboxylic acid (33 mg, 0.24 mmol) and purified by SCX ion exchange chromatography to afford the title compound as a white solid (40 mg, 46%).

¹H NMR (399.99 MHz, CD3OD) δ 7.82 (dd, J=3.9, 1.9 Hz, 1H), 7.23-7.16 (m, 2H), 6.81-6.73 (m, 2H), 6.69-6.64 (m, 1H), 3.77-3.70 (m, 2H), 3.66 (s, 2H), 3.57 (s, 2H), 3.38-3.32 (m, 2H), 2.57 (s, 4H), 1.64 (s, 6H), 1.63-1.57 (m, 6H), 1.51-1.43 (m, 2H)

APCI-MS m/z: 437.1 [MH+]

HPLC (Method A) Retention time: 4.82 min

### EXAMPLE 93

### 2-[2-({9-[(2,2-dimethyl-1,3-benzodioxol-4-yl)methyl]-3,9-diazaspiro[5.5]undec-3-yl}carbonyl)phenyl]acetamide Trifluoroacetate

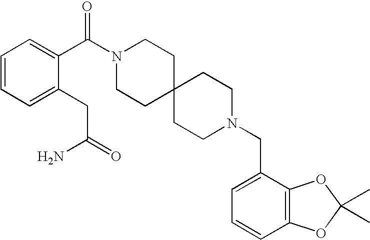

The title compound was prepared by the synthetic procedure of Intermediate A using Intermediate V and 2,2-dimethyl-1,3-benzodioxole-4-carbaldehyde as starting materials.

The crude product was purified by preparative HPLC (RP-18, gradient acetonitrile/water/TFA from 10/90/0.1 to 95/5/0.1) to give the product as a white solid (43 mg, 28%).

¹H NMR (399.99 MHz, DMSO-D6) δ 9.44 (s, 1H), 7.47-7.22 (m, 3H), 7.16 (t, J=7.1 Hz, 1H), 6.97-6.81 (m, 3H), 4.23 (dd, 2H), 4.11-2.94 (m, H2O, 12H), 1.97-1.75 (m, 2H), 1.74-1.14 (m, 12H)

APCI-MS m/z: [MH+] 478.3

HPLC (Method A) Retention time: 5.96 min

HPLC (Method B) Retention time: 8.30 min

### EXAMPLE 94

### 4-({9-[(2,2-dimethyl-1,3-benzodioxol-4-yl)methyl]-3,9-diazaspiro[5.5]undec-3-yl}carbonyl)pyridazin-3-amine Trifluoroacetate

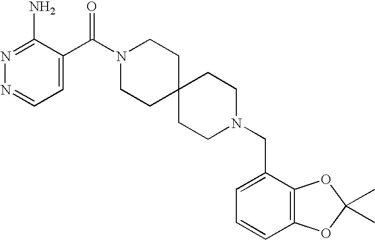

4-(3,9-diazaspiro[5.5]undec-3-ylcarbonyl)pyridazin-3-amine was prepared using the amide coupling procedure of Example 8 and the Boc cleaving procedure of Intermediate A using tert-butyl 3,9-diazaspiro[5.5]undecane-3-carboxylate hydrochloric acid salt and 3-aminopyridazine-4-carboxylic acid as starting materials. This intermediate was reacted with 2,2-dimethyl-1,3-benzodioxole-4-carbaldehyde using the synthetic procedure of Intermediate A. The crude product was purified by preparative HPLC (RP-18, gradient acetonitrile/water/TFA from 10/90/0.1 to 95/5/0.1) to afford the product as a white solid (268 mg, 75%).

¹H NMR (399.99 MHz, DMSO-D6) δ 9.55 (s, 1H), 8.57 (s, 1H), 7.83-7.49 (m, 3H), 7.06-6.78 (m, 3H), 4.93-3.66 (m, 4H), 3.64-3.50 (m, 2H), 3.35-2.96 (m, 6H), 1.95-1.27 (m, 14H)

APCI-MS m/z: [MH+] 438.3

HPLC (Method A) Retention time: 4.41 min

HPLC (Method B) Retention time: 7.24 min

### EXAMPLE 95

### 4-({9-[(2-ethyl-2-methyl-1,3-benzodioxol-4-yl)methyl]-3,9-diazaspiro[5.5]undec-3-yl}carbonyl)pyridin-3-amine Trifluoroacetate

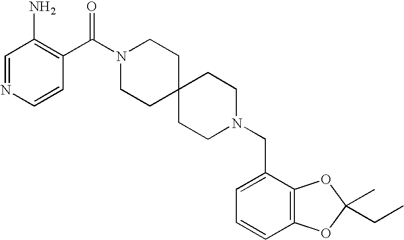

### (a) 2-ethyl-2,4-dimethyl-1,3-benzodioxole

A mixture of 3-methylbenzene-1,2-diol (4.73 g, 38.3 mmol), phosphorus pentoxide (6.00 g, 42.3 mmol), 2-butanone (5 ml, 55.4 mmol) and toluene was stirred at 75° C. for 16 h and filtered through silica. The crude product was purified by distillation to yield the product as a yellow oil (6.16 g, 91%)

¹H NMR (399.99 MHz, DMSO-D6) δ 6.70-6.57 (m, 3H), 2.13 (s, 3H), 1.90 (q, 2H), 1.56 (s, 3H), 0.92 (t, 3H)

GC-MS m/z: [M+] 178.1

### (b) 4-(bromomethyl)-2-ethyl-2-methyl-1,3-benzodioxole

A mixture of 2-ethyl-2,4-dimethyl-1,3-benzodioxole (0.59 g, 3.33 mmol), N-bromosuccinimide (580 mg, 3.26 mmol), AIBN (30 mg, 0.18 mmol) and tetrachloromethane (10 ml) was heated under a strong UV irradiation at 60° C. for 1 h, filtered and evaporated. The crude product was directly used in next step.

GC-MS m/z: [M+] 255.9

### (c) 4-({9-[(2-ethyl-2-methyl-1,3-benzodioxol-4-yl)methyl]-3,9-diazaspiro[5.5]undec-3-yl}carbonyl)pyridin-3-amine Trifluoroacetate

A mixture of Intermediate M (89 mg, 0.35 mmol), potassium carbonate (91 mg, 0.66 mmol) and DMF was stirred at room temperature and crude 4-(bromomethyl)-2-ethyl-2-methyl-1,3-benzodioxole was added portionwise until LC-MS showed full consumption of Intermediate M. The mixture was acidified and purified by preparative HPLC (RP-18, gradient acetonitrile/water/TFA from 10/90/0.1 to 95/5/0.1) to give the product as a white solid (48 mg, 34%).

¹H NMR (399.99 MHz, DMSO-D6) δ 8.12 (s, 1H), 7.97 (d, 1H), 7.38 (d, 1H), 6.98-6.84 (m, 3H), 4.25 (s, 2H), 3.72-3.00 (m, 8H), 2.03-1.80 (m, 4H), 1.79-1.20 (m, 9H), 0.94 (s, 3H)

APCI-MS m/z: [MH+] 451.4

HPLC (Method A) Retention time: 5.72 min

HPLC (Method B) Retention time: 8.20 min

### EXAMPLE 96

### 4-{[9-(spiro[1,3-benzodioxole-2,1′-cyclobutan]-4-ylmethyl)-3,9-diazaspiro[5.5]undec-3-yl]carbonyl}pyridin-3-amine Trifluoroacetate

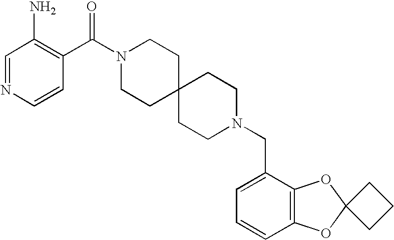

The title compound was prepared by the synthetic procedure of example 95 using cyclobutanone to give the product as a white solid (26 mg, 32%).

APCI-MS m/z: [MH+] 449.3

HPLC (Method A) Retention time: 5.21 min

HPLC (Method B) Retention time: 8.58 min

### EXAMPLE 97

### 4-{[9-(spiro[1,3-benzodioxole-2,1′-cyclopentan]-4-ylmethyl)-3,9-diazaspiro[5.5]undec-3-yl]carbonyl}pyridin-3-amine

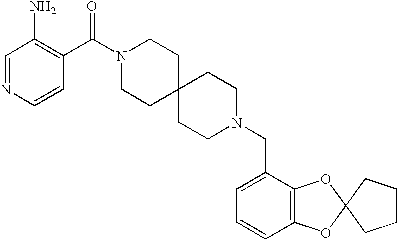

The title compound was prepared by the synthetic procedure of Intermediate A using Intermediate M and spiro[1,3-benzodioxole-2,1′-cyclopentane]-4-carbaldehyde. The crude product was purified by preparative HPLC (RP-18, gradient acetonitrile/water/aqueous NH₃ from 10:90:0.1 to 95:5:0.1) to give the product as a white solid (30 mg, 15%).

¹H NMR (399.99 MHz, DMSO-D6) δ 8.05 (s, 1H), 7.76 (d, 1H), 6.92 (d, H), 6.79-6.69 (m, 3H), 3.65-3.46 (m, 2H), 3.40 (s, 2H), 3.26-3.09 (m, 2H), 2.42-2.27 (m, 4H), 2.09-1.94 (m, 4H), 1.81-1.70 (m, 4H), 1.53-1.26 (m, 8H)

APCI-MS m/z: [MH+] 463.4

HPLC (Method A) Retention time: 5.98 min

HPLC (Method B) Retention time: 8.65 min

### EXAMPLE 98

### 4-{[9-(spiro[1,3-benzodioxole-2,1′-cyclopentan]-4-ylmethyl)-3,9-diazaspiro[5.5]undec-3-yl]carbonyl}pyridin-2-amine Trifluoroacetate

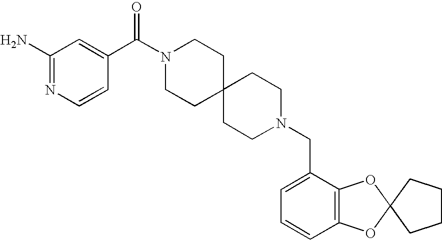

The title compound was prepared by the synthetic procedure of Intermediate A using Intermediate L and spiro[1,3-benzodioxole-2,1′-cyclopentane]-4-carbaldehyde. The crude product was purified by preparative HPLC (RP-18, gradient acetonitrile/water/aqueous NH₃ from 10:90:0.1 to 95:5:0.1) to give the product as a white solid (45 mg, 22%).

¹H NMR (399.99 MHz, DMSO-D6) δ 8.03-7.95 (m, 1H), 7.00-6.86 (m, 3H), 6.79-6.68 (m, 2H), 4.26 (d, 2H), 3.57 (s, 2H), 3.30-2.99 (m, 6H), 2.08 (d, 4H), 1.90 (d, 2H), 1.84-1.75 (m, 4H), 1.68 (s, 1H), 1.62-1.47 (m, 3H), 1.41 (s, 1H), 1.34-1.27 (m, 1H)

APCI-MS m/z: [MH+] 434.2

HPLC (Method A) Retention time: 6.11 min

HPLC (Method B) Retention time: 8.67 min

### EXAMPLE 99

### 4-{[9-(spiro[1,3-benzodioxole-2,1′-cycloheptan]-4-ylmethyl)-3,9-diazaspiro[5.5]undec-3-yl]carbonyl}pyridin-3-amine Trifluoroacetate

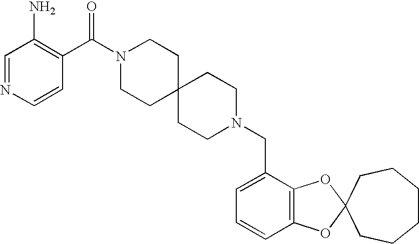

The title compound was prepared by the synthetic procedure of example 95 using D Intermediate M and cycloheptanone to give the product as a white solid (70 mg, 33%).

¹H NMR (399.99 MHz, DMSO-D6) δ 9.53 (s, 1H), 8.13 (s, 1H), 8.00 (d, 1H), 7.43 (d, 1H), 6.96-6.84 (m, 3H), 4.24 (s, 2H), 3.99-3.35 (m, H₂O, 2H), 3.32-2.99 (m, 6H), 2.09 (s, 4H), 1.86 (d, 2H), 1.77-1.26 (m, 14H)

APCI-MS m/z: [MH+] 491.4

HPLC (Method A) Retention time: 6.70 min

HPLC (Method B) Retention time: 10.10 min

### EXAMPLE 100

### 3-[(2-ethyl-2-methyl-1,3-benzodioxol-4-yl)methyl]-9-[(1-oxidopyridin-2-yl)carbonyl]-3,9-diazaspiro[5.5]undecane Trifluoroacetate

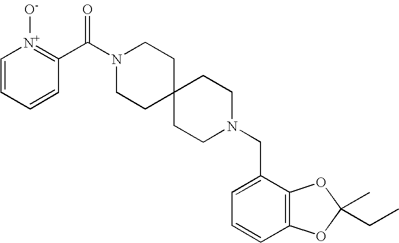

The title compound was prepared by the synthetic procedure of example 95 using Intermediate N to give the product as a white solid (18 mg, 16%).

¹H NMR (399.99 MHz, DMSO-D6) δ 9.40 (s, 1H), 8.26 (t, 1H), 7.51-7.35 (m, 3H), 6.96-6.84 (m, 3H), 4.29-4.19 (m, 2H), 3.72-2.96 (m, 8H), 2.02-1.89 (m, 4H), 1.89-1.19 (m, 9H), 0.94 (dd, 3H)

APCI-MS m/z: [MH+] 452.3

HPLC (Method A) Retention time: 5.68 min

HPLC (Method B) Retention time: 7.67 min

### EXAMPLE 101

### 3-[(1-oxidopyridin-2-yl)carbonyl]-9-(spiro[1,3-benzodioxole-2,1′-cyclobutan]-4-ylmethyl)-3,9-diazaspiro[5.5]undecane Trifluoroacetate

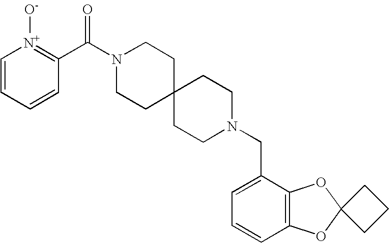

The title compound was prepared by the synthetic procedure of example 95 using Intermediate N and cyclobutanone to give the product as a white solid (22 mg, 20%).

¹H NMR (399.99 MHz, DMSO-D6) δ 9.39 (d, 1H), 8.27 (t, 1H), 7.53-7.37 (m, 3H), 7.03-6.87 (m, 3H), 4.27 (dd, J=13.0, 4.4 Hz, 2H), 3.73-2.94 (m, 8H), 2.62 (q, 4H), 2.01-1.17 (m, 10H)

APCI-MS m/z: [MH+] 450.3

HPLC (Method A) Retention time: 5.47 min

HPLC (Method B) Retention time: 7.55 min

### EXAMPLE 102

### 3-[(1-oxidopyridin-2-yl)carbonyl]-9-(spiro[1,3-benzodioxole-2,11-cyclooctan]-4-ylmethyl)-3,9-diazaspiro[5.5]undecane Trifluoroacetate

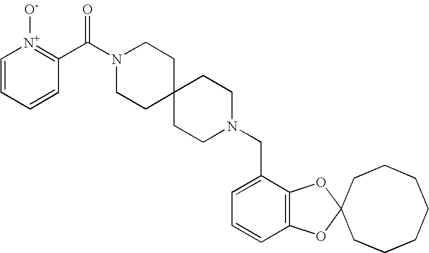

The title compound was prepared by the synthetic procedure of example 95 using Intermediate N and cyclooctanone to give the product as a white solid (73 mg, 58%).

¹H NMR (399.99 MHz, DMSO-D6) δ 9.35 (s, 1H), 8.30-8.23 (m, 1H), 7.51-7.36 (m, 3H), 6.95-6.85 (m, 3H), 4.27-4.18 (m, 2H), 3.71-3.18 (m, 8H), 3.19-2.91 (m, 4H), 2.14-2.00 (m, 4H), 2.00-1.20 (m, 14H)

APCI-MS m/z: [MH+] 506.4

HPLC (Method A) Retention time: 7.93 min

HPLC (Method B) Retention time: 9.66 min

### EXAMPLE 103

### 3-[(2-methyl-2-phenyl-1,3-benzodioxol-4-yl)methyl]-9-[(1-oxidopyridin-2-yl)carbonyl]-3,9-diazaspiro[5.5]undecane Acetate

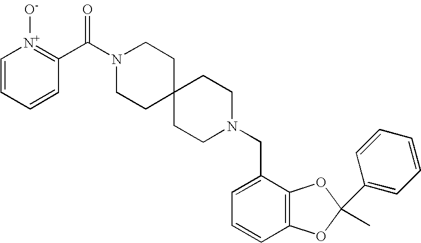

The title compound was prepared by the synthetic procedure of example 95 using Intermediate N and acetophenone and converted to an acetic acid salt to give the product as a white solid (6 mg, 5%).

¹H NMR (399.99 MHz, DMSO-D6) δ 8.26 (d, 1H), 7.63-7.56 (m, 2H), 7.51-7.34 (m, 6H), 6.77 (s, 3H), 3.69-3.44 (m, 2H), 3.15-2.91 (m, 4H), 2.44-2.25 (m, 4H), 1.98 (s, 3H), 1.91 (s, 3H), 1.54-1.20 (m, 8H)

APCI-MS m/z: [MH+] 500.4

HPLC (Method A) Retention time: 6.90 min

HPLC (Method B) Retention time: 8.68 min

### EXAMPLE 104

### 3-[(2-cyclopropyl-2-methyl-1,3-benzodioxol-4-yl)methyl]-9-[(1-oxidopyridin-2-yl)carbonyl]-3,9-diazaspiro[5.5]undecane Trifluoroacetate

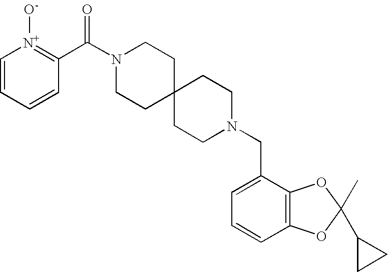

The title compound was prepared by the synthetic procedure of example 95 using Intermediate N and 1-cyclopropylethanone to give the product as a white solid (9 mg, 10%).

¹H NMR (399.99 MHz, DMSO-D6) δ 9.29 (s, 1H), 8.27 (t, 1H), 7.52-7.37 (m, 3H), 6.95-6.83 (m, 3H), 4.29-4.20 (m, 2H), 3.72-2.95 (m, 6H), 2.00-1.79 (m, 2H), 1.77-1.16 (m, 12H), 0.61-0.43 (m, 4H)

APCI-MS m/z: [MH+] 464.2

HPLC (Method A) Retention time: 7.82 min

HPLC (Method B) Retention time: 5.90 min

### EXAMPLE 105

### 3-[(2,2-dimethyl-2H-chromen-8-yl)methyl]-9-isonicotinoyl-3,9-diazaspiro[5.5]undecane Trifluoroacetate

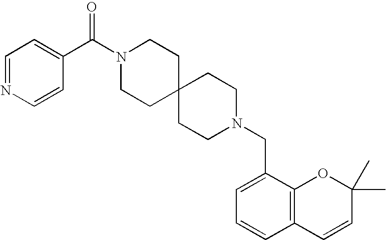

3-isonicotinoyl-3,9-diazaspiro[5.5]undecane (52 mg, 0.20 mmol), 2,2-dimethyl-2H-chromene-8-carbaldehyde (41 mg, 0.22 mmol) and sodium triacetoxyborohydride (85 mg, 0.40 mmol) were dissolved in dichloromethane (5 ml) and acetic acid (300 μl) was added. The reaction was stirred overnight at room temperature after which it was diluted with dichloromethane and washed with saturated aqueous sodium bicarbonate. The organic layer was evaporated and the crude product purified by preparative HPLC (RP-18, gradient acetonitrile/water/TFA 10/90/0.1 to 70/30/0.1) to afford the title compound as a white solid (100 mg, 66%).

¹H NMR (399.99 MHz, CD3OD) δ 8.77 (d, J=5.1 Hz, 2H), 7.68 (d, J=6.3 Hz, 2H), 7.27 (t, J=6.3 Hz, 1H), 7.17 (d, J=7.2 Hz, 1H), 6.99-6.91 (m, 1H), 6.43 (dd, J=9.8, 3.7 Hz, 1H), 5.79 (dd, J=9.8, 5.8 Hz, 1H), 4.31 (d, J=11.3 Hz, 2H), 3.78 (t, J=9.8 Hz, 2H), 3.47-3.33 (m, 4H), 3.26-3.11 (m, 2H), 2.04 (d, J=15.1 Hz, 2H), 1.87-1.77 (m, 1H), 1.75-1.56 (m, 5H), 1.48 (d, J=11.8 Hz, 6H)

APCI-MS m/z: 432.2 [MH+]

HPLC (Method A) Retention time: 5.91 min

HPLC (Method B) Retention time: 8.96 min

### EXAMPLE 106

### 4-({9-[(2,2-dimethyl-2H-chromen-8-yl)methyl]-3,9-diazaspiro[5.5]undec-3-yl}carbonyl)pyridin-3-amine Trifluoroacetate

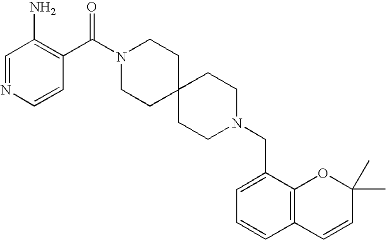

The compound was prepared by the amide coupling procedure of Intermediate S, using Intermediate K and 3-aminoisonicotinic acid as starting materials to give the product as a white solid (27 mg, 23%).

¹H NMR (399.99 MHz, CD3OD) δ 8.14 (s, 1H), 7.98 (d, J=4.9 Hz, 1H), 7.61 (d, J=5.1 Hz, 1H), 7.27 (d, J=7.0 Hz, 1H), 7.16 (d, J=7.2 Hz, 1H), 6.96 (t, J=7.4 Hz, 1H), 6.43 (d, J=9.9 Hz, 1H), 5.79 (d, J=9.5 Hz, 1H), 4.37-4.26 (m, 2H), 3.88-3.68 (m, 2H), 3.50-3.33 (m, 4H), 3.28-3.13 (m, 4H), 2.12-1.97 (m, 2H), 1.91-1.56 (m, 6H), 1.51 (s, 6H)

APCI-MS m/z: 447.3 [MH+]

HPLC (Method A) Retention time: 5.67 min

HPLC (Method B) Retention time: 9.00 min

### EXAMPLE 107

### 6-({9-[(2,2-dimethyl-2H-chromen-8-yl)methyl]-3,9-diazaspiro[5.5]undec-3-yl}carbonyl)pyridin-3-amine Trifluoroacetate

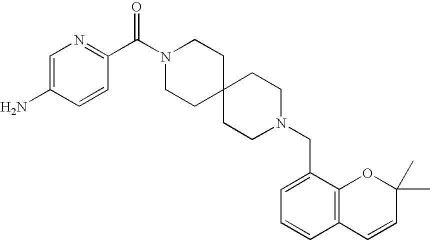

The compound was prepared by the amide coupling procedure of Intermediate S, using Intermediate K and 5-aminopyridine-2-carboxylic acid as starting materials to give the product as a white solid (10 mg, 8%).

¹H NMR (399.99 MHz, CD3OD) δ 7.99 (d, J=2.6 Hz, 1H), 7.59 (d, J=8.8 Hz, 1H), 7.42-7.33 (m, 1H), 7.27 (d, J=7.6 Hz, 1H), 7.17 (d, J=7.3 Hz, 1H), 6.96 (t, J=7.6 Hz, 1H), 6.43 (d, J=9.9 Hz, 1H), 5.80 (d, J=9.9 Hz, 1H), 4.32 (s, 2H), 3.66 (bs, 4H), 3.43 (d, J=13.4 Hz, 2H), 3.28-3.17 (m, 2H), 2.05 (d, J=14.7 Hz, 2H), 1.78 (s, 1H), 1.71-1.61 (m, 3H), 1.58-1.51 (m, 1H), 1.50 (s, 6H)

APCI-MS m/z: 447.3 [MH+]

HPLC (Method A) Retention time: 5.96 min

HPLC (Method B) Retention time: 8.57 min

### EXAMPLE 108

### 2-[2-({9-[(2,2-dimethyl-2H-chromen-8-yl)methyl]-3,9-diazaspiro[5.5]undec-3-yl}carbonyl)phenyl]acetamide

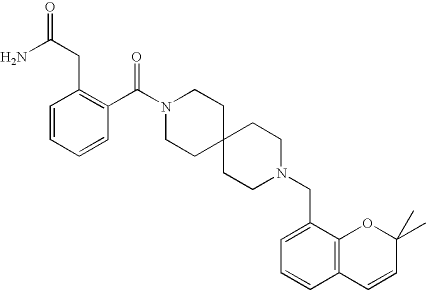

2-[2-(3,9-diazaspiro[5.5]undec-3-ylcarbonyl)phenyl]acetamide (80 mg, 0.25 mmol), 2,2-dimethyl-2H-chromene-8-carbaldehyde (53 mg, 0.28 mmol) and sodium triacetoxyborohydride (106 mg, 0.50 mmol) were dissolved in CH₃CN (5 ml) and stirred at room temperature overnight. The mixture was dissolved in dichloromethane and washed with saturated aqueous sodium bicarbonate. The organic layer was evaporated and the crude product purified by preparative HPLC (RP-18, gradient acetonitrile/water/TFA 10/90/0.1 to 70/30/0.1) then eluted through a SCX ion exchange column to afford the title compound as a white solid (45 mg, 37%).

¹H NMR (399.99 MHz, CD3OD) δ 7.41-7.37 (m, 2H), 7.35-7.30 (m, 1H), 7.23 (d, J=7.3 Hz, 1H), 7.16 (d, J=7.6 Hz, 1H), 6.94 (d, J=6.9 Hz, 1H), 6.81 (t, J=7.5 Hz, 1H), 6.36 (d, J=9.8 Hz, 1H), 5.68 (d, J=9.7 Hz, 1H), 3.82-3.68 (m, 2H), 3.66 (s, 2H), 3.61 (s, 2H), 3.28-3.22 (m, 2H), 2.57 (s, 4H), 1.65-1.54 (m, 6H), 1.46-1.39 (m, 2H), 1.40 (s, 6H)

APCI-MS m/z: 488.4 [MH+]

HPLC (Method A) Retention time: 6.97 min

HPLC (Method B) Retention time: 9.38 min

### EXAMPLE 109

### 3-(2,3-dihydro-1,4-benzodioxin-5-ylmethyl)-9-isonicotinoyl-3,9-diazaspiro[5.5]undecane Trifluoroacetate

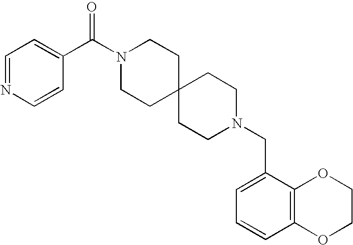

The title compound was prepared by the procedure of Example 1 using 2,3-dihydro-1,4-benzodioxin-5-carbaldehyde and Intermediate S to give the product as a white solid (85 mg, 42%).

¹H NMR (399.99 MHz, CD3OD) δ 8.79 (d, J=4.7 Hz, 2H), 7.71 (d, J=6.1 Hz, 2H), 7.05-6.88 (m, 3H), 4.43-4.23 (m, 4H), 3.85-3.71 (m, 2H), 3.46-3.33 (m, 6H), 3.28-3.10 (m, 4H), 2.09-1.97 (m, 2H), 1.88-1.79 (m, 1H), 1.75-1.42 (m, 5H)

APCI-MS m/z: 408.4 [MH+]

HPLC (Method A) Retention time: 3.74 min

HPLC (Method B) Retention time: 6.79 min

### EXAMPLE 110

### 3-[(2,2-dimethyl-2,3-dihydro-1,4-benzodioxin-5-yl)methyl]-9-isonicotinoyl-3,9-diazaspiro[5.5]undecane Trifluoroacetate

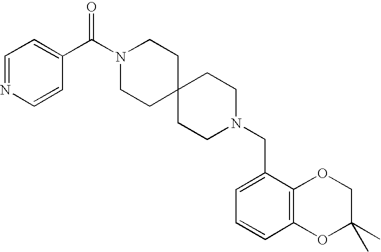

The title compound was prepared by the procedure of Example 77 using Intermediate J and Intermediate S to give the product as a white solid (120 mg, 70%).

APCI-MS m/z: 436.3 [MH+]

HPLC (Method A) Retention time: 4.71 min

HPLC (Method B) Retention time: 7.78 min

### EXAMPLE 111

### 3-[(2,2-dimethyl-2,3-dihydro-1-benzofuran-7-yl)methyl]-9-[3-(3-pyridin-2-yl-1,2,4-oxadiazol-5-yl)propanoyl]-3,9-diazaspiro[5.5]undecane

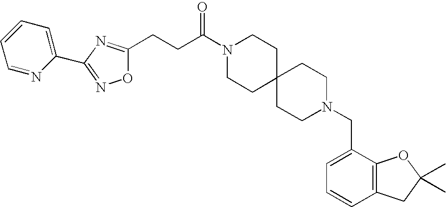

The title compound was prepared according to Example 24 using 3-[(2,2-dimethyl-2,3-dihydro-1-benzofuran-7-yl)methyl]-3,9-diazaspiro[5.5]undecane dihydrochloride (60 mg, 0.16 mmol) and 3-(3-pyridin-2-yl-1,2,4-oxadiazol-5-yl)propanoic acid (42 mg, 0.19 mmol). The crude product was purified by preparative HPLC (RP-18, gradient acetonitrile/water/TFA 10/90/0.1 to 70/30/0.1) then eluted through a SCX ion exchange column to afford the title compound as a white solid (25 mg, 31%).

¹H NMR (499.881 MHz, CD3OD) δ 8.70 (d, J=4.4 Hz, 1H), 8.15 (d, J=7.7 Hz, 1H), 8.02 (t, J=7.7 Hz, 1H), 7.59 (t, J=6.3 Hz, 1H), 7.30 (dd, J=7.3, 2.0 Hz, 1H), 7.20 (dd, J=7.2, 3.8 Hz, 1H), 6.93 (td, J=7.5, 3.5 Hz, 1H), 4.27 (d, J=6.6 Hz, 2H), 3.62-3.52 (m, 4H), 3.44-3.36 (m, 2H), 3.29-3.27 (m, 2H), 3.23-3.15 (m, 2H), 3.13-3.05 (m, 4H), 2.01 (d, J=15.7 Hz, 2H), 1.76 (t, J=5.7 Hz, 1H), 1.67-1.58 (m, 3H), 1.54 (t, J=5.7 Hz, 1H), 1.50 (d, J=5.3 Hz, 6H), 1.42 (t, J=5.7 Hz, 1H)

APCI-MS m/z: 516.2 [MH+]

HPLC (Method A) Retention time: 7.20 min

### EXAMPLE 112

### 4-({9-[(2,2-dimethyl-2H-chromen-8-yl)methyl]-3,9-diazaspiro[5.5]undec-3-yl}carbonyl)pyridin-2-amine (Trifluoroacetate)

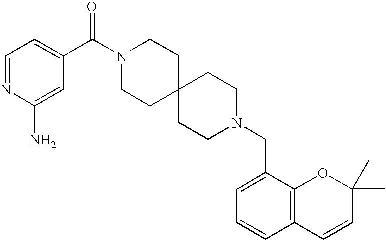

The title compound was prepared by the amide coupling procedure of Intermediate S, using Intermediate K and 2-aminoisonicotinic acid as starting materials to give the product as a white solid (46 mg, 33%).

¹H NMR (399.99 MHz, CD3OD) δ 7.97-7.89 (m, 1H), 7.27 (d, J=7.5 Hz, 1H), 7.17 (d, J=7.4 Hz, 1H), 7.01-6.91 (m, 2H), 6.85 (t, J=5.2 Hz, 1H), 6.43 (d, J=10.0 Hz, 1H), 5.84-5.74 (m, 1H), 4.32 (d, J=7.8 Hz, 2H), 3.74 (bs, 2H), 3.52-3.35 (m, 4H), 3.27-3.11 (m, 4H), 2.10-1.97 (m, 2H), 1.90-1.76 (m, 2H), 1.76-1.53 (m, 4H), 1.53 (s, 6H)

APCI-MS m/z: 447.3 [MH+]

HPLC (Method A) Retention time: 5.85 min

HPLC (Method B) Retention time: 8.99 min

### EXAMPLE 113

### 6-({9-[(2,2-dimethyl-2H-chromen-8-yl)methyl]-3,9-diazaspiro[5.5]undec-3-yl}carbonyl)pyridin-2(1H)-one

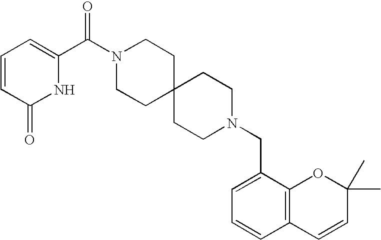

The title compound was prepared by the amide coupling procedure of Intermediate S, using Intermediate K and 6-hydroxypyridine-2-carboxylic acid as starting materials to give the product as a white solid (15 mg, 13%).

¹H NMR (399.99 MHz, CD3OD) δ 7.67-7.57 (m, 1H), 7.54 (s, 1H), 7.27 (d, J=7.7 Hz, 1H), 7.17 (d, J=6.4 Hz, 1H), 6.96 (t, J=7.5 Hz, 1H), 6.61 (d, J=9.2 Hz, 1H), 6.50 (d, J=6.9 Hz, 1H), 6.43 (d, J=9.8 Hz, 1H), 5.80 (d, J=9.9 Hz, 1H), 4.31 (s, 2H), 3.82-3.35 (m, 2H), 3.27-3.15 (m, 4H), 2.11-1.92 (m, 4H), 1.84-1.59 (m, 4H), 1.55 (s, 6H)

APCI-MS m/z: 448.2 [MH+]

HPLC (Method A) Retention time: 6.29 min

HPLC (Method B) Retention time: 7.92 min

### EXAMPLE 114

### 2-({9-[(2,2-dimethyl-2,3-dihydro-1-benzofuran-7-yl)methyl]-3,9-diazaspiro[5.5]undec-3-yl}carbonyl)benzonitrile Trifluoroacetate

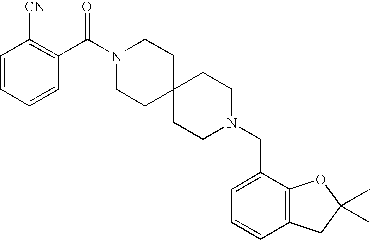

The title compound was prepared by the amide coupling procedure of Intermediate S, using Intermediate A and 2-cyanobenzoic acid as starting materials to give the product as a white solid (63 mg, 58%).

¹H NMR (399.99 MHz, CD3OD) δ 7.89-7.82 (m, 1H), 7.82-7.73 (m, 1H), 7.69-7.60 (m, 1H), 7.57-7.49 (m, 1H), 7.33-7.25 (m, 1H), 7.22-7.15 (m, 1H), 6.97-6.88 (m, 1H), 4.26 (d, J=14.4 Hz, 2H), 3.88-3.76 (m, 2H), 3.46-3.36 (m, 2H), 3.28-3.06 (m, 6H), 2.12-1.98 (m, 2H), 1.88-1.78 (m, 1H), 1.74-1.56 (m, 5H), 1.53 (s, 3H), 1.50 (s, 3H)

APCI-MS m/z: 444.3 [MH+]

HPLC (Method A) Retention time: 7.69 min

HPLC (Method B) Retention time: 10.38 min

### EXAMPLE 115

### 4-({9-[(2,2-dimethyl-2,3-dihydro-1-benzofuran-7-yl)methyl]-3,9-diazaspiro[5.5]undec-3-yl}carbonyl)pyridine-2,6-diol Trifluoroacetate

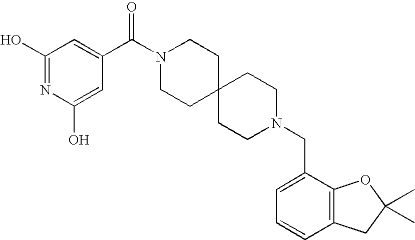

The title compound was prepared by the amide coupling procedure of Intermediate S, using Intermediate A and 2,6-dihydroisonicotinic acid as starting materials to give the product as a white solid (10 mg, 10%).

¹H NMR (399.99 MHz, CD3OD) δ 7.30 (d, J=7.5 Hz, 1H), 7.20 (d, J=7.3 Hz, 1H), 6.93 (t, J=7.5 Hz, 1H), 5.74 (d, J=4.0 Hz, 1H), 4.26 (d, J=8.5 Hz, 2H), 3.78-3.65 (m, 2H), 3.51-3.35 (m, 4H), 3.26-3.05 (m, 4H), 2.13-1.97 (m, 2H), 1.82-1.72 (m, 1H), 1.71-1.43 (m, 5H), 1.51 (s, 3H), 1.49 (s, 3H)

APCI-MS m/z: 453.3 [MH+]

HPLC (Method A) Retention time: 5.36 min

HPLC (Method B) Retention time: 5.05 min

### EXAMPLE 116

### 3-[(6-fluoro-4H-1,3-benzodioxin-8-yl)methyl]-9-isonicotinoyl-3,9-diazaspiro[5.5]undecane Trifluoroacetate

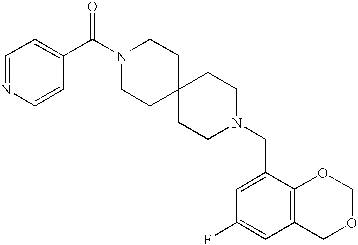

The title compound was prepared by the procedure of Example 1 using 6-fluoro-4H-1,3-benzodioxin-8-carbaldehyde and Intermediate S to give the product as a white solid (58 mg, 28%).

¹H NMR (399.99 MHz, CD3OD) δ 8.88 (s, 2H), 7.90 (d, J=6.4 Hz, 2H), 7.17 (d, J=5.2 Hz, 1H), 7.00 (d, J=8.0 Hz, 1H), 5.46-5.16 (m, 2H), 4.92-4.89 (m, 2H), 4.32 (m, 2H), 3.78 (bs, 2H), 3.51-3.07 (m, 8H), 2.13-1.93 (m, 2H), 1.84 (s, 1H), 1.79-1.65 (m, 3H), 1.59 (s, 1H), 1.47 (s, 1H)

APCI-MS m/z: 426.3 [MH+]

HPLC (Method A) Retention time: 3.47 min

HPLC (Method B) Retention time: 7.09 min

### EXAMPLE 117

### 4-({9-[(2,2-dimethyl-2H-chromen-8-yl)methyl]-3,9-diazaspiro[5.5]undec-3-yl}carbonyl)pyridazin-3-amine

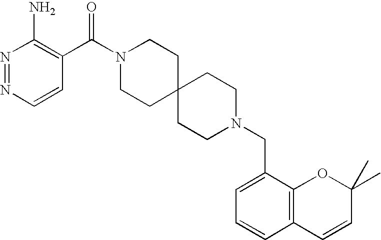

3-[(2,2-dimethyl-2H-chromen-8-yl)methyl]-3,9-diazaspiro[5.5]undecane dihydrochloride (75 mg, 0.19 mmol), 3-aminopyridazine-4-carboxylic acid (32 mg, 0.23 mmol), PYBOP (120 mg, 0.23 mmol) and triethylamine (106 μl, 0.76 mmol) were dissolved in dichloromethane (5 ml) and stirred at room temperature for 1 h. The mixture was diluted with dichloromethane (10 ml) and washed with saturated aqueous sodium bicarbonate. The organic layer was evaporated and the crude product purified by preparative HPLC (RP-18, gradient acetonitrile/water/TFA 5/95/0.1 to 60/40/0.1) then eluted through a SCX ion exchange column to afford the title compound as a white solid (20 mg, 24%).

¹H NMR (399.99 MHz, CD3OD) δ 8.52 (d, J=4.7 Hz, 1H), 7.29 (d, J=4.8 Hz, 1H), 7.16 (d, J=6.4 Hz, 1H), 6.94 (d, J=6.4 Hz, 1H), 6.82 (t, J=7.5 Hz, 1H), 6.36 (d, J=9.9 Hz, 1H), 5.68 (d, J=9.8 Hz, 1H), 3.72 (s, 2H), 3.62 (s, 2H), 2.59 (s, 4H), 1.67-1.54 (m, 6H), 1.49 (s, 2H), 1.41 (s, 6H)

APCI-MS m/z: 448.2 [MH+]

HPLC (Method A) Retention time: 5.48 min

HPLC (Method B) Retention time: 7.55 min

### EXAMPLE 118

### 5-chloro-4-({9-[(2,2-dimethyl-1,3-benzodioxol-4-yl)methyl]-1-3,9-diazaspiro[5.5]undec-3-yl}carbonyl)pyrimidin-2-amine

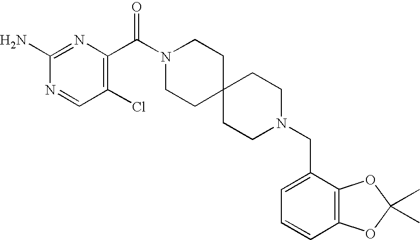

The title compound was prepared according to procedure of Example 64 using Intermediate B and 2-amino-5-chloropyrimidine-4-carboxylic acid as starting materials to give the product as a white solid (18 mg, 14%).

¹H NMR (399.99 MHz, DMSO-D6) δ 8.34 (s, 1H), 7.10 (s, 2H), 6.80-6.65 (m, 3H), 3.56 (s, 2H), 3.14 (s, 2H), 3.42-3.37 (m, 2H), 2.40-2.25 (m, 4H), 1.61 (s, 6H), 1.53-1.31 (m, 8H)

APCI-MS mm/z: [MH+] 472.4

HPLC (Method A) Retention time: 6.15 min

HPLC (Method B) Retention time: 8.68 min

### EXAMPLE 119

### 4-({9-[(2,2-dimethyl-2,3-dihydro-1-benzofuran-7-yl)methyl]-3,9-diazaspiro[5.5]undec-3-yl}carbonyl)pyrimidin-2-amine

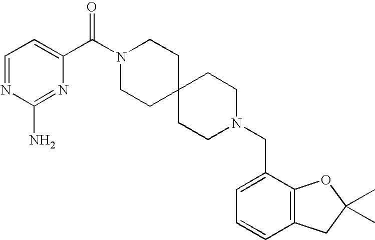

The title compound was prepared with the procedure of Example 64 using Intermediate A and 2-aminopyrimidine-4-carboxylic acid as starting materials and THF/NMP (10:1) as a solvent to give the product as a white solid (38 mg, 28%).

¹H NMR (399.99 MHz, DMSO-D6) δ 9.17 (s, 1H), 8.33 (t, 1H), 7.26 (dd, 2H), 6.97-6.79 (m, 2H), 6.57 (t, 1H), 4.20 (d, 2H), 3.32-2.97 (m, 10H), 1.88 (d, 2H), 1.68-1.26 (m, 12H)

APCI-MS m/z: 436.2 [MH+]

HPLC (Method A) Retention time: 5.20 min

HPLC (Method B) Retention time: 7.58 min

### EXAMPLE 120

### 4-({9-[(2,2-dimethyl-1,3-benzodioxol-4-yl)methyl]-3,9-diazaspiro[5.5]undec-3-yl}carbonyl)pyrimidin-2-amine

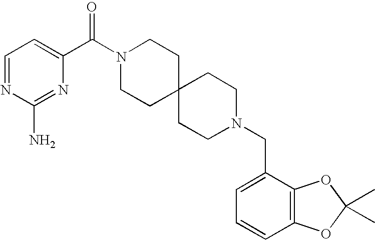

The title compound was prepared with the procedure of Example 119 using Intermediate B and 2-aminopyrimidine-4-carboxylic acid as starting materials to give the product as a white solid (106 mg, 27%).

¹H NMR (399.99 MHz, DMSO-D6) δ 9.29 (s, 1H), 8.34 (t, 1H), 6.98-6.80 (m, 3H), 6.60 (t, 1H), 4.25 (d, 2H), 3.62-3.47 (m, 4H), 3.20-2.99 (m, 4H), 1.96-1.80 (m, 2H), 1.75-1.44 (m, 10H), 1.43-1.24 (m, 2H)

APCI-MS m/z: 438.3 [MH+]

HPLC (Method A) Retention time: 5.00 min

HPLC (Method B) Retention time: 7.55 min

### EXAMPLE 121

### 4-({9-[(2,2-dimethyl-2H-chromen-8-yl)methyl]-3,9-diazaspiro[5.5]undec-3-yl}carbonyl)pyrimidin-2-amine

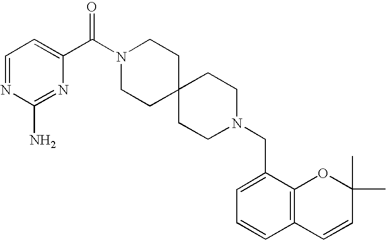

The title compound was prepared with the procedure of Example 119 using Intermediate K and 2-aminopyrimidine-4-carboxylic acid as starting materials to give the product as a white solid (14 mg, 8%).

¹H NMR (399.99 MHz, DMSO-D6) δ 9.20-8.94 (m, 1H), 8.33 (t, 1H), 7.30 (d, J=7.5 Hz, 1H), 7.18 (d, 1H), 6.98-6.89 (m, 2H), 6.89-6.78 (m, 1H), 6.57 (t, 1H), 6.45 (dd, 1H), 5.82 (dd, 1H), 4.24 (d, 2H), 3.64-3.51 (m, 4H), 3.31-2.95 (m, 4H), 1.89 (d, 2H), 1.78-1.11 (m, 12H)

APCI-MS m/z: 448.3 [MH+]

HPLC (Method A) Retention time: 6.06 min

HPLC (Method B) Retention time: 8.70 min

### EXAMPLE 122

### 8-[(2,2-dimethyl-2,3-dihydro-1-benzofuran-7-yl)methyl]-2-(pyridin-4-ylacetyl)-2,8-diazaspiro[4.5]decane

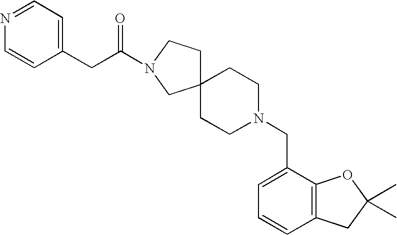

The title compound was prepared by the procedure of Example 64 using 8-[(2,2-dimethyl-2,3-dihydro-1-benzofuran-7-yl)methyl]-2,8-diazaspiro[4.5]decane and pyridin-4-ylacetic acid as starting materials to give the product as a white solid (5 mg, 5%).

¹H NMR (399.99 MHz, CD3OD) δ 8.77 (s, 2H), 7.97 (d, 2H), 7.25 (dd, 2H), 6.96-6.89 (m, 1H), 4.28 (d, 2H), 4.17-4.05 (m, 2H), 3.83-3.64 (m, 2H), 3.62-3.44 (m, 4H), 3.25-3.04 (m, 4H), 2.18-1.78 (m, 4H), 1.56-1.44 (m, 6H)

APCI-MS m/z: 420.3 [MH+]

HPLC (Method A) Retention time: 4.91 min

HPLC (Method B) Retention time: 8.08 min

### EXAMPLE 123

### 4-({9-[(2,2-dimethyl-2,3-dihydro-1-benzofuran-4-yl)methyl]-3,9-diazaspiro[5.5]undec-3-yl}carbonyl)pyridazin-3-amine

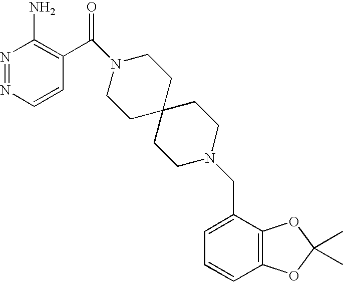

The title compound was prepared by the procedure of Example 64 using Intermediate C and 3-aminopyridazine-4-carboxylic acid as starting materials to give the product as a white solid (34 mg, 13%).

¹H NMR (399.99 MHz, CD3OD) δ 8.53 (s, 1H), 7.80 (s, 1H), 7.23 (t, 1H), 6.95 (d, 1H), 6.78 (d, 1H), 4.25 (d, 2H), 3.78 (s, 2H), 3.42 (d, 4H), 3.27-3.11 (m, 4H), 2.05 (d, 2H), 1.93-1.57 (m, 6H), 1.48 (s, 6H)

APCI-MS m/z: 436.3 [MH+]

HPLC (Method A) Retention time: 4.83 min

HPLC (Method B) Retention time: 7.54 min

### EXAMPLE 124

### 4-({9-[(2,2-dimethyl-2,3-dihydro-1-benzofuran-4-yl)methyl]-3,9-diazaspiro[5.5]undec-3-yl}carbonyl)pyrimidin-2-amine

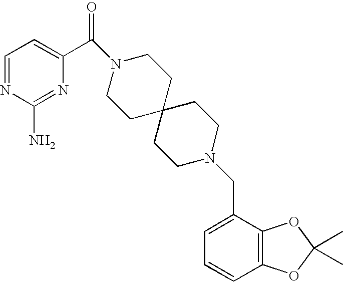

The title compound was prepared by the procedure of Example 64 using Intermediate C and 2-aminopyrimidine-4-carboxylic acid as starting materials to give the product as a white solid (40 mg, 15%).

¹H NMR (399.99 MHz, CD3OD) δ 8.39 (t, 1H), 7.23 (t, 1H), 6.95 (dd, 1H), 6.89 (t, 1H), 6.78 (d, 1H), 4.25 (d, 2H), 3.79-3.65 (m, 2H), 3.51-3.37 (m, 4H), 3.28-3.10 (m, 4H), 2.05 (d, 2H), 1.88-1.74 (m, 2H), 1.73-1.41 (m, 10H)

APCI-MS m/z: 436.3 [MH+]

HPLC (Method A) Retention time: 5.15 min

HPLC (Method B) Retention time: 7.83 min

### EXAMPLE 125

### 4-({9-[(3,3-dimethyl-2,3-dihydro-1,4-benzodioxin-5-yl)methyl]-3,9-diazaspiro[5.5]undec-3-yl}carbonyl)pyridin-3-amine Trifluoroacetate

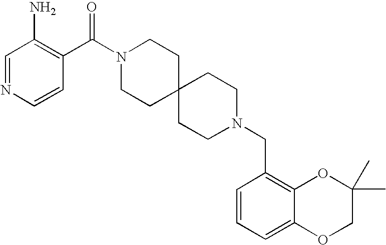

The title compound was prepared by the procedure of Example 77 using intermediate M and intermediate Z as starting materials to give the product as a white solid (120 mg, 60%).

APCI-MS m/z: 451.3 [MH+]

HPLC (Method A) Retention time: 4.71 min

HPLC (Method B) Retention time: 8.13 min

¹H NMR (399.99 MHz, CD3OD) δ 8.17 (s, 1H), 8.00 (d, J=5.1 Hz, 1H), 7.64-7.61 (m, 1H), 7.06-6.98 (m, 2H), 6.92 (t, J=7.8 Hz, 1H), 4.31 (s, 2H), 3.97 (s, 2H), 3.78 (s, 2H), 3.48-3.33 (m, 4H), 3.28-3.09 (m, 2H), 2.02 (d, J=14.5 Hz, 2H), 1.89-1.56 (m, 5H), 1.52-1.44 (m, 3H), 1.39 (s, 6H)

### EXAMPLE 126

### 4-({9-[(3,3-dimethyl-2,3-dihydro-1,4-benzodioxin-5-yl)methyl]-3,9-diazaspiro[5.5]undec-3-yl}carbonyl)pyridin-2-amine Trifluoroacetate

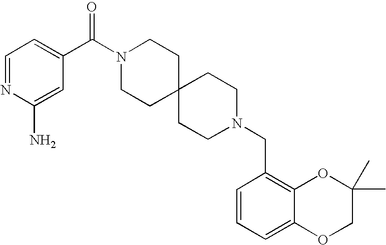

The title compound was prepared by the procedure of Example 77 using intermediate L and intermediate Z as starting materials to give the product as a white solid (102 mg, 58%).

APCI-MS m/z: 451.0 [MH+]

HPLC (Method A) Retention time: 4.81 min

HPLC (Method B) Retention time: 8.16 min

¹H NMR (399.99 MHz, CD3OD) δ 7.92 (t, J=5.4 Hz, 1H), 7.06-6.95 (m, 3H), 6.92 (t, J=7.8 Hz, 1H), 6.87-6.82 (m, 1H), 4.31 (d, J=6.8 Hz, 2H), 3.96 (d, J=3.6 Hz, 2H), 3.78-3.69 (m, 2H), 3.46-3.35 (m, 5H), 3.27-3.10 (m, 2H), 2.08-1.97 (m, 2H), 1.82-1.64 (m, 5H), 1.60-1.53 (m, 1H), 1.51-1.44 (m, 1H), 1.40 (s, 3H), 1.38 (s, 3H)

### EXAMPLE 127

### 4-({9-[(2,2,3,3-tetramethyl-2,3-dihydro-1-benzofuran-7-yl)methyl]-3,9-diazaspiro[5.5]undec-3-yl}carbonyl)pyrimidin-2-amine Trifluoroacetate

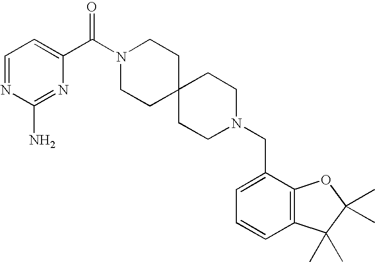

The title compound was prepared by the procedure of Example 119 using intermediate Q and 2-aminopyrimidine-4-carboxylic acid as starting materials to give the product as a white solid (50 mg, 36%).

APCI-MS m/z: 464.3 [MH+]

HPLC (Method A) Retention time: 6.43 min

HPLC (Method B) Retention time: 8.89 min

¹H NMR (399.99 MHz, CD3OD) δ 8.39 (t, J=5.2 Hz, 1H), 7.28-7.18 (m, 2H), 7.02-6.95 (m, 1H), 6.89-6.82 (m, 1H), 4.28 (d, J=8.2 Hz, 2H), 3.77-3.68 (m, 2H), 3.49-3.35 (m, 4H), 3.26-3.10 (m, 2H), 2.10-2.00 (m, 2H), 1.81-1.48 (m, 6H), 1.36 (d, J=7.4 Hz, 6H), 1.23 (d, J=4.2 Hz, 6H)

### EXAMPLE 128

### 6-({9-[(2,2,3,3-tetramethyl-2,3-dihydro-1-benzofuran-7-yl)methyl]-3,9-diazaspiro[5.5]undec-3-yl}carbonyl)pyridin-3-amine Trifluoroacetate

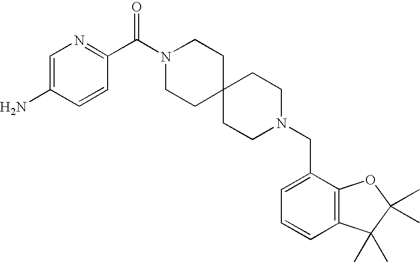

The title compound was prepared by the procedure of Example 64 using intermediate Q and 5-aminopyridine-2-carboxylic acid as starting materials to give the product as a white solid (60 mg, 43%).

APCI-MS m/z: 463.1 [MH+]

HPLC (Method A) Retention time: 6.23 min

HPLC (Method B) Retention time: 8.98 min

¹H NMR (399.99 MHz, CD3OD) δ 7.99 (s, 1H), 7.64-7.56 (m, 1H), 7.44-7.34 (m, 1H), 7.28-7.18 (m, 2H), 6.98 (t, J=7.5 Hz, 1H), 4.28 (s, 2H), 3.82-3.52 (m, 4H), 3.44-3.34 (m, 2H), 3.25-3.10 (m, 2H), 2.04 (d, J=14.4 Hz, 2H), 1.81-1.72 (m, 2H), 1.71-1.59 (m, 2H), 1.58-1.48 (m, 2H), 1.36 (s, 6H), 1.23 (s, 6H)

### EXAMPLE 129

### 4-({9-[(2,2,3,3-tetramethyl-2,3-dihydro-1-benzofuran-7-yl)methyl]-3,9-diazaspiro[5.5]undec-3-yl}carbonyl)pyridin-2-amine Trifluoroacetate

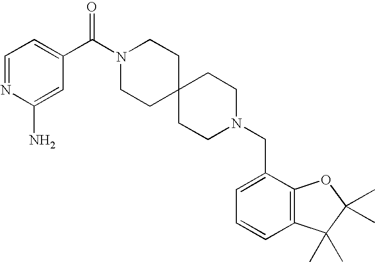

The title compound was prepared by the procedure of Example 64 using intermediate Q and 2-aminoisonicotinic acid as starting materials to give the product as a white solid (90 mg, 65%).

APCI-MS m/z: 463.4 [MH+]

HPLC (Method A) Retention time: 6.07 min

HPLC (Method B) Retention time: 9.26 min

¹H NMR (399.99 MHz, CD3OD) δ 7.90 (t, J=5.4 Hz, 1H), 7.27-7.18 (m, 2H), 7.01-6.93 (m, 2H), 6.86 (t, J=5.1 Hz, 1H), 4.27 (d, J=9.2 Hz, 2H), 3.77-3.68 (m, 2H), 3.44-3.34 (m, 4H), 3.26-3.09 (m, 2H), 2.05 (d, J=14.9 Hz, 2H), 1.82-1.75 (m, 1H), 1.74-1.59 (m, 3H), 1.59-1.52 (m, 1H), 1.51-1.44 (m, 1H), 1.36 (d, J=8.3 Hz, 6H), 1.22 (d, J=4.2 Hz, 6H)

### EXAMPLE 130

### 4-({9-[(2,2,3,3-tetramethyl-2,3-dihydro-1-benzofuran-7-yl)methyl]-3,9-diazaspiro[5.5]undec-3-yl}carbonyl)pyridin-3-amine Trifluoroacetate

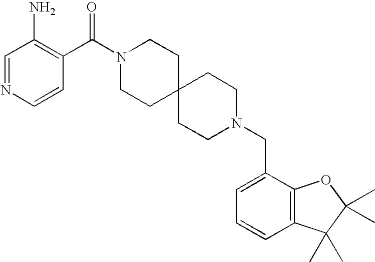

The title compound was prepared by the procedure of Example 64 using intermediate Q and 3-aminoisonicotinic acid as starting materials to give the product as a white solid (55 mg, 44%).

APCI-MS m/z: 463.1 [MH+]

HPLC (Method A) Retention time: 5.99 min

HPLC (Method B) Retention time: 9.24 min

¹H NMR (399.99 MHz, CD3OD) δ 8.15 (s, 1H), 7.98 (d, J=5.3 Hz, 1H), 7.62 (d, J=5.5 Hz, 1H), 7.28-7.18 (m, 2H), 6.97 (t, J=7.5 Hz, 1H), 4.32-4.23 (m, 2H), 3.84-3.72 (m, 2H), 3.45-3.33 (m, 4H), 3.27-3.08 (m, 2H), 2.04 (d, J=14.4 Hz, 2H), 1.87-1.78 (m, 1H), 1.74-1.56 (m, 4H), 1.53-1.45 (m, 1H), 1.36 (d, J=5.0 Hz, 6H), 1.23 (s, 6H)

### EXAMPLE 131

### 4-({9-[(2,2-dimethyl-2H-chromen-5-yl)methyl]-3,9-diazaspiro[5.5]undec-3-yl}carbonyl)pyrimidin-2-amine

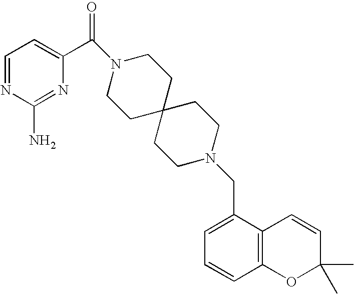

The title compound was prepared by the procedure of Example 1 using Intermediate X and Intermediate P as starting materials to give the product as a white solid (8 mg, 3%).

¹H NMR (399.99 MHz, DMSO-D6) δ 9.00 (s, 1H), 8.33 (dd, 1H), 7.25-7.13 (m, 1H), 7.10-6.98 (m, 1H), 6.93-6.75 (m, 3H), 6.57 (dd, H), 5.93 (dd, 1H), 4.42-4.28 (m, 2H), 3.63-3.50 (m, 4H), 3.29-3.06 (m, 4H), 2.00-1.76 (m, 2H), 1.74-1.17 (m, 12H)

APCI-MS m/z: 448.3 [MH+]

HPLC (Method A) Retention time: 5.83 min

HPLC (Method B) Retention time: 8.93 min

### EXAMPLE 132

### 4-({9-[(2,2-dimethyl-3,4-dihydro-2H-chromen-8-yl)methyl]-3,9-diazaspiro[5.5]undec-3-yl}carbonyl)pyrimidin-2-amine

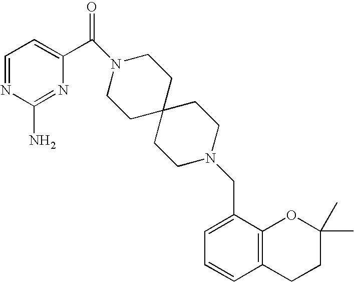

The title compound was prepared by the procedure of Example 1 using Intermediate Y and Intermediate P as starting materials to give the product as a white solid (49 mg, 21%).

¹H NMR (399.99 MHz, DMSO-D6) δ 8.94 (d, 1H), 8.33 (t, 1H), 7.27 (d, J=7.3 Hz, 1H), 7.20 (d, 1H), 6.92-6.78 (m, 3H), 6.60-6.50 (m, 1H), 4.22 (dd, 2H), 3.62-3.49 (m, 4H), 3.31-2.99 (m, 4H), 2.83-2.72 (m, 2H), 1.89 (d, J=14.5 Hz, 2H), 1.83-1.76 (m, 2H), 1.67-1.46 (m, 4H), 1.43-1.22 (m, 8H)

APCI-MS m/z: 450.3 [MH+]

HPLC (Method A) Retention time: 6.11 min

HPLC (Method B) Retention time: 8.85 min

### EXAMPLE 133

### 6-amino-3-({9-[(2,2-dimethyl-1,3-benzodioxol-4-yl)methyl]-3,9-diazaspiro[5.5]undec-3-yl}carbonyl)pyridin-2(1H)-one

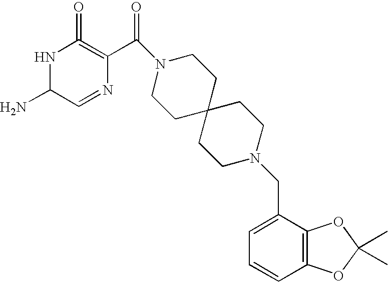

The title compound was prepared by the procedure of Example 119 using Intermediate B and 6-amino-2-oxo-1,2-dihydropyridine-3-carboxylic acid as starting materials to give the product as a white solid (36 mg, 28%).

¹H NMR (399.99 MHz, DMSO-D6) δ 11.36-10.24 (m, 1H), 9.31 (s, 1H), 7.29 (d, H), 7.01-6.83 (m, 3H), 6.62-6.10 (m, 2H), 5.44-5.29 (m, 1H), 4.24 (d, 2H), 3.42-3.19 (m, 6H), 3.17-3.01 (m, 2H), 1.87 (d, 2H), 1.68 (s, 6H), 1.61-1.45 (m, 4H), 1.31 (s, 2H)

APCI-MS m/z: 453.3 [MH+]

HPLC (Method A) Retention time: 4.97 min

HPLC (Method B) Retention time: 6.67 min

### EXAMPLE 134

### 2-({9-[(2,2-dimethyl-2,3-dihydro-1-benzofuran-4-yl)methyl]-3,9-diazaspiro[5.5]undec-3-yl}carbonyl)pyridin-3-amine

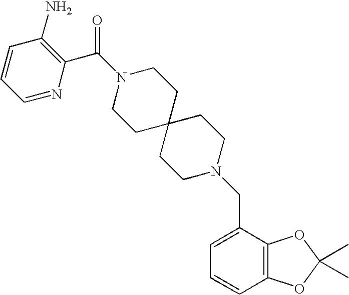

The title compound was prepared by the procedure of Example 119 using Intermediate C and 3-aminopyridine-2-carboxylic acid as starting materials to give the product as a white solid (38 mg, 30%).

¹H NMR (399.99 MHz, DMSO-D6) δ 9.18 (s, 1H), 7.79 (s, 1H), 7.24-7.14 (m, 3H), 6.95 (d, 1H), 6.78 (d, 1H), 4.22 (s, 2H), 3.40-2.98 (m, 8H), 1.89 (d, 2H), 1.78-1.24 (m, 12H)

APCI-MS mm/z: 435.3 [MH+]

HPLC (Method A) Retention time: 4.55 min

HPLC (Method B) Retention time: 8.33 min

### EXAMPLE 135

### 8-[(2,2-dimethyl-2,3-dihydro-1-benzofuran-7-yl)methyl]-2-isonicotinoyl-2,8-diazaspiro[4.5]decane

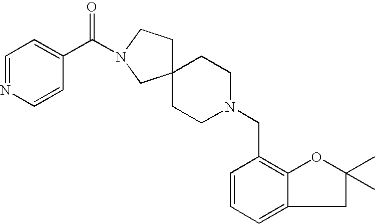

### a) 8-[(2,2-dimethyl-2,3-dihydro-1-benzofuran-7-yl)methyl]-2,8-diazaspiro[4.5]decane Hydrochloric Acid Salt

The title compound was prepared by the procedure of intermediate A using tert-butyl 2,8-diazaspiro[4.5]decane-2-carboxylate hydrochloric acid salt and 2,2-dimethyl-2,3-dihydro-1-benzofuran-7-carbaldehyde as starting material to give the product (0.2 g, 42%) as white solid.

LCMS (ESI): m/z 301 (M+1).

### b) 8-[(2,2-dimethyl-2,3-dihydro-1-benzofuran-7-yl)methyl]-2-isonicotinoyl-2,8-diazaspiro[4.5]decane

To a solution of 8-[(2,2-dimethyl-2,3-dihydro-1-benzofuran-7-yl)methyl]-2,8-diazaspiro[4.5]decane hydrochloric acid salt (0.1 g, 0.3 mmol) in CH₂Cl₂ (15 ml) were added isonicotinic acid (0.04 g, 0.33 mmol), EDCI (0.057 g, 0.036 mmol), HOBT (0.006 g, 0.04 mmol) and Et₃N (0.061 g, 0.6 mmol) under N₂. Reaction mixture was stirred at RT for 16 h. Reaction mixture was diluted with CH₂Cl₂, washed with saturated NaHCO₃ solution followed by water and brine, dried over Na₂SO₄. Filtrate was concentrated and crude was purified by preparative HPLC to yield the product (0.02 g, 15%) as pale yellow liquid.

¹H NMR (400 MHz, CDCl₃): δ 1.27 (s, 6H), 1.46 (m, 3H), 1.82 (m, 3H), 2.64 (brs, 3H), 3.02 (m, 3H), 3.21 (s, 2H), 3.45 (m, 2H), 3.71 (s, 2H), 3.84 (s, 1H), 6.82 (m, 1H), 7.10 (m, 2H), 7.37 (brs, 2H), 8.72 (brs, 2H).

LCMS (ESI): m/z 406 (M+1).

HPLC (Method C) RT: 2.65 min

### EXAMPLE 136

### 8-[(2,2-dimethyl-2H-chromen-8-yl)methyl]-2-isonicotinoyl-2,8-diazaspiro[4.5]decane

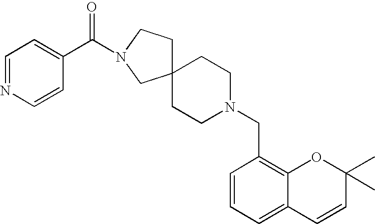

### a) 8-[(2,2-dimethyl-2H-chromen-8-yl)methyl]-2,8-diazaspiro[4.5]decane Hydrochloric Acid Salt

The title compound was prepared by the procedure of intermediate A using tert-butyl 2,8-diazaspiro[4.5]decane-2-carboxylate hydrochloric acid salt and 2,2-dimethyl-2H-chromene-8-carbaldehyde as starting material to give the product (0.05 g, 34%) as white solid.

LCMS (ESI): m/z 313 (M+1).

### b) 8-[(2,2-dimethyl-2H-chromen-8-yl)methyl]-2-isonicotinoyl-2,8-diazaspiro[4.5]decane

To a solution of 8-[(2,2-dimethyl-2H-chromen-8-yl)methyl]-2,8-diazaspiro[4.5]decane hydrochloric salt (0.05 g, 0.14 mmol) in CH₂Cl₂ (15 ml) were added isonicotinic acid (0.02 g, 15 mmol), EDCI (0.032 g, 16 mmol), HOBt (0.003 g, 0.2 mmol) and Et₃N (0.028 g, 28 mmol) under N₂. Reaction mixture was stirred at RT for 10 h. Reaction mixture was diluted with CH₂Cl₂, washed with saturated NaHCO₃ solution followed by water and brine, dried over Na₂SO₄. Filtrate was concentrated and crude was purified by column chromatography over silica gel using methanol and dichloromethane as eluent to yield the product (0.05 g, 84%) as pale yellow liquid.

¹H NMR (400 MHz, CDCl₃): δ 1.41 (s, 3H), 1.47 (s, 3H), 1.79 (m, 3H), 3.21 (s, 1H), 3.46 (t, 2H, J=7.0 Hz), 3.53 (s, 1H), 3.71 (t, 2H, J=7.3 Hz), 5.63 (t, 1H, J=9.3 Hz), 6.35 (t, 1H, J=7.8 Hz), 6.88 (m, 2H), 7.37 (m, 2H), 8.72 (m, 2H).

LCMS (ESI): m/z 418 (M+1).

HPLC (Method C) RT: 6.46 min

### EXAMPLE 137

### 2-[(2,2-dimethyl-2,3-dihydro-1-benzofuran-7-yl)methyl]-8-isonicotinoyl-2,8-diazaspiro[4.5]decane

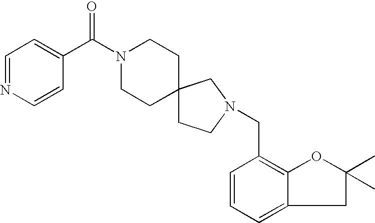

### a) 2-[(2,2-dimethyl-2,3-dihydro-1-benzofuran-7-yl)methyl]-2,8-diazaspiro[4.5]decane Hydrochloric Acid Salt

The title compound was prepared by the procedure of intermediate A using tert-butyl 2,8-diazaspiro[4.5]decane-8-carboxylate hydrochloric acid salt and 2,2-dimethyl-2,3-dihydro-1-benzofuran-7-carbaldehyde as starting material to give the product (0.26 g, 57%) as yellow liquid.

LCMS (ESI): m/z 401 (M+1).

### b) 2-[(2,2-dimethyl-2,3-dihydro-1-benzofuran-7-yl)methyl]-8-isonicotinoyl-2,8-diazaspiro[4.5]decane

To a solution of 2-[(2,2-dimethyl-2,3-dihydro-1-benzofuran-7-yl)methyl]-2,8-diazaspiro[4.5]decane hydrochloric acid salt (0.05 g, 0.14 mmol) in CH₂Cl₂ (15 ml) were added isonicotinic acid (0.02 g, 15 mmol), EDCI (0.032 g, 16 mmol), HOBt (0.003 g, 0.2 mmol) and Et₃N (0.028 g, 28 mmol) under N₂. Reaction mixture was stirred at RT for 10 h. Reaction mixture was diluted with CH₂Cl₂, washed with saturated NaHCO₃ solution followed by water and brine, dried over Na₂SO₄. Filtrate was concentrated and crude was purified by column chromatography over silica gel using methanol and dichloromethane as eluent to yield the product (0.05 g, 84%) as pale yellow liquid.

¹H NMR (400 MHz, CDCl₃): δ 1.47 (s, 6H), 1.55 (brs, 2H), 1.69 (m, 2H), 2.4-2.9 (m, 4H), 3.02 (s, 2H), 3.36 (t, 2H, J=5.5 Hz), 3.57 (m, 2H), 3.67 (s, 2H), 3.71 (s, 2H), 6.81 (t, 1H, J=7.5 Hz), 7.06 (d, 1H, J=7.2 Hz), 7.13 (d, 1H, J=7.2 Hz), 7.20 (d, 1H, J=5.6 Hz), 8.55 (brs, 2H).

LCMS (ESI): m/z 420 (M+1).

HPLC (Method C) RT: 5.56 min

### EXAMPLE 138

### 7-[(2,2-dimethyl-2,3-dihydro-1-benzofuran-7-yl)methyl]-2-isonicotinoyl-2,7-diazaspiro[3.5]nonane

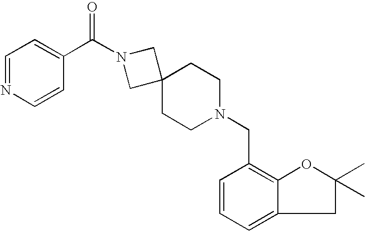

### a) 7-[(2,2-dimethyl-2,3-dihydro-1-benzofuran-7-yl)methyl]-2,7-diazaspiro[3.5]nonane Hydrochloric Acid

The title compound was prepared by the procedure of intermediate A tert-butyl 2,7-diazaspiro[3.5]nonane-2-carboxylate hydrochloric acid salt and 2,2-dimethyl-2,3-dihydro-1-benzofuran-7-carbaldehyde as starting material to give the product (0.2 g, 57%) as a white solid.

### b) 7-[(2,2-dimethyl-2,3-dihydro-1-benzofuran-7-yl)methyl]-2-isonicotinoyl-2,7-diazaspiro[3.5]nonane

To a solution of 7-[(2,2-dimethyl-2,3-dihydro-1-benzofuran-7-yl)methyl]-2,7-diazaspiro[3.5]nonane hydrochloric acid (0.1 g, 0.3 mmol) in CH₂Cl₂ (15 ml) were added isonicotinic acid (0.042 g, 0.33 mmol), EDCI (0.069 g, 0.36 mmol), HOBt (0.006 g, 0.04 mmol) and Et₃N (0.06 g, 0.6 mmol) under N₂. Reaction mixture was stirred at RT for 16 h. Reaction mixture was diluted with CH₂Cl₂, washed with saturated NaHCO₃ solution followed by water and brine, dried over Na₂SO₄. Filtrate was concentrated and crude was purified by preparative HPLC to yield the product (40 mg) as pale yellow liquid.

¹H NMR (400 MHz, CDCl₃): δ 1.47 (s, 6H), 1.87 (brs, 4H), 3.04 (s, 2H), 3.74 (s, 2H), 3.90 (s, 2H), 3.97 (s, 2H), 6.83 (t, 1H, J=7.4 Hz), 7.12 (t, 2H, J=6.4 Hz), 7.50 (d, 2H, J=5.2 Hz), 8.73 (d, 2H, J=5.7 Hz).

LCMS (ESI): m/z 392 (M+1).

HPLC (Method C) RT: 7.25 min

### EXAMPLE 139

### 7-[(2,2-dimethyl-2H-chromen-8-yl)methyl]-2-isonicotinoyl-2,7-diazaspiro[3.5]nonane

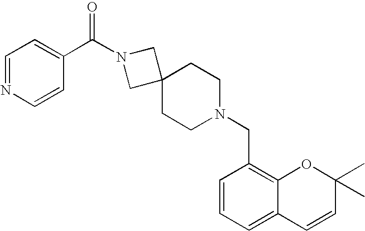

### a) 7-[(2,2-dimethyl-2H-chromen-8-yl)methyl]-2,7-diazaspiro[3.5]nonane Hydrochloride Salt

The title compound was prepared by the procedure of intermediate A tert-butyl 2,7-diazaspiro[3.5]nonane-2-carboxylate hydrochloric acid salt and 2,2-dimethyl-2H-chromene-8-carbaldehyde as starting material to give the product (0.3 g, 57%) as white solid.

LCMS (ESI): m/z 299 (M+1).

### b) 7-[(2,2-dimethyl-2H-chromen-8-yl)methyl]-2-isonicotinoyl-2,7-diazaspiro[3.5]nonane

To a solution of 7-[(2,2-dimethyl-2H-chromen-8-yl)methyl]-2,7-diazaspiro[3.5]nonane hydrochloride salt (0.15 g, 0.44 mmol) in CH₂Cl₂ (15 ml) were added isonicotinic acid (0.06 g, 0.48 mmol), EDCI (0.1 g, 0.52 mmol), HOBt (0.008 g, 0.06 mmol) and Et₃N (0.089 g, 0.88 mmol) under N₂. Reaction mixture was stirred at RT for 10 h. Reaction mixture was diluted with CH₂Cl₂, washed with saturated NaHCO₃ solution followed by water and brine, dried over Na₂SO₄. Filtrate was concentrated and crude was purified by column chromatography over silica gel using methanol and dichloromethane as eluent to yield the product (50 g, 28%) as pale yellow liquid.

¹H NMR (400 MHz, CDCl₃): δ 1.44 (s, 6H), 2.44 (m, 4H), 3.47 (s, 2H), 3.54 (s, 2H), 3.72 (s, 2H), 3.81 (s, 2H), 5.62 (d, 1H, J=9.8 Hz), 6.34 (d, 1H, J=9.8 Hz), 6.83 (t, 1H, J=7.52 Hz), 6.92 (m, 1H), 7.22 (m, 3H), 8.55 (m, 2H).

LCMS (ESI): m/z 390 (M+1).

HPLC (Method C) RT: 7.44 min

### EXAMPLE 140

### 2-[(2,2-dimethyl-2,3-dihydro-1-benzofuran-7-yl)methyl]-8-(pyridin-4-ylacetyl)-2,8-diazaspiro[4.5]decane

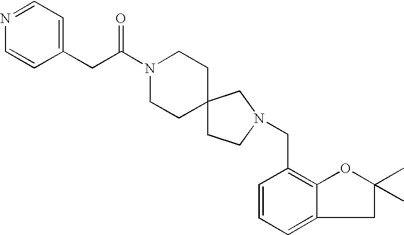

The title compound was prepared by the procedure of example 137 using 2-[(2,2-dimethyl-2,3-dihydro-1-benzofuran-7-yl)methyl]-2,8-diazaspiro[4.5]decane hydrochloric acid salt (0.1 g, 0.29 mmol) and 2-(4-pyridyl)acetic acid (0.053 g, 0.31 mmol) to give the product (0.01 g, 8%) as yellow liquid.

¹H NMR (400 MHz, CDCl₃): δ 1.47 (s, 6H), 1.54 (br, 2H), 2.20 (br, 4H), 2.50 (m, 2H), 2.69 (m, 2H), 3.02 (s, 2H), 3.27 (brs, 2H), 3.62 (s, 2H), 3.70 (m, 2H), 6.80 (t, 1H, J=7.5 Hz), 7.04 (d, 1H, J=7.2 Hz), 7.13 (d, 1H, J=7.2 Hz), 7.27 (d, 2H, J=5.6 Hz), 8.69 (d, 2H, J=5.3 Hz).

LCMS (ESI): m/z 406 (M+1).

HPLC (Method C) RT: 5.68 min

### EXAMPLE 141

### 7-[(2,2-dimethyl-2H-chromen-8-yl)methyl]-2-(pyridin-4-ylacetyl)-2,7-diazaspiro[3.5]nonane

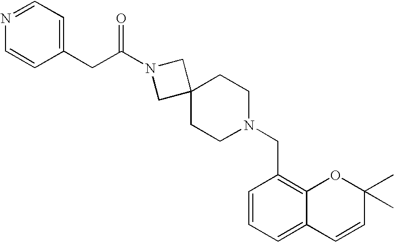

The title compound was prepared by the procedure of example 139 using 7-[(2,2-dimethyl-2H-chromen-8-yl)methyl]-2,7-diazaspiro[3.5]nonane hydrochloride salt (0.1 g, 0.29 mmol) and 2-(4-pyridyl)acetic acid (0.053 g, 0.31 mmol) to give the product (0.05 g, 28%) as pale yellow liquid.

¹H NMR (400 MHz, CDCl₃): δ 1.43 (s, 6H), 2.53 (m, 4H), 3.52 (s, 6H), 3.64 (s, 2H), 3.92 (s, 2H), 3.97 (s, 2H), 5.63 (d, 1H, J=9.8 Hz), 6.34 (d, 1H, J=9.8 Hz), 6.84 (t, 1H, J=7.52 Hz), 6.93 (m, 1H), 7.18 (m, 1H), 7.70 (m, 2H), 8.74 (m, 2H).

LCMS (ESI): m/z 404 (M+1).

HPLC (Method C) RT: 7.65 min

### EXAMPLE 142

### 7-[(2,2-dimethyl-2,3-dihydro-1-benzofuran-7-yl)methyl]-2-(pyridin-4-ylacetyl)-2,7-diazaspiro[3.5]nonane

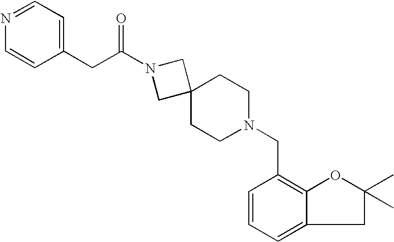

The title compound was prepared by the procedure of example 138 using 7-[(2,2-dimethyl-2,3-dihydro-1-benzofuran-7-yl)methyl]-2,7-diazaspiro[3.5]nonane hydrochloric acid (0.1 g, 0.3 mmol) and 2-(4-pyridyl)acetic acid (0.057 g, 0.33 mmol) to give the product (40 g, 31%) as pale yellow liquid. ¹H NMR (400 MHz, CDCl₃): δ 1.47 (s, 6H), 1.77 (m, 4H), 2.42 (brs, 3H), 3.02 (s, 2H), 3.46 (s, 2H), 3.52 (s, 2H), 3.71 (s, 2H), 3.79 (s, 2H), 6.81 (t, 1H, J=7.4 Hz), 7.06 (d, 1H, J=7.2 Hz), 7.12 (d, 1H, J=7.2 Hz), 7.23 (d, 2H, J=5.7 Hz), 8.56 (d, 2H, J=5.9 Hz).

LCMS (ESI): m/z 406 (M+1).

HPLC (Method C) RT: 7.51 min

### EXAMPLE 143

### 2-[4-({9-[(3,3-dimethyl-2,3-dihydro-1,4-benzodioxin-5-yl)methyl]-3,9-diazaspiro[5.5]undec-3-yl}carbonyl)pyridin-3-yl]acetamide Ditrifluoroacetate

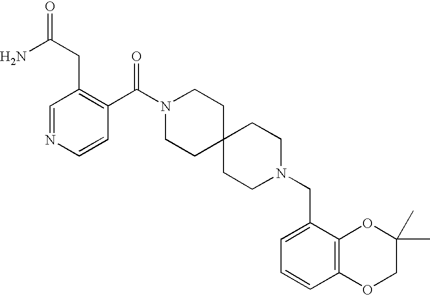

[4-({9-[(3,3-dimethyl-2,3-dihydro-1,4-benzodioxin-5-yl)methyl]-3,9-diazaspiro[5.5]undec-3-yl}carbonyl)pyridin-3-yl]acetic acid (Example 162) (90 mg, 0.18 mmol), HBTU (76 mg, 0.20 mmol) and Triethylamine (50 μl, 0.36 mmol) were dissolved in NMP (10 ml). The mixture was stirred at rt for 1 hr after which it was cooled to 0° C. on an ice-bath. Ammonia gas was bubbled through the mixture and it was stirred for an additional 15 min. The mixture was diluted with EtOAc and washed with H₂O. The organic layer was evaporated and purified by preparative HPLC (RP-18, gradient acetonitrile/water/TFA 5/95/0.1 to 60/40/0.1) to afford 55 mg (42%) of the title compound as a white solid.

¹H NMR (299.945 MHz, CD3OD) δ 8.69 (s, 1H), 8.64 (dd, J=5.3, 2.3 Hz, 1H), 7.57 (d, J=5.4 Hz, 1H), 7.05-7.02 (m, 1H), 7.01 (d, J=3.7 Hz, 1H), 6.97-6.89 (m, 1H), 4.31 (d, J=9.0 Hz, 2H), 3.97 (d, J=5.0 Hz, 2H), 3.84-3.69 (m, 4H), 3.47-3.36 (m, 4H), 3.26-3.10 (m, 2H), 2.12-1.95 (m, 2H), 1.86-1.42 (m, 6H), 1.40 (s, 3H), 1.38 (s, 3H)

APCI-MS m/z: 493.4 [MH+]

HPLC (Method A) RT: 4.50 min

HPLC (Method B) RT: 7.19 min

### EXAMPLE 144

### 2-[(2,2-dimethyl-2H-chromen-8-yl)methyl]-8-isonicotinoyl-2,8-diazaspiro[4.5]decane

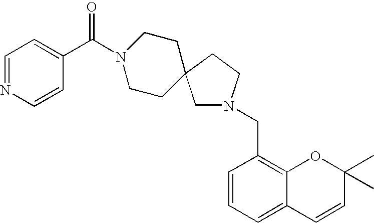

### a) 2-[(2,2-dimethyl-2H-chromen-8-yl)methyl]-2,8-diazaspiro[4.5]decane Hydrochloric Acid

The title compound was prepared by the procedure of intermediate A tert-butyl 2,8-diazaspiro[4.5]decane-8-carboxylate hydrochloric acid salt and 2,2-dimethyl-2H-chromene-8-carbaldehyde as starting material to give the product (0.2 g, 53%) as white solid.

LCMS (ESI): m/z 313 (M+1).

### b) 2-[(2,2-dimethyl-2H-chromen-8-yl)methyl]-8-isonicotinoyl-2,8-diazaspiro[4.5]decane

To a solution of 2-[(2,2-dimethyl-2H-chromen-8-yl)methyl]-2,8-diazaspiro[4.5]decane hydrochloric acid (0.1 g, 0.28 mmol) in CH₂Cl₂ (15 ml) were added isonicotinic acid (0.04 g, 0.31 mmol), EDCI (0.06 g, 0.33 mmol), HOBt (0.005 g, 0.04 mmol) and Et₃N (0.056 g, 0.56 mmol) under N₂. Reaction mixture was stirred at RT for 10 h. Reaction mixture was diluted with CH₂Cl₂, washed with saturated NaHCO₃ solution followed by water and brine, dried over Na₂SO₄. Filtrate was concentrated and crude was purified by column chromatography over silica gel using methanol and dichloromethane as eluent to yield the product (0.06 g, 51%) as yellow liquid.

¹H NMR (400 MHz, CDCl₃): δ 1.41 (s, 6H), 3.29 (brs, 2H), 3.75 (m, 4H), 5.62 (d, 1H, J=9.78 Hz), 6.34 (d, 1H, J=9.78 Hz), 6.84 (t, 1H, J=7.4 Hz), 6.92 (m, 1H), 7.27 (m, 2H), 8.71 (m, 2H).

LCMS (ESI): m/z 418 (M+1).

HPLC (Method C) RT: 7.59 min

### EXAMPLE 145

### 8-[(2,2-dimethyl-2H-chromen-8-yl)methyl]-2-(pyridin-4-ylacetyl)-2,8-diazaspiro[4.5]decane

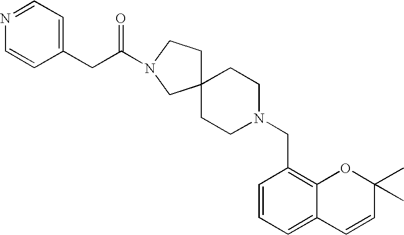

The title compound was prepared by the procedure of example 136 using 8-[(2,2-dimethyl-2H-chromen-8-yl)methyl]-2,8-diazaspiro[4.5]decane hydrochloric acid (0.1 g, 0.28 mmol) and 2-(4-pyridyl)acetic acid hydrochloride (0.06 g, 0.34 mmol) to give the product (0.06 g, 50%) as pale yellow liquid.

¹H NMR (400 MHz, CDCl₃): δ 1.44 (s, 6H), 1.55 (m, 2H), 1.84 (m, 2H), 3.26 (s, 1H), 3.37 (s, 2H), 3.51 (m, 3H), 3.64 (s, 1H), 5.64 (d, 1H, J=9.8 Hz), 6.34 (d, 1H, 9.8 Hz), 6.85 (m, 1H), 6.93 (m, 1H), 7.23 (s, 2H), 8.56 (m, 2H)

LCMS (ESI): m/z 432 (M+1).

HPLC (Method C) RT: 7.56 min

### EXAMPLE 146

### 3-[4-({9-[(2,2-dimethyl-2,3-dihydro-1-benzofuran-4-yl)methyl]-3,9-diazaspiro[5.5]undec-3-yl}carbonyl)pyridin-3-yl]propanamide Trifluoroacetate

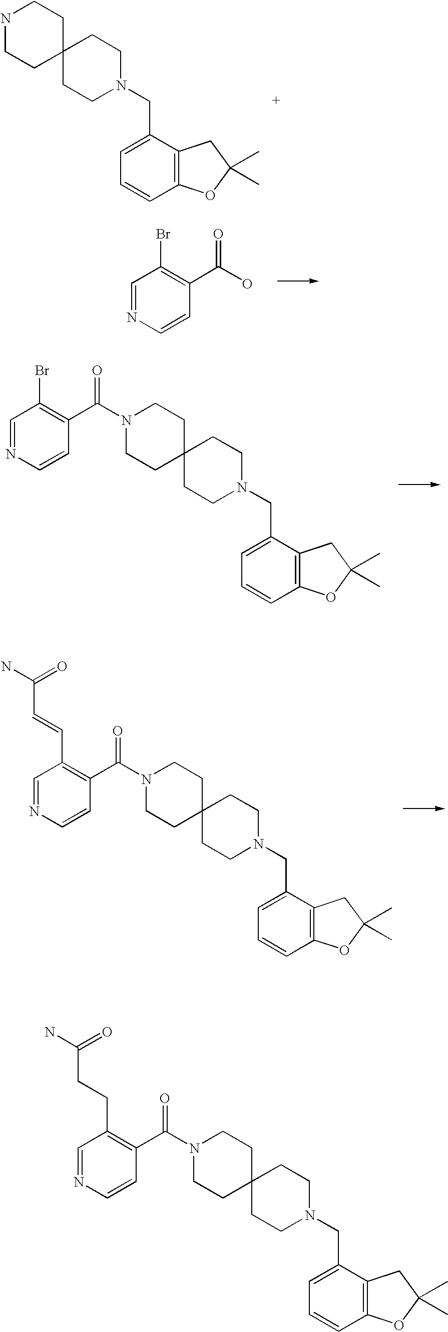

### a) 3-(3-bromoisonicotinoyl)-9-[(2,2-dimethyl-2,3-dihydro-1-benzofuran-4-yl)methyl]-3,9-diazaspiro[5.5]undecane

3-[(2,2-dimethyl-2,3-dihydro-1-benzofuran-4-yl)methyl]-3,9-diazaspiro[5.5]undecane (Intermediate C) (200 mg, 0.64 mmol), 3-bromoisonicotinic acid (156 mg, 0.77 mmol), HBTU (292 mg, 0.77 mmol) and triethylamine (178 μl, 1.28 mmol) were dissolved in dichloromethane (10 ml). The mixture was stirred for 1 hr after which it was washed with NaHCO₃ (sat.) and the organic layer was dried over Na₂SO₄ and evaporated, affording 400 mg of a yellow oil which was used directly in the next step.

APCI-MS m/z: 498.2, 500.2 [MH+]

### b) (2E)-3-[4-({9-[(2,2-dimethyl-2,3-dihydro-1-benzofuran-4-yl)methyl]-3,9-diazaspiro[5.5]undec-3-yl}carbonyl)pyridin-3-yl] acrylamide

3-(3-bromoisonicotinoyl)-9-[(2,2-dimethyl-2,3-dihydro-1-benzofuran-4-yl)methyl]-3,9-diazaspiro[5.5]undecane (all material from previous step, 0.64 mmol), acrylamide (136 mg, 1.92 mmol), and triethylamine (267 μl, 1.92 mmol) were dissolved in dry CH₃CN (4 ml). Argon was bubbled through the mixture and Palladium (II) Acetate (7 mg, 0.03 mmol) and tri-o-tolylphosphine (18 mg, 0.06 mmol) were added. The mixture was heated in a microwave oven (CEM Explorer) at 100° C. for 10 minutes. Silca-SH (Pd scavenger) was added and the mixture was stirred at room temperature for an additional 10 minutes. The mixture was filtered through celite, the filtrate diluted with EtOAc (25 ml) and washed with H₂O and brine. The organic layer was dried over Na₂SO₄ and evaporated affording 400 mg of a yellow oil, which was used directly in the next step.

APCI-MS m/z: 489.4 [MH+]

### c) 3-[4-({9-[(2,2-dimethyl-2,3-dihydro-1-benzofuran-4-yl)methyl]-3,9-diazaspiro[5.5]undec-3-yl}carbonyl)pyridin-3-yl]propanamide Trifluoroacetate

(2E)-3-[4-({9-[(2,2-dimethyl-2,3-dihydro-1-benzofuran-4-yl)methyl]-3,9-diazaspiro[5.5]undec-3-yl}carbonyl)pyridin-3-yl]acrylamide (from the previous step, 0.64 mmol) was dissolved in Methanol (10 ml) and 10% Pd/C (40 mg) was added. The mixture was hydrogenated over night at room temperature and atmospheric pressure. The catalyst was filtered off using celite and the filtrate was evaporated. The residue was purified by preparative HPLC (RP-18, gradient acetonitrile/water/TFA 5/95/0.1 to 60/40/0.1) to afford 240 mg (62%) of the title compound as a white solid.

¹H NMR (399.99 MHz, CD3OD) δ 8.78 (s, 1H), 8.69 (dd, J=5.3, 3.5 Hz, 1H), 7.75 (d, J=5.5 Hz, 1H), 7.22 (td, J=7.9, 2.3 Hz, 1H), 6.95 (t, J=7.1 Hz, 1H), 6.78 (dd, J=8.0, 2.3 Hz, 1H), 4.25 (d, J=12.2 Hz, 2H), 3.97-3.68 (m, 2H), 3.48-3.36 (m, 4H), 3.24-2.88 (m, 8H), 2.64 (t, J=7.0 Hz, 2H), 2.17-1.56 (m, 8H), 1.47 (d, J=7.8 Hz, 6H)

APCI-MS m/z: 491.4 [MH+]

HPLC (Method A) RT: 4.46 min

HPLC (Method B) RT: 7.49 min

### EXAMPLE 147

### 4-({9-[(3,3-dimethyl-2,3-dihydro-1,4-benzodioxin-5-yl)methyl]-3,9-diazaspiro[5.5]undec-3-yl}carbonyl)pyridine-2-carbonitrile Trifluoroacetate

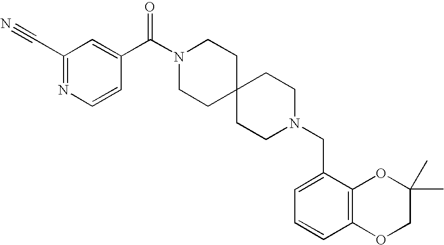

3-[(3,3-dimethyl-2,3-dihydro-1,4-benzodioxin-5-yl)methyl]-3,9-diazaspiro[5.5]undecane (Intermediate AA) (75 mg, 0.23 mmol), 2-cyanoisonicotinic acid (38 mg, 0.27 mmol), HBTU (102 mg, 0.27 mmol) and triethylamine (62 μl, 0.45 mmol) was dissolved in dichloromethane (10 ml) and NMP (2 ml) and stirred at room temperature over night. The solution was diluted with additional dichloromethane (10 ml) and washed with NaHCO₃ (sat.) and brine. The organic layer was dried over Na₂SO₄, evaporated and purified twice by preparative HPLC (RP-18, gradient acetonitrile/water/TFA 5/95/0.1 to 65/35/0.1) to afford 50 mg (41%) of the title compound as a white solid.

¹H NMR (399.99 MHz, CD3OD) δ 8.80 (d, J=4.4 Hz, 1H), 7.93 (d, J=3.2 Hz, 1H), 7.67 (s, 1H), 7.06-6.99 (m, 2H), 6.93 (t, J=7.8 Hz, 1H), 4.31 (d, J=10.1 Hz, 2H), 3.97 (d, J=5.0 Hz, 2H), 3.81-3.72 (m, 2H), 3.47-3.33 (m, 2H), 3.28-3.09 (m, 2H), 2.10-1.97 (m, 3H), 1.88-1.44 (m, 7H), 1.40 (s, 3H), 1.38 (s, 3H)

APCI-MS m/z: 461.2 [MH+]

HPLC (Method A) RT: 6.74 min

HPLC (Method B) RT: 9.47 min

### EXAMPLE 148

### 4-({9-[(3,3-dimethyl-2,3-dihydro-1,4-benzodioxin-5-yl)methyl]-3,9-diazaspiro[5.5]undec-3-yl}carbonyl)pyridine-2-carboxamide Trifluoroacetate

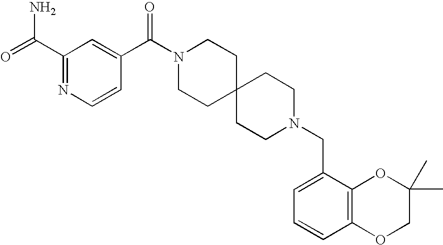

4-({9-[(3,3-dimethyl-2,3-dihydro-1,4-benzodioxin-5-yl)methyl]-3,9-diazaspiro[5.5]undec-3-yl}carbonyl)pyridine-2-carbonitrile trifluoroacetate (Example 147) (100 mg, 0.22 mmol) was dissolved in DMSO (2 ml) and cooled to 0° C. Potassium carbonate (36 mg, 0.26 mmol) was added followed by dropwise addition of Hydrogen Peroxide (35% in H2O) (24 μl, 0.24 mmol). The cool-bath was removed and the mixture stirred at room temperature over night. The mixture was diluted with EtOAc and washed with aqueous Na₂S₂O₃ (10%) and brine. The organic layer was evaporated and purified by preparative HPLC (RP-18, gradient acetonitrile/water/TFA 5/9510.1 to 65/35/0.1) to afford 60 mg (46%) of the title compound as a white solid.

¹H NMR (399.99 MHz, CD3OD) δ 8.75 (s, 1H), 8.07 (s, 1H), 7.56 (s, 1H), 7.06-6.99 (m, 2H), 6.96-6.90 (m, 1H), 4.31 (d, J=12.9 Hz, 2H), 3.97 (d, J=6.4 Hz, 2H), 3.83-3.73 (m, 2H), 3.46-3.35 (m, 4H), 3.28-3.11 (m, 2H), 2.11-2.01 (m, 2H), 1.85-1.78 (m, 1H), 1.74-1.55 (m, 4H), 1.51-1.43 (m, 1H), 1.41 (s, 3H), 1.38 (s, 3H)

APCI-MS m/z: 479.3 [MH+]

HPLC (Method A) RT: 4.97 min

HPLC (Method B) RT: 7.98 min

### EXAMPLE 149

### (2E)-3-[2-({9-[(3,3-dimethyl-2,3-dihydro-1,4-benzodioxin-5-yl)methyl]-3,9-diazaspiro[5.5]undec-3-yl}carbonyl)phenyl]acrylamide Trifluoroacetate

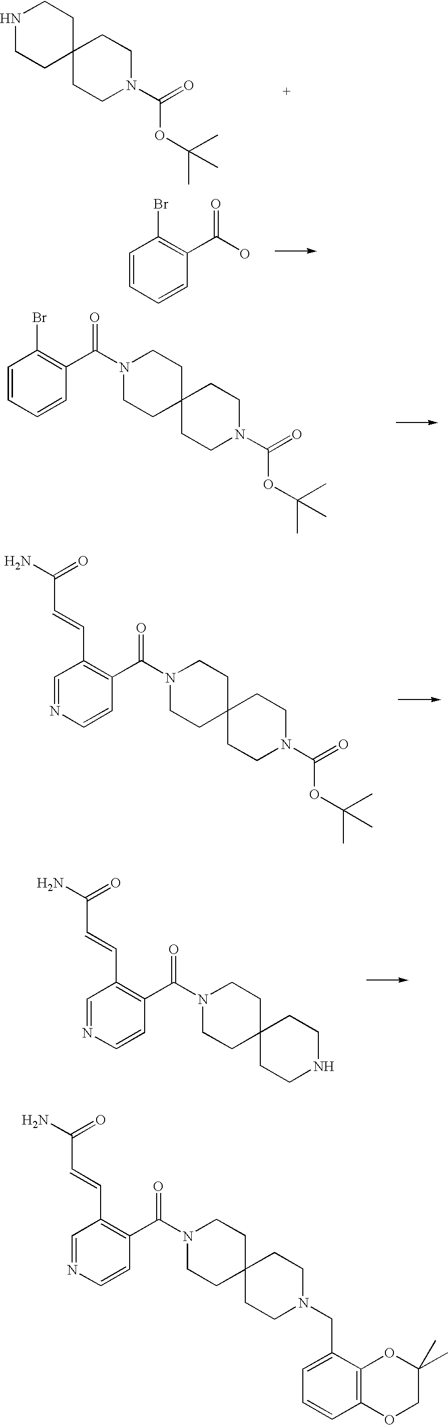

### a) tert-butyl 9-(2-bromobenzoyl)-3,9-diazaspiro[5.5]undecane-3-carboxylate

tert-butyl 3,9-diazaspiro[5.5]undecane-3-carboxylate hydrochloride (1.0 g, 3.44 mmol), 2-bromobenzoic acid (0.83 g, 4.13 mmol), HBTU (1.57 g, 4.13 mmol) and triethylamine (1.44 ml, 10.3 mmol) were dissolved in dichloromethane (20 ml) and stirred at room temperature over night. The mixture was disluted with dichloromethane and washed with aqueous NaHCO₃ (sat.). The organic layer was dried over Na₂SO₄ and evaporated. The residue was purified using column chromatography on SiO₂ eluting with Heptane:EtOAc 10:1 to 1:2 affording 1.38 g (92%) of the title compound as a colourless oil.

¹H NMR (299.944 MHz, CDCl3) δ 7.61-7.55 (m, 1H), 7.39-7.32 (m, 1H), 7.27-7.21 (m, 2H), 3.86-3.72 (m, 2H), 3.47-3.31 (m, 4H), 3.31-3.10 (m, 2H), 1.71-1.33 (m, 17H)

### b) tert-butyl 9-{2-[(1E)-3-amino-3-oxoprop-1-en-1-yl]benzoyl}-3,9-diazaspiro[5.5]undecane-3-carboxylate

Synthesised according to Example 146b from the product of Example 149a and purified by preparative HPLC (RP-18, gradient acetonitrile/water/TFA 15/85/0.1 to 90/10/0.1) to afford 120 mg (61%) of the title compound as a yellow oil.

APCI-MS m/z: 428.2 [MH+]

### c) (2E)-3-[2-(3,9-diazaspiro[5.5]undec-3-ylcarbonyl)phenyl]acrylamide

tert-butyl 9-{2-[(1E)-3-amino-3-oxoprop-1-en-1-yl]benzoyl}-3,9-diazaspiro[5.5]undecane-3-carboxylate (120 mg, 0.28 mmol) was dissolved in Methanol (10 ml) and conc. HCl (3 ml) was added. The mixture was stirred at room temperature over night and evaporated. The residue was neutralized on an SCX-ion exchange column affording 34 mg (37%) of title compound as a colourless oil.

APCI-MS m/z: 328.1 [MH+]

### d) (2E)-3-[2-({9-[(3,3-dimethyl-2,3-dihydro-1,4-benzodioxin-5-yl)methyl]-3,9-diazaspiro[5.5]undec-3-yl}carbonyl)phenyl]acrylamide Trifluoroacetate

(2E)-3-[2-(3,9-diazaspiro[5.5]undec-3-ylcarbonyl)phenyl]acrylamide (34 mg, 0.10 mmol), 3,3-dimethyl-2,3-dihydro-1,4-benzodioxin-5-carbaldehyde (Intermediate Z) (21 mg, 0.11 mmol) was dissolved in dry CH₃CN (3 ml) and Sodium triacetoxyborohydride (42 mg, 0.20 mmol) was added. The mixture was stirred at room temperature over night, after which it was diluted with EtOAc, washed with aqueous NaHCO₃ (sat.), evaporated and purified by preparative HPLC (RP-18, gradient acetonitrile/water/TFA 5/95/0.1 to 75/25/0.1) to afford 8 mg (13%) of the title compound as a white solid.

¹H NMR (399.99 MHz, CD3OD) δ 7.82-7.77 (m, 1H), 7.56 (d, J=15.6 Hz, 1H), 7.53-7.45 (m, 2H), 7.34-7.29 (m, 1H), 7.05-6.98 (m, 2H), 6.96-6.90 (m, 1H), 6.70 (dd, J=15.7, 3.1 Hz, 1H), 4.29 (d, J=20.0 Hz, 2H), 4.04-3.90 (m, 1H), 3.97 (d, J=9.7 Hz, 2H), 3.75-3.63 (m, 1H), 3.44-3.35 (m, 2H), 3.26-3.06 (m, 4H), 2.14-1.96 (m, 2H), 1.88-1.80 (m, 1H), 1.73-1.53 (m, 4H), 1.41 (s, 3H), 1.37 (s, 3H)

APCI-MS m/z: 504.2 [MH+]

HPLC (Method A) RT: 6.71 min

HPLC (Method B) RT: 8.75 min

### EXAMPLE 150

### 6-({9-[(2,2-dimethyl-2,3-dihydro-1-benzofuran-4-yl)methyl]-3,9-diazaspiro[5.5]undec-3-yl}carbonyl)pyridazin-3(2H)-one Trifluoroacetate

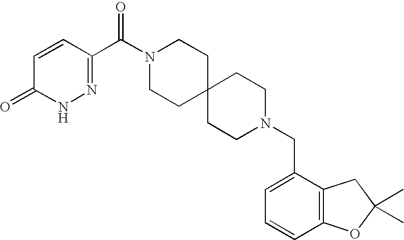

3-[(2,2-dimethyl-2,3-dihydro-1-benzofuran-4-yl)methyl]-3,9-diazaspiro[5.5]undecane (Intermediate C) (72 mg, 0.23 mmol), 6-oxo-1,6-dihydropyridazine-3-carboxylic acid (44 mg, 0.28 mmol), HBTU (106 mg, 0.28 mmol) and triethylamine (64 μl, 0.46 mmol) were dissolved in THF (3 ml) and NMP (0.5 ml). The mixture was stirred at room temperature over night after which it was diluted with dichloromethane and washed with aqueous NaHCO₃ (sat.), evaporated and purified by preparative HPLC (RP-18, gradient acetonitrile/water/TFA 5/95/0.1 to 65/35/0.1) to afford 55 mg (43%) of the title compound as a white solid.

¹H NMR (399.99 MHz, CD3OD) δ 7.64 (d, J=9.3 Hz, 1H), 7.23 (t, J=7.9 Hz, 1H), 7.04 (dd, J=9.7, 3.1 Hz, 1H), 6.95 (d, J=7.7 Hz, 1H), 6.79 (d, J=8.1 Hz, 1H), 4.26 (d, J=3.9 Hz, 2H), 3.81-3.63 (m, 4H), 3.42 (d, J=13.4 Hz, 2H), 3.29-3.13 (m, 2H), 3.16 (s, 2H), 2.05 (d, J=15.4 Hz, 2H), 1.85-1.75 (m, 2H), 1.73-1.60 (m, 2H), 1.58-1.50 (m, 2H), 1.48 (s, 6H)

APCI-MS m/z: 437.2 [MH+]

HPLC (Method A) RT: 5.31 min

HPLC (Method B) RT: 7.07 min

### EXAMPLE 151

### 5-({9-[(2,2-dimethyl-2,3-dihydro-1-benzofuran-4-yl)methyl]-3,9-diazaspiro[5.5]undec-3-yl}carbonyl)pyridin-3-ol Trifluoroacetate

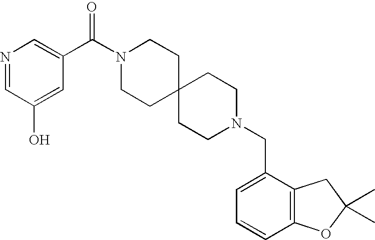

3-[(2,2-dimethyl-2,3-dihydro-1-benzofuran-4-yl)methyl]-3,9-diazaspiro[5.5]undecane dihydrochloride (Intermediate C) (77 mg, 0.20 mmol), 5-hydroxynicotinic acid (33 mg, 0.22 mmol), PyBOP (125 mg, 0.24 mmol) and triethylamine (111 μl, 0.80 mmol) were dissolved in THF (3 ml) and NMP (0.5 ml). The mixture was stirred at room temperature over night after which it was diluted with dichloromethane and washed with aqueous NaHCO₃ (sat.), evaporated and purified by preparative HPLC (RP-18, gradient acetonitrile/water/TFA 5/95/0.1 to 75/25/0.1) to afford 65 mg (59%) of the title compound as a white solid.

¹H NMR (399.99 MHz, CD3OD) δ 8.23 (s, 1H), 8.12 (s, 1H), 7.36 (s, 1H), 7.23 (t, J=7.8 Hz, 1H), 6.95 (d, J=7.6 Hz, 1H), 6.79 (d, J=7.9 Hz, 1H), 4.25 (s, 2H), 3.77 (s, 2H), 3.50-3.38 (m, 4H), 3.28-3.13 (m, 2H), 3.15 (s, 2H), 2.05 (d, J=15.4 Hz, 2H), 1.91-1.53 (m, 6H), 1.44 (s, 6H)

APCI-MS m/z: 436.3 [MH+]

HPLC (Method A) RT: 4.89 min

HPLC (Method B) RT: 5.29 min

### EXAMPLE 152

### 3-({9-[(2,2-dimethyl-2,3-dihydro-1-benzofuran-4-yl)methyl]-3,9-diazaspiro[5.5]undec-3-yl}carbonyl)pyridin-4(1H)-one Trifluoroacetate

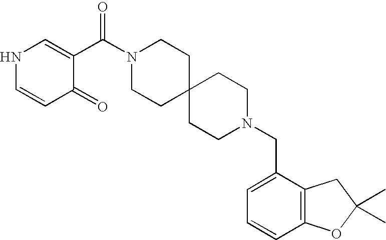

Synthesised according to Example 151 using Intermediate C (72 mg, 0.23 mmol) and 4-oxo-1,4-dihydropyridine-3-carboxylic acid (39 mg, 0.28 mmol). The crude product was purified by preparative HPLC (RP-18, gradient acetonitrile/water/TFA 5/95/0.1 to 65/35/0.1) to afford 17 mg (13%) of the title compound as a white solid.

¹H NMR (399.99 MHz, CD3OD) δ 7.98 (d, J=1.1 Hz, 1H), 7.89 (d, J=7.1 Hz, 1H), 7.23 (t, J=7.9 Hz, 1H), 6.94 (d, J=7.7 Hz, 1H), 6.78 (d, J=8.1 Hz, 1H), 6.59 (d, J=7.3 Hz, 1H), 4.25 (d, J=4.4 Hz, 2H), 3.80-3.69 (m, 2H), 3.46-3.34 (m, 4H), 3.28-3.13 (m, 2H), 3.10 (s, 2H), 2.08-1.98 (m, 2H), 1.85-1.72 (m, 2H), 1.71-1.58 (m, 2H), 1.58-1.44 (m, 2H), 1.48 (s, 6H)

APCI-MS m/z: 436.2 [MH+]

HPLC (Method A) RT: 4.75 min

HPLC (Method B) RT: 6.18 min

### EXAMPLE 153

### 3-({9-[(2,2-dimethyl-2,3-dihydro-1-benzofuran-4-yl)methyl]-3,9-diazaspiro[5.5]undec-3-yl}carbonyl)pyrazin-2(1H)-one Trifluoroacetate

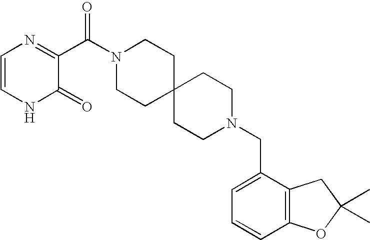

Synthesised according to Example 150 using Intermediate C (63 mg, 0.20 mmol) and 3-oxo-3,4-dihydropyrazine-2-carboxylic acid (34 mg, 0.24 mmol). The crude product was purified by preparative HPLC (RP-18, gradient acetonitrile/water/TFA 5/95/0.1 to 65/35/0.1) to afford 55 mg (50%) of the title compound as a white solid.

¹H NMR (399.99 MHz, CD3OD) δ 7.48 (s, 2H), 7.23 (t, J=7.7 Hz, 1H), 6.95 (d, J=7.5 Hz, 1H), 6.78 (d, J=7.9 Hz, 1H), 4.25 (d, J=9.1 Hz, 2H), 3.80-3.71 (m, 2H), 3.45-3.33 (m, 4H), 3.28-3.19 (m, 2H), 3.15 (d, J=9.0 Hz, 2H), 2.05 (d, J=12.7 Hz, 2H), 1.88-1.53 (m, 6H), 1.48 (s, 6H)

APCI-MS m/z: 437.2 [MH+]

HPLC (Method A) RT: 4.95 min

HPLC (Method B) RT: 4.48 min

### EXAMPLE 154

### 6-({9-[(2,2-dimethyl-2,3-dihydro-1-benzofuran-4-yl)methyl]-3,9-diazaspiro[5.5]undec-3-yl}carbonyl)pyridin-2-amine Trifluoroacetate

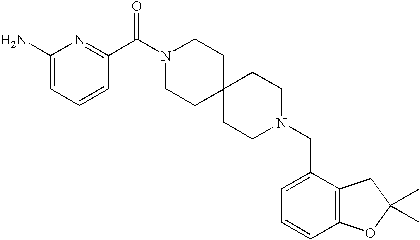

Synthesised essentially according to Example 150 using Intermediate C (dihydrochloride) (77 mg, 0.20 mmol) and 6-aminopyridine-2-carboxylic acid (33 mg, 0.24 mmol). The crude product was purified by preparative HPLC (RP-18, gradient acetonitrile/water/TFA 5/95/0.1 to 60/40/0.1) to afford 24 mg (22%) of the title compound as a white solid.

¹H NMR (399.99 MHz, CD3OD) δ 7.78 (dd, J=8.7, 7.3 Hz, 1H), 7.23 (t, J=7.8 Hz, 1H), 6.95 (d, J=7.7 Hz, 1H), 6.90 (d, J=8.8 Hz, 1H), 6.86 (d, J=7.3 Hz, 1H), 6.79 (d, J=7.9 Hz, 1H), 4.25 (s, 2H), 3.80-3.63 (m, 2H), 3.59-3.48 (m, 2H), 3.46-3.38 (m, 2H), 3.28-3.18 (m, 2H), 3.16 (s, 2H), 2.09-2.00 (m, 2H), 1.87-1.74 (m, 2H), 1.73-1.61 (m, 2H), 1.58-1.50 (m, 2H), 1.48 (s, 6H)

APCI-MS m/z: 435.3 [MH+]

HPLC (Method B) RT: 8.41 min

### EXAMPLE 155

### 6-({9-[(2,2-dimethyl-2,3-dihydro-1-benzofuran-4-yl)methyl]-3,9-diazaspiro[5.5]undec-3-yl}carbonyl)pyridin-3-ol Trifluoroacetate

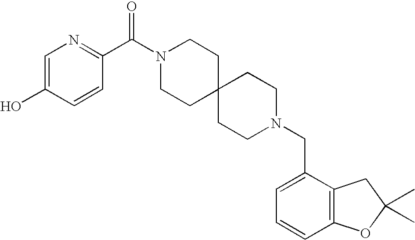

Synthesised according to Example 151 using Intermediate C (dihydrochloride) (77 mg, 0.20 mmol) and 5-hydroxypyridine-2-carboxylic acid monohydrate (38 mg, 0.24 mmol). The crude product was purified by preparative HPLC (RP-18, gradient acetonitrile/water/TFA 5/95/0.1 to 65/35/0.1) to afford 45 mg (41%) of the title compound as a white solid.

¹H NMR (299.945 MHz, CD3OD) δ 8.11 (s, 1H), 7.49 (d, J=8.4 Hz, 1H), 7.30 (dd, J=8.7, 2.5 Hz, 1H), 7.23 (t, J=7.8 Hz, 1H), 6.94 (d, J=7.7 Hz, 1H), 6.79 (d, J=8.3 Hz, 1H), 4.25 (s, 2H), 3.75 (s, 2H), 3.54 (s, 2H), 3.45-3.35 (m, 2H), 3.27-3.13 (m, 2H), 3.16 (s, 2H), 2.13-1.99 (m, 2H), 1.88-1.53 (m, 6H), 1.48 (s, 6H)

APCI-MS m/z: 436.3 [MH+]

HPLC (Method A) RT: 5.61 min

HPLC (Method B) RT: 5.25 min

### EXAMPLE 156

### 6-({9-[(3,3-dimethyl-2,3-dihydro-1,4-benzodioxin-5-yl)methyl]-3,9-diazaspiro[5.5]undec-3-yl}carbonyl)pyridin-2-amine Trifluoroacetate

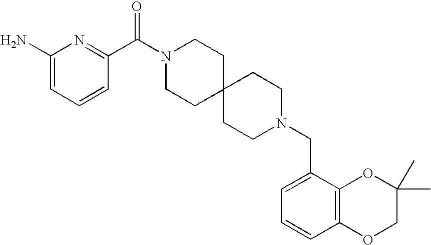

Synthesised essentially according to Example 151 using Intermediate AA (50 mg, 0.15 mmol) and 6-aminopyridine-2-carboxylic acid (25 mg, 0.18 mmol). The crude product was purified by preparative HPLC (RP-18, gradient acetonitrile/water/TFA 5/95/0.1 to 60/40/0.1) to afford 42 mg (49%) of the title compound as a white solid.

¹H NMR (399.99 MHz, CD3OD) δ 7.78 (dd, J=8.7, 7.2 Hz, 1H), 7.02 (d, J=7.3 Hz, 2H), 6.96-6.84 (m, 3H), 4.31 (s, 2H), 3.97 (s, 2H), 3.80-3.66 (m, 2H), 3.58-3.47 (m, 2H), 3.46-3.38 (m, 2H), 3.27-3.15 (m, 2H), 2.10-2.00 (m, 2H), 1.84-1.61 (m, 5H), 1.60-1.47 (m, 3H), 1.39 (s, 6H)

APCI-MS m/z: 451.3 [MH+]

HPLC (Method A) RT: 5.14 min

HPLC (Method B) RT: 8.31 min

### EXAMPLE 157

### 4-({7-[(2,2-dimethyl-2,3-dihydro-1-benzofuran-4-yl)methyl]-2,7-diazaspiro[3.5]non-2-yl}carbonyl)pyrimidin-2-amine

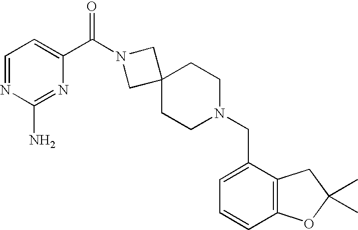

Synthesised according to Example 151 using Intermediate AD (29 mg, 0.10 mmol) and 2-aminopyrimidine-4-carboxylic acid (16 mg, 0.12 mmol) to afford 8 mg (20%) of the title compound as a white solid.

¹H NMR (499.88 MHz, CD3OD) δ 8.39 (d, J=5.0 Hz, 1H), 7.07 (d, J=5.1 Hz, 1H), 7.04 (t, J=7.8 Hz, 1H), 6.77 (d, J=7.7 Hz, 1H), 6.57 (d, J=7.6 Hz, 1H), 4.39 (s, 2H), 3.84 (s, 2H), 3.41 (s, 2H), 3.05 (s, 2H), 2.42 (s, 4H), 1.83 (t, J=5.4 Hz, 4H), 1.44 (s, 6H)

APCI-MS m/z: 408.2 [MH+]

HPLC (Method A) RT: 4.97 min

HPLC (Method B) RT: 7.43 min

### EXAMPLE 158

### 6-({7-[(2,2-dimethyl-2,3-dihydro-1-benzofuran-4-yl)methyl]-2,7-diazaspiro[3.5]non-2-yl}carbonyl)pyridin-3-amine

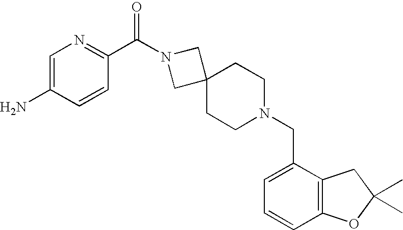

Synthesised according to Example 151 using Intermediate AD (58 mg, 0.20 mmol) and 5-aminopyridine-2-carboxylic acid (33 mg, 0.24 mmol) to afford 45 mg (55%) of the title compound as a white solid.

¹H NMR (399.99 MHz, CD3OD) δ 7.96 (d, J=2.3 Hz, 1H), 7.71 (d, J=8.5 Hz, 1H), 7.07-6.98 (m, 2H), 6.78 (d, J=7.6 Hz, 1H), 6.57 (d, J=8.0 Hz, 1H), 4.33 (s, 2H), 3.83 (s, 2H), 3.41 (s, 2H), 3.05 (s, 2H), 2.43 (s, 4H), 1.82 (t, J=5.3 Hz, 4H), 1.44 (s, 6H)

APCI-MS m/z: 407.2 [MH+]

HPLC (Method A) RT: 4.75 min

HPLC (Method B) RT: 7.88 min

### EXAMPLE 159

### 2-[4-({9-[(2,2-dimethyl-2,3-dihydro-1-benzofuran-4-yl)methyl]-3,9-diazaspiro[5.5]undec-3-yl}carbonyl)pyridin-3-yl]acetamide

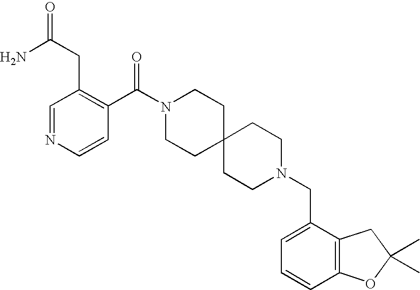

Synthesized according to Example 143 using [4-({9-[(2,2-dimethyl-2,3-dihydro-1-benzofuran-4-yl)methyl]-3,9-diazaspiro[5.5]undec-3-yl}carbonyl)pyridin-3-yl]acetic acid (Example 164) to afford 15 mg (15%) of the title compound as a white solid.

¹H NMR (399.99 MHz, CD3OD) δ 8.56 (s, 1H), 8.51 (d, J=5.1 Hz, 2H), 7.31 (d, J=5.0 Hz, 1H), 7.04 (t, J=7.8 Hz, 1H), 6.78 (d, J=7.6 Hz, 1H), 6.56 (d, J=7.8 Hz, 1H), 3.77-3.57 (m, 4H), 3.44 (s, 2H), 3.28-3.21 (m, 2H), 3.04 (s, 2H), 2.55-2.39 (m, 4H), 1.66-1.55 (m, 6H), 1.51-1.46 (m, 2H), 1.43 (s, 6H)

APCI-MS m/z: 477.3 [MH+]

HPLC (Method A) RT: 4.59 min

HPLC (Method B) RT: 6.97 min

### EXAMPLE 160

### 2-[4-({9-[(2,2,3,3-tetramethyl-2,3-dihydro-1-benzofuran-7-yl)methyl]-3,9-diazaspiro[5.5]undec-3-yl}carbonyl)pyridin-3-yl]acetamide

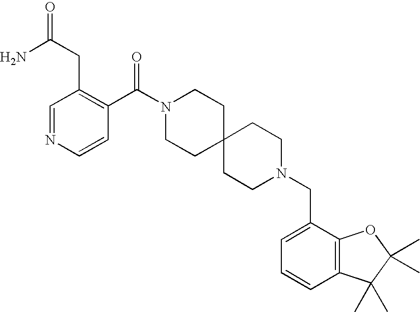

Synthesized according to Example 143 using [4-({9-[(2,2,3,3-tetramethyl-2,3-dihydro-1-benzofuran-7-yl)methyl]-3,9-diazaspiro[5.5]undec-3-yl}carbonyl)pyridin-3-yl]acetic acid (Example 183) to afford 25 mg (25%) of the title compound as a white solid.

¹H NMR (399.99 MHz, DMSO-D6) δ 8.49 (s, 1H), 8.46 (d, J=4.8 Hz, 1H), 7.49 (s, 1H), 7.20 (d, J=4.8 Hz, 1H), 7.05-6.95 (m, 2H), 6.78 (t, J=7.4 Hz, 1H), 3.57 (s, 2H), 3.43 (s, 2H), 3.36 (s, 2H), 3.06 (s, 2H), 2.38-2.26 (m, 2H), 1.44 (s, 6H), 1.32 (s, 2H), 1.23 (s, 6H), 1.12 (s, 6H)

APCI-MS m/z: 505.2 [MH+]

HPLC (Method A) RT: 5.94 min

HPLC (Method B) RT: 7.90 min

### EXAMPLE 161

### N-cyclopropyl-2-[4-({9-[(2,2-dimethyl-2,3-dihydro-1-benzofuran-4-yl)methyl]-3,9-diazaspiro[5.5]undec-3-yl}carbonyl)pyridin-3-yl]acetamide

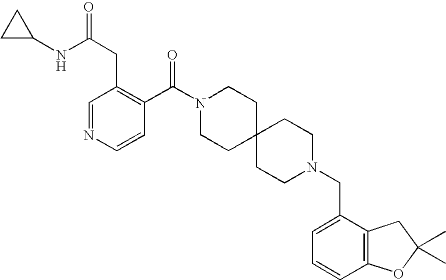

[4-({9-[(2,2-dimethyl-2,3-dihydro-1-benzofuran-4-yl)methyl]-3,9-diazaspiro[5.5]undec-3-yl}carbonyl)pyridin-3-yl]acetic acid (Example 164) (75 mg, 0.16 mmol), HBTU (68 mg, 0.18 mmol), cyclopropylamine (17 μl, 0.24 mmol) and triethylamine (45 μl, 0.32 mmol) were dissolved in CH₃CN (4 ml) and NMP (2 ml). The mixture was stirred at room temperature over night. The mixture was diluted with EtOAc and washed with aqueous NaHCO₃ (sat.). The organic layer was evaporated and purified by preparative HPLC (XTerra, gradient acetonitrile/water/NH₄OH 20/80/0.2 to 55/45/0.2) to afford 25 mg (30%) of the title compound as a white solid.

¹H NMR (399.99 MHz, CD3OD) δ 8.54 (s, 1H), 8.50 (d, J=5.0 Hz, 1H), 7.31 (d, J=5.0 Hz, 1H), 7.04 (t, J=7.8 Hz, 1H), 6.78 (d, J=7.4 Hz, 1H), 6.56 (d, J=7.8 Hz, 1H), 3.80-3.50 (m, 4H), 3.44 (s, 2H), 3.25 (s, 2H), 3.04 (s, 2H), 2.66 (dquintet, J=7.2, 3.7 Hz, 1H), 2.54-2.40 (m, 4H), 1.60 (s, 6H), 1.49 (t, J=5.3 Hz, 2H), 1.43 (s, 6H), 0.71 (td, J=6.9, 5.3 Hz, 2H), 0.51-0.46 (m, 2H)

APCI-MS m/z: 517.2 [MH+]

HPLC (Method A) RT: 5.24 min

HPLC (Method B) RT: 5.56 min

### EXAMPLE 162

### [4-({9-[(3,3-dimethyl-2,3-dihydro-1,4-benzodioxin-5-yl)methyl]-3,9-diazaspiro[5.5]undec-3-yl}carbonyl)pyridin-3-yl]acetic Acid

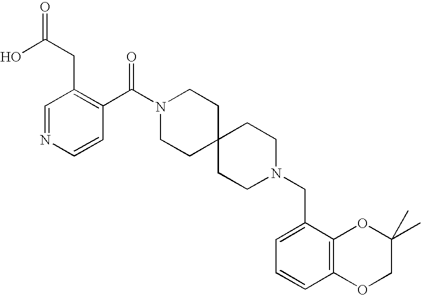

3-[(3,3-dimethyl-2,3-dihydro-1,4-benzodioxin-5-yl)methyl]-3,9-diazaspiro[5.5]undecane (Intermediate AA) (200 mg, 0.60 mmol), 3-[2-ethoxy-1-(ethoxycarbonyl)-2-oxoethyl]isonicotinic acid (Intermediate AB) (185 mg, 0.66 mmol), HBTU (250 mg, 0.66 mmol) and triethylamine (167 μl, 1.20 mmol) were dissolved in dichloromethane (10 ml) and stirred at room temperature over night. The reaction mixture was diluted with dichloromethane and washed with aqueous NaHCO₃ (sat.), dried over Na2SO4 and evaporated. The residue was dissolved in Methanol (20 ml) and H2O (5 ml) and Lithium hydroxide monohydrate (125 mg, 3.00 mmol) was added and refluxed for 2 hrs. After cooling the solids were filtered off and the filtrate evaporated and purified by preparative HPLC (RP-18, gradient acetonitrile/water/NH₄OAc (2 g/L) 5/95 to 55/45) to afford 90 mg (30%) of the title compound as a offwhite solid.

APCI-MS m/z: 494.3 [MH+]

### EXAMPLE 163

### [4-({9-[(2,2,3,3-tetramethyl-2,3-dihydro-1-benzofuran-7-yl)methyl]-3,9-diazaspiro[5.5]undec-3-yl}carbonyl)pyridin-3-yl]acetic Acid

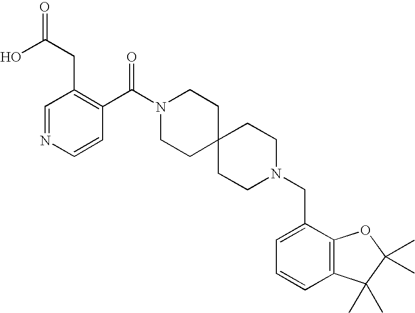

Synthesized according to example 164 from 3-[(2,2,3,3-tetramethyl-2,3-dihydro-1-benzofuran-7-yl)methyl]-3,9-diazaspiro[5.5]undecane (Intermediate Q) and 3-[2-ethoxy-1-(ethoxycarbonyl)-2-oxoethyl]isonicotinic acid (Intermediate AB) to afford 190 mg (43%) of the title compound as a yellow solid.

¹H NMR (399.99 MHz, DMSO-D6) δ 8.49 (s, 1H), 8.42 (d, J=4.8 Hz, 1H), 7.17 (d, J=4.8 Hz, 1H), 7.03 (d, J=7.6 Hz, 1H), 6.99 (d, J=7.3 Hz, 1H), 6.78 (t, J=7.4 Hz, 1H), 3.55 (s, 2H), 3.44 (s, 2H), 3.36 (s, 2H), 3.07 (s, 2H), 2.37-2.25 (m, 4H), 1.47-1.38 (m, 6H), 1.31 (s, 2H), 1.23 (s, 6H), 1.12 (s, 6H)

APCI-MS m/z: 506.3 [MH+]

HPLC (Method A) RT: 6.19 min

HPLC (Method B) RT: 5.84 min

### EXAMPLE 164

### [4-({9-[(2,2-dimethyl-2,3-dihydro-1-benzofuran-4-yl)methyl]-3,9-diazaspiro[5.5]undec-3-yl}carbonyl)pyridin-3-yl]acetic Acid

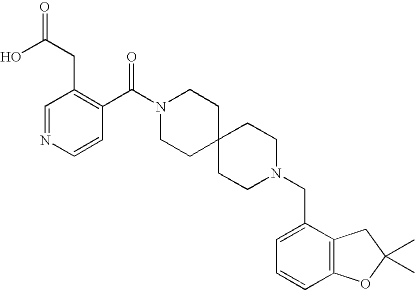

Synthesized according to example 164 from 3-[(2,2-dimethyl-2,3-dihydro-1-benzofuran-4-yl)methyl]-3,9-diazaspiro[5.5]undecane (Intermediate C) and 3-[2-ethoxy-1-(ethoxycarbonyl)-2-oxoethyl]isonicotinic acid (Intermediate AB) to afford 250 mg (41%) of the title compound as a yellow solid.

¹H NMR (399.99 MHz, DMSO-D6) δ 8.50 (s, 1H), 8.44 (d, J=5.0 Hz, 1H), 7.19 (d, J=4.8 Hz, 1H), 7.00 (t, J=7.8 Hz, 1H), 6.70 (d, J=7.4 Hz, 1H), 6.56 (d, J=7.8 Hz, 1H), 3.56 (s, 2H), 3.48 (s, 2H), 3.34 (s, 2H), 3.09 (s, 2H), 2.97 (s, 2H), 2.34-2.26 (m, 4H), 1.44 (s, 6H), 1.38 (s, 6H), 1.33 (s, 2H)

APCI-MS m/z: 478.3 [MH+]

### EXAMPLE 165

### 6-({7-[(2,2-dimethyl-2,3-dihydro-1-benzofuran-4-yl)methyl]-2,7-diazaspiro[4.4]non-2-yl}carbonyl)pyridin-3-amine Di Trifluoroacetate

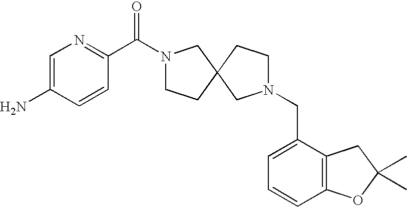

Synthesised according to Example 151 using Intermediate AC (80 mg, 0.28 mmol) and 5-aminopyridine-2-carboxylic acid (47 mg, 0.34 mmol) to afford 110 mg (62%) of the title compound as a white solid.

¹H NMR (399.99 MHz, CD3OD) δ 7.99 (d, J=2.3 Hz, 1H), 7.70 (d, J=8.7 Hz, 1H), 7.26-7.17 (m, 2H), 6.95 (dd, J=17.5, 8.0 Hz, 1H), 6.77 (t, J=7.9 Hz, 1H), 4.36 (d, J=21.9 Hz, 2H), 3.98-3.85 (m, 2H), 3.78-3.58 (m, 4H), 3.16 (d, J=15.9 Hz, 2H), 2.37-1.99 (m, 6H), 1.48 (d, J=7.6 Hz, 6H)

APCI-MS m/z: 407.2 [MH+]

HPLC (Method A) RT: 4.68 min

HPLC (Method B) RT: 7.67 min

### EXAMPLE 166

### 5-chloro-4-({9-[(2,2-dimethyl-2,3-dihydro-1-benzofuran-4-yl)methyl]-3,9-diazaspiro[5.5]undec-3-yl}carbonyl)pyridin-2-amine Di Trifluoroacetate

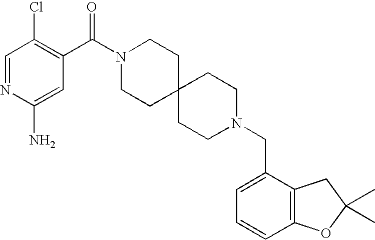

Synthesised according to Example 151 using Intermediate C (100 mg, 0.32 mmol) and 2-amino-5-chloroisonicotinic acid (66 mg, 0.38 mmol) to afford 150 mg (67%) of the title compound as a yellowish solid.

¹H NMR (399.99 MHz, CD3OD) δ 8.01 (d, J=3.9 Hz, 1H), 7.23 (t, J=8.0 Hz, 1H), 6.94 (dd, J=7.4, 3.9 Hz, 1H), 6.79 (d, J=8.0 Hz, 1H), 6.63 (s, 1H), 4.25 (d, J=9.9 Hz, 2H), 3.85-3.66 (m, 2H), 3.46-3.37 (m, 2H), 3.25-3.11 (m, 6H), 2.10-1.98 (m, 2H), 1.87-1.53 (m, 6H), 1.48 (d, J=5.1 Hz, 6H)

APCI-MS m/z: 469.2 [MH+]

HPLC (Method A) RT: 5.42 min

HPLC (Method B) RT: 9.22 min

### EXAMPLE 167

### 2-[3-({9-[(2,2-dimethyl-2,3-dihydro-1-benzofuran-4-yl)methyl]-3,9-diazaspiro[5.5]undec-3-yl}carbonyl)phenyl]acetamide Trifluoroacetate

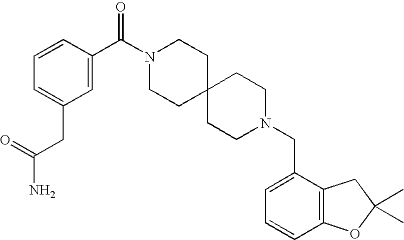

The title compound was prepared by the procedure of Example 119 using Intermediate C and Intermediate AF as starting materials to give the product as a white solid (60 mg, 44%).

¹H NMR (399.99 MHz, CD3OD) δ 7.42 (d, J=4.0 Hz, 2H), 7.36 (s, 1H), 7.32-7.27 (m, 1H), 7.22 (t, J=7.9 Hz, 1H), 6.95 (d, J=7.6 Hz, 1H), 6.78 (d, J=8.0 Hz, 1H), 4.25 (s, 2H), 3.75 (s, 2H), 3.57 (s, 2H), 3.50-3.36 (m, 4H), 3.28-3.11 (m, 4H), 2.03 (d, J=14.6 Hz, 2H), 1.87-1.77 (m, 1H), 1.75-1.59 (m, 3H), 1.58-1.39 (m, 8H)

APCI-MS m/z: 476.3 [MH+]

HPLC (Method A) Retention time: 5.80 min

HPLC (Method B) Retention time: 8.29 min

### EXAMPLE 168

### 4-({9-[(2,2-dimethyl-2,3-dihydro-1-benzofuran-4-yl)methyl]-3,9-diazaspiro[5.5]undec-3-yl}carbonyl)benzamide Trifluoroacetate

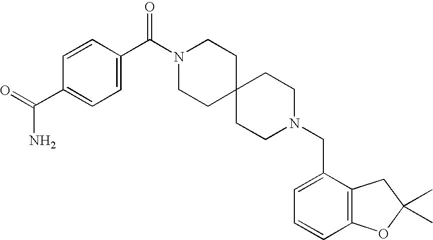

The title compound was prepared by the procedure of Example 119 using Intermediate C and 4-(aminocarbonyl)benzoic acid as starting materials to give the product as a white solid (60 mg, 45%).

¹H NMR (399.99 MHz, CD3OD) δ 7.96 (d, J=7.7 Hz, 2H), 7.50 (d, J=8.0 Hz, 2H), 7.22 (t, J=7.8 Hz, 1H), 6.95 (d, J=7.3 Hz, 1H), 6.78 (d, J=7.9 Hz, 1H), 4.24 (d, J=7.9 Hz, 2H), 3.78 (s, 2H), 3.49-3.34 (m, 4H), 3.29-3.08 (m, 4H), 2.05 (d, J=14.2 Hz, 2H), 1.90-1.77 (m, 1H), 1.77-1.53 (m, 4H), 1.52-1.38 (m, 7H)

APCI-MS m/z: 462.3 [MH+]

HPLC (Method A) Retention time: 5.59 min

HPLC (Method B) Retention time: 8.13 min

### EXAMPLE 169

### 2-[4-({9-[(3,3-dimethyl-2,3-dihydro-1,4-benzodioxin-5-yl)methyl]-3,9-diazaspiro[5.5]undec-3-yl}carbonyl)phenyl]acetamide Trifluoroacetate

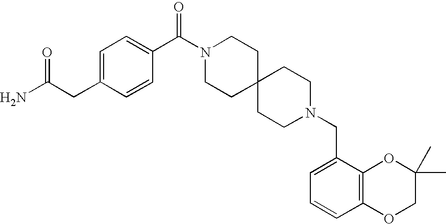

The title compound was prepared by the procedure of Example 119 using Intermediate AA and Intermediate AG as starting materials and DCM/NMP (5:2) as solvent to give the product as a white solid (50 mg, 31%).

¹H NMR (399.99 MHz, CD3OD) δ 7.38 (q, J=8.2 Hz, 4H), 7.01 (d, J=8.3 Hz, 2H), 6.92 (t, J=7.8 Hz, 1H), 4.30 (s, 2H), 3.97 (s, 2H), 3.75 (s, 2H), 3.57 (s, 2H), 3.52-3.35 (m, 4H), 3.29-3.09 (m, 2H), 2.03 (d, J=14.3 Hz, 2H), 1.83-1.74 (m, 1H), 1.74-1.59 (m, 3H), 1.59-1.51 (m, 1H), 1.48-1.34 (m, 7H)

APCI-MS m/z: 492.2 [MH+]

HPLC (Method A) Retention time: 5.73 min

HPLC (Method B) Retention time: 8.03 min

### EXAMPLE 170

### 5-chloro-4-({9-[(3,3-dimethyl-2,3-dihydro-1,4-benzodioxin-5-yl)methyl]-3,9-diazaspiro[5.5]undec-3-yl}carbonyl)pyrimidin-2-amine Trifluoroacetate

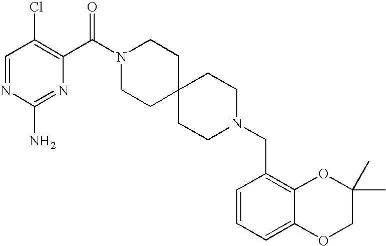

The title compound was prepared by the procedure of Example 64 using Intermediate AA and 2-amino-5-chloropyrimidine-4-carboxylic acid as starting materials and THF as solvent to give the product as a white solid (30 mg, 17%).

¹H NMR (399.99 MHz, CD3OD) δ 8.32 (d, J=5.7 Hz, 1H), 7.06-6.98 (m, 2H), 6.97-6.89 (m, 1H), 4.30 (d, J=9.7 Hz, 2H), 3.97 (d, J=4.9 Hz, 2H), 3.78-3.71 (m, 2H), 3.45-3.36 (m, 2H), 3.28-3.11 (m, 2H), 2.09-1.98 (m, 2H), 1.97-1.91 (m, 2H), 1.79 (t, J=5.7 Hz, 1H), 1.76-1.62 (m, 3H), 1.59-1.49 (m, 2H), 1.40 (s, 3H), 1.38 (s, 3H)

APCI-MS m/z: 486.3/488.3 [MH+]

HPLC (Method A) Retention time: 6.45 min

HPLC (Method B) Retention time: 8.66 min

### EXAMPLE 171

### 6-({9-[(3,3-dimethyl-2,3-dihydro-1,4-benzodioxin-5-yl)methyl]-3,9-diazaspiro[5.5]undec-3-yl}carbonyl)pyridin-3-amine Trifluoroacetate

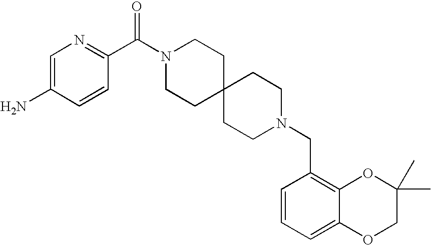

The title compound was prepared by the procedure of Example 64 using Intermediate AA and 5-aminopyridine-2-carboxylic acid as starting materials to give the product as a white solid (97 mg, 57%).

¹H NMR (399.99 MHz, CD3OD) δ 8.00 (d, J=2.5 Hz, 1H), 7.65 (d, J=8.8 Hz, 1H), 7.47-7.43 (m, 1H), 7.05-6.99 (m, 2H), 6.96-6.89 (m, 1H), 4.31 (s, 2H), 3.97 (s, 2H), 3.73-3.59 (m, 4H), 3.46-3.37 (m, 2H), 3.27-3.15 (m, 2H), 2.04 (d, J=14.9 Hz, 2H), 1.82-1.74 (m, 2H), 1.72-1.63 (m, 2H), 1.58-1.52 (m, 2H), 1.39 (s, 6H)

APCI-MS m/z: 451.3 [MH+]

HPLC (Method A) Retention time: 5.02 min

HPLC (Method B) Retention time: 7.66 min

### EXAMPLE 172

### 2-({9-[(3,3-dimethyl-2,3-dihydro-1,4-benzodioxin-5-yl)methyl]-3,9-diazaspiro[5.5]undec-3-yl}carbonyl)pyridin-3-amine Trifluoroacetate

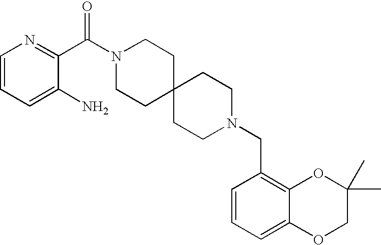

The title compound was prepared by the procedure of Example 64 using Intermediate AA and 3-aminopyridine-2-carboxylic acid as starting materials to give the product as a white solid (111 mg, 66%).

¹H NMR (399.99 MHz, CD3OD) δ 7.97-7.95 (m, 1H), 7.63-7.59 (m, 1H), 7.57-7.52 (m, 1H), 7.04-6.99 (m, 2H), 6.93 (d, J=7.4 Hz, 1H), 4.31 (s, 2H), 3.97 (s, 2H), 3.73-3.49 (m, 4H), 3.45-3.37 (m, 2H), 3.27-3.15 (m, 2H), 2.03 (d, J=14.9 Hz, 2H), 1.83-1.74 (m, 2H), 1.74-1.62 (m, 2H), 1.59-1.51 (m, 2H), 1.39 (s, 6H)

APCI-MS m/z: 451.1 [MH+]

HPLC (Method A) Retention time: 4.96 ml

HPLC (Method B) Retention time: 7.97 min

### EXAMPLE 173

### 6-({9-[(2,2-dimethyl-2,3-dihydro-1,4-benzodioxin-5-yl)methyl]-3,9-diazaspiro[5.5]undec-3-yl}carbonyl)pyridin-3-amine Trifluoroacetate

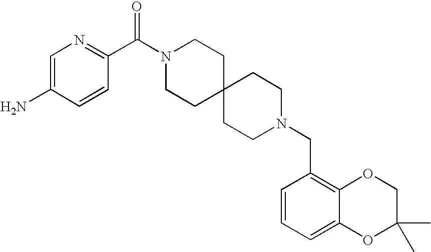

The title compound was prepared by the procedure of Example 64 using Intermediate AH and 5-aminopyridine-2-carboxylic acid as starting materials to give the product as a white solid (70 mg, 41%).

¹H NMR (399.99 MHz, CD3OD) δ 8.00 (d, J=2.5 Hz, 1H), 7.71-7.64 (m, 1H), 7.51-7.44 (m, 1H), 7.01-6.92 (m, 3H), 4.35 (s, 2H), 4.04 (s, 2H), 3.75-3.55 (m, 4H), 3.46-3.36 (m, 2H), 3.26-3.16 (m, 2H), 2.09-1.99 (m, 2H), 1.83-1.74 (m, 2H), 1.73-1.63 (m, 2H), 1.59-1.51 (m, 2H), 1.37-1.32 (m, 6H)

APCI-MS m/z: 451.0 [MH+]

HPLC (Method A) Retention time: 5.15 min

HPLC (Method B) Retention time: 7.61 min

### EXAMPLE 174

### 4-({9-[(2,2-dimethyl-2,3-dihydro-1,4-benzodioxin-5-yl)methyl]-3,9-diazaspiro[5.5]undec-3-yl}carbonyl)pyridin-2-amine Trifluoroacetate

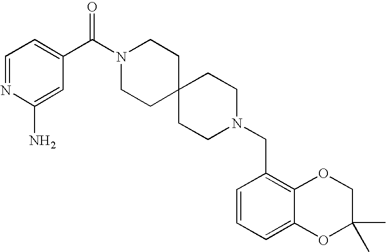

The title compound was prepared by the procedure of Example 64 using Intermediate AH and 2-aminoisonicotinic acid as starting materials to give the product as a white solid (50 mg, 300%).

¹H NMR (399.99 MHz, CD3OD) δ 7.91 (t, J=5.9 Hz, 1H), 7.01-6.90 (m, 4H), 6.85 (t, J=5.1 Hz, 1H), 4.34 (d, J=8.3 Hz, 2H), 4.03 (d, J=5.3 Hz, 2H), 3.78-3.69 (m, 2H), 3.46-3.35 (m, 4H), 3.28-3.12 (m, 2H), 2.03 (d, J=14.7 Hz, 2H), 1.85-1.62 (m, 4H), 1.61-1.45 (m, 2H), 1.34 (d, J=2.6 Hz, 6H)

APCI-MS m/z: 451.0 [MH+]

HPLC (Method A) Retention time: 5.09 min

HPLC (Method B) Retention time: 7.77 min

### EXAMPLE 175

### 4-({9-[(2,2-dimethyl-2,3-dihydro-1,4-benzodioxin-5-yl)methyl]-3,9-diazaspiro[5.5]undec-3-yl}carbonyl)pyridin-3-amine Trifluoroacetate

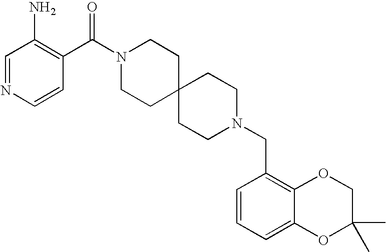

The title compound was prepared by the procedure of Example 64 using Intermediate AH and 3-aminoisonicotinic acid as starting materials to give the product as a white solid (120 mg, 71%).

¹H NMR (399.99 MHz, CD3OD) δ 8.16 (s, 1H), 7.99 (d, J=3.1 Hz, 1H), 7.65 (d, J=5.1 Hz, 1H), 7.02-6.90 (m, 3H), 4.34 (d, J=9.2 Hz, 2H), 4.03 (s, 2H), 3.78 (s, 2H), 3.45-3.33 (m, 4H), 3.29-3.10 (m, 2H), 2.02 (d, J=14.8 Hz, 2H), 1.89-1.63 (m, 4H), 1.63-1.44 (m, 2H), 1.34 (s, 6H)

APCI-MS m/z: 451.0 [MH+]

HPLC (Method A) Retention time: 4.97 min

HPLC (Method B) Retention time: 7.68 min

### EXAMPLE 176

### 4-({9-[(2,2-dimethyl-2,3-dihydro-1,4-benzodioxin-5-yl)methyl]-3,9-diazaspiro[5.5]undec-3-yl}carbonyl)pyrimidin-2-amine Trifluoroacetate

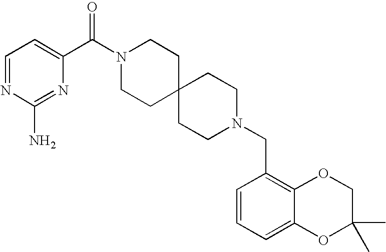

The title compound was prepared by the procedure of Example 64 using Intermediate AH and 2-aminopyrimidine-4-carboxylic acid as starting materials and THF as solvent to give the product as a white solid (60 mg, 35%).

¹H NMR (399.99 MHz, CD3OD) δ 8.39 (t, J=5.3 Hz, 1H), 7.02-6.91 (m, 3H), 6.86-6.79 (m, 1H), 4.34 (d, J=7.9 Hz, 2H), 4.03 (d, J=5.4 Hz, 2H), 3.76-3.67 (m, 2H), 3.47-3.36 (m, 4H), 3.28-3.12 (m, 2H), 2.03 (d, J=15.0 Hz, 2H), 1.83-1.61 (m, 4H), 1.59-1.47 (m, 2H), 1.35 (d, J=3.1 Hz, 6H)

APCI-MS m/z: 452.0 [MH+]

HPLC (Method A) Retention time: 5.37 min

HPLC (Method B) Retention time: 7.40 min

### EXAMPLE 177

### 4-({9-[(3,3-dimethyl-2,3-dihydro-1,4-benzodioxin-5-yl)methyl]-3,9-diazaspiro[5.5]undec-3-yl}carbonyl)pyrimidin-2-amine Trifluoroacetate

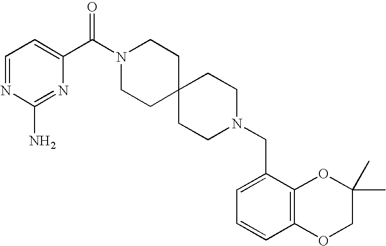

The title compound was prepared by the procedure of Example 77 using Intermediate P and Intermediate Z as starting materials to give the product as a white solid (65 mg, 32%).

¹H NMR (399.99 MHz, CD3OD) δ 8.38 (t, J=4.8 Hz, 1H), 7.05-6.99 (m, 2H), 6.96-6.89 (m, 1H), 6.78 (d, J=5.2 Hz, 1H), 4.31 (d, J=7.7 Hz, 2H), 3.97 (d, J=4.4 Hz, 2H), 3.76-3.68 (m, 2H), 3.48-3.37 (m, 4H), 3.27-3.11 (m, 2H), 2.03 (d, J=14.9 Hz, 2H), 1.81-1.61 (m, 4H), 1.59-1.47 (m, 2H), 1.40 (s, 3H), 1.38 (s, 3H)

APCI-MS m/z: 452.2 [MH+]

HPLC (Method A) Retention time: 5.15 min

HPLC (Method B) Retention time: 7.82 min

### EXAMPLE 178

### 8-[(2,2-dimethyl-2,3-dihydro-1-benzofuran-7-yl)methyl]-2-(pyridin-4-ylacetyl)-2,8-diazaspiro[4.5]decane

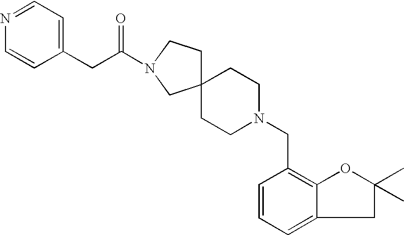

The title compound was prepared by the procedure of example 135 using 8-[(2,2-dimethyl-2,3-dihydro-1-benzofuran-7-yl)methyl]-2,8-diazaspiro[4.5]decane hydrochloric acid (0.1 g, 0.3 mmol) and 2-(4-pyridyl)acetic acid (0.057 g, 0.33 mmol) to give the product (0.05 g, 41%) as pale yellow liquid.

¹H NMR (400 MHz, CDCl₃): δ 1.43 (s, 6H), 1.88 (m, 2H), 3.04 (s, 3H), 3.37 (s, 1H), 3.54 (m, 3H), 3.65 (s, 2H), 6.85 (t, 1H, J=7.5 Hz), 7.0-7.25 (m, 4H), 8.56 (m, 2H).

LCMS (ESI): m/z 420 (M+1).

HPLC (Method C) RT: 6.23 min

### EXAMPLE 179

### 6-({9-[(2,2-dimethyl-1,3-benzodioxol-4-yl)methyl]-3,9-diazaspiro[5.5]undec-3-yl}carbonyl)pyrimidin-4-amine

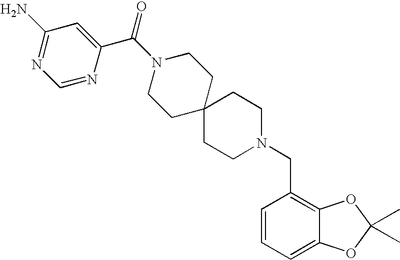

A mixture of 3-[(2,2-dimethyl-1,3-benzodioxol-4-yl)methyl]-3,9-diazaspiro[5.5]undecane (100 mg, 0.32 mmol), (benzotriazol-1-yloxy)tripyrrolidinophosphonium hexafluorophosphate (PYBOP, 165 mg, 0.32 mmol), 6-aminopyrimidine-4-carboxylic (56 mg, 0.33 mmol), triethylamine (200 μl, 1.4 mmol), THF (3 ml) and NMP (0.5 ml) was stirred at ambient temperature for one hour, evaporated and acidified with TFA. Product was purified with preparative HPLC (RP-18, gradient acetonitrile/water/TFA from 10/90/0.1 to 60/40/0.1) to give the title compound as a white solid (29 mg, 17%).

¹H NMR (399.99 MHz, DMSO-D₆) δ 9.30 (s, 1H), 8.41 (d, 1H), 7.45 (s, 2H), 6.99-6.83 (m, 3H), 6.49 (d, 1H), 4.32-4.18 (m, 2H), 3.40-2.96 (m, 8H), 1.90 (d, 2H), 1.68 (d, 6H), 1.53 (dd, 4H), 1.36 (d, 2H)

APCI-MS m/z: 438.0 [MH+]

HPLC (Method A) RT: 4.78 min

HPLC (Method B) RT: 7.32 min

### EXAMPLE 180

### 6-({9-[(3,3-dimethyl-2,3-dihydro-1,4-benzodioxin-5-yl)methyl]-3,9-diazaspiro[5.5]undec-3-yl}carbonyl)pyrimidin-4-amine

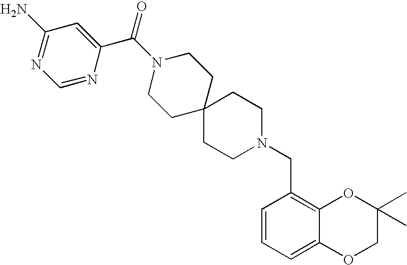

The title compound was prepared with the procedure of Example 181 using 3-[(3,3-dimethyl-2,3-dihydro-1,4-benzodioxin-5-yl)methyl]-3,9-diazaspiro[5.5]undecane as starting material to give the product as a white solid. (38 mg, 28%).

¹H NMR (399.99 MHz, DMSO-D₆) δ 9.09 (d, 1H), 8.43 (d, 1H), 7.54 (s, 1H), 7.03 (s, 2H), 6.91 (t, 1H), 6.50 (d, 1H), 6.50 (d, 1H), 4.24 (dd, 2H), 3.97 (d, 2H), 3.39-2.96 (m, 8H), 1.89 (d, 2H), 1.72-1.46 (m, 4H), 1.44-1.28 (m, 8H)

APCI-MS m/z: 452.0 [MH+]

HPLC (Method A) RT: 5.54 min

HPLC (Method B) RT: 7.14 min

### EXAMPLE 181

### Methyl [2-({9-[(2,2-dimethyl-2,3-dihydro-1-benzofuran-7-yl)methyl]-3,9-diazaspiro[5.5]undec-3-yl}carbonyl)phenyl]acetate

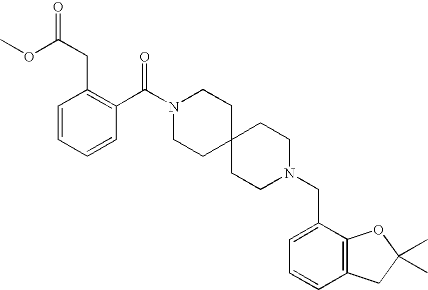

The title compound was prepared with the procedure of Example 194 to give the product as a white solid (24 mg, 35%).

¹H NMR (499.881 MHz, DMSO-D₆) δ 9.18 (s, 1H), 7.41-7.18 (m, 6H), 6.90 (td, 1H), 4.19 (dd, 2H), 3.82-3.54 (m, 2H), 3.59 (s, 3H), 3.23 (d, 2H), 3.18-2.98 (m, 8H), 1.92-1.82 (m, 2H), 1.68-1.21 (m, 12H)

APCI-MS m/z: 491.1 [MH+]

HPLC (Method A) RT: 8.13 min

HPLC (Method B) RT: 10.04 min

CCL1 SPA Binding Assay

Membranes from CHO-K1 cells transfected with human recombinant chemokine CCR8 receptor (ES-136-M) were purchased from Euroscreen. Membrane preparations are stored at −70 C in 7.5 mM Tris-Cl pH 7.5, 12.5 mM MgCl₂, 0.3 mM EDTA, 1 mM EGTA, 250 mM sucrose until used.

The CCR8 membranes (50.6 mg/ml) were preincubated with Wheat Germ Agglutinin SPA beads (4.05 mg/ml) in assay buffer (50 mM HEPES, 1 mM CaCl₂×2H₂O, 5 mM MgCl₂×6H₂O, 75 mM NaCl, 0.1% BSA) at pH=7.4 for 2 hours on ice. A 10-point dose-response curve (final concentrations 50 μM, 16.7 μM, 5.6 μM, 1.9 μM, 0.62 μM, 0.21 μM, 0.069 μM, 0.023 μM) was prepared by diluting compounds by serial dilution 1:3 in DMSO. In the screening plate (Polystyrene NBS plates, Costar Corning 3604) 1 μl from the DMSO solutions of compounds was transferred into each well. 1 μl of DMSO was added to the blank control wells and 1 μl unlabeled CCL1 (300 nM) was added to background control wells. 50 μl of the SPA bead-membrane mixture was added into each well. Finally, 50 μl (30 μM) ¹²⁵I CCL1 (2000 Ci/mM) was added to each well. Plates were then incubated at RT with shaking (700 rpm) for 90 minutes followed by 30 minutes at RT without shaking. The plate was read in a Wallac MicroBeta counter for 2 minutes/well.

All the compounds of the examples (with the exception of examples 162 and 164 for which IC50 values have not been determined) have an IC₅₀ of less than 2 μM. The results obtained for a representative selection of the compounds of the Examples are shown in Table 1 below.

TABLE 1

| Example number | IC₅₀ (nM) |
|---|---|
| 1 | 12 |
| 5 | 9 |
| 18 | 57 |
| 31 | 10 |
| 49 | 38 |
| 63 | 143 |
| 68 | 8 |
| 86 | 8 |
| 92 | 23 |
| 110 | 53 |
| 111 | 104 |
| 124 | 12 |
| 133 | 84 |
| 139 | 70 |
| 143 | 32 |
| 157 | 50 |
| 159 | 87 |
| 173 | 21 |
| 181 | 72 |

1. A compound of general formula 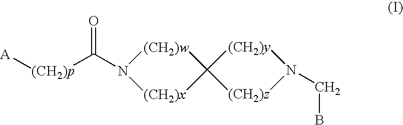
wherein
B represents the group 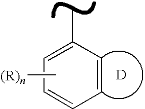
wherein
ring D, together with the two benzene carbon atoms to which it is fused, is a 5- or 6-membered, non-aromatic ring containing one or two ring-oxygen atoms, and optionally containing a carbon-carbon double bond between two ring carbon atoms other than said benzene carbon atoms, ring D being optionally substituted with one or more substituents independently selected from C₁-C₆ alkyl, C₃-C₆ cycloalkyl, or phenyl (said phenyl being optionally substituted with one or more substituents independently selected from halogen, hydroxyl or C₁-C₄ alkoxy);
and additionally wherein when ring D is a 5-membered, non-aromatic ring containing two ring-oxygen atoms that are 1,3 disposed, ring D may be optionally substituted with group E, wherein group E together with a single carbon atom on ring D, represents a 4- to 8-membered cycloalkyl ring, such that group E forms a spiro structure with ring D;
each R represents a group independently selected from halogen or C₁-C₄ alkyl; and
n is 0, 1 or 2;
w x, v and z are independently 1, 2 or 3;
p is 0, 1 or 2:
A represents a group selected from phenyl, a 5- or 6-membered heteroaromatic ring containing at least one ring heteroatom independently selected from nitrogen, oxygen or sulphur, or pyridine-N-oxide, each group being optionally substituted with one or more substituents independently selected from hydroxyl, —CN, halogen, oxo (═O), C₁-C₆ aminoalkyl, C₁-C₆ alkylamino-C₁-C₆ alkyl, N,N-di(C₁-C₆)alkylamino-C₁-C₆ alkyl, C₁-C₆ alkoxy, C₁-C₆ alkylcarbonyl, —NR¹R², —C(O)—NR³R⁴, —C₁-C₆ alkylenyl-C(O)—NR³R⁴, —C₁-C₄ alkyl-C(O)—NR⁵R⁶, —NHSO₂—R⁷, —NHC(O)R⁸, —SO₂NH₂, carboxyl, carboxyl-C₁-C₆ alkyl, C₁-C₆ alkoxycarbonyl, C₁-C₄ alkoxycarbonyl-C₁-C₄ alkyl, C₃-C₆ cycloalkylamino, phenyl, pyridyl (said phenyl and pyridyl being optionally further substituted with one or more groups independently selected from halogen, hydroxyl, carboxy or C₁-C₄ alkyl), C₁-C₆ alkyl or C₃-C₆ cycloalkyl (said latter two C₁-C₆ alkyl and C₃-C₆ cycloalkyl substituents being optionally further substituted with one or more substituents independently selected from halogen, hydroxyl, or —CN);
or A represents a 9- or 10-membered bicyclic ring system containing one or more ring heteroatoms independently selected from nitrogen, oxygen or sulphur and which is optionally substituted with one or more substituents independently selected from hydroxyl, —CN, halogen, oxo, C₁-C₆ alkoxy, —NR⁹R¹⁰, carboxyl, or C₁-C₆ alkyl;
wherein:
R¹ and R² each independently represent a hydrogen atom, a C₁-C₆ alkyl, C₃-C₆ cycloalkyl or R¹ and R² together with the nitrogen atom to which they are attached form a hydantoin group or form a 4- to 7-membered saturated heterocycle, said heterocycle being optionally substituted with hydroxyl, C₁-C₄ alkoxy, or C₁-C₄ alkoxy-C₁-C₄ alkyl
R³ and R⁴ each independently represent a hydrogen atom, C₁-C₆ alkyl, or C₃-C₆ cycloalkyl, or R³ and R⁴ together with the nitrogen atom to which they are attached form a 4- to 7-membered saturated heterocycle, said heterocycle being optionally substituted with aminocarbonyl;
R⁵ and R⁶ each independently represent a hydrogen atom, C₁-C₆ alkyl, or C₃-C₆ cycloalkyl, or R⁵ and R⁶ together with the nitrogen atom to which they are attached form a 4- to 7-membered saturated heterocycle, said heterocycle being optionally substituted with aminocarbonyl;
R⁷ represents C₁-C₆ alkyl, or a 6-membered saturated or unsaturated heterocyclic ring, the ring containing at least one nitrogen atom, the ring being optionally substituted with one or more substituents independently selected from halogen, oxo, C₁-C₆ alkoxy, or C₁-C₆ alkyl;
R⁸ represents pyridine-N-oxide optionally substituted with one or more substituents independently selected from halogen, or C₁-C₆ alkyl, or R⁸ represents C₁-C₆ alkyl, C₁-C₆ hydroxyalkyl, or a 5- or 6-membered saturated heterocyclic ring containing at least one heteroatom independently selected from nitrogen and oxygen, which ring being optionally substituted with one or more substituents independently selected from halogen, C₁-C₆ alkoxy, oxo, or C₁-C₆ alkyl; and
R⁹ and R¹⁰ each independently represent a hydrogen atom or C₁-C₆ alkyl;
or a pharmaceutically acceptable salt thereof.

2. The compound or pharmaceutically acceptable salt according to claim 1, wherein ring D is substituted with one or more C₁-C₄ alkyl groups.

3. The compound or pharmaceutically acceptable salt according to claim 1, wherein B represents the group 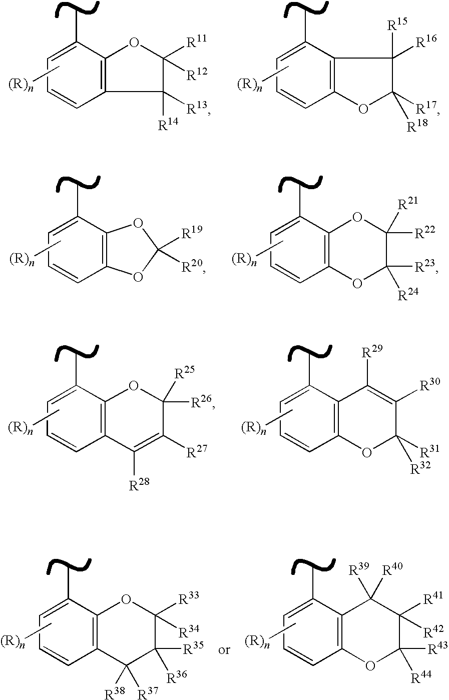
wherein each R¹¹, R¹², R¹³, R¹⁴, R¹⁵, R¹⁶, R¹⁷, R¹⁸, R²¹, R²², R²³, R²⁴, R²⁵, R²⁶, R²⁷, R²⁸, R²⁹, R³⁰ R³¹, R³², R³³, R³⁴, R³⁵, R³⁶, R³⁷, R³⁸, R³⁹, R⁴⁰, R⁴¹, R⁴², R⁴³, and R⁴⁴ independently represent a hydrogen atom or C₁-C₆ alkyl;
R¹⁹ and R²⁰ each independently represent hydrogen, C₁-C₆ alkyl, C₃-C₆ cycloalkyl, or phenyl; or R¹⁹ and R²⁰ together with the carbon to which they are attached form a 4- to 8-membered cycloalkyl ring;
n is 0, 1 or 2 and each R represents a group independently selected from halogen or C₁-C₄ alkyl.

4. The compound or pharmaceutically acceptable salt according to claim 3, wherein each R¹¹, R¹², R¹³, R¹⁴, R¹⁵, R¹⁶, R¹⁷R¹⁸, R²¹, R²², R²³, R²⁴, R²⁵, R²⁶, R²⁷, R²⁸, R²⁹, R³⁰ R³¹, R³², R³³, R³⁴, R³⁵, R³⁶, R³⁷, R³⁸, R³⁹, R⁴⁰, R⁴¹, R⁴², R⁴³ and R⁴⁴ independently represent hydrogen or C₁-C₄ alkyl.

5. The compound or pharmaceutically acceptable salt according to claim 4, wherein each R¹¹, R¹², R¹³, R¹⁴, R¹⁵, R¹⁶, R¹⁷, R¹⁸, R²¹, R²², R²³, R²⁴, R²⁵, R²⁶, R²⁷, R²⁸, R²⁹, R³⁰ R³¹, R³², R³³, R³⁴, R³⁵, R³⁶, R³⁷, R³⁸, R³⁹, R⁴⁰, R⁴¹, R⁴², R⁴³, and R⁴⁴ independently represent hydrogen or methyl.

6. The compound or pharmaceutically acceptable salt according to claim 3, wherein B represents the group 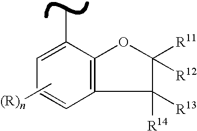
wherein n is 0, 1, or 2 and each R represents a group independently selected from halogen or C₁-C₄ alkyl, and R¹¹, R¹², R¹³, and R¹⁴ are as defined in claim 3.

7. The compound or pharmaceutically acceptable salt according to claim 3, wherein, B represents the group 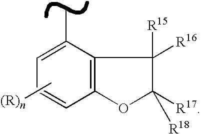
wherein n is 0, 1, or 2 and each R represents a group independently selected from halogen or C₁-C₄ alkyl, and R¹⁵, R¹⁶, R¹⁷, and R¹⁸ are as defined in claim 3.

8. The compound or pharmaceutically acceptable salt according to claim 3, wherein, B represents the group 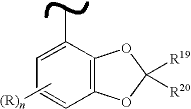
wherein n is 0, 1, or 2 and each R represents a group independently selected from halogen or C₁-C₄ alkyl, and R¹⁹ and R²⁰ are as defined in claims 3.

9. The compound or pharmaceutically acceptable salt according to claim 3, wherein, B represents the group 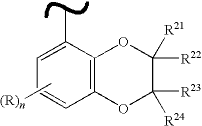
wherein n is 0, 1, or 2 and each R represents a group independently selected from halogen or C₁-C₄ alkyl, and R²¹, R²², R²³, and R²⁴ are as defined in claim 3.

10. The compound or pharmaceutically acceptable salt according to claim 3, wherein, B represents the group 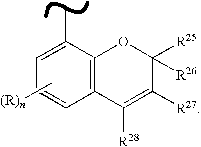
wherein n is 0, 1, or 2 and each R represents a group independently selected from halogen or C₁-C₄ alkyl, and R²⁵, R²⁶, R²⁷, and R²⁸ are as defined in claim 3.

11. The compound or pharmaceutically acceptable salt according to claim 1, wherein the group B is selected from the following: 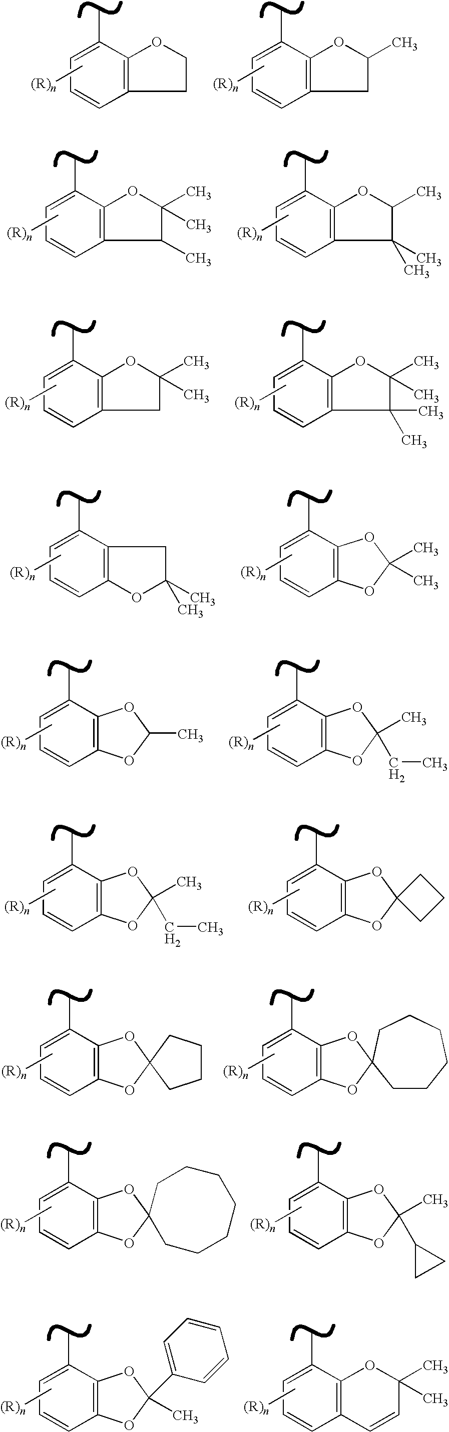 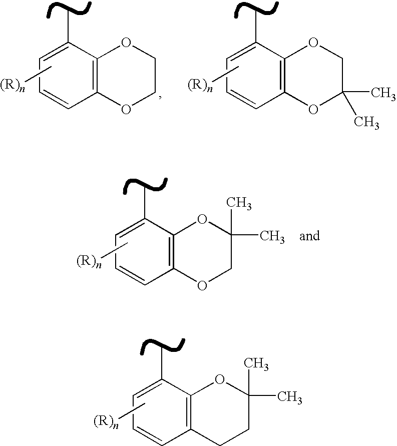
and wherein n is 0, 1, or 2 and each R represents a group independently selected from halogen or C₁-C₄ alkyl.

12. The compound or pharmaceutically acceptable salt according to claim 11, wherein the group B is: 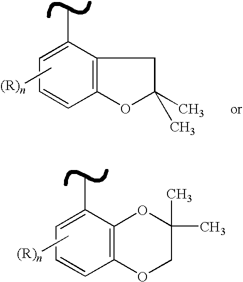
and wherein n is 0, 1, or 2 and each R represents a group independently selected from halogen or C₁-C₄ alkyl.

13. The compound or pharmaceutically acceptable salt according to claim 1, wherein n is 0.

14. The compound or pharmaceutically acceptable salt according to claim 1, wherein the w+x is not greater than 5 and y+z is not greater than 5, and w+x+y+z is greater than 5.

15. The compound or pharmaceutically acceptable salt according to claim 1, wherein each of w, x, y and z is equal to 2.

16. The compound or pharmaceutically acceptable salt according to claim 1, wherein w and x are each equal to 1, and y and z are each equal to 2.

17. The compound or pharmaceutically acceptable salt according to claim 1, wherein p is 0.

18. The compound or pharmaceutically acceptable salt according to claim 1, wherein A is phenyl, pyridyl, or pyrimidinyl each being substituted with 0, 1 or 2 substituents independently selected from hydroxyl, —CN, halogen, oxo (═O), C₁-C₆ aminoalkyl, C₁-C₆ alkoxy, C₁-C₆ alkylcarbonyl, —NR¹R², —C(O)—NR³R⁴, —C₁-C₄ alkyl-C(O)—NR⁵R⁶, —NHSO₂—R⁷, —NHC(O)R⁸, —SO₂NH₂, carboxyl, carboxyl-C₁-C₆ alkyl, C₁-C₆ alkoxycarbonyl, C₁-C₄ alkoxycarbonyl-C₁-C₄ alkyl, C₃-C₆ cycloalkylamino, phenyl, pyridyl (said phenyl and pyridyl being optionally further substituted with one or more groups independently selected from halogen, hydroxyl, carboxy or C₁-C₄ alkyl), C₁-C₆ alkyl or C₃-C₆ cycloalkyl; said latter two C₁-C₆ alkyl and C₃-C₆ cycloalkyl substituents being optionally further substituted with one or more substituents independently selected from halogen, hydroxyl, or —CN, and wherein R¹, R², R³, R⁴, R⁵, R⁶, R⁷, and R⁸ are as defined in claim 1.

19. The compound or pharmaceutically acceptable salt according to claim 18, wherein A is phenyl, pyridyl, or pyrimidinyl, each of which is substituted with one or two substituents independently selected from hydroxyl, cyano, halogen, C₁-C₆ alkyl, NH₂, C₁-C₄ alkoxycarbonyl, C₁-C₄ alkoxycarbonyl-C₁-C₄ alkyl, —C(O)—NR³R⁴; —C₁-C₄ alkyl-C(O)—NR⁵R⁶, or —NHC(O)R⁸, and wherein R³, R⁴, R⁵, R⁶, and R⁸ are as defined in claim 18.

20. The compound or pharmaceutically acceptable salt according to claim 18, wherein A is pyridyl or pyrimidinyl, each substituted with NH₂.

21. The compound or pharmaceutically acceptable salt according to claim 1, wherein A is pyridyl substituted with at least one group independently selected from NR¹R², or —C₁-C₂-alkyl-C(O)—NR³R⁴; R¹ and R² each independently represent hydrogen or —C₁-C₄-alkyl; R³ and R⁴ each independently represent hydrogen or —C₁-C₄-alkyl.

22. The compound as defined in claim 1 selected from:
3-[(2,2-dimethyl-2,3-dihydro-1-benzofuran-7-yl)methyl]-9-isonicotinoyl-3,9-diazaspiro[5.5]undecane;
3-[(2,2-dimethyl-2,3-dihydro-1-benzofuran-7-yl)methyl]-9-[(1-oxidopyridin-2-yl)carbonyl]-3,9-diazaspiro[5.5]undecane;
3-({9-[(2,2-dimethyl-2,3-dihydro-1-benzofuran-7-yl)methyl]-3,9-diazaspiro[5.5]undec-3-yl}carbonyl)pyridin-2(1H)-one;
[2-({9-[(2,2-dimethyl-2,3-dihydro-1-benzofuran-7-yl)methyl]-3,9-diazaspiro[5.5]undec-3-yl}carbonyl)phenyl]acetic acid;
methyl [2-({9-[(2,2-dimethyl-2,3-dihydro-1-benzofuran-7-yl)methyl]-3,9-diazaspiro[5.5]undec-3-yl}carbonyl)phenyl]acetate;
3-({9-[(2,2-dimethyl-2,3-dihydro-1-benzofuran-7-yl)methyl]-3,9-diazaspiro[5.5]undec-3-yl}carbonyl)-1-methylpyridin-2(1H)-one;
3-[(2,2-dimethyl-2,3-dihydro-1-benzofuran-7-yl)methyl]-9-(pyrimidin-4-ylcarbonyl)-3,9-diazaspiro[5.5]undecane;
4-({9-[(2,2-dimethyl-2,3-dihydro-1-benzofuran-7-yl)methyl]-3,9-diazaspiro[5.5]undec-3-yl}carbonyl)pyridin-3-ol;
2-({9-[(2,2-dimethyl-2,3-dihydro-1-benzofuran-7-yl)methyl]-3,9-diazaspiro[5.5]undec-3-yl}carbonyl)pyridin-3-ol;
5-({9-[(2,2-dimethyl-2,3-dihydro-1-benzofuran-7-yl)methyl]-3,9-diazaspiro[5.5]undec-3-yl}carbonyl)pyridin-2-amine;
3-({9-[(2,2-dimethyl-2,3-dihydro-1-benzofuran-7-yl)methyl]-3,9-diazaspiro[5.5]undec-3-yl}carbonyl)pyridin-2-amine;
2-({9-[(2,2-dimethyl-2,3-dihydro-1-benzofuran-7-yl)methyl]-3,9-diazaspiro[5.5]undec-3-yl}carbonyl)pyridin-4-ol;
3-({9-[(2,2-dimethyl-2,3-dihydro-1-benzofuran-7-yl)methyl]-3,9-diazaspiro[5.5]undec-3-yl}carbonyl)pyridin-4-ol;
3-(1H-1,2,3-benzotriazol-5-ylcarbonyl)-9-[(2,2-dimethyl-2,3-dihydro-1-benzofuran-7-yl)methyl]-3,9-diazaspiro[5.5]undecane;
5-({9-[(2,2-dimethyl-2,3-dihydro-1-benzofuran-7-yl)methyl]-3,9-diazaspiro[5.5]undec-3-yl}carbonyl)pyridine-2-carbonitrile;
2′-({9-[(2,2-dimethyl-2,3-dihydro-1-benzofuran-7-yl)methyl]-3,9-diazaspiro[5.5]undec-3-yl}carbonyl)biphenyl-2-carboxylic acid;
2-[2-({9-[(2,2-dimethyl-2,3-dihydro-1-benzofuran-7-yl)methyl]-3,9-diazaspiro[5.5]undec-3-yl}carbonyl)phenyl]acetamide;
1-{[2-({9-[(2,2-dimethyl-2,3-dihydro-1-benzofuran-7-yl)methyl]-3,9-diazaspiro[5.5]undec-3-yl}carbonyl)phenyl]acetyl}-D-prolinamide;
N-cyclopropyl-2-[2-({9-[(2,2-dimethyl-2,3-dihydro-1-benzofuran-7-yl)methyl]-3,9-diazaspiro[5.5]undec-3-yl}carbonyl)phenyl]acetamide;
3-[2-(2-azetidin-1-yl-2-oxoethyl)benzoyl]-9-[(2,2-dimethyl-2,3-dihydro-1-benzofuran-7-yl)methyl]-3,9-diazaspiro[5.5]undecane;
[5-chloro-2-({9-[(2,2-dimethyl-2,3-dihydro-1-benzofuran-7-yl)methyl]-3,9-diazaspiro[5.5]undec-3-yl}carbonyl)phenyl]acetic acid;
3-[2-({9-[(2,2-dimethyl-2,3-dihydro-1-benzofuran-7-yl)methyl]-3,9-diazaspiro[5.5]undec-3-yl}carbonyl)phenyl]propanoic acid;
4-({9-[(2,2-dimethyl-2,3-dihydro-1-benzofuran-7-yl)methyl]-3,9-diazaspiro[5.5]undec-3-yl}carbonyl)pyridin-2-amine;
4-({9-[(2,2-dimethyl-2,3-dihydro-1-benzofuran-7-yl)methyl]-3,9-diazaspiro[5.5]undec-3-yl}carbonyl)pyridin-3-amine;
N-[2-({9-[(2,2-dimethyl-2,3-dihydro-1-benzofuran-7-yl)methyl]-3,9-diazaspiro[5.5]undec-3-yl}carbonyl)phenyl]methanesulfonamide;
4-({9-[(2,2-dimethyl-2,3-dihydro-1-benzofuran-7-yl)methyl]-3,9-diazaspiro[5.5]undec-3-yl}carbonyl)-1H-pyrazol-3-amine;
3-[(2,2-dimethyl-2,3-dihydro-1-benzofuran-7-yl)methyl]-9-(1,2,3-thiadiazol-4-ylcarbonyl)-3,9-diazaspiro[5.5]undecane;
3-[(2,2-dimethyl-2,3-dihydro-1-benzofuran-7-yl)methyl]-9-[(3-methylisoxazol-4-yl)carbonyl]-3,9-diazaspiro[5.5]undecane;
3-[(2,2-dimethyl-2,3-dihydro-1-benzofuran-7-yl)methyl]-9-(1H-pyrazol-4-ylcarbonyl)-3,9-diazaspiro[5.5]undecane;
3-[(2,2-dimethyl-2,3-dihydro-1-benzofuran-7-yl)methyl]-9-(3-furoyl)-3,9-diazaspiro[5.5]undecane;
3-[(2,2-dimethyl-2,3-dihydro-1-benzofuran-7-yl)methyl]-9-(isoxazol-5-ylcarbonyl)-3,9-diazaspiro[5.5]undecane;
3-[(2,2-dimethyl-2,3-dihydro-1-benzofuran-7-yl)methyl]-9-[(1-methyl-1H-imidazol-4-yl)carbonyl]-3,9-diazaspiro[5.5]undecane;
1-[5-({9-[(2,2-dimethyl-2,3-dihydro-1-benzofuran-7-yl)methyl]-3,9-diazaspiro[5.5]undec-3-yl}carbonyl)-1H-pyrrol-3-yl]ethanone;
3-[(2,2-dimethyl-2,3-dihydro-1-benzofuran-7-yl)methyl]-9-(1H-pyrazol-3-ylcarbonyl)-3,9-diazaspiro[5.5]undecane;
3-[(2,2-dimethyl-2,3-dihydro-1-benzofuran-7-yl)methyl]-9-(1H-indol-3-ylcarbonyl)-3,9-diazaspiro[5.5]undecane;
3-[(2,2-dimethyl-2,3-dihydro-1-benzofuran-7-yl)methyl]-9-(1H-indazol-3-ylcarbonyl)-3,9-diazaspiro[5.5]undecane;
3-[(2,2-dimethyl-2,3-dihydro-1-benzofuran-7-yl)methyl]-9-(1H-indol-2-ylcarbonyl)-3,9-diazaspiro[5.5]undecane;
3-(2-chloroisonicotinoyl)-9-[(2,2-dimethyl-2,3-dihydro-1-benzofuran-7-yl)methyl]-3,9-diazaspiro[5.5]undecane;
[2-({9-[(2,2-dimethyl-2,3-dihydro-1-benzofuran-7-yl)methyl]-3,9-diazaspiro[5.5]undec-3-yl}carbonyl)phenyl]amine;
N-[2-({9-[(2,2-dimethyl-2,3-dihydro-1-benzofuran-7-yl)methyl]-3,9-diazaspiro[5.5]undec-3-yl}carbonyl)phenyl]acetamide;
N-[2-({9-[(2,2-dimethyl-2,3-dihydro-1-benzofuran-7-yl)methyl]-3,9-diazaspiro[5.5]undec-3-yl}carbonyl)phenyl]-2-hydroxyacetamide;
1-[4-({9-[(2,2-dimethyl-2,3-dihydro-1-benzofuran-7-yl)methyl]-3,9-diazaspiro[5.5]undec-3-yl}carbonyl)pyridin-2-yl]pyrrolidin-3-ol;
3-[(2,2-dimethyl-2,3-dihydro-1-benzofuran-7-yl)methyl]-9-{2-[(2S)-2-(methoxymethyl)pyrrolidin-1-yl]isonicotinoyl}-3,9-diazaspiro[5.5]undecane;
4-({9-[(2,2-dimethyl-2,3-dihydro-1-benzofuran-7-yl)methyl]-3,9-diazaspiro[5.5]undec-3-yl}carbonyl)-N-methylpyridin-2-amine;
N-[2-({9-[(2,2-dimethyl-2,3-dihydro-1-benzofuran-7-yl)methyl]-3,9-diazaspiro[5.5]undec-3-yl}carbonyl)phenyl]-6-methyl-2,4-dioxo-1,2,3,4-tetrahydropyrimidine-5-sulfonamide;
1-[2-({9-[(2,2-dimethyl-2,3-dihydro-1-benzofuran-7-yl)methyl]-3,9-diazaspiro[5.5]undec-3-yl}carbonyl)phenyl]imidazolidine-2,4-dione;
N-[2-({9-[(2,2-dimethyl-2,3-dihydro-1-benzofuran-7-yl)methyl]-3,9-diazaspiro[5.5]undec-3-yl}carbonyl)phenyl]nicotinamide 1-oxide;
N-[2-({9-[(2,2-dimethyl-2,3-dihydro-1-benzofuran-7-yl)methyl]-3,9-diazaspiro[5.5]undec-3-yl}carbonyl)phenyl]-1-methyl-L-prolinamide;
N-[2-({9-[(2,2-dimethyl-2,3-dihydro-1-benzofuran-7-yl)methyl]-3,9-diazaspiro[5.5]undec-3-yl}carbonyl)phenyl]tetrahydrofuran-2-carboxamide;
N-[2-({9-[(2,2-dimethyl-2,3-dihydro-1-benzofuran-7-yl)methyl]-3,9-diazaspiro[5.5]undec-3-yl}carbonyl)phenyl]-5-oxoprolinamide;
[4-({9-[(2,2-dimethyl-2,3-dihydro-1-benzofuran-7-yl)methyl]-3,9-diazaspiro[5.5]undec-3-yl}carbonyl)phenyl]amine;
3-[(2,2-dimethyl-2,3-dihydro-1-benzofuran-7-yl)methyl]-9-(3-methylisonicotinoyl)-3,9-diazaspiro[5.5]undecane;
2-({9-[(2,2-dimethyl-2,3-dihydro-1-benzofuran-7-yl)methyl]-3,9-diazaspiro[5.5]undec-3-yl}carbonyl)pyridin-4-amine;
3-[(2,2-dimethyl-2,3-dihydro-1-benzofuran-7-yl)methyl]-9-(2-methylisonicotinoyl)-3,9-diazaspiro[5.5]undecane;
6-({9-[(2,2-dimethyl-2,3-dihydro-1-benzofuran-7-yl)methyl]-3,9-diazaspiro[5.5]undec-3-yl}carbonyl)pyridin-3-amine;
4-({9-[(2,2-dimethyl-2,3-dihydro-1-benzofuran-7-yl)methyl]-3,9-diazaspiro[5.5]undec-3-yl}carbonyl)pyridazin-3-amine;
{[2-({9-[(2,2-dimethyl-2,3-dihydro-1-benzofuran-7-yl)methyl]-3,9-diazaspiro[5.5]undec-3-yl}carbonyl)pyridin-4-yl]methyl}amine;
4-({9-[(2,2-dimethyl-2,3-dihydro-1-benzofuran-7-yl)methyl]-3,9-diazaspiro[5.5]undec-3-yl}carbonyl)quinolin-2-ol;
3-[(2,2-dimethyl-2,3-dihydro-1-benzofuran-7-yl)methyl]-9-(1,8-naphthyridin-2-ylcarbonyl)-3,9-diazaspiro[5.5]undecane;
3-[(2,2-dimethyl-2,3-dihydro-1-benzofuran-7-yl)methyl]-9-(1,6-naphthyridin-2-ylcarbonyl)-3,9-diazaspiro[5.5]undecane;
4-({9-[(2,2-dimethyl-2,3-dihydro-1-benzofuran-7-yl)methyl]-3,9-diazaspiro[5.5]undec-3-yl}carbonyl)-6-methoxypyridin-3-amine;
4-({9-[(2,2-dimethyl-2,3-dihydro-1-benzofuran-7-yl)methyl]-3,9-diazaspiro[5.5]undec-3-yl}carbonyl)-2-methylquinolin-3-amine;
7-({9-[(2,2-dimethyl-2,3-dihydro-1-benzofuran-7-yl)methyl]-3,9-diazaspiro[5.5]undec-3-yl}carbonyl)-1H-indole-2,3-dione;
3-({9-[(2,2-dimethyl-2,3-dihydro-1-benzofuran-7-yl)methyl]-3,9-diazaspiro[5.5]undec-3-yl}carbonyl)pyridin-4-amine;
5-({9-[(2,2-dimethyl-2,3-dihydro-1-benzofuran-7-yl)methyl]-3,9-diazaspiro[5.5]undec-3-yl}carbonyl)pyridin-3-amine;
3-[(2,2-dimethyl-2,3-dihydro-1-benzofuran-7-yl)methyl]-9-(1H-indol-7-ylcarbonyl)-3,9-diazaspiro[5.5]undecane;
3-[(2,2-dimethyl-2,3-dihydro-1-benzofuran-7-yl)methyl]-9-(1H-indol-5-ylcarbonyl)-3,9-diazaspiro[5.5]undecane;
3-[(2,2-dimethyl-2,3-dihydro-1-benzofuran-7-yl)methyl]-9-(1H-indol-6-ylcarbonyl)-3,9-diazaspiro[5.5]undecane;
3-(1H-benzimidazol-6-ylcarbonyl)-9-[(2,2-dimethyl-2,3-dihydro-1-benzofuran-7-yl)methyl]-3,9-diazaspiro[5.5]undecane;
4-({9-[(3,3-dimethyl-2,3-dihydro-1,4-benzodioxin-5-yl)methyl]-3,9-diazaspiro[5.5]undec-3-yl}carbonyl)pyrimidin-2-amine;
3-({9-[(2,2-dimethyl-2,3-dihydro-1-benzofuran-7-yl)methyl]-3,9-diazaspiro[5.5]undec-3-yl}carbonyl)benzonitrile;
4-({9-[(2,2-dimethyl-2,3-dihydro-1-benzofuran-7-yl)methyl]-3,9-diazaspiro[5.5]undec-3-yl}carbonyl)benzonitrile;
4-({9-[(2,2-dimethyl-2,3-dihydro-1-benzofuran-7-yl)methyl]-3,9-diazaspiro[5.5]undec-3-yl}carbonyl)benzenesulfonamide;
[3-({9-[(2,2-dimethyl-2,3-dihydro-1-benzofuran-7-yl)methyl]-3,9-diazaspiro[5.5]undec-3-yl}carbonyl)phenyl]amine;
5-({9-[(2,2-dimethyl-2,3-dihydro-1-benzofuran-7-yl)methyl]-3,9-diazaspiro[5.5]undec-3-yl}carbonyl)pyrazin-2(1H)-one;
5-({9-[(2,2-dimethyl-2,3-dihydro-1-benzofuran-7-yl)methyl]-3,9-diazaspiro[5.5]undec-3-yl}carbonyl)pyridin-2(1H)-one;
3-isonicotinoyl-9-[(2-methyl-2,3-dihydro-1-benzofuran-7-yl)methyl]-3,9-diazaspiro[5.5]undecane;
3-isonicotinoyl-9-[(2,3,3-trimethyl-2,3-dihydro-1-benzofuran-7-yl)methyl]-3,9-diazaspiro[5.5]undecane;
3-isonicotinoyl-9-[(2,2,3-trimethyl-2,3-dihydro-1-benzofuran-7-yl)methyl]-3,9-diazaspiro[5.5]undecane;
3-(2,3-dihydro-1-benzofuran-7-ylmethyl)-9-isonicotinoyl-3,9-diazaspiro[5.5]undecane;
3-isonicotinoyl-9-[(2,2,3,3-tetramethyl-2,3-dihydro-1-benzofuran-7-yl)methyl]-3,9-diazaspiro[5.5]undecane;
3-[(5-chloro-2,2-dimethyl-2,3-dihydro-1-benzofuran-7-yl)methyl]-9-isonicotinoyl-3,9-diazaspiro[5.5]undecane;
3-isonicotinoyl-9-[(2,2,4-trimethyl-2,3-dihydro-1-benzofuran-7-yl)methyl]-3,9-diazaspiro[5.5]undecane;
3-[(4-chloro-2,2-dimethyl-2,3-dihydro-1-benzofuran-7-yl)methyl]-9-isonicotinoyl-3,9-diazaspiro[5.5]undecane;
3-[(2,2-dimethyl-2,3-dihydro-1-benzofuran-4-yl)methyl]-9-isonicotinoyl-3,9-diazaspiro[5.5]undecane;
4-({9-[(2,2-dimethyl-2,3-dihydro-1-benzofuran-4-yl)methyl]-3,9-diazaspiro[5.5]undec-3-yl}carbonyl)pyridin-3-amine;
6-({9-[(2,2-dimethyl-2,3-dihydro-1-benzofuran-4-yl)methyl]-3,9-diazaspiro[5.5]undec-3-yl}carbonyl)pyridin-3-amine;
2-[2-({9-[(2,2-dimethyl-2,3-dihydro-1-benzofuran-4-yl)methyl]-3,9-diazaspiro[5.5]undec-3-yl}carbonyl)phenyl]acetamide;
3-(1,3-benzodioxol-4-ylmethyl)-9-isonicotinoyl-3,9-diazaspiro[5.5]undecane;
3-[(2,2-dimethyl-1,3-benzodioxol-4-yl)methyl]-9-isonicotinoyl-3,9-diazaspiro[5. S]undecane;
4-({9-[(2,2-dimethyl-1,3-benzodioxol-4-yl)methyl]-3,9-diazaspiro[5.5]undec-3-yl}carbonyl)pyridin-3-amine;
4-({9-[(2,2-dimethyl-1,3-benzodioxol-4-yl)methyl]-3,9-diazaspiro[5.5]undec-3-yl}carbonyl)pyridin-2-amine;
2-({9-[(2,2-dimethyl-1,3-benzodioxol-4-yl)methyl]-3,9-diazaspiro[5.5]undec-3-yl}carbonyl)pyridin-3-amine;
2-[2-({9-[(2,2-dimethyl-1,3-benzodioxol-4-yl)methyl]-3,9-diazaspiro[5.5]undec-3-yl}carbonyl)phenyl]acetamide;
4-({9-[(2,2-dimethyl-1,3-benzodioxol-4-yl)methyl]-3,9-diazaspiro[5.5]undec-3-yl}carbonyl)pyridazin-3-amine;
4-({9-[(2-ethyl-2-methyl-1,3-benzodioxol-4-yl)methyl]-3,9-diazaspiro[5.5]undec-3-yl}carbonyl)pyridin-3-amine;
4-{[9-(spiro[1,3-benzodioxole-2,1′-cyclobutan]-4-ylmethyl)-3,9-diazaspiro[5.5]undec-3-yl]carbonyl}pyridin-3-amine;
4-{[9-(spiro[1,3-benzodioxole-2,1′-cyclopentan]-4-ylmethyl)-3,9-diazaspiro[5.5]undec-3-yl]carbonyl}pyridin-3-amine;
4-{[9-(spiro[1,3-benzodioxole-2,1′-cyclopentan]-4-ylmethyl)-3,9-diazaspiro[5.5]undec-3-yl]carbonyl}pyridin-2-amine;
4-{[9-(spiro[1,3-benzodioxole-2,1′-cycloheptan]-4-ylmethyl)-3,9-diazaspiro[5.5]undec-3-yl]carbonyl}pyridin-3-amine;
3-[(2-ethyl-2-methyl-1,3-benzodioxol-4-yl)methyl]-9-[(1-oxidopyridin-2-yl)carbonyl]-3,9-diazaspiro[5.5]undecane;
3-[(1-oxidopyridin-2-yl)carbonyl]-9-(spiro[1,3-benzodioxole-2,1′-cyclobutan]-4-ylmethyl)-3,9-diazaspiro[5.5]undecane;
3-[(1-oxidopyridin-2-yl)carbonyl]-9-(spiro[1,3-benzodioxole-2,1′-cyclooctan]-4-ylmethyl)-3,9-diazaspiro[5.5]undecane;
3-[(2-methyl-2-phenyl-1,3-benzodioxol-4-yl)methyl]-9-[(1-oxidopyridin-2-yl)carbonyl]-3,9-diazaspiro[5.5]undecane;
3-[(2-cyclopropyl-2-methyl-1,3-benzodioxol-4-yl)methyl]-9-[(1-oxidopyridin-2-yl)carbonyl]-3,9-diazaspiro[5.5]undecane;
3-[(2,2-dimethyl-2H-chromen-8-yl)methyl]-9-isonicotinoyl-3,9-diazaspiro[5.5]undecane;
4-({9-[(2,2-dimethyl-2H-chromen-8-yl)methyl]-3,9-diazaspiro[5.5]undec-3-yl}carbonyl)pyridin-3-amine;
6-({9-[(2,2-dimethyl-2H-chromen-8-yl)methyl]-3,9-diazaspiro[5.5]undec-3-yl}carbonyl)pyridin-3-amine;
2-[2-({9-[(2,2-dimethyl-2H-chromen-8-yl)methyl]-3,9-diazaspiro[5.5]undec-3-yl}carbonyl)phenyl]acetamide;
3-(2,3-dihydro-1,4-benzodioxin-5-ylmethyl)-9-isonicotinoyl-3,9-diazaspiro[5.5]undecane;
3-[(2,2-dimethyl-2,3-dihydro-1,4-benzodioxin-5-yl)methyl]-9-isonicotinoyl-3,9-diazaspiro[5.5]undecane;
3-[(2,2-dimethyl-2,3-dihydro-1-benzofuran-7-yl)methyl]-9-[3-(3-pyridin-2-yl-1,2,4-oxadiazol-5-yl)propanoyl]-3,9-diazaspiro[5.5]undecane;
4-({9-[(2,2-dimethyl-2H-chromen-8-yl)methyl]-3,9-diazaspiro[5.5]undec-3-yl}carbonyl)pyridin-2-amine;
6-({9-[(2,2-dimethyl-2H-chromen-8-yl)methyl]-3,9-diazaspiro[5.5]undec-3-yl}carbonyl)pyridin-2(1H)-one;
2-({9-[(2,2-dimethyl-2,3-dihydro-1-benzofuran-7-yl)methyl]-3,9-diazaspiro[5.5]undec-3-yl}carbonyl)benzonitrile;
4-({9-[(2,2-dimethyl-2,3-dihydro-1-benzofuran-7-yl)methyl]-3,9-diazaspiro[5.5]undec-3-yl}carbonyl)pyridine-2,6-diol;
3-[(6-fluoro-4H-1,3-benzodioxin-8-yl)methyl]-9-isonicotinoyl-3,9-diazaspiro[5.5]undecane;
4-({9-[(2,2-dimethyl-2H-chromen-8-yl)methyl]-3,9-diazaspiro[5.5]undec-3-yl}carbonyl)pyridazin-3-amine;
5-chloro-4-({9-[(2,2-dimethyl-1,3-benzodioxol-4-yl)methyl]-3,9-diazaspiro[5.5]undec-3-yl}carbonyl)pyrimidin-2-amine;
4-({9-[(2,2-dimethyl-2,3-dihydro-1-benzofuran-7-yl)methyl]-3,9-diazaspiro[5.5]undec-3-yl}carbonyl)pyrimidin-2-amine;
4-({9-[(2,2-dimethyl-1,3-benzodioxol-4-yl)methyl]-3,9-diazaspiro[5.5]undec-3-yl}carbonyl)pyrimidin-2-amine;
4-({9-[(2,2-dimethyl-2H-chromen-8-yl)methyl]-3,9-diazaspiro[5.5]undec-3-yl}carbonyl)pyrimidin-2-amine;
8-[(2,2-dimethyl-2,3-dihydro-1-benzofuran-7-yl)methyl]-2-(pyridin-4-ylacetyl)-2,8-diazaspiro[4.5]decane;
4-({9-[(2,2-dimethyl-2,3-dihydro-1-benzofuran-4-yl)methyl]-3,9-diazaspiro[5.5]undec-3-yl}carbonyl)pyridazin-3-amine;
4-({9-[(2,2-dimethyl-2,3-dihydro-1-benzofuran-4-yl)methyl]-3,9-diazaspiro[5.5]undec-3-yl}carbonyl)pyrimidin-2-amine;
4-({9-[(3,3-dimethyl-2,3-dihydro-1,4-benzodioxin-5-yl)methyl]-3,9-diazaspiro[5.5]undec-3-yl}carbonyl)pyridin-3-amine;
4-({9-[(3,3-dimethyl-2,3-dihydro-1,4-benzodioxin-5-yl)methyl]-3,9-diazaspiro[5.5]undec-3-yl}carbonyl)pyridin-2-amine;
4-({9-[(2,2,3,3-tetramethyl-2,3-dihydro-1-benzofuran-7-yl)methyl]-3,9-diazaspiro[5.5]undec-3-yl}carbonyl)pyrimidin-2-amine;
6-({9-[(2,2,3,3-tetramethyl-2,3-dihydro-1-benzofuran-7-yl)methyl]-3,9-diazaspiro[5.5]undec-3-yl}carbonyl)pyridin-3-amine;
4-({9-[(2,2,3,3-tetramethyl-2,3-dihydro-1-benzofuran-7-yl)methyl]-3,9-diazaspiro[5.5]undec-3-yl}carbonyl)pyridin-2-amine;
4-({9-[(2,2,3,3-tetramethyl-2,3-dihydro-1-benzofuran-7-yl)methyl]-3,9-diazaspiro[5.5]undec-3-yl}carbonyl)pyridin-3-amine;
4-({9-[(2,2-dimethyl-2H-chromen-5-yl)methyl]-3,9-diazaspiro[5.5]undec-3-yl}carbonyl)pyrimidin-2-amine;
4-({9-[(2,2-dimethyl-3,4-dihydro-2H-chromen-8-yl)methyl]-3,9-diazaspiro[5.5]undec-3-yl}carbonyl)pyrimidin-2-amine;
6-amino-3-({9-[(2,2-dimethyl-1,3-benzodioxol-4-yl)methyl]-3,9-diazaspiro[5.5]undec-3-yl}carbonyl)pyridin-2(1H)-one;
2-({9-[(2,2-dimethyl-2,3-dihydro-1-benzofuran-4-yl)methyl]-3,9-diazaspiro[5.5]undec-3-yl}carbonyl)pyridin-3-amine;
8-[(2,2-dimethyl-2,3-dihydro-1-benzofuran-7-yl)methyl]-2-isonicotinoyl-2,8-diazaspiro[4.5]decane;
8-[(2,2-dimethyl-2H-chromen-8-yl)methyl]-2-isonicotinoyl-2,8-diazaspiro[4.5]decane;
2-[(2,2-dimethyl-2,3-dihydro-1-benzofuran-7-yl)methyl]-8-isonicotinoyl-2,8-diazaspiro[4.5]decane;
7-[(2,2-dimethyl-2,3-dihydro-1-benzofuran-7-yl)methyl]-2-isonicotinoyl-2,7-diazaspiro[3.5]nonane;
7-[(2,2-dimethyl-2H-chromen-8-yl)methyl]-2-isonicotinoyl-2,7-diazaspiro[3.5]nonane;
2-[(2,2-dimethyl-2,3-dihydro-1-benzofuran-7-yl)methyl]-8-(pyridin-4-ylacetyl)-2,8-diazaspiro[4.5]decane;
7-[(2,2-dimethyl-2H-chromen-8-yl)methyl]-2-(pyridin-4-ylacetyl)-2,7-diazaspiro[3.5]nonane;
7-[(2,2-dimethyl-2,3-dihydro-1-benzofuran-7-yl)methyl]-2-(pyridin-4-ylacetyl)-2,7-diazaspiro[3.5]nonane;
2-[4-({9-[(3,3-dimethyl-2,3-dihydro-1,4-benzodioxin-5-yl)methyl]-3,9-diazaspiro[5.5]undec-3-yl}carbonyl)pyridin-3-yl]acetamide;
2-[(2,2-dimethyl-2H-chromen-8-yl)methyl]-8-isonicotinoyl-2,8-diazaspiro[4.5]decane;
8-[(2,2-dimethyl-2H-chromen-8-yl)methyl]-2-(pyridin-4-ylacetyl)-2,8-diazaspiro[4.5]decane;
3-[4-({9-[(2,2-dimethyl-2,3-dihydro-1-benzofuran-4-yl)methyl]-3,9-diazaspiro[5.5]undec-3-yl}carbonyl)pyridin-3-yl]propanamide;
4-({9-[(3,3-dimethyl-2,3-dihydro-1,4-benzodioxin-5-yl)methyl]-3,9-diazaspiro[5.5]undec-3-yl}carbonyl)pyridine-2-carbonitrile;
4-({9-[(3,3-dimethyl-2,3-dihydro-1,4-benzodioxin-5-yl)methyl]-3,9-diazaspiro[5.5]undec-3-yl}carbonyl)pyridine-2-carboxamide;
(2E)-3-[2-({9-[(3,3-dimethyl-2,3-dihydro-1,4-benzodioxin-5-yl)methyl]-3,9-diazaspiro[5.5]undec-3-yl}carbonyl)phenyl]acrylamide;
6-({9-[(2,2-dimethyl-2,3-dihydro-1-benzofuran-4-yl)methyl]-3,9-diazaspiro[5.5]undec-3-yl}carbonyl)pyridazin-3(2H)-one;
5-({9-[(2,2-dimethyl-2,3-dihydro-1-benzofuran-4-yl)methyl]-3,9-diazaspiro[5.5]undec-3-yl}carbonyl)pyridin-3-ol;
3-({9-[(2,2-dimethyl-2,3-dihydro-1-benzofuran-4-yl)methyl]-3,9-diazaspiro[5.5]undec-3-yl}carbonyl)pyridin-4(1H)-one;
3-({9-[(2,2-dimethyl-2,3-dihydro-1-benzofuran-4-yl)methyl]-3,9-diazaspiro[5.5]undec-3-yl}carbonyl)pyrazin-2(1H)-one;
6-({9-[(2,2-dimethyl-2,3-dihydro-1-benzofuran-4-yl)methyl]-3,9-diazaspiro[5.5]undec-3-yl}carbonyl)pyridin-2-amine;
6-({9-[(2,2-dimethyl-2,3-dihydro-1-benzofuran-4-yl)methyl]-3,9-diazaspiro[5.5]undec-3-yl}carbonyl)pyridin-3-ol;
6-({9-[(3,3-dimethyl-2,3-dihydro-1,4-benzodioxin-5-yl)methyl]-3,9-diazaspiro[5.5]undec-3-yl}carbonyl)pyridin-2-amine;
4-({7-[(2,2-dimethyl-2,3-dihydro-1-benzofuran-4-yl)methyl]-2,7-diazaspiro[3.5]non-2-yl}carbonyl)pyrimidin-2-amine;
6-({7-[(2,2-dimethyl-2,3-dihydro-1-benzofuran-4-yl)methyl]-2,7-diazaspiro[3.5]non-2-yl}carbonyl)pyridin-3-amine;
2-[4-({9-[(2,2-dimethyl-2,3-dihydro-1-benzofuran-4-yl)methyl]-3,9-diazaspiro[5.5]undec-3-yl}carbonyl)pyridin-3-yl]acetamide;
2-[4-({9-[(2,2,3,3-tetramethyl-2,3-dihydro-1-benzofuran-7-yl)methyl]-3,9-diazaspiro[5.5]undec-3-yl}carbonyl)pyridin-3-yl]acetamide;
N-cyclopropyl-2-[4-({9-[(2,2-dimethyl-2,3-dihydro-1-benzofuran-4-yl)methyl]-3,9-diazaspiro[5.5]undec-3-yl}carbonyl)pyridin-3-yl]acetamide;
[4-({9-[(3,3-dimethyl-2,3-dihydro-1,4-benzodioxin-5-yl)methyl]-3,9-diazaspiro[5.5]undec-3-yl}carbonyl)pyridin-3-yl]acetic acid;
[4-({9-[(2,2,3,3-tetramethyl-2,3-dihydro-1-benzofuran-7-yl)methyl]-3,9-diazaspiro[5.5]undec-3-yl}carbonyl)pyridin-3-yl]acetic acid;
[4-({9-[(2,2-dimethyl-2,3-dihydro-1-benzofuran-4-yl)methyl]-3,9-diazaspiro[5.5]undec-3-yl}carbonyl)pyridin-3-yl]acetic acid;
6-({7-[(2,2-dimethyl-2,3-dihydro-1-benzofuran-4-yl)methyl]-2,7-diazaspiro[4.4]non-2-yl}carbonyl)pyridin-3-amine;
5-chloro-4-({9-[(2,2-dimethyl-2,3-dihydro-1-benzofuran-4-yl)methyl]-3,9-diazaspiro[5.5]undec-3-yl}carbonyl)pyridin-2-amine;
2-[3-({9-[(2,2-dimethyl-2,3-dihydro-1-benzofuran-4-yl)methyl]-3,9-diazaspiro[5.5]undec-3-yl}carbonyl)phenyl]acetamide;
4-({9-[(2,2-dimethyl-2,3-dihydro-1-benzofuran-4-yl)methyl]-3,9-diazaspiro[5.5]undec-3-yl}carbonyl)benzamide;
2-[4-({9-[(3,3-dimethyl-2,3-dihydro-1,4-benzodioxin-5-yl)methyl]-3,9-diazaspiro[5.5]undec-3-yl}carbonyl)phenyl]acetamide;
5-chloro-4-({9-[(3,3-dimethyl-2,3-dihydro-1,4-benzodioxin-5-yl)methyl]-3,9-diazaspiro[5.5]undec-3-yl}carbonyl)pyrimidin-2-amine;
6-({9-[(3,3-dimethyl-2,3-dihydro-1,4-benzodioxin-5-yl)methyl]-3,9-diazaspiro[5.5]undec-3-yl}carbonyl)pyridin-3-amine;
2-({9-[(3,3-dimethyl-2,3-dihydro-1,4-benzodioxin-5-yl)methyl]-3,9-diazaspiro[5.5]undec-3-yl}carbonyl)pyridin-3-amine;
6-({9-[(2,2-dimethyl-2,3-dihydro-1,4-benzodioxin-5-yl)methyl]-3,9-diazaspiro[5.5]undec-3-yl}carbonyl)pyridin-3-amine;
4-({9-[(2,2-dimethyl-2,3-dihydro-1,4-benzodioxin-5-yl)methyl]-3,9-diazaspiro[5.5]undec-3-yl}carbonyl)pyridin-2-amine;
4-({9-[(2,2-dimethyl-2,3-dihydro-1,4-benzodioxin-5-yl)methyl]-3,9-diazaspiro[5.5]undec-3-yl}carbonyl)pyridin-3-amine;
4-({9-[(2,2-dimethyl-2,3-dihydro-1,4-benzodioxin-5-yl)methyl]-3,9-diazaspiro[5.5]undec-3-yl}carbonyl)pyrimidin-2-amine;
4-({9-[(3,3-dimethyl-2,3-dihydro-1,4-benzodioxin-5-yl)methyl]-3,9-diazaspiro[5.5]undec-3-yl}carbonyl)pyrimidin-2-amine;
8-[(2,2-dimethyl-2,3-dihydro-1-benzofuran-7-yl)methyl]-2-(pyridin-4-ylacetyl)-2,8-diazaspiro[4.5]decane;
6-({9-[(2,2-dimethyl-1,3-benzodioxol-4-yl)methyl]-3,9-diazaspiro[5.5]undec-3-yl}carbonyl)pyrimidin-4-amine;
6-({9-[(3,3-dimethyl-2,3-dihydro-1,4-benzodioxin-5-yl)methyl]-3,9-diazaspiro[5.5]undec-3-yl}carbonyl)pyrimidin-4-amine; and
methyl [2-({9-[(2,2-dimethyl-2,3-dihydro-1-benzofuran-7-yl)methyl]-3,9-diazaspiro[5.5]undec-3-yl}carbonyl)phenyl]acetate;
and pharmaceutically acceptable salts thereof.

23. A pharmaceutical composition comprising a compound as claimed in claim 1 or claim 22 or a pharmaceutically acceptable salt thereof, in association with a pharmaceutically acceptable adjuvant, diluent or carrier.

24. A process for the preparation of a pharmaceutical composition as claimed in claim 23 which comprises mixing a compound as claimed in claim 1 or claim 22 or a pharmaceutically acceptable salt thereof, with a pharmaceutically acceptable adjuvant, diluent or carrier.

25. (canceled)

26. A method for treating a respiratory disease, said method comprising administering to a patient in need thereof a therapeutically effective amount of a compound of claim 1 or claim 22 or a pharmaceutically acceptable salt thereof.

27. The method of claim 26 wherein said respiratory disease is asthma, COPD or rhinitis.

28. A process for the preparation of a compound as defined in claim 1 or pharmaceutically acceptable salt thereof which comprises:
(a) reaction of a compound of formula (II): 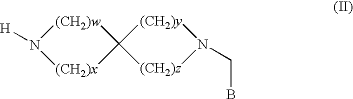
where w, x, y, z and B are as defined in claim 1, with a compound of formula (III) 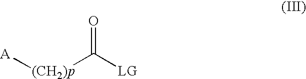
wherein p is as defined in claim 1 and A is as defined in claim 1 or a protected derivative thereof, and LG is a leaving group, or
(b) reaction of a compound of formula (IV): 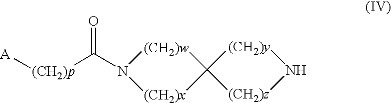
wherein p, w, x, y, and Z are as defined in claim 1 and A is as defined in claim 1 or a protected derivative thereof, with an aldehyde compound of formula (V): 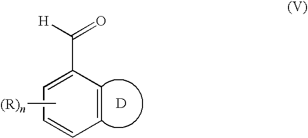
wherein D, n, and R are as defined claim 1, or
(c) reaction of a compound of formula (IV) defined above with a compound of formula (VI) 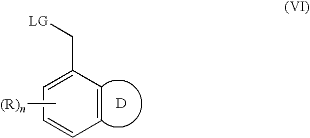
wherein D, n and R are as defined in claim 1, and LG is a suitable leaving group and optionally thereafter (a), (b) or (c):
converting a compound of formula (I) into another compound of formula (I),
removing any protecting groups, and/or
forming a pharmaceutically acceptable salt.

29. A compound of formula (II) or salt thereof 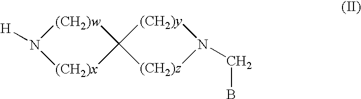
or a compound of formula (II)′ or salt thereof 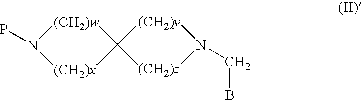
wherein
B represents the group 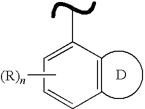
wherein
ring D, together with the two benzene carbon atoms to which it is fused, is a 5- or 6-membered, non-aromatic ring containing one or two ring-oxygen atoms, and optionally containing a carbon-carbon double bond between two ring carbon atoms other than said benzene carbon atoms, ring D being optionally substituted with one or more substituents independently selected from C₁-C₆ alkyl, C₃-C₆ cycloalkyl, or phenyl (said phenyl being optionally substituted with one or more substituents independently selected from halogen, hydroxyl or C₁-C₄ alkoxy);
and additionally wherein when ring D is a 5-membered, non-aromatic ring containing two ring-oxygen atoms that are 1,3 disposed, ring D may be optionally substituted with group E, wherein group E together with a single carbon atom on ring D, represents a 4- to 8-membered cycloalkyl ring, such that group E forms a spiro structure with ring D;
each R represents a group independently selected from halogen or C₁-C₄ alkyl; and
n is 0, 1 or 2;
w, x, v and z are independently 1, 2 or 3;
A represents a group selected from phenyl, a 5- or 6-membered heteroaromatic ring containing at least one ring heteroatom independently selected from nitrogen, oxygen or sulphur, or pyridine-N-oxide, each group being optionally substituted with one or more substituents independently selected from hydroxyl, —CN, halogen, oxo (═O), C₁-C₆ aminoalkyl, C₁-C₆ alkylamino, C₁-C₆ alkyl, N,N-di(C₁-C₆)alkylamino-C₁-C₆ alkyl, C₁-C₆ alkoxy, C₁-C₆ alkylcarbonyl, —NR¹R², —C(O)—NR³R⁴, —C₁-C₆ alkylenyl-C(O)—NR³R⁴, —C₁-C₄ alkyl-C(O)—NR⁵R⁶, —NHSO₂—R⁷, —NHC(O)R⁸, —SO₂NH₂, carboxyl, carboxyl-C₁-C₆ alkyl, C₁-C₆ alkoxycarbonyl, C₁-C₄ alkoxycarbonyl-C₁-C₄ alkyl, C₃-C₆ cycloalkylamino, phenyl, pyridyl (said phenyl and pyridyl being optionally further substituted with one or more groups independently selected from halogen, hydroxyl, carboxy or C₁-C₄ alkyl), C₁-C₆ alkyl or C₃-C₆ cycloalkyl (said latter two C₁-C₆ alkyl and C₃-C₆cycloalkyl substituents being optionally further substituted with one or more substituents independently selected from halogen, hydroxyl, or —CN);
or A represents a 9- or 10-membered bicyclic ring system containing one or more ring heteroatoms independently selected from nitrogen, oxygen or sulphur and which is optionally substituted with one or more substituents independently selected from hydroxyl, —CN, halogen, oxo, C₁-C₆ alkoxy, —NR⁹R¹⁰, carboxyl, or C₁-C₆ alkyl;
wherein:
R¹ and R² each independently represent a hydrogen atom, a C₁-C₆ alkyl, C₃-C₆ cycloalkyl or R¹ and R² together with the nitrogen atom to which they are attached form a hydantoin group or form a 4- to 7-membered saturated heterocycle, said heterocycle being optionally substituted with hydroxyl, C₁-C₄ alkoxy, or C₁-C₄ alkoxy-C₁-C₄ alkyl;
R³ and R⁴ each independently represent a hydrogen atom, C₁-C₆ alkyl, or C₁-C₆ cycloalkyl, or R³ and R⁴ together with the nitrogen atom to which they are attached form a 4- to 7-membered saturated heterocycle, said heterocycle being optionally substituted with aminocarbonyl;
R⁵ and R⁶ each independently represent a hydrogen atom, C₁-C₆ alkyl, or C₃-C₆ cycloalkyl, or R⁵ and R⁶ together with the nitrogen atom to which they are attached form a 4- to 7-membered saturated heterocycle, said heterocycle being optionally substituted with aminocarbonyl;
R⁷ represents C₁-C₆ alkyl, or a 6-membered saturated or unsaturated heterocyclic ring, the ring containing at least one nitrogen atom, the ring being optionally substituted with one or more substituents independently selected from halogen, oxo, C₁-C₆ alkoxy, or C₁-C₆ alkyl;
R⁸ represents pyridine-N-oxide optionally substituted with one or more substituents independently selected from halogen, or C₁-C₆ alkyl, or R⁸ represents C₁-C₆ alkyl, C₁-C₆ hydroxyalkyl, or a 5- or 6-membered saturated heterocyclic ring containing at least one heteroatom independently selected from nitrogen and oxygen, which ring being optionally substituted with one or more substituents independently selected from halogen, C₁-C₆ alkoxy, oxo, or C₁-C₆alkyl; and
R⁹ and R¹⁰ each independently represent a hydrogen atom or C₁-C₆ alkyl;
and P is an amine protecting group.
